SEQUENCE LISTING (203 sequences)

SEQ ID NO: 1 - protein, 117 aa, synthetic construct
DVQLVESGGGLVQPGGSLRLSCAASRSIGRLDRMGWYRHRTGEPRELVATITGGSSINYGDFVKGRFTISRDNAKNTVYLQMNSLRPEDTAVYYCNFNKYVTSRDTWGQGTLVTVSS

SEQ ID NO: 2 - protein, 117 aa, synthetic construct
DVQLVESGGGLVQPGGSLRLSCAASRSIGRLDRMGWYRHRTGEPRELVATITGGSSINYGDFVKGRFTISRDNSKNTVYLQMNNLRPEDTAVYYCNFNKYVTSRDTWGQGTLVTVSS

SEQ ID NO: 3 - protein, 117 aa, synthetic construct
DVQLVESGGGLVQPGGSLRLSCAASRSIGRLDRMGWYRHRPGKPRELVATITGGSSINYADSVKGRFTISRDNSKNTVYLQMNSLRPEDTAVYYCNFNKYVTSRDTWGQGTLVTVSS

SEQ ID NO: 4 - protein, 117 aa, synthetic construct
DVQLVESGGGLVQPGGSLRLSCAASRSIGRLDRMGWYRHRPGKPRELVATITGGSSINYADSVKGRFTISIDNSKNTVYLQMNSLRPEDTAVYYCNFNKYVTSRDTWGQGTLVTVSS

SEQ ID NO: 5 - protein, 116 aa, synthetic construct
DVQLVESGGGLVQPGGSLRLSCAASRSIGRLDRMGWYRHRPGKPRELVATITGGSSINYADSVKGRFTISRDNSKNTVYLQMNLRPEDTAVYYCNFNKYVTSRDTWGQGTLVTVSS

SEQ ID NO: 6 - protein, 116 aa, synthetic construct
DVQLVESGGGLVQPGGSLRLSCAASRSIGRLDRMGWYRHRPGKPRELVATITGGSSINYADSVKGRFTISIDNSKNTVYLQMNLRPEDTAVYYCNFNKYVTSRDTWGQGTLVTVSS

SEQ ID NO: 7 - protein, 117 aa, synthetic construct
EVQLVESGGGLVQPGGSLRLSCAASRSIGRLDRMGWYRHRPGEPRELVATITGGSSINYGDSVKGRFTISIDNSKNTVYLQMNSLRPEDTAVYYCNFNKYVTSRDTWGQGTLVTVSS

SEQ ID NO: 8 - protein, 117 aa, synthetic construct
EVQLVESGGGLVQPGGSLRLSCAASRSIGRLDRMGWYRHRPGKPRELVATITGGSSINYGDSVKGRFTISIDNSKNTVYLQMNSLRPEDTAVYYCNFNKYVTSRDTWGQGTLVTVSS

SEQ ID NO: 9 - protein, 449 aa, synthetic construct
EVQLLESGGGLVQPGGSLRLSCAASGFTFNSYAMSWVRQAPGKGLEWVSLISGSGGFTYYADSVKGRFTISRDNSRTTLYLQMNSLRAEDTAVYYCAKDRLVAPGTFDYWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENYKTTPPVLDSDGSFFLYSKLTVDKSRWQQGNVFSCSVMHEALHNHYTQKSLSLSPGK

SEQ ID NO: 10 - protein, 214 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 12 - protein, 120 aa, synthetic construct
EVQLLESGGGLVQPGGSLRLSCAASGFTFNSYAMSWVRQAPGKGLEWVSIISGSGGFTYYADSVKGRFTISRDNSRTTLYLQMNSLRAEDTAVYYCAKDRLVAPGTFDYWGQGALVTVSS

SEQ ID NO: 13 - protein, 107 aa, synthetic construct
EIVLTQSPGTLSLSPGERATLSCRASQSVSSNYLAWYQQKPGQAPRLLIYGASSRATGIPDRFSGSGSGTDFTLTISRLEPEDFAVYYCQQYGSSFTFGPGTKVDIK

SEQ ID NO: 14 - protein, 120 aa, synthetic construct
QVQLVESGGGVVQPGRSLRLSCAASGFTFSNFGMHWVRQAPGKGLEWVAAIWYDGHDKYYSYYVKGRFTISRDNSKNTLFLQMNSLRAEDTAVYYCARDSSSWYRYFDYWGQGTLVTVSS

SEQ ID NO: 15 - protein, 107 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSFTFGPGTKVDIK

SEQ ID NO: 16 - protein, 120 aa, synthetic construct
QVQLVESGGGVVQPGRSLRLSCAASGFTFSNFGMHWVRQAPGKGLEWVAAIWYDGHDKYYAYYVKGRFTISRDNSKNTLFLQMNSLRAEDTAVYYCARDSSSWYRYFDYWGQGTLVTVSS

SEQ ID NO: 17 - protein, 58 aa, synthetic construct
MLHPLCKVGSHQGSVAVDLGQISLSPSAACSLKILQLITVNSIIVSLTFGGGTKVEIK

SEQ ID NO: 18 - protein, 120 aa, synthetic construct; identical to SEQ ID NO: 14

SEQ ID NO: 19 - protein, 57 aa, synthetic construct
MPPVWKVGSHQGSAAVDLGQISLSPSAACSLKILQLITVNSLIVTLTFGGGTKVEIK

SEQ ID NO: 20 - protein, 116 aa, synthetic construct
QVQLVESGGGVVQPGRSLRLSCAASGFTFSSYTMHWVRQAPGKGLEWVAVIWYDGSNKYYVDSVKGRFTISRDNSKNTLYLQMNSLRAEDTAVYYCARKNWSFDFWGQGTLVTVSS

SEQ ID NO: 21 - protein, 106 aa, synthetic construct
EIVLTQSPATLSLSPGERATLSCRASQGVSRYLAWYQQKPGQAPRLLIYDASNRATGIPARVSGSGPGTDFTLTISSLEPEDFAVDYCQQRSNWQYTFGQGTKLEI

SEQ ID NO: 22 - protein, 121 aa, synthetic construct
QKQLVEFGGGVVQPGRSLRLSCAASGFTFSNYGMHWVRQAPGKGLEWVAVIWNDGSNKYYVDSVKGRFIISRDNSKNTLYLQMNSLRAEDTAVYYCARDRMGIYYYGMDVWGQGTTVTVSS

SEQ ID NO: 23 - protein, 104 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWTFGQGTKVEI

SEQ ID NO: 24 - protein, 120 aa, synthetic construct
EVQLLESGGGLVQPGGSLRLSCAASGFTFSSYAMNWVRQAPGKGLEWVSIISGSGGFTYYADSVKGRFTISRDNSKNTLYLQMNRLRAEDTAIYFCAKDDIPAAGTFDPWGQGTLVTVSS

SEQ ID NO: 25 - protein, 106 aa, synthetic construct
AIQLTQSPSSLSASVGDRVTITCRASQGISSALAWYQQKPGKAPKLLIYDVSSLESGVPSRFSGSGSGTDFTLTISSLQPEDFATYYCQQFNSYWTFGQGTKVEIK

SEQ ID NO: 26 - protein, 119 aa, synthetic construct
EVQLLESGGGLVQPGGSLRLSCAASGFTFSSYAMSWVRQAPGKGLEWVSLISGSGGLTKYADSVKGRFTISRDNSKRTLYLQMNSLRAEDTAVYYCAKDILVTGALDYWGQGTLVTVSS

SEQ ID NO: 27 - protein, 120 aa, synthetic construct
EVQLLESGGGLVQPGGSLRLSCAASGFTFSSYAMNWVRQAPGKGLEWVSIISGSGGFTYYADSVKGRFTISRDNSKKTLYLQMSRLRAEDTAIYFCAKDDIPAAGTFDPWGQGTLVTVSS

SEQ ID NO: 28 - protein, 113 aa, synthetic construct
DILMTQTPLSLPVSLGDQASISCRSSQSIVHGNGNTYLEWHLQKPGQSPKLLIYRVSNRFSGVPDRFSGSGSGTDFTLKINRVEAEDLGVYYCFQGSHVPYTFGGGTKVEIKR

SEQ ID NO: 29 - protein, 113 aa, synthetic construct
DIVMTQTPLSLPVSLGDQASMYCRSSQSPVHSNGNTYLHWYLQKPGQSPKLLIYKVSNRFSGVPDRFSGSGSGTDFTLKISRVEAEDLGVYFCSQSTHIPWTFGGGTKVEIKR

SEQ ID NO: 30 - protein, 123 aa, synthetic construct
QVQLQQPGAELVRPGASVKLSCKASGYTFTSYWLNWVKQRPGQGLEWIVMIDPSDSETHYNQVFKDKATLTVDKSSSTAYMQLSSLTSEDSAVYYCIRGRGNFYGGSHAMEYWGQGTLLTVSS

SEQ ID NO: 31 - protein, 118 aa, synthetic construct
QVQLQQPGAELVKPGTSVKLSCKASGYSFTSYWMHGVRQRPGQGLEWIGEIDPSNGRTNYNEKFKSKATLTVDKSSSTAYIQLSSLTSEDSAVYYCTRERSPRYFDVWGAGTTLTVSS

SEQ ID NO: 32 - protein, 7 aa, synthetic construct
  VARIANT 4: note = wherein X is any amino acid.
VRGXYYY

SEQ ID NO: 33 - DNA, 381 bp, synthetic construct
  source 1..381: mol_type = other DNA
gaggtgcagttggtggagtctgggggaggcttggtacagcctggggggtccctgagactctcctgtgcagcctctggattcacttttagcaactatgccatgaactgggtccgccaggctccagggaaggggctggagtgggtctcaactattagcggaagtggtggtgccacaaggtatgcagactccgtgaagggccgattcaccatatccagagacaattccaggaacacggtgtatctgcaaatgaacagcctgagagtcgaggacacggccgttttttactgtacgaaagatcggctcattatggctacggttcggggaccctattactacggtatggacgtctggggccaagggaccacggtcaccgtctcctca

SEQ ID NO: 34 - protein, 127 aa, synthetic construct
EVQLVESGGGLVQPGGSLRLSCAASGFTFSNYAMNWVRQAPGKGLEWVSTISGSGGATRYADSVKGRFTISRDNSRNTVYLQMNSLRVEDTAVFYCTKDRLIMATVRGPYYYGMDVWGQGTTVTVSS

SEQ ID NO: 35 - DNA, 24 bp, synthetic construct
  source 1..24: mol_type = other DNA
ggattcacttttagcaactatgcc

SEQ ID NO: 36 - protein, 8 aa, synthetic construct
GFTFSNYA

SEQ ID NO: 37 - DNA, 24 bp, synthetic construct
  source 1..24: mol_type = other DNA
attagcggaagtggtggtgccaca

SEQ ID NO: 38 - protein, 8 aa, synthetic construct
ISGSGGAT

SEQ ID NO: 39 - DNA, 60 bp, synthetic construct
  source 1..60: mol_type = other DNA
acgaaagatcggctcattatggctacggttcggggaccctattactacggtatggacgtc

SEQ ID NO: 40 - protein, 20 aa, synthetic construct
TKDRLIMATVRGPYYYGMDV

SEQ ID NO: 41 - DNA, 15 bp, synthetic construct
  source 1..15: mol_type = other DNA
aactatgccatgaac

SEQ ID NO: 42 - protein, 5 aa, synthetic construct
NYAMN

SEQ ID NO: 43 - DNA, 51 bp, synthetic construct
  source 1..51: mol_type = other DNA
actattagcggaagtggtggtgccacaaggtatgcagactccgtgaagggc

SEQ ID NO: 44 - protein, 17 aa, synthetic construct
TISGSGGATRYADSVKG

SEQ ID NO: 45 - DNA, 54 bp, synthetic construct
  source 1..54: mol_type = other DNA
gatcggctcattatggctacggttcggggaccctattactacggtatggacgtc

SEQ ID NO: 46 - protein, 18 aa, synthetic construct
DRLIMATVRGPYYYGMDV

SEQ ID NO: 47 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaacctcctgatctatgctgcatccagtttgcaaagtggggtcccatcaaggttcagtggcagtggatctgagacagatttcactctcaccatcagcagtctgcaacctgaagattttgcaacttactactgtcaacagagtcacagtgtctcattcactttcggccctgggaccaaagtggatatcaaa

SEQ ID NO: 48 - protein, 107 aa, synthetic construct
DIQMTQSPSSLSASVGDRVTITCRASQSISSYLNWYQQKPGKAPNLLIYAASSLQSGVPSRFSGSGSETDFTLTISSLQPEDFATYYCQQSHSVSFTFGPGTKVDIK

SEQ ID NO: 49 - DNA, 18 bp, synthetic construct
  source 1..18: mol_type = other DNA
cagagcattagcagctat

SEQ ID NO: 50 - protein, 6 aa, synthetic construct
QSISSY

SEQ ID NO: 51-52 - skipped (2 entries)

SEQ ID NO: 53 - DNA, 27 bp, synthetic construct
  source 1..27: mol_type = other DNA
caacagagtcacagtgtctcattcact

SEQ ID NO: 54 - protein, 9 aa, synthetic construct
QQSHSVSFT

SEQ ID NO: 55 - DNA, 33 bp, synthetic construct
  source 1..33: mol_type = other DNA
cgggcaagtcagagcattagcagctatttaaat

SEQ ID NO: 56 - protein, 11 aa, synthetic construct
RASQSISSYLN

SEQ ID NO: 57 - DNA, 21 bp, synthetic construct
  source 1..21: mol_type = other DNA
gctgcatccagtttgcaaagt

SEQ ID NO: 58 - protein, 7 aa, synthetic construct
AASSLQS

SEQ ID NO: 59 - DNA, 27 bp, synthetic construct
  source 1..27: mol_type = other DNA
caacagagtcacagtgtctcattcact

SEQ ID NO: 60 - protein, 9 aa, synthetic construct
QQSHSVSFT

SEQ ID NO: 61 - DNA, 1362 bp, synthetic construct
  source 1..1362: mol_type = other DNA
gaagtgcaactggtggagtccggaggaggcctggtgcagcctggaggaagcctgaggctgagctgtgccgccagcggcttcaccttcagcaactacgccatgaactgggtgaggcaggcccctggcaagggactggagtgggtctccaccatcagcggctccggaggcgctacacggtacgccgatagcgtgaagggccggtttaccatttcccgggacaactcccggaacaccgtgtacctccagatgaacagcctgagggtggaggataccgccgtgttctactgcaccaaggacaggctgattatggccaccgtgaggggaccttactactatggcatggatgtgtggggccagggcacaaccgtcaccgtgtcctccgcctccaccaagggacctagcgtgttccctctcgccccctgttccaggtccacaagcgagtccaccgctgccctcggctgtctggtgaaagactactttcccgagcccgtgaccgtctcctggaatagcggagccctgacctccggcgtgcacacatttcccgccgtgctgcagagcagcggactgtatagcctgagcagcgtggtgaccgtgcccagctccagcctcggcaccaaaacctacacctgcaacgtggaccacaagccctccaacaccaaggtggacaagcgggtggagagcaagtacggccccccttgccctccttgtcctgcccctgagttcgagggaggaccctccgtgttcctgtttccccccaaacccaaggacaccctgatgatctcccggacacccgaggtgacctgtgtggtcgtggacgtcagccaggaggaccccgaggtgcagttcaactggtatgtggacggcgtggaggtgcacaatgccaaaaccaagcccagggaggagcagttcaattccacctacagggtggtgagcgtgctgaccgtcctgcatcaggattggctgaacggcaaggagtacaagtgcaaggtgtccaacaagggactgcccagctccatcgagaagaccatcagcaaggctaagggccagccgagggagccccaggtgtataccctgcctcctagccaggaagagatgaccaagaaccaagtgtccctgacctgcctggtgaagggattctacccctccgacatcgccgtggagtgggagagcaatggccagcccgagaacaactacaaaacaacccctcccgtgctcgatagcgacggcagcttctttctctacagccggctgacagtggacaagagcaggtggcaggagggcaacgtgttctcctgttccgtgatgcacgaggccctgcacaatcactacacccagaagagcctctccctgtccctgggcaag

SEQ ID NO: 62 - protein, 454 aa, synthetic construct
EVQLVESGGGLVQPGGSLRLSCAASGFTFSNYAMNWVRQAPGKGLEWVSTISGSGGATRYADSVKGRFTISRDNSRNTVYLQMNSLRVEDTAVFYCTKDRLIMATVRGPYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - DNA, 642 bp, synthetic construct
  source 1..642: mol_type = other DNA
gacatccagatgacccagtccccttcctccctgagcgctagcgtgggagatagggtgaccatcacctgcagggcctcccaaagcatctcctcctacctgaactggtaccagcagaaacccggcaaggcccccaacctgctgatctacgctgcctcctccctccagtccggcgtgcctagcaggtttagcggctccggaagcgagaccgacttcaccctgaccatctcctccctgcagcccgaggacttcgccacctactactgccagcaatcccacagcgtgtccttcaccttcggccccggcaccaaggtggacatcaagaggaccgtggccgccccctccgtgttcatctttcccccctccgatgaacagctgaagagcggcaccgctagcgtggtgtgcctgctgaacaacttctaccccagggaggccaaggtgcagtggaaggtggacaatgccctgcagtccggcaacagccaggagagcgtgaccgagcaggactccaaggacagcacctacagcctgtcctccaccctgaccctgtccaaggccgactacgagaagcacaaagtgtacgcctgcgaagtgacccatcagggcctgagctcccccgtgaccaagtcctttaacaggggcgagtgc

SEQ ID NO: 64 - protein, 214 aa, synthetic construct
DIQMTQSPSSLSASVGDRVTITCRASQSISSYLNWYQQKPGKAPNLLIYAASSLQSGVPSRFSGSGSETDFTLTISSLQPEDFATYYCQQSHSVSFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - DNA, 372 bp, synthetic construct
  source 1..372: mol_type = other DNA
caggtgcagctggtggagtctgggggaggcttggtcaagcctggagggtccctgagactctcctgtgcagcctctcgattcaccctcagtgactactacatgacctggatccgccaggctccagggaaggggctggagtgggtttcatacattagtagtagtggtaataccatatactacgcagactctgtgaagggccgattcaccatctccagggacaacgccaagaactcactgtatctgcaaatgaacagcctgagagccgaggacacggccgtgtattactgtgcgagagatctgagtgggagctactgggactactactacggtatggacgtctggggccaagggaccacggtcaccgtctcctca

SEQ ID NO: 66 - protein, 124 aa, synthetic construct
QVQLVESGGGLVKPGGSLRLSCAASRFTLSDYYMTWIRQAPGKGLEWVSYISSSGNTIYYADSVKGRFTISRDNAKNSLYLQMNSLRAEDTAVYYCARDLSGSYWDYYYGMDVWGQGTTVTVSS

SEQ ID NO: 67 - DNA, 24 bp, synthetic construct
  source 1..24: mol_type = other DNA
cgattcaccctcagtgactactac

SEQ ID NO: 68 - protein, 8 aa, synthetic construct
RFTLSDYY

SEQ ID NO: 69 - DNA, 24 bp, synthetic construct
  source 1..24: mol_type = other DNA
attagtagtagtggtaataccata

SEQ ID NO: 70 - protein, 8 aa, synthetic construct
ISSSGNTI

SEQ ID NO: 71 - DNA, 51 bp, synthetic construct
  source 1..51: mol_type = other DNA
gcgagagatctgagtgggagctactgggactactactacggtatggacgtc

SEQ ID NO: 72 - protein, 17 aa, synthetic construct
ARDLSGSYWDYYYGMDV

SEQ ID NO: 73 - DNA, 15 bp, synthetic construct
  source 1..15: mol_type = other DNA
gactactacatgacc

SEQ ID NO: 74 - protein, 5 aa, synthetic construct
DYYMT

SEQ ID NO: 75 - DNA, 51 bp, synthetic construct
  source 1..51: mol_type = other DNA
tacattagtagtagtggtaataccatatactacgcagactctgtgaagggc

SEQ ID NO: 76 - protein, 17 aa, synthetic construct
YISSSGNTIYYADSVKG

SEQ ID NO: 77 - DNA, 45 bp, synthetic construct
  source 1..45: mol_type = other DNA
gatctgagtgggagctactgggactactactacggtatggacgtc

SEQ ID NO: 78 - protein, 15 aa, synthetic construct
DLSGSYWDYYYGMDV

SEQ ID NO: 79 - DNA, 321 bp, synthetic construct
  source 1..321: mol_type = other DNA
gccatccagttgacccagtctccatcctccctgtctacatctgtaggagacagagtcaccatcgcttgccgggcaagtcagggcattaacaatgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttaccctcggacgttcggccaagggaccaaggtggaaatcaaa

SEQ ID NO: 80 - protein, 107 aa, synthetic construct
AIQLTQSPSSLSTSVGDRVTIACRASQGINNALAWYQQKPGKAPKLLIYDASSLESGVPSRFSGSGSGTDFTLTISSLQPEDFATYYCQQFNSYPRTFGQGTKVEIK

SEQ ID NO: 81 - DNA, 18 bp, synthetic construct
  source 1..18: mol_type = other DNA
cagggcattaacaatgct

SEQ ID NO: 82 - protein, 6 aa, synthetic construct
QGINNA

SEQ ID NO: 83-84 - skipped (2 entries)

SEQ ID NO: 85 - DNA, 27 bp, synthetic construct
  source 1..27: mol_type = other DNA
caacagtttaatagttaccctcggacg

SEQ ID NO: 86 - protein, 9 aa, synthetic construct
QQFNSYPRT

SEQ ID NO: 87 - DNA, 33 bp, synthetic construct
  source 1..33: mol_type = other DNA
cgggcaagtcagggcattaacaatgctttagcc

SEQ ID NO: 88 - protein, 11 aa, synthetic construct
RASQGINNALA

SEQ ID NO: 89 - DNA, 21 bp, synthetic construct
  source 1..21: mol_type = other DNA
gatgcctccagtttggaaagt

SEQ ID NO: 90 - protein, 7 aa, synthetic construct
DASSLES

SEQ ID NO: 91 - DNA, 27 bp, synthetic construct
  source 1..27: mol_type = other DNA
caacagtttaatagttaccctcggacg

SEQ ID NO: 92 - protein, 9 aa, synthetic construct
QQFNSYPRT

SEQ ID NO: 93 - DNA, 369 bp, synthetic construct
  source 1..369: mol_type = other DNA
gaggtgcagctggtggagtctgggggaggcttggtaaagcctggggggtcccttagactctcctgtgcagcctctggattcactttcagtaacgcctggatgagctgggtccgccaggctccagggaaggggctggagtgggttggccgtattaaaagcaaaactgaaggtgggacaacagactacgctgcacccgtgaaaggcagattcaccatctcaagagatgattcaaaaaacacgctgtatctgcaaatgaacagcctgaaaaccgaggacacagccgtgtattactgtaccacagattttctatggttcggggagttcccttttgactactggggccagggaaccctggtcaccgtctcctca

SEQ ID NO: 94 - protein, 123 aa, synthetic construct
EVQLVESGGGLVKPGGSLRLSCAASGFTFSNAWMSWVRQAPGKGLEWVGRIKSKTEGGTTDYAAPVKGRFTISRDDSKNTLYLQMNSLKTEDTAVYYCTTDFLWFGEFPFDYWGQGTLVTVSS

SEQ ID NO: 95 - DNA, 24 bp, synthetic construct
  source 1..24: mol_type = other DNA
ggattcactttcagtaacgcctgg

SEQ ID NO: 96 - protein, 8 aa, synthetic construct
GFTFSNAW

SEQ ID NO: 97 - DNA, 30 bp, synthetic construct
  source 1..30: mol_type = other DNA
attaaaagcaaaactgaaggtgggacaaca

SEQ ID NO: 98 - protein, 10 aa, synthetic construct
IKSKTEGGTT

SEQ ID NO: 99 - DNA, 42 bp, synthetic construct
  source 1..42: mol_type = other DNA
accacagattttctatggttcggggagttcccttttgactac

SEQ ID NO: 100 - protein, 14 aa, synthetic construct
TTDFLWFGEFPFDY

SEQ ID NO: 101 - DNA, 15 bp, synthetic construct
  source 1..15: mol_type = other DNA
aacgcctggatgagc

SEQ ID NO: 102 - protein, 5 aa, synthetic construct
NAWMS

SEQ ID NO: 103 - DNA, 57 bp, synthetic construct
  source 1..57: mol_type = other DNA
cgtattaaaagcaaaactgaaggtgggacaacagactacgctgcacccgtgaaaggc

SEQ ID NO: 104 - protein, 19 aa, synthetic construct
RIKSKTEGGTTDYAAPVKG

SEQ ID NO: 105 - DNA, 36 bp, synthetic construct
  source 1..36: mol_type = other DNA
gattttctatggttcggggagttcccttttgactac

SEQ ID NO: 106 - protein, 12 aa, synthetic construct
DFLWFGEFPFDY

SEQ ID NO: 107 - DNA, 321 bp, synthetic construct
  source 1..321: mol_type = other DNA
gacatccagatgacccagtctccatcctccctgtctgcatctgtaggagacagagtcaccatcacttgccgggcgagtcagggcattagcaattatttagcctggtatcagcagaaaccagggaaaattcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtataacagtgcccctcggacgttcggccaagggaccaaggtggaaatcaaa

SEQ ID NO: 108 - protein, 107 aa, synthetic construct
DIQMTQSPSSLSASVGDRVTITCRASQGISNYLAWYQQKPGKIPKLLIYAASTLQSGVPSRFSGSGSGTDFTLTISSLQPEDVATYYCQKYNSAPRTFGQGTKVEIK

SEQ ID NO: 109 - DNA, 18 bp, synthetic construct
  source 1..18: mol_type = other DNA
cagggcattagcaattat

SEQ ID NO: 110 - protein, 6 aa, synthetic construct
QGISNY

SEQ ID NO: 111-112 - skipped (2 entries)

SEQ ID NO: 113 - DNA, 27 bp, synthetic construct
  source 1..27: mol_type = other DNA
caaaagtataacagtgcccctcggacg

SEQ ID NO: 114 - protein, 9 aa, synthetic construct
QKYNSAPRT

SEQ ID NO: 115 - DNA, 33 bp, synthetic construct
  source 1..33: mol_type = other DNA
cgggcgagtcagggcattagcaattatttagcc

SEQ ID NO: 116 - protein, 11 aa, synthetic construct
RASQGISNYLA

SEQ ID NO: 117 - DNA, 21 bp, synthetic construct
  source 1..21: mol_type = other DNA
gctgcatccactttgcaatca

SEQ ID NO: 118 - protein, 7 aa, synthetic construct
AASTLQS

SEQ ID NO: 119 - DNA, 27 bp, synthetic construct
  source 1..27: mol_type = other DNA
caaaagtataacagtgcccctcggacg

SEQ ID NO: 120 - protein, 9 aa, synthetic construct
QKYNSAPRT

SEQ ID NO: 121 - DNA, 981 bp, Homo sapiens
  source 1..981: mol_type = other DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 122 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 123 - DNA, 981 bp, Homo sapiens
  source 1..981: mol_type = other DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccgtgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcgtgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacgcagaagagcctctccctgtctctgggtaaa

SEQ ID NO: 124 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 125 - DNA, 981 bp, Homo sapiens
  source 1..981: mol_type = other DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctcaccgtggacaagagcaggtggcaggaggggaacgtcttctcatgctccgtgatgcatgaggctctgcacaaccactacacgcagaagagcctctccctgtctctgggtaaa

SEQ ID NO: 126 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 127 - DNA, 981 bp, synthetic construct
  source 1..981: mol_type = other DNA
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctgaatttgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 128 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 129 - DNA, 981 bp, synthetic construct
  source 1..981: mol_type = other DNA
gcctccaccaagggacctagcgtgttccctctcgccccctgttccaggtccacaagcgagtccaccgctgccctcggctgtctggtgaaagactactttcccgagcccgtgaccgtctcctggaatagcggagccctgacctccggcgtgcacacatttcccgccgtgctgcagagcagcggactgtatagcctgagcagcgtggtgaccgtgcccagctccagcctcggcaccaaaacctacacctgcaacgtggaccacaagccctccaacaccaaggtggacaagcgggtggagagcaagtacggccccccttgccctccttgtcctgcccctgagttcgagggaggaccctccgtgttcctgtttccccccaaacccaaggacaccctgatgatctcccggacacccgaggtgacctgtgtggtcgtggacgtcagccaggaggaccccgaggtgcagttcaactggtatgtggacggcgtggaggtgcacaatgccaaaaccaagcccagggaggagcagttcaattccacctacagggtggtgagcgtgctgaccgtcctgcatcaggattggctgaacggcaaggagtacaagtgcaaggtgtccaacaagggactgcccagctccatcgagaagaccatcagcaaggctaagggccagccgagggagccccaggtgtataccctgcctcctagccaggaagagatgaccaagaaccaagtgtccctgacctgcctggtgaagggattctacccctccgacatcgccgtggagtgggagagcaatggccagcccgagaacaactacaaaacaacccctcccgtgctcgatagcgacggcagcttctttctctacagccggctgacagtggacaagagcaggtggcaggagggcaacgtgttctcctgttccgtgatgcacgaggccctgcacaatcactacacccagaagagcctctccctgtccctgggcaag

SEQ ID NO: 130 - DNA, 981 bp, synthetic construct
  source 1..981: mol_type = other DNA
gccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 131 - DNA, 981 bp, synthetic construct
  source 1..981: mol_type = other DNA
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctccagttgcggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 132 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 133 - DNA, 990 bp, Homo sapiens
  source 1..990: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtggagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 134 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - DNA, 321 bp, Homo sapiens
  source 1..321: mol_type = other DNA
cgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 136 - protein, 107 aa, Homo sapiens [sequence omitted]

SEQ ID NO: 137 - DNA, 321 bp, Homo sapiens
  source 1..321: mol_type = other DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggagagcaaggacagcacctacagcctcagcagcaccctgacgctgagcaaagcagactacgagaaacacaaagtctacgccggcgaagtcacccatcagggcctgagctcgcccgtcacaaagagcttcaacaggggagagtgt

SEQ ID NO: 138 - protein, 107 aa, Homo sapiens
RTVAAPSVFIFPPSDEQLKSGTASVVCLLNNFYPREAKVQWKVDNALQSGNSQESVTEQESKDSTYSLSSTLTLSKADYEKHKVYAGEVTHQGLSSPVTKSFNRGEC

SEQ ID NO: 139 - DNA, 321 bp, Homo sapiens
  source 1..321: mol_type = other DNA
cgaactgtggctgcaccatctgtcttcatcttcccgccatctgatgagcagttgaaatctggaactgcctctgttgtgtgcctgctgaataacttctatcccagagaggccaaagtacagcggaaggtggataacgccctccaatcgggtaactcccaggagagtgtcacagagcaggagagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 140 - protein, 107 aa, Homo sapiens
RTVAAPSVFIFPPSDEQLKSGTASVVCLLNNFYPREAKVQRKVDNALQSGNSQESVTEQESKDSTYSLSSTLTLSKADYEKHKVYACEVTHQGLSSPVTKSFNRGEC

SEQ ID NO: 141 - DNA, 321 bp, Homo sapiens
  source 1..321: mol_type = other DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 142 - protein, 107 aa, Homo sapiens
RTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 143 - DNA, 321 bp, Homo sapiens
  source 1..321: mol_type = other DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcaacaccctgacgctgagcaaagcagactacgagaaacacaaagtctacgcctgcgaagtcacccatcagggcctgagctcgcccgtcacaaagagcttcaacaggggagagtgc

SEQ ID NO: 144 - protein, 107 aa, Homo sapiens
RTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 145 - DNA, 312 bp, Homo sapiens
  source 1..312: mol_type = other DNA
cccaaggccaaccccacggtcactctgttcccgccctcctctgaggagctccaagccaacaaggccacactagtgtgtctgatcagtgacttctacccgggagctgtgacagtggcttggaaggcagatggcagccccgtcaaggcgggagtggagacgaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 146 - protein, 104 aa, Homo sapiens
PKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 147 - DNA, 318 bp, Homo sapiens
  source 1..318: mol_type = other DNA
ggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 148 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 149 - DNA, 318 bp, Homo sapiens; identical to SEQ ID NO: 147
  source 1..318: mol_type = other DNA

SEQ ID NO: 150 - DNA, 318 bp, Homo sapiens
  source 1..318: mol_type = other DNA
ggccagcctaaggccgctccttctgtgaccctgttccccccatcctccgaggaactgcaggctaacaaggccaccctcgtgtgcctgatcagcgacttctaccctggcgccgtgaccgtggcctggaaggctgatagctctcctgtgaaggccggcgtggaaaccaccaccccttccaagcagtccaacaacaaatacgccgcctcctcctacctgtccctgacccctgagcagtggaagtcccaccggtcctacagctgccaagtgacccacgagggctccaccgtggaaaagaccgtggctcctaccgagtgctcc

SEQ ID NO: 151 - DNA, 318 bp, Homo sapiens
  source 1..318: mol_type = other DNA
ggccagcctaaagctgcccccagcgtcaccctgtttcctccctccagcgaggagctccaggccaacaaggccaccctcgtgtgcctgatctccgacttctatcccggcgctgtgaccgtggcttggaaagccgactccagccctgtcaaagccggcgtggagaccaccacaccctccaagcagtccaacaacaagtacgccgcctccagctatctctccctgacccctgagcagtggaagtcccaccggtcctactcctgtcaggtgacccacgagggctccaccgtggaaaagaccgtcgcccccaccgagtgctcc

SEQ ID NO: 152 - protein, 106 aa, Homo sapiens; identical to SEQ ID NO: 148

SEQ ID NO: 153 - DNA, 318 bp, Homo sapiens
  source 1..318: mol_type = other DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 154 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 155 - DNA, 312 bp, Homo sapiens
  source 1..312: mol_type = other DNA
cccaaggctgccccctcggtcactctgttcccaccctcctctgaggagcttcaagccaacaaggccacactggtgtgtctcataagtgacttctacccgggagccgtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagttgcccctacggaatgttca

SEQ ID NO: 156 - protein, 104 aa, Homo sapiens
PKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 157 - DNA, 318 bp, Homo sapiens
  source 1..318: mol_type = other DNA
ggtcagcccaaggctgccccctcggtcactctgttcccaccctcctctgaggagcttcaagccaacaaggccacactggtgtgtctcataagtgacttctacccggggccagtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacggaatgttca

SEQ ID NO: 158 - protein, 106 aa, Homo sapiens
GQPKAAPSVTLFPPSSEELQANKATLVCLISDFYPGPVTVAWKADSSPVKAGVETTTPSKQSNNKYAASSYLSLTPEQWKSHKSYSCQVTHEGSTVEKTVAPTECS

SEQ ID NO: 159 - DNA, 318 bp, Homo sapiens
  source 1..318: mol_type = other DNA
ggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 160 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 161 - DNA, 318 bp, Homo sapiens
  source 1..318: mol_type = other DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 162 - protein, 106 aa, Homo sapiens; identical to SEQ ID NO: 154

SEQ ID NO: 163 - DNA, 318 bp, Homo sapiens
  source 1..318: mol_type = other DNA
ggtcagcccaaggctgccccatcggtcactctgttcccgccctcctctgaggagcttcaagccaacaaggccacactggtgtgcctgatcagtgacttctacccgggagctgtgaaagtggcctggaaggcagatggcagccccgtcaacacgggagtggagaccaccacaccctccaaacagagcaacaacaagtacgcggccagcagctacctgagcctgacgcctgagcagtggaagtcccacagaagctacagctgccaggtcacgcatgaagggagcaccgtggagaagacagtggcccctgcagaatgttca

SEQ ID NO: 164 - protein, 106 aa, Homo sapiens
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 165 - DNA, 318 bp, Homo sapiens
  source 1..318: mol_type = other DNA
ggtcagcccaaggctgccccatcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 166 - protein, 106 aa, Homo sapiens
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 167 - DNA, 615 bp, synthetic construct
  source 1..615: mol_type = other DNA
atgggctggtcctgcatcatcctgtttctggtggccaccgccaccggcgtgcacagcgattacaaggatgacgacgataagcgtatgaaacagatcgaagataaaattgaagagatcttgagcaaaatctatcatatcgaaaacgaaattgcgcgtatcaaaaagctgattggcgaacgtggcggtggcagcggtggcggtagcggcggtggcagccaggtgtcccaccgataccccaggatccagtccatcaaggtccagttcaccgagtacaaaaaggagaagggattcatcctgacctcccaaaaggaggacgagatcatgaaggtgcaaaacaactccgtgatcatcaactgcgacggcttctacctgatctccctgaagggctacttctcccaggaggtgaacatctccctgcactaccagaaggacgaggagcccctgttccagctgaagaaggtgaggtccgtgaattccctgatggtggccagcctgacctacaaggacaaggtctacctgaacgtgaccaccgacaacaccagcctggacgacttccatgtcaacggcggcgagctgatcctgatccatcagaaccccggcgagttttgcgtcctg

SEQ ID NO: 168 - protein, 205 aa, synthetic construct
MGWSCIILFLVATATGVHSDYKDDDDKRMKQIEDKIEEILSKIYHIENEIARIKKLIGERGGGSGGGSGGGSQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 169 - DNA, 615 bp, synthetic construct
  source 1..615: mol_type = other DNA
atgggctggtcctgcatcatcctgtttctggtggccaccgccaccggcgtgcacagcgattacaaggatgacgacgataagcgtatgaaacagatcgaagataaaattgaagagatcttgagcaaaatctatcatatcgaaaacgaaattgcgcgtatcaaaaagctgattggcgaacgtggcggtggcagcggtggcggtagcggcggtggcagccaggtgtcccaccaataccccaggatccagtccatcaaggtccagttcaccgagtacaaaaaggaggagggattcatcctgacctcccaaaaggaggacgagatcatgaaggtgcaaaacaactccgtgatcatcaactgcgacggcttctacctgatctccctgaagggctacttctcccaggaggtgaacatctccctgcactaccagaaggacgaggagcccctgttccagctgaagaaggtgaggtccgtgaattccctgatggtggccagcctgacctacaaggacaaggtctacctgaacgtgaccaccgacaacaccagcctggacgacttccatgtcaacggcggcgagctgatcctgatccatcagaaccccggcgagttttgcgtcctg

SEQ ID NO: 170 - protein, 205 aa, synthetic construct
MGWSCIILFLVATATGVHSDYKDDDDKRMKQIEDKIEEILSKIYHIENEIARIKKLIGERGGGSGGGSGGGSQVSHQYPRIQSIKVQFTEYKKEEGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 171 - DNA, 1332 bp, synthetic construct
  source 1..1332: mol_type = other DNA
atgggctggtcctgcatcatcctgtttctggtggccaccgccaccggcgtgcacagcctgcattgcgtgggcgacacctatccctccaacgacaggtgctgccacgagtgcaggcctggaaacggcatggtgagcaggtgcagccggtcccagaataccgtgtgtaggccctgcggccccggcttttacaacgacgtggtgtcctccaagccctgcaagccctgcacatggtgcaacctgcggtccggcagcgagaggaagcagctctgcacagccacccaggacaccgtctgtaggtgtagggctggcacccagcctctggactcctacaagcccggcgtggattgtgctccttgccctcccggccatttctcccctggcgacaaccaggcttgcaagccctggaccaactgtaccctggccggcaagcatacactgcagcctgcttccaactcctccgacgctatctgcgaggatagggacccccctgccacacaaccccaggagacacagggccctcctgctaggcccatcacagtccaacccaccgaagcctggcccaggacatcccaaggcccttccaccaggcctgtggaagtgcctggaggaagggctgtggccattgaaggtcgtatggatgaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagcccctga

SEQ ID NO: 172 - protein, 443 aa, synthetic construct
MGWSCIILFLVATATGVHSLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAIEGRMDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 173 - DNA, 552 bp, Homo sapiens
  source 1..552: mol_type = other DNA
atggagagggtgcagcccctcgaggagaacgtgggaaacgccgccaggcctaggttcgagaggaacaagctgctgctggtggcttccgtgatccaaggactcggcctgctgctctgcttcacctacatctgcctccacttcagcgccctgcaggtgtcccaccgataccccaggatccagtccatcaaggtccagttcaccgagtacaaaaaggagaagggattcatcctgacctcccaaaaggaggacgagatcatgaaggtgcaaaacaactccgtgatcatcaactgcgacggcttctacctgatctccctgaagggctacttctcccaggaggtgaacatctccctgcactaccagaaggacgaggagcccctgttccagctgaagaaggtgaggtccgtgaattccctgatggtggccagcctgacctacaaggacaaggtctacctgaacgtgaccaccgacaacaccagcctggacgacttccatgtcaacggcggcgagctgatcctgatccatcagaaccccggcgagttttgcgtcctgtaa

SEQ ID NO: 174 - protein, 181 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFC

SEQ ID NO: 175 - DNA, 834 bp, Homo sapiens
  source 1..834: mol_type = other DNA
atgtgcgtgggggctcggcggctgggccgcgggccgtgtgcggctctgctcctcctgggcctggggctgagcaccgtgacggggctccactgtgtcggggacacctaccccagcaacgaccggtgctgccacgagtgcaggccaggcaacgggatggtgagccgctgcagccgctcccagaacacggtgtgccgtccgtgcgggccgggcttctacaacgacgtggtcagctccaagccgtgcaagccctgcacgtggtgtaacctcagaagtgggagtgagcggaagcagctgtgcacggccacacaggacacagtctgccgctgccgggcgggcacccagcccctggacagctacaagcctggagttgactgtgccccctgccctccagggcacttctccccaggcgacaaccaggcctgcaagccctggaccaactgcaccttggctgggaagcacaccctgcagccggccagcaatagctcggacgcaatctgtgaggacagggaccccccagccacgcagccccaggagacccagggccccccggccaggcccatcactgtccagcccactgaagcctggcccagaacctcacagggaccctccacccggcccgtggaggtccccgggggccgtgcggttgccgccatcctgggcctgggcctggtgctggggctgctgggccccctggccatcctgctggccctgtacctgctccggagggaccagaggctgccccccgatgcccacaagccccctgggggaggcagtttccggacccccatccaagaggagcaggccgacgcccactccaccctggccaagatctga

SEQ ID NO: 176 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 177 - protein, 117 aa, synthetic construct
EVQLVESGGGLVQAGGSLRLSCAASRSIGRLDRMGWYRHRTGEPRELVATITGGSSINYGDFVKGRFTISIDNAKNTVYLQMNNLKPEDTAVYYCNFNKYVTSRDTWGQGTQVTVSS

SEQ ID NO: 178 - protein, 128 aa, synthetic construct
EVQLVESGGGLVQAGGSLRLSCVASGRSFSTYIMGWFRQAPGKEREFVATISRSGITIRSADSVKGRFTISRDNAKNTVYLQMNSLKPEDTAVYYCAAGPYVEQTLGLYQTLGPWDYWGQGTQVTVSS

SEQ ID NO: 179 - protein, 124 aa, synthetic construct
EVQLVESGGGLVQAGGSLRLSCAASGRTFSSIYAKGWFRQAPGKEREFVAAISRSGRSTSYADSVKGRFTISRDNAKNTVYLQMNSLKPEDTAVYYCAAVGGATTVTASEWDYWGLGTQVTVSS

SEQ ID NO: 180 - protein, 125 aa, synthetic construct
EVQLVESGGGLVQAGDSLRLSCAASGLTFSSFAMGWFRQAPGKEREFVAAISRSGYGTSEADSVRDRFIISRDNAKNTVTLHLSRLKPEDTAVYYCAAEHTLGRPSRSQINYLYWGQGTQVTVSS

SEQ ID NO: 181 - protein, 117 aa, synthetic construct
EVQLVESGGGLVQAGGSLRLSCAASRNILSLNTMGWYRHAPGKPRELVARISSNSKTDYADSVKGRFTISRDNAKNTVLLQMNSLKPEDTGVYYCNLNVWRTSSDYWGQGTQVTVSS

SEQ ID NO: 182 - protein, 128 aa, synthetic construct
EVQLVESGGGLVQAGGSLRLSCAASGFTLDDYAIAWFRQAPGKEREGVSRIKISNGRTTYAGSVKGRFTISSDNAKNTVYLQMNSLNAEDTAVYYCAADRSSLLFGSNWDRKARYDYWGQGTQVTVSS

SEQ ID NO: 183 - protein, 125 aa, synthetic construct
EVQLVESGGGLVQAGASLRLSCAASGRRFISNYAMGWFRQAPGQERAFVAAISRSGSITYYTDSVKGRFSISRDYAKSTVYLQMDNLKPEDTAVYYCAADGGAVRDLTTNLPDYWGRGTQVTVSS

SEQ ID NO: 184 - protein, 128 aa, synthetic construct
EVQLVESGGGLVQPGGSLRLSCAASGRSFSTYIMGWFRQAPGKEREFVATISRSGITTRSADSVKGRFTISRDNSKNTVYLQMNSLRPEDTAVYYCAAGPYVEQTLGLYQTLGPWDYWGQGTLVTVSS

SEQ ID NO: 185 - protein, 124 aa, synthetic construct
EVQLVESGGGLVQPGGSLRLSCAASGRTFSSIYAKGWFRQAPGKEREFVAAISRSGRSTSYADSVKGRFTISRDNAKNTVYLQMNSLRPEDTAVYYCAAVGGATTVTASEWDYWGLGTLVTVSS

SEQ ID NO: 186 - protein, 124 aa, synthetic construct
EVQLVESGGGLVQPGGSLRLSCAASGRTFSSIYAKGWFRQAPGKEREFVAAISRSGRSTSYADSVKGRFTISRDNSKNTVYLQMNSLRPEDTAVYYCAAVGGATTVTASEWDYWGLGTLVTVSS

SEQ ID NO: 187 - protein, 124 aa, synthetic construct
EVQLVESGGGLVQPGGSLRLSCAASGRTFSSIYAKGWFRQAPGKEREFVAAISRSGRSTSYADSVKGRFTISRDNAKNTVYLQMNSLRPEDTAVYYCAAVGGATTVTASEWDYWGQGTLVTVSS

SEQ ID NO: 188 - protein, 124 aa, synthetic construct
EVQLVESGGGLVQPGGSLRLSCAASGRTFSSIYAKGWFRQAPGKEREFVAAISRSGRSTSYADSVKGRFTISRDNSKNTVYLQMNSLRPEDTAVYYCAAVGGATTVTASEWDYWGQGTLVTVSS

SEQ ID NO: 189 - protein, 124 aa, synthetic construct
DVQLVESGGGLVQPGGSLRLSCAASGRTFSSIYAKGWFRQAPGKEREFVAAISRSGRSTSYADSVKGRFTISRDNAKNTVYLQMNSLRPEDTAVYYCAAVGGATTVTASEWDYWGLGTLVTVSS

SEQ ID NO: 190 - protein, 124 aa, synthetic construct
DVQLVESGGGLVQPGGSLRLSCAASGRTFSSIYAKGWFRQAPGKEREFVAAISRSGRSTSYADSVKGRFTISRDNSKNTVYLQMNSLRPEDTAVYYCAAVGGATTVTASEWDYWGQGTLVTVSS

SEQ ID NO: 191 - protein, 116 aa, synthetic construct
DVQLVESGGGLVQAGGSLRLSCAASRSIGRLDRMGWYRHRTGEPRELVATITGGSSINYGDFVKGRFTISIDNAKNTVYLQMNLKPEDTAVYYCNFNKYVTSRDTWGQGTQVTVSS

SEQ ID NO: 192 - protein, 117 aa, synthetic construct
DVQLVESGGGLVQPGGSLRLSCAASRSIGRLDRMGWYRHRTGEPRELVATITGGSSINYGDFVKGRFTISRDNSKNTVYLQMNSLRPEDTAVYYCNFNKYVTSRDTWGQGTLVTVSS

SEQ ID NO: 193 - protein, 117 aa, synthetic construct
DVQLVESGGGLVQPGGSLRLSCAASRSIGRLDRMGWYRHATGEPRELVATITGGSSINYGDFVKGRFTISRDNSKNTVYLQMNSLRPEDTAVYYCNFNKYVTSRDTWGQGTLVTVSS

SEQ ID NO: 194 - protein, 117 aa, synthetic construct
DVQLVESGGGLVQPGGSLRLSCAASRSIGRLDRMGWYRHRPGEPRELVATITGGSSINYGDFVKGRFTISRDNSKNTVYLQMNSLRPEDTAVYYCNFNKYVTSRDTWGQGTLVTVSS

SEQ ID NO: 195 - protein, 117 aa, synthetic construct
DVQLVESGGGLVQPGGSLRLSCAASRSIGRLDRMGWYRHRTGKPRELVATITGGSSINYGDFVKGRFTISRDNSKNTVYLQMNSLRPEDTAVYYCNFNKYVTSRDTWGQGTLVTVSS

SEQ ID NO: 196 - protein, 117 aa, synthetic construct
DVQLVESGGGLVQPGGSLRLSCAASRSIGRLDRMGWYRHAPGEPRELVATITGGSSINYGDFVKGRFTISRDNSKNTVYLQMNSLRPEDTAVYYCNFNKYVTSRDTWGQGTLVTVSS

SEQ ID NO: 197 - protein, 117 aa, synthetic construct
DVQLVESGGGLVQPGGSLRLSCAASRSIGRLDRMGWYRHATGKPRELVATITGGSSINYGDFVKGRFTISRDNSKNTVYLQMNSLRPEDTAVYYCNFNKYVTSRDTWGQGTLVTVSS

SEQ ID NO: 198 - protein, 117 aa, synthetic construct
DVQLVESGGGLVQPGGSLRLSCAASRSIGRLDRMGWYRHRPGKPRELVATITGGSSINYGDFVKGRFTISRDNSKNTVYLQMNSLRPEDTAVYYCNFNKYVTSRDTWGQGTLVTVSS

SEQ ID NO: 199 - protein, 98 aa, synthetic construct
DVQLVESGGGLVQPGGSLRLSCAASRSIGRLDRMGWYRHAPGKPRELVATITGGSSINYGDFVKGRFTISRDNSKNTVYLQMNSLRPEDTAVYYCNFN

SEQ ID NO: 200 - protein, 117 aa, synthetic construct
DVQLVESGGGLVQPGGSLRLSCAASRSIGRLDRMGWYRHRTGEPRELVATITGGSSINYADFVKGRFTISRDNSKNTVYLQMNSLRPEDTAVYYCNFNKYVTSRDTWGQGTLVTVSS

SEQ ID NO: 201 - protein, 117 aa, synthetic construct
DVQLVESGGGLVQPGGSLRLSCAASRSIGRLDRMGWYRHRTGEPRELVATITGGSSINYGDSVKGRFTISRDNSKNTVYLQMNSLRPEDTAVYYCNFNKYVTSRDTWGQGTLVTVSS

SEQ ID NO: 202 - protein, 117 aa, synthetic construct
DVQLVESGGGLVQPGGSLRLSCAASRSIGRLDRMGWYRHRTGEPRELVATITGGSSINYADSVKGRFTISRDNSKNTVYLQMNSLRPEDTAVYYCNFNKYVTSRDTWGQGTLVTVSS

SEQ ID NO: 203 - protein, 117 aa, synthetic construct
DVQLVESGGGLVQPGGSLRLSCAASRSIGRLDRMGWYRHRTGEPRELVATITGGSSINYGDFVKGRFTISIDNSKNTVYLQMNSLRPEDTAVYYCNFNKYVTSRDTWGQGTLVTVSS

(19) United States
(12) Patent Application Publication
(10) Pub. No.: US 2024/0392021 A1
(43) Pub. Date: Nov. 28, 2024

(54) PHARMACEUTICAL FORMULATIONS CONTAINING ANTI-OX40L ANTIBODIES

(71) Applicant: KYMAB LIMITED, Cambridge (GB)

(72) Inventors: Chen ZHU, Cambridge, MA (US); Ashi GUPTA, Cambridge, MA (US); Tamera GOODING, Cambridge, MA (US); Sanket PATKE, Cambridge, MA (US); Luca BADIALI, Paris (FR); Susanne JÖRG, Basel (CH); Laura ENGELKE, Basel (CH)

(21) Appl. No.: 18/634,191

(22) Filed: Apr. 12, 2024

(30) Foreign Application Priority Data
Apr. 14, 2023 (EP) 23168134.7
Apr. 14, 2023 (EP) 23168136.2

(51) Int. Cl. C07K 16/28; A61K 47/18; A61K 47/22; A61K 47/26

(52) U.S. Cl. CPC C07K 16/2875; A61K 47/183; A61K 47/22; A61K 47/26; C07K 2317/565

(57) ABSTRACT

The present disclosure provides aqueous pharmaceutical formulations comprising anti-OX40L antibodies or antigen binding fragment thereof for treating or preventing OX40L-mediated diseases or conditions in humans. The present disclosure also provides containers and kits comprising the aqueous pharmaceutical formulations and the use of these aqueous pharmaceutical formulations in treating or preventing OX40L-mediated diseases or conditions in humans.

### RELATED APPLICATIONS

This application claims the benefit of priority to European Patent Application Nos. 23168136.2, and 23168134.7, filed Apr. 14, 2023, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Aug. 15, 2024, is named 753707_SA9-649_ST26.xml and is 207,760 bytes in size.

### BACKGROUND

OX40 ligand (OX40L) is a TNF family member and is a 34 kDa type II transmembrane protein. The crystallized complex of human OX40 and OX40L is a trimeric configuration of one OX40L (trimer) and three OX40 monomers. The human extracellular domain is 42% homologous to mouse OX40L.

OX40L is not constitutively expressed but can be induced on professional APCs such as B-cells, dendritic cells (DCs) and macrophages. Other cell types such as Langerhans cells, endothelial cells, smooth muscle cells, mast cells and natural killer (NK) cells can be induced to express OX40L. T-cells can also express OX40L. The OX40L receptor, OX40, is expressed on activated T cells (CD4⁺ and CD8⁺ T cells, Th2, Th1 and Th17 cells) and CD4⁺Foxp3⁺ cells, even in the absence of activation.

The interaction between OX40 and OX40L occurs during the T-cell-DC interaction 2 or 3 days after antigen recognition. After leaving DCs, the OX40-expressing T-cell may interact with an OX40L-expressing cell other than a DC and receive an OX40 signal from this cell, which may provide essential signals for the generation of memory T-cells, the enhancement of Th2 response and the prolongation of the inflammatory responses. OX40 signals into responder T-cells render them resistant to Treg mediated suppression.

Anti-OX40L monoclonal antibodies (Anti-OX40L mAb) may be clinically useful for the treatment or prevention of OX40L-mediated diseases or conditions. Exemplary anti-human OX40L (hOX40L) antibodies and fragments and medical applications for treating or preventing hOX40L-mediated diseases or conditions in humans are described, inter alia, in WO2015/132580 and U.S. Pat. No. 9,139,653.

As such, there is a demand for anti-OX40L antibody pharmaceutical formulations that are suitable for administration to a subject in need thereof, while at the same time maintain the appropriate protein structure throughout its shelf-life and ensure proper and accurate dosage to achieve desired efficacy for the treatment or prevention of OX40L-mediated diseases or conditions upon administration.

An aqueous (liquid)-based formulation is typically an exemplary type of drug formulation for therapeutic antibodies, e.g., monoclonal antibodies (mAbs) and mAb-based therapeutics, which can be used for intravenous (IV) or subcutaneous (SC) delivery of the mAbs or mAb-based therapeutics to achieve maximum bioavailability. However, therapeutic antibodies in an aqueous solution are prone to degradation, aggregation, or undesired chemical modifications unless the solution is formulated properly. The stability of an antibody in aqueous formulation depends not only on the kinds of excipients used in the formulation, but also on the amounts and proportions of the excipients relative to one another. Aside from the stability, other factors, such as suitable viscosity and turbidity of the formulation, the concentration of antibody that can be accommodated by a particular formulation, the visual quality or appearance of the formulation, as well as other properties which enable the formulation to be conveniently administered to a subject are factors that need to be considered in formulating a therapeutic antibody. As such, it is a challenge to develop properly formulated mAbs, such as a properly formulated anti-OX40L mAb.

The present disclosure addresses the need of stable aqueous pharmaceutical formulations comprising high concentrations or low concentrations of anti-OX40L antibodies and drug products comprising the formulations.

### SUMMARY

The present disclosure is based on the discovery of specific aqueous pharmaceutical formulations that work particularly well to stabilize an anti-human OX40L (hOX40L) antibody at a wide range of concentrations, including high concentrations of an anti-human OX40L (hOX40L) antibody. The formulations described herein advantageously reduce turbidity and provide increased stabilization for an hOX40L antibody when exposed to metals and/or when exposed to agitation-induced stress. The aqueous pharmaceutical formulations disclosed herein are stable when packaged in containers such as vials or injection devices.

The present disclosure provides aqueous pharmaceutical formulations comprising an anti-hOX40 ligand (OX40L) antagonist antibody or antigen binding fragments thereof, which is suitable for delivery via injectable routes to a subject in need thereof. The present disclosure also relates to pharmaceutical unit dosages, containers, and kits comprising the aqueous pharmaceutical formulations, as well as the use of the aqueous formulations for treating hOX40-mediated diseases or conditions in a subject in need thereof.

The present disclosure provides aqueous pharmaceutical formulations comprising anti-hOX40L antibodies or antigen binding fragments thereof, which are suitable for delivery via injectable routes to a subject in need thereof. The present disclosure also relates to pharmaceutical unit dosages, containers, and kits comprising the aqueous pharmaceutical formulations, as well as the use of the aqueous formulations for treating hOX40L-mediated diseases or conditions in a subject in need thereof.

In one aspect, embodiments of the present disclosure pertain to an aqueous pharmaceutical formulation which comprises: (a) an antibody or antigen binding fragment thereof that specifically binds to OX40 ligand (OX40L); (b) a stabilizer; (c) a surfactant; and (d) a buffer comprising histidine or a histidine salt; wherein the pH of the aqueous pharmaceutical formulation ranges from 5.5±0.2 to about 6.5±0.2, wherein the antibody or antigen binding fragment thereof comprises: a heavy chain complementarity determining region (HCDR) 1 of SEQ ID NO: 42, an HCDR2 of SEQ ID NO:44, and an HCDR3 of SEQ ID NO:46; and a light chain complementarity determining region (LCDR) 1 of SEQ ID NO:56, an LCDR2 of SEQ ID NO:58, and an LCDR3 of SEQ ID NO:60; and wherein the aqueous pharmaceutical formulation is suitable for parenteral administration to a mammalian subject.

In one aspect, embodiments of the present disclosure pertain to an aqueous pharmaceutical formulation which comprises: (a) an anti-OX40 ligand (OX40L) antagonist antibody or antigen binding fragment; (b) a stabilizer; (c) a surfactant; and (d) a buffer comprising histidine or a histidine salt; wherein the pH of the aqueous pharmaceutical formulation ranges from 5.5±0.2 to about 6.5±0.2, wherein the antibody or antigen binding fragment thereof comprises: a heavy chain complementarity determining region (HCDR) 1 of SEQ ID NO: 42, an HCDR2 of SEQ ID NO:44, and an HCDR3 of SEQ ID NO:46; and a light chain complementarity determining region (LCDR) 1 of SEQ ID NO:56, an LCDR2 of SEQ ID NO:58, and an LCDR3 of SEQ ID NO:60; and wherein the aqueous pharmaceutical formulation is suitable for parenteral administration to a mammalian subject.

In certain embodiments, the antibody or antigen binding fragment thereof is amlitelimab or variants thereof.

In one aspect, embodiments of the present disclosure pertain to an aqueous pharmaceutical formulation which comprises: (a) an anti-OX40 ligand (OX40L) antagonist antibody or antigen binding fragment; (b) a stabilizer; (c) a chelator; (d) a surfactant; and (e) a buffer comprising histidine or a histidine salt; wherein the pH of the aqueous pharmaceutical formulation ranges from 5.5±0.2 to about 6.5±0.2, wherein the antibody or antigen binding fragment thereof comprises: a heavy chain complementarity determining region (HCDR) 1 of SEQ ID NO: 42, an HCDR2 of SEQ ID NO:44, and an HCDR3 of SEQ ID NO:46; and a light chain complementarity determining region (LCDR) 1 of SEQ ID NO:56, an LCDR2 of SEQ ID NO:58, and an LCDR3 of SEQ ID NO:60; and wherein the aqueous pharmaceutical formulation is suitable for parenteral administration to a mammalian subject. In certain embodiments, the antibody or antigen binding fragment thereof is amlitelimab or variants thereof.

In another aspect, embodiments of the present disclosure pertain to an aqueous pharmaceutical formulation which comprises: (a) an antibody or antigen binding fragment thereof that specifically binds to OX40 ligand (OX40L); (b) a stabilizer; (c) a chelator; (d) a surfactant; and (e) a buffer comprising histidine or a histidine salt; wherein the pH of the aqueous pharmaceutical formulation ranges from 5.5±0.2 to about 6.5±0.2, wherein the antibody or antigen binding fragment thereof comprises: a heavy chain complementarity determining region (HCDR) 1 of SEQ ID NO: 42, an HCDR2 of SEQ ID NO:44, and an HCDR3 of SEQ ID NO:46; and a light chain complementarity determining region (LCDR) 1 of SEQ ID NO:56, an LCDR2 of SEQ ID NO:58, and an LCDR3 of SEQ ID NO:60; and wherein the aqueous pharmaceutical formulation is suitable for parenteral administration to a mammalian subject. In certain embodiments, the antibody or antigen binding fragment thereof is amlitelimab or variants thereof.

In certain embodiments, the antibody or antigen binding fragment thereof comprised in the aqueous pharmaceutical formulation is amlitelimab or variants thereof. In certain embodiments, the antibody or antigen binding fragment thereof is present in a concentration ranging from about 28 mg/mL to about 138 mg/mL, or from 31 mg/mL to 125 mg/mL. In one embodiment, the antibody or antigen binding fragment thereof is present in a concentration of 28 mg/mL, 31 mg/mL, 62.5 mg/mL, 125 mg/mL, or 138 mg/mL. In one embodiment, the antibody or antigen binding fragment thereof is present in a concentration of 28 mg/mL, 31 mg/mL, 125 mg/mL, or 138 mg/mL. In certain embodiments, the antibody or antigen binding fragment thereof is present in a concentration ranging from 75 mg/mL to 125 mg/mL. In some embodiments, the antibody or antigen binding fragment thereof is present in the aqueous pharmaceutical formulation in a concentration of 125 mg/mL to 138 mg/. In one embodiment, the antibody or antigen binding fragment thereof is present in a concentration of 125 mg/mL.

In certain embodiments, the antibody or antigen binding fragment thereof is administered to a subject as an initial dose of 500 mg, followed by one or more secondary doses of 250 mg each q4w. In certain embodiments, the loading dose comprises 2×2 mL injections of 250 mg. In certain embodiments, the one or more secondary doses each comprises a 1×2 mL injection of 250 mg.

In certain embodiments, the antibody or antigen binding fragment thereof is administered to a subject as an initial dose of 500 mg, followed by one or more secondary doses of 250 mg every 12 weeks (q12w). In certain embodiments, the antibody or antigen binding fragment thereof is administered to a subject as an initial dose of 250 mg, followed by one or more secondary doses of 125 mg every 12 weeks (q12w).

In certain embodiments, the antibody or antigen binding fragment thereof is administered to a subject as an initial dose of 250 mg, followed by one or more secondary doses of 250 mg each q4w. In certain embodiments, the loading dose comprises a 1×2 mL injection of 250 mg antibody or antigen-binding fragment thereof and 1×2 mL placebo. In certain embodiments, the one or more secondary doses each comprises a 1×2 mL injection of 250 mg.

In certain embodiments, the antibody or antigen binding fragment thereof is administered to a subject as an initial dose of 15 mg, followed by one or more secondary doses of 125 mg each q4w. In certain embodiments, the loading dose comprises a 1×2 mL injection of 125 mg antibody or antigen-binding fragment thereof and 1×2 mL placebo. In certain embodiments, the one or more secondary doses each comprises a 1×2 mL injection of 125 mg.

In certain embodiments, the stabilizer in the pharmaceutical formulation is mannitol and/or sucrose. In certain embodiments, the stabilizer in the pharmaceutical formulation is sucrose. In one embodiment, the at least one stabilizer is sucrose, which is present in an amount of 220 mM±33 mM. In one embodiment, the at least one stabilizer is sucrose, which is present in an amount of 220 mM.

In certain embodiments, the surfactant in the pharmaceutical formulation is selected from polysorbate 80. In one embodiment, the aqueous pharmaceutical formulation comprises 0.01% (w/v) to 0.1% (w/v) polysorbate 80. In one embodiment, the aqueous pharmaceutical formulation comprises 0.02% (w/v) to 0.1% (w/v) polysorbate 80. In one embodiment, the aqueous pharmaceutical formulation comprises 0.02% (w/v) to 0.06% (w/v) polysorbate 80. In another embodiment, the aqueous pharmaceutical formulation comprises 0.04% (w/v) polysorbate 80. In yet another embodiment, the aqueous pharmaceutical formulation comprises 0.06% (w/v) polysorbate 80.

In certain embodiments, the buffer comprised in the pharmaceutical formulation comprises 10 mM±1.5 mM L-histidine or histidine hydrochloride. In certain embodiments, the buffer comprised in the pharmaceutical formulation comprises 20 mM L-histidine or histidine hydrochloride.

In certain embodiments, the pharmaceutical formulation further comprises a chelator selected from the group consisting of ethylenediaminetetraacetic acid (EDTA), diethylenetriamene pentaacetate (DTPA) and salts thereof, and any combinations thereof. In certain embodiments, the chelator comprises 10 μM EDTA or 10 μM DTPA.

In one embodiment, the aqueous pharmaceutical formulation comprises 28±4.2 mg/mL to 138 mg/mL±20.7 mg/mL of the antibody or antigen binding fragment thereof; 10 mM±1.5 mM L-histidine or histidine hydrochloride; 220 mM sucrose, 0.06% (w/v) polysorbate 80; optionally, 10 μM EDTA or 10 μM DTPA, and water; with the pH of the aqueous pharmaceutical formulation being about 5.8 to about 6.2.

In one embodiment, the aqueous pharmaceutical formulation comprises 125 mg/mL to 138 mg/mL of the antibody or antigen binding fragment thereof; 20 mM L-histidine or histidine hydrochloride; 220 mM sucrose, 0.04% (w/v) polysorbate 80; and water; with the pH of the aqueous pharmaceutical formulation being about 5.8 to about 6.2.

In some embodiments, the aqueous pharmaceutical formulation is free or essentially free of particles. In some embodiments, the aqueous pharmaceutical formulation is free or substantially free of sodium chloride. In some embodiments, the aqueous pharmaceutical formulation is free or substantially free of arginine.

In some embodiments, less than 5% of the antibody is detected in an aggregated form after 28 days of storage at 40° C., detected by size exclusion high performance liquid chromatography.

In some embodiments, at least 91% of the antibody comprised in the aqueous pharmaceutical formulation has native conformation after 28 days at 40° C., measured by size exclusion chromatography. In some embodiments, at least 94% of the antibody in the aqueous pharmaceutical formulation has native conformation after 8 weeks at 25° C., measured by size exclusion chromatography. In some embodiments, at least 98% of the antibody has native conformation after 16 weeks at 5° C. or at −65° C., measured by size exclusion chromatography.

In some embodiments, at least 55% of the antibody in the aqueous pharmaceutical formulation is the main charge variant of the antibody after 28 days at 40° C., measured by imaged capillary isoelectric focusing (iCIEF). In some embodiments, at least 65% of the antibody is the main charge variant of the antibody after 16 weeks at 25° C., measured by imaged capillary isoelectric focusing (iCIEF). In some embodiments, at least 70% of the antibody is the main charge variant of the antibody after 16 weeks at 5° C., measured by imaged capillary isoelectric focusing (iCIEF). In some embodiments, at least 70% of the antibody is the main charge variant of the antibody after 16 weeks at −65° C., measured by imaged capillary isoelectric focusing (iCIEF).

In certain embodiments, the aqueous pharmaceutical formulation is stable upon storage at 5° C. or 25° C. for at least 3 months. In one embodiment, the aqueous pharmaceutical formulation is stable upon storage at 40° C. for at least 2 months. In one embodiment, the aqueous pharmaceutical formulation is stable upon storage at 5° C. for at least 1 year.

In certain embodiments, the aqueous pharmaceutical formulation is stable upon storage at −65° C., 5° C., or 25° C. for at least 16 weeks. In one embodiment, the aqueous pharmaceutical formulation is stable upon storage at 40° C. for at least 8 weeks. In one embodiment, the aqueous pharmaceutical formulation is stable upon storage at 5° C. for at least 1 year. In certain embodiments, the aqueous pharmaceutical formulation is stable upon freezing and thawing.

In certain embodiments, the aqueous pharmaceutical formulation according to the present disclosure is contained in a container. In certain embodiments, the aqueous pharmaceutical formulation is suitable for subcutaneous delivery.

In an aspect, the application pertains to a pharmaceutical unit dosage form suitable for parenteral administration to a mammalian subject which comprises the aqueous pharmaceutical formulation disclosed herein in a suitable container. In certain embodiments, the aqueous pharmaceutical formulation is suitable for intravenous, subcutaneous, or intramuscular administration. In one embodiment, the suitable container is a pre-filled syringe. In one embodiment, the suitable container is a pre-filled pen or an autoinjector.

In another aspect, embodiments of the present disclosure pertain to a sealed container comprising the aqueous pharmaceutical formulation disclosed herein. In some embodiments, the sealed container is a vial, a syringe, a microinfusor, a pen delivery device, or an autoinjector. In another embodiment, the sealed container is a single or multi-chambered syringe. In yet another embodiment, the sealed container is a pre-filled syringe containing 2.25 ml of the aqueous pharmaceutical formulation.

In still another aspect, embodiments of the present disclosure pertain to a pre-filled syringe comprising an aqueous pharmaceutical formulation comprising: about 28 mg/mL to about 138 mg/mL of an antibody or antigen binding fragment thereof that specifically binds to OX40 ligand (OX40L); 10 mM L-histidine or histidine hydrochloride; 220 mM sucrose; 0.06% (w/v) polysorbate 80, and water; wherein the pH of the aqueous pharmaceutical formulation is 6.0. In some embodiments, the aqueous pharmaceutical formulation contained in the pre-filled syringe may further comprise 10 μM EDTA or 10 μM DTPA. In some embodiments, the pre-filled syringe comprises a safety system, e.g. such as a needle guard, such as the BD UltraSafe™ or BD UltraSafe Plus™ needle guard. In some embodiments, the aqueous pharmaceutical formulation contained in the pre-filled syringe comprises from 31 mg/mL to 125 mg/mL antibody or antigen binding fragment thereof that specifically binds to OX40 ligand (OX40L). In certain embodiments, the aqueous pharmaceutical formulation contained in the pre-filled syringe comprises about 31 mg/mL to about 125 mg/mL amlitelimab or a variant thereof. In certain embodiments, the aqueous pharmaceutical formulation contained in the pre-filled syringe comprises 125 mg/mL±18.75 mg/mL or about 125 mg/mL amlitelimab or a variant thereof. In one embodiment, the pre-filled syringe comprises about 125 mg of amlitelimab in a 1 mL solution (125 mg/mL±18.75 mg/mL). In one embodiment, the pre-filled syringe comprises about 250 mg of amlitelimab in a 2 mL solution (125 mg/mL±18.75 mg/mL).

In still another aspect, embodiments of the present disclosure pertain to a pre-filled pen or an autoinjector comprising an aqueous pharmaceutical formulation comprising: about 28 mg/mL to about 138 mg/mL of an antibody or antigen binding fragment thereof that specifically binds to OX40 ligand (OX40L); 10 mM L-histidine or histidine hydrochloride; 220 mM sucrose; 0.06% (w/v) polysorbate 80, and water; wherein the pH of the aqueous pharmaceutical formulation is 6.0. In some embodiments, the aqueous pharmaceutical formulation contained in the pre-filled pen or autoinjector may further comprise 10 μM EDTA or 10 μM DTPA. In some embodiments, the aqueous pharmaceutical formulation contained in the pre-filled pen or autoinjector comprises from 31 mg/mL to 125 mg/mL antibody or antigen binding fragment thereof that specifically binds to OX40 ligand (OX40L). In certain embodiments, the aqueous pharmaceutical formulation contained in the pre-filled pen or autoinjector comprises about 31 mg/mL to about 125 mg/mL amlitelimab or a variant thereof. In certain embodiments, the aqueous pharmaceutical formulation contained in the pre-filled pen or autoinjector comprises 125 mg/mL±18.75 mg/mL amlitelimab or a variant thereof. In one embodiment, the pre-filled pen or autoinjector comprises about 125 mg of amlitelimab in a 1 mL solution (125 mg/mL±18.75 mg/mL). In one embodiment, the pre-filled pen or autoinjector comprises about 250 mg of amlitelimab in a 2 mL solution (125 mg/mL±18.75 mg/mL).

In still another aspect, embodiments of the present disclosure pertain to a pre-filled syringe comprising an aqueous pharmaceutical formulation comprising: 125 mg/mL mg/mL to 138 mg/mL of amlitelimab or a variant thereof; 20 mM L-histidine or histidine hydrochloride; 220 mM sucrose; 0.04% (w/v) polysorbate 80, and water; wherein the pH of the aqueous pharmaceutical formulation is 6.0.

In one embodiment, the pre-filled pen or autoinjector includes a single dose of the formulation. In one embodiment, the pre-filled pen or autoinjector is sleeve-triggered. In one embodiment, the pre-filled pen or autoinjector comprises a fixedly arranged syringe. In one embodiment, the pre-filled pen or autoinjector provides audible feedback at the end of injection and/or following injection. In one embodiment, the pre-filled pen or autoinjector comprises an anti-roll feature.

In still another aspect, embodiments of the present disclosure pertain to a kit comprising a sealed container comprising an aqueous pharmaceutical formulation disclosed herein. In one embodiment, the sealed container is a pre-filled syringe comprising an aqueous pharmaceutical formulation disclosed herein. In one embodiment, the sealed container is a pre-filled pen or an autoinjector comprising an aqueous pharmaceutical formulation disclosed herein. In one embodiment, the pre-filled pen or autoinjector includes a single dose of the formulation. In one embodiment, the pre-filled pen or autoinjector is sleeve-triggered. In one embodiment, the pre-filled pen or autoinjector comprises a fixedly arranged syringe. In one embodiment, the pre-filled pen or autoinjector provides audible feedback at the end of injection and/or following injection. In one embodiment, the pre-filled pen or autoinjector comprises an anti-roll feature.

In yet another aspect, embodiments of the present disclosure pertain to a kit comprising: a sealed container comprising any aqueous pharmaceutical formulation disclosed herein and at least one separate injection device for delivery of the aqueous pharmaceutical formulation to a mammalian subject in need thereof. In one embodiment, the injection device is a syringe, a microinfusor, a pen delivery device, or an autoinjector. In one embodiment, the injection device is a single or multi-chambered syringes.

In yet another aspect, embodiments of the present disclosure pertain to the use of an aqueous pharmaceutical formulation disclosed herein, a pharmaceutical unit dosage form disclosed herein, a sealed container disclosed herein, a pre-filled syringe, a prefilled pen or autoinjector, or a kit disclosed herein in treating a disease or condition in a subject selected from the group consisting of an autoimmune disease or condition, an inflammatory disease or condition, a systemic inflammatory disease or condition, and a transplant rejection disease or condition mediated by hOX40L.

In yet another aspect, embodiments of the present disclosure pertain to a method of treating a disease or condition in a subject selected from the group consisting of an autoimmune disease or condition, an inflammatory disease or condition, a systemic inflammatory disease or condition, and a transplant rejection disease or condition, where the method comprises administering to a subject in need thereof an effective amount of an aqueous pharmaceutical formulation described herein.

In certain embodiments, a method of treating atopic dermatitis is provided wherein the method comprises administering to a subject in need thereof an effective amount of an aqueous pharmaceutical formulation described herein.

In certain embodiments, a method of treating asthma is provided wherein the method comprises administering to a subject in need thereof an effective amount of an aqueous pharmaceutical formulation described herein.

In another aspect, an aqueous pharmaceutical formulation is provided comprising: (a) an anti-OX40 ligand (OX40L) antagonist antibody or antigen binding fragment; (b) a stabilizer; (c) a chelator; (d) a surfactant; and (e) a buffer comprising histidine or a histidine salt; wherein the pH of the aqueous pharmaceutical formulation ranges from 5.5 to about 6.5, wherein the antibody or antigen binding fragment thereof comprises: a VH domain which comprises the HCDR1 sequence of SEQ ID NO:36 or 42, the HCDR2 sequence of SEQ ID NO:38 or 44, and the HCDR3 sequence of SEQ ID NO:40 or 46, and a VL domain which comprises the LCDR1 sequence of SEQ ID NO:50 or 56, the LCDR2 sequence of SEQ ID NO:52 or 58, and the LCDR3 sequence of SEQ ID NO:54 or 60; and wherein the aqueous pharmaceutical formulation is suitable for parenteral administration to a mammalian subject.

In another aspect, an aqueous pharmaceutical formulation is provided comprising: (a) an anti-OX40 ligand (OX40L) antagonist antibody or antigen binding fragment; (b) a stabilizer; (c) a surfactant; and (d) a buffer comprising histidine or a histidine salt; wherein the pH of the aqueous pharmaceutical formulation ranges from 5.5 to about 6.5, wherein the antibody or antigen binding fragment thereof comprises: a VH domain which comprises the HCDR1 sequence of SEQ ID NO:36 or 42, the HCDR2 sequence of SEQ ID NO:38 or 44, and the HCDR3 sequence of SEQ ID NO:40 or 46, and a VL domain which comprises the LCDR1 sequence of SEQ ID NO:50 or 56, the LCDR2 sequence of SEQ ID NO:52 or 58, and the LCDR3 sequence of SEQ ID NO:54 or 60; and wherein the aqueous pharmaceutical formulation is suitable for parenteral administration to a mammalian subject.

In certain embodiments, the VL domain comprises the amino acid sequence of SEQ ID NO:48 and the VH domain comprises the amino acid sequence of SEQ ID NO:34.

In certain embodiments, the antibody comprises a heavy chain and a light chain, wherein the heavy chain amino acid sequence consists of the amino acid sequence of SEQ ID No: 62 and the light chain amino acid sequence consists of the amino acid sequence of SEQ ID No: 64

### BRIEF DESCRIPTION OF THE DRAWINGS

The accompanying drawings, which are incorporated herein and constitute part of this specification, illustrate exemplary embodiments of the disclosure, and, together with the general description given above and the detailed description given below, serve to explain the features of the disclosure.

FIGS. 1A, 1B, and 1C graphically depict HMWS evolution of the formulations contained in OMPI syringes upon storage over 12 weeks at 5° C. (FIG. 1A), 25° C. (FIG. 1B), and 40° C. (FIG. 1C), respectively, evaluated by SEC-HPLC in a buffer/pH screening study in Example 1.

FIGS. 2A, 2B, 2C graphically depict the evolution of ≥2 μm subvisible particles in the example formulations contained in OMPI syringes upon storage over 12 weeks at 5° C. (FIG. 3A), 25° C. (FIG. 2B), and 40° C. (FIG. 2C), evaluated by a particle counter in a buffer/pH screening study in Example 1.

FIGS. 3A, 3B, and 3C graphically depict the evolution of ≥10 μm subvisible particles in the example formulations contained in OMPI syringes upon storage over 12 weeks at 5° C. (FIG. 3A), 25° C. (FIG. 3B), and 40° C. (FIG. 3C), evaluated by a particle counter in a buffer/pH screening study in Example 1.

FIGS. 4A, 4B, and 4C graphically depict the evolution of ≥25 μm subvisible particles in the example formulations contained in the OMPI syringes upon storage over 12 weeks at 5° C. (FIG. 4A), 25° C. (FIG. 4B), and 40° C. (FIG. 4C), evaluated by a particle counter in a buffer/pH screening study in Example 1.

FIGS. 5A, 5B, and 5C graphically depict the results of turbidity of the formulation samples contained in the OMPI syringes upon storage over 12 weeks at 5° C. (FIG. 5A), 25° C. (FIG. 5B), and 40° C. (FIG. 5C), evaluated by a particle counter in a buffer/pH screening study in Example 1.

FIGS. 6A, 6B, and 6C graphically depict the purity and fragmentation measured by capillary electrophoresis of the formulation samples contained in the OMPI syringes upon storage over 12 weeks at 5° C. (FIG. 6A), 25° C. (FIG. 6B), and 40° C. (FIG. 6C), evaluated in the buffer/pH screening study in Example 1.

FIGS. 7A, 7B, and 7C graphically depict the PS80 quantification of the example formulations stored in Syringe Ref #1_s to 6_s and 11_s to 16_s over 12 weeks at 5° C. (FIG. 7A), 25° C. (FIG. 7B), and 40° C. (FIG. 7C), evaluated in the buffer/pH screening study in Example 1.

FIGS. 8A, 8B, and 8C graphically depict the charge variants analyzed by capillary isoelectric focusing (cIEF) for the example formulations stored in Syringe Ref #1_s to 6_s and 11_s to 16_s over 8 weeks at 5° C. (FIG. 8A), 25° C. (FIG. 8B), and 40° C. (FIG. 8C), evaluated in the buffer/pH screening study in Example 1.

FIG. 9 graphically depicts deamidation at HC N332 in the antibody in the example formulations contained in Syringe Ref #1_s to 6_s and 11_s to 16_s after 3-month storage at 40° C. evaluated in the buffer/pH screening study in Example 1.

FIG. 10 graphically depicts oxidation at HC M103 in the antibody in the example formulations contained in Syringe Ref #1_s to 6_s and 11_s to 16_s after 3-month storage at 40° C., evaluated in the buffer/pH screening study in Example 1.

FIG. 11 graphically depicts the pH measurements of the formulations contained in Syringe Ref #1_s to 6_s and 11_s to 16_s at t0 and after 12 weeks of storage at 25° C. and 40° C., evaluated in the buffer/pH screening study in Example 1.

FIG. 12 graphically depicts the osmolality measurements of the formulations contained in Syringe Ref #1_s to 6_s and 11_s to 16_s at t0 and after 12 weeks of storage at 40° C., as described in the buffer/pH screening study in Example 1.

FIGS. 13A, 13B, and 13C graphically depict aggregation measurements evaluated by SEC-HPLC for the formulations contained in Syringe Ref #1_s, 7_s, 8_s, 11_s, 17_x, and 18_s over 12 weeks of storage at 5° C. (FIG. 13A), 25° C. (FIG. 13B), and 40° C. (FIG. 13C), as described in the stabilizer study in Example 1.

FIGS. 14A, 14B, and 14C graphically illustrate the evolution of ≥2 μm subvisible particles in example formulations contained in Syringe Ref #1_s, 7_s, 8_s, 11_s, 17_x, and 18_s over 12 weeks of storage at 5° C. (FIG. 14A), 25° C. (FIG. 14B), and 40° C. (FIG. 14C), as described in the stabilizer study in Example 1.

FIGS. 15A, 15B, and 15C graphically depict the evolution of ≥10 μm subvisible particles in the example formulations contained in Syringe Ref #1_s, 7_s, 8_s, 11_s, 17_x, and 18_s over 12 weeks of storage at 5° C. (FIG. 15A), 25° C. (FIG. 15B), and 40° C. (FIG. 15C), as described in the stabilizer study in Example 1.

FIGS. 16A, 16B, and 16C graphically depict the evolution of ≥25 μm subvisible particles in example formulations contained in Syringe Ref #1_s, 7_s, 8_s, 11_s, 17_x, and 18_s over 12 weeks of storage at 5° C. (FIG. 16A), 25° C. (FIG. 16B), and 40° C. (FIG. 16C), as described in the stabilizer study in Example 1.

FIGS. 17A, 17B, and 17C graphically depict turbidity measurements of the example formulations contained in Syringe Ref #1_s, 7_s, 8_s, 11_s, 17_x, and 18_s at TO and after 12 weeks of storage at 5° C. (FIG. 17A), 25° C. (FIG. 17B), and 40° C. (FIG. 17C), as described in the stabilizer study in Example 1.

FIGS. 18A, 18B, and 18C graphically depict the PS80 quantification of the example formulations contained in Syringe Ref #1_s, 7_s, 8_s, 11_s, 17_x, and 18_s after 12 weeks pf storage at 5° C. (FIG. 18A), 25° C. (FIG. 18B), and 40° C. (FIG. 18C), as described in the stabilizer study in Example 1.

FIG. 19 graphically depicts deamidation at HC N332 in the antibody in the example formulations contained in Syringe Ref #1_s, 7_s, 8_s, 11_s, 17_x, and 18_s after 3-month storage at 40° C., as described in the stabilizer study in Example 1.

FIG. 20 graphically depicts oxidation at HC M103 in the antibody in the example formulations contained in Syringe Ref #1_s, 7_s, 8_s, 11_s, 17_x, and 18_s after 3-month storage at 40° C., as described in the stabilizer study in Example 1.

FIGS. 21A, 21B, and 21C graphically depict HMWS evolution of the formulations in syringes 1_s_Omp, 9_s_BD, 10_s_BD, and 11_s_Omp over 12 weeks of storage at 5° C. (FIG. 21A), 25° C. (FIG. 21B), and 40° C. (FIG. 21C), as described in the syringe comparability study in Example 1.

FIGS. 22A, 22B, and 22C graphically depict the concentration of subvisible particles of ≥2 μm measured by HIAC in the example formulations contained in syringes 1_s_Omp, 9_s_BD, 10_s_BD, and 11_s_Omp over 12 weeks of storage at 5° C. (FIG. 22A), 25° C. (FIG. 22B), and 40° C. (FIG. 22C), respectively, as described in syringe comparability study in Example 1.

FIGS. 23A, 23B, and 23C graphically depict the concentration of subvisible particles of ≥10 μm measured by HIAC in the example formulations contained in syringes 1_s_Omp, 9_s_BD, 10_s_BD, and 11_s_Omp over 12 weeks of storage at 5° C. (FIG. 23A), 25° C. (FIG. 23B), and 40° C. (FIG. 23C), respectively, as described in the syringe comparability study in Example 1.

FIGS. 24A, 24B, and 24C graphically depict the concentration of subvisible particles of ≥25 μm measured by HIAC in the example formulations contained in syringes 1_s_Omp, 9_s_BD, 10_s_BD, and 11_s_Omp over 12 weeks of storage at 5° C. (FIG. 24A), 25° C. (FIG. 24B), and 40° C. (FIG. 24C), respectively, as described in the syringe comparability study in Example 1.

FIGS. 25A, 25B, and 25C graphically depict the turbidity measurements for the example formulations contained in syringes 1_s_Omp, 9_s_BD, 10_s_BD, and 11_s_Omp at t) and after 12 weeks of storage at 5° C. (FIG. 25A), 25° C. (FIG. 25B), and 40° C. (FIG. 25C), respectively, as described in the syringe comparability study in Example 1.

FIGS. 26A, 26B, and 26C graphically depict the charge variants of the antibody measured by cIEF in the example formulations contained in syringes 1_s_Omp, 9_s_BD, 10_s_BD, and 11_s_Omp at t) over 12 weeks of storage at 5° C. (FIG. 26A), 25° C. (FIG. 26B), and 40° C. (FIG. 26C), respectively, as described in the syringe comparability study in Example 1.

FIGS. 27A, 27B, and 27C graphically depict the evolution of HMWS % measured by SEC-HPLC in the sample formulations in the syringes with Ref No. C-1 to C_6 over 12 weeks of storage at 5° C. (FIG. 27A), 25° C. (FIG. 27B), and 40° C. (FIG. 27C), as described in the chelator justification study in Example 1.

FIG. 28A graphically depicts the PS80 measurements analyzed by CAD in the formulations of Ref. No. C_1 to C_6 over 2 months of storage at 5° C., 25° C., and 40° C., as described in the chelator justification study in Example 1.

FIGS. 28B, 28C, and 28D graphically depict the changes in PS80 in the formulations of Ref. No. C_1 to C_6 over 3 months of storage at 5° C. (FIG. 29A), 25° C. (FIG. 29B), and 40° C. (FIG. 29C) (Dotted line indicates the method variability), as described in the chelator justification study in Example 1.

FIG. 29 depicts the percent change per week of HC N332 deamidation of the antibody in the formulations of Ref. No. C_1 to C_6 over 8 weeks of storage at 40° C., as described in the chelator justification study in Example 1.

FIG. 30 graphically depicts the percent change per week of HC M103 oxidation of the antibody in the formulations over 8 weeks of storage at 40° C., as described in the chelator justification study in Example 1.

Charge variants were analyzed at TO, T2 wk (two weeks), T4 wk (4 weeks), and T8 wk (8 weeks) by CIEF for formulations of Ref. Nos. C-1 to C_6 stored over 8 weeks at 5° C., 25° C., and 40° C., as described in the chelator justification study in Example 1.

FIGS. 31A, 31B, and 31C graphically depict the charge variants measured by cIEF for the antibody in the formulations of Ref. No. C_1 to C_6 over 8 weeks of storage at 5° C., 25° C., and 40° C. where acidic peak is illustrated in FIG. 31A, the basic peak is illustrated in FIG. 31B, and the main peak is illustrated in FIG. 31C, as described in the chelator justification study in Example 1.

FIGS. 32A, 32B, and 32C are graphs depicting the measurements of HMWS evolution for formulations with 31 mg/mL and 125 mg/mL antibody at timepoints of T0, T1 hr, T3 hr, and T6 hr over the duration of the wrist-action study, as described in the PS80 justification study in Example 1.

FIGS. 33A, 33B, and 33C graphically depict the turbidity analysis for the example formulations with 31 mg/mL and 125 mg/mL antibody at timepoints of T0, T1 hr, T3 hr, and T6 hr over the duration of the wrist-action study, as described in the PS80 justification study in Example 1.

FIGS. 34A, 34B, 34C, 34D, 34E, and 34F graphically depict subvisible particles of at least 2 μm, at least 10 μm, or at least 25 μm measured by HIAC in the example formulations for the example formulations with 31 mg/mL and 125 mg/mL antibody at timepoints of T0, T1 hr, T3 hr, and T6 hr over the duration of the wrist-action study, as described in the PS80 justification study in Example 1.

FIGS. 35A and 35B are graphs depicting the results of HMWS evolution analyzed by SEC-HPLC for 31 mg/mL and 125 mg/mL formulations over the duration of the orbital shaking study, as described in the PS80 justification study in Example 1.

FIGS. 36A, 36B, 36C, 36D, 36E, and 36F graphically depict subvisible particles of at least 2 μm, at least 10 μm, or at least 25 μm measured by HIAC in the example formulations for the example formulations with 31 mg/mL and 125 mg/mL antibody at timepoints of T0, T1 hr, T3 hr, and T6 hr over the duration of the orbital shaking study, as described in the PS80 justification study in Example 1.

FIGS. 37A and 37B graphically depict turbidity for 31 mg/mL and 125 mg/mL formulations over the duration of the study with wrist-action and silicone oil, as described in the PS80 justification study in Example 1.

FIG. 38 is a graph depicting the evolution of HMWS analyzed by SEC-HPLC for the formulations F2-1, F2-2, and F2-3 over the 3 months of storage at 5° C., 25° C., and 40° C., as described in Example 2.

FIG. 39 is a graph depicting the PS80 loss of the formulations F2-1, F2-2, and F2-3 over 3 months of storage at 5° C., 25° C., and 40° C., as detailed in Example 2.

FIG. 40 is a graph depicting the percent change per week of deamidation at HC D332 of the antibody in formulations F2-1, F2-2, and F2-3 after 1 month of storage at 40° C., measured by peptide mapping in Example 2.

FIG. 41 is a graph depicting the percent change per week of oxidation at HC M103 of the antibody in formulations F2-1, F2-2, and F2-3 after 1 month of storage at 40° C., measured by peptide mapping, as detailed in Example 2.

FIG. 42 is a graph depicting the charge variants measured by capillary isoelectric focusing (cIEF) for formulations F2-1, F2-2, and F2-3 over two months of storage at 5° C., 25° C., and 40° C., as detailed in Example 2.

FIG. 43 is a table showing the stability results analyzed for formulations F3-1, F3-2, and F3-3 in containers over 3 months of storage at 5° C., 25° C., and 40° C. according to the study in Example 3.

FIG. 44 graphically depicts a result of gliding forces of 2.23 mL BD and OMPI PFS according to a study in Example 4.

FIG. 45 graphically depicts viscoelastic behavior of amlitelimab in a concentration of 263 mg/ml in 25 mM histidine/histidine hydrochloride buffer (HisHCl) at pH 6.2, analyzed by shear rate ramping from 0-4000 s−1 at 25° C. as described in Example 5.

FIG. 46 graphically depicts a concentration-dependent viscosity profile of amlitelimab in 25 mM histidine/histidine hydrochloride buffer (HisHCl) at pH 6.2 at 5° C. and 25° C. as described in Example 5.

FIG. 47 graphically depicts the protein contents of the formulation samples at initial timepoint T0 and timepoints after being subject to freeze-thaw stress (−65° C. to 25° C.) or shaking stress at ambient temperature and at cool temperature, measured by UV/Vis.

FIG. 48 graphically depicts the pH values of the formulation samples at initial timepoint T0 and timepoints after being subject to freeze-thaw stress (−65° C. to 25° C.) or shaking stress at ambient temperature and at cool temperature.

FIG. 49 graphically depicts the surfactant contents of the formulation samples at initial timepoint T0 and timepoints after being subject to freeze-thaw stress (−65° C. to 25° C.) or shaking stress at ambient temperature and at cool temperature.

FIG. 50 graphically depicts the clarity and opalescence of solution (turbidity) of the formulation samples at initial timepoint T0 or at timepoints after being subject to freeze-thaw stress (−65° C. to 25° C.) or shaking stress at ambient temperature and at cool temperature.

FIGS. 51A, 51B, and 51C graphically depict the differences in the percentages of high molecular weight species (HMWS), main peak, and low molecular weight species (LMWS) for all formulations samples subject to freeze-thaw stress (−65° C. to 25° C.) or shaking stress at ambient temperature and at cool temperature, analyzed by size exclusion chromatography (SE-HPLC) in Example 6, where FIG. 51A graphically depicts the percent of main peak obtained from the SE-HPLC analysis, FIG. 51B graphically depicts the HMW species obtained from the SE-HPLC analysis, and FIG. 51C graphically depicts the HMW species obtained from the SE-HPLC analysis.

FIGS. 52A and 52B graphically depict subvisible particles of all formulation samples analyzed by HIAC in Example 2. FIG. 52A includes panel (a) and panel (b) which graphically depict the counts of subvisible particles having a size of at least 2 μm for all formulation samples, where panel (a) illustrates the counts in a range from 0 to 12000 (counts/mL), while panel (b) illustrates the counts in the range of 0-2000 (counts/mL). FIG. 52B includes panel (a), panel (b), and panel (c) which respectively graphically depict the counts of subvisible particles having a size of at least 5 μm (panel a), at least 10 (panel b), and at least 25 (panel (c) for all formulation samples.

FIGS. 53A, 53B, and 53C graphically depict charged variants assessment for all formulation samples stored for over 8 weeks or 16 weeks at −65° C., 5° C., 25° C., and 40° C., or subject to freeze-thaw stress (−65° C. to 25° C.) or shaking stress at ambient temperature and at cool temperature. FIG. 53A graphically depicts the main peak of protein measured by imaged capillary isoelectric focusing (iCIEF). FIG. 53B graphically depicts the acidic variant measured by imaged capillary isoelectric focusing (iCIEF). FIG. 53C graphically depicts the basic variant measured by imaged capillary isoelectric focusing (iCIEF).

### DETAILED DESCRIPTION

Embodiments of the present disclosure provide aqueous pharmaceutical formulations comprising anti-OX40L antibodies or antigen binding fragments thereof for treating or preventing OX40L-mediated diseases or conditions in humans. Embodiments also provide packaged drug products comprising a container comprising the formulations disclosed herein. In some embodiments, the anti-OX40L antibodies are amlitelimab, amlitelimab variants, or antigen binding fragments thereof.

Before the present disclosure is described in detail, it is to be understood that the embodiments described herein are not limited to the particular methods and experimental conditions described, as such methods and conditions may vary. It is also to be understood that the terminology used herein is for the purpose of describing particular embodiments only, and is not intended to be limiting in scope. Other than in the operating examples, or where otherwise indicated, all numbers expressing quantities of ingredients or reaction conditions used herein should be understood as disclosing those numbers, as well as those numbers modified in all instances by the term “about.”

### Definitions

Listed below are definitions of various terms used to describe the embodiments disclosed herein. These definitions apply to the terms as they are used throughout this specification and claims, unless otherwise limited in specific instances, either individually or as part of a larger group.

Unless otherwise defined, all scientific and technical terms used herein shall have the meanings that are commonly understood by those of ordinary skill in the art. Further, unless otherwise required by context, singular terms shall include pluralities and plural terms shall include the singular. Generally, nomenclature utilized in connection with, and techniques of, cell and tissue culture, molecular biology, and protein and oligo- or polynucleotide chemistry and hybridization described herein are those well-known and commonly used in the art.

As used in this specification and the appended claims, the singular forms “a,” “an” and “the” include plural referents unless the content clearly dictates otherwise. Thus, for example, reference to “a molecule” optionally includes a combination of two or more such molecules, and the like. The use of “or” herein is the inclusive or.

As used herein, the term “about” or “approximately” will be understood by persons of ordinary skill in the art and will vary to some extent on the context in which it is used. When referring to a measurable value such as an amount, a temporal duration, and the like, the term “about” or “approximately” is meant to encompass variations of 20%, ±15%, or ±10%, including ±5%, ±1%, and ±0.1% from the specified value, as such variations are appropriate to perform the disclosed methods.

As used in this specification and claim(s), the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the term “consisting essentially of” refers to those elements required for a given embodiment. The term permits the presence of elements that do not materially affect the basic and novel or functional characteristic(s) of that embodiment.

The abbreviation, “e.g.” is derived from the Latin exempli gratia, and is used herein to indicate a non-limiting example. Thus, the abbreviation “e.g.” is synonymous with the term “for example.”

As used herein, the term “amino acid” includes alanine (Ala or A); arginine (Arg or R); asparagine (Asn or N); aspartic acid (Asp or D); cysteine (Cys or C); glutamine (Gin or Q); glutamic acid (Glu or E); glycine (Gly or G); histidine (His or H); isoleucine (lie or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V). Non-traditional amino acids are also within the scope of the disclosure and include norleucine, ornithine, norvaline, homoserine, and other amino acid residue analogues such as those described in Ellman et al. Meth. Enzymol. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA. Introduction of the non-traditional amino acid can also be achieved using peptide chemistries known in the art. As used herein, the term “polar amino acid” includes amino acids that have net zero charge, but have non-zero partial charges in different portions of their side chains (e.g., M, F, W, S, Y, N, Q, C). These amino acids can participate in hydrophobic interactions and electrostatic interactions. As used herein, the term “charged amino acid” includes amino acids that can have non-zero net charge on their side chains (e.g., R, K, H, E, D). These amino acids can participate in hydrophobic interactions and electrostatic interactions.

As used herein, the term “conservative amino acid substitutions” refers to amino acid substitutions result from replacing one amino acid with another having similar structural and/or chemical properties, such as the replacement of a leucine with an isoleucine or valine, an aspartate with a glutamate, or a threonine with a serine. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not reduce the activity of the peptide, (i.e. the ability of the peptide to penetrate the blood brain barrier (BBB)). Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984), incorporated by reference in its entirety.) In some embodiments, individual substitutions, deletions or additions that alter, add or delete a single amino acid or a small percentage of amino acids can also be considered “conservative substitutions” if the change does not reduce the activity of the peptide. Insertions or deletions are typically in the range of about 1 to 5 amino acids. The choice of conservative amino acids may be selected based on the location of the amino acid to be substituted in the peptide, for example if the amino acid is on the exterior of the peptide and expose to solvents, or on the interior and not exposed to solvents.

In alternative embodiments, one can select the amino acid which will substitute an existing amino acid based on the location of the existing amino acid, i.e. its exposure to solvents (i.e. if the amino acid is exposed to solvents or is present on the outer surface of the peptide or polypeptide as compared to internally localized amino acids not exposed to solvents). Selection of such conservative amino acid substitutions are well known in the art, for example as disclosed in Dordo et al, J. Mol Biol, 1999, 217, 721-739 and Taylor et al., J. Theor. Biol. 119(1986); 205-218 and S. French and B. Robson, J. Mol. Evol., 19(1983)171. Accordingly, one can select conservative amino acid substitutions suitable for amino acids on the exterior of a protein or peptide (i.e. amino acids exposed to a solvent), for example, but not limited to, the following substitutions can be used: substitution of Y with F, T with S or K, P with A, E with D or Q, N with D or G, R with K, G with N or A, T with S or K, D with N or E, I with L or V, F with Y, S with T or A, R with K, G with N or A, K with R, A with S, K or P.

In alternative embodiments, one can also select conservative amino acid substitutions encompassed suitable for amino acids on the interior of a protein or peptide, for example one can use suitable conservative substitutions for amino acids is on the interior of a protein or peptide (i.e. the amino acids are not exposed to a solvent), for example but not limited to, one can use the following conservative substitutions: where Y is substituted with F, T with A or S, I with L or V, W with Y, M with L, N with D, G with A, T with A or S, D with N, I with L or V, F with Y or L, S with A or T and A with S, G, T or V. In some embodiments, non-conservative amino acid substitutions are also encompassed within the term of variants.

As used herein, the term “administering,” refers to dispensing, delivering, or applying an active compound, i.e., an antibody or antigen-binding fragment thereof, according to the present disclosure, in a pharmaceutical formulation to a subject by any suitable route for delivery of the active compound to the subject. Examples of routes of administration include, but are not limited to, subcutaneous, intravenous, e.g., intravenous injection and intravenous infusion, e.g., via central venous access, intramuscular, oral, nasal, and pulmonary administration.

As used herein, the term “antibody” generally refers to immunoglobulin molecules comprising four polypeptide chains, two heavy (H) chains and two light (L) chains inter-connected by disulfide bonds, as well as multimers thereof (e.g., IgM); however, immunoglobulin molecules consisting of only heavy chains (i.e., lacking light chains) are also encompassed within the definition of the term “antibody.” Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementary determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

As used herein, the terms “antagonistic antibody” or “antagonist antibody” are used herein equivalently and include an antibody that is capable of inhibiting and/or neutralizing the biological signalling activity of OX40, for example by blocking binding or substantially reducing binding of OX40 to OX40 ligand (OX40L) and thus inhibiting or reducing the signalling pathway triggered by OX40 and/or inhibiting or reducing an OX40-mediated cell response like lymphocyte proliferation, cytokine expression, or lymphocyte survival. Such antibodies may include for example, OX40L antagonistic antibodies, or OX40L antagonist antibodies or OX40 antagonistic antibodies or OX40 antagonist antibodies.

Unless specifically indicated otherwise, the term “antibody,” as used herein, shall be understood to encompass complete antibody molecules as well as antigen-binding fragments thereof. The term “antigen-binding portion” or “antigen-binding fragment” of an antibody (or simply “antibody portion” or “antibody fragment”), as used herein, refers to one or more fragments of an antibody that retain the ability to specifically bind to a target antigen such as human OX40L or an epitope thereof.

As used herein, the term “antibody fragment” refers to a polypeptide that includes at least one immunoglobulin variable domain or immunoglobulin variable domain sequence and which specifically binds a given antigen. An antibody fragment can comprise an antibody or a polypeptide comprising an antigen-binding domain of an antibody. In some embodiments, an antibody fragment can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody fragment” encompasses antigen-binding fragments of antibodies (e.g., single chain antibodies, Fab and sFab fragments, F(ab′)2, Fd fragments, Fv fragments, scFv, and domain antibodies (dAb) fragments (see, e.g. de Wildt et al., Eur J. Immunol., 1996; 26(3):629-39; which is incorporated by reference herein in its entirety)) as well as complete antibodies. An antibody can have the structural features of IgA, IgG, IgE, IgD, IgM (as well as subtypes and combinations thereof).

Antibodies can be from any source, including mouse, rabbit, pig, rat, and primate (human and non-human primate) and primatized antibodies. Antibodies also include minibodies, humanized antibodies, chimeric antibodies, and the like.

As used herein, “antibody variable domain” refers to the portions of the light and heavy chains of antibody molecules that include amino acid sequences of complementarity determining regions (CDRs; i.e., CDR1, CDR2, and CDR3), and framework regions (FRs). VH refers to the variable domain of the heavy chain. VL refers to the variable domain of the Light chain. According to the methods used in this disclosure, the amino acid positions assigned to CDRs and FRs may be defined according to Kabat (Sequences of Proteins of Immunological Interest (National Institutes of Health, Bethesda, Md., 1987 and 1991)) or according to IMGT nomenclature.

As used herein, the term “antibody binding site” refers to a polypeptide or domain that comprises one or more CDRs of an antibody and is capable of binding an antigen. For example, the polypeptide comprises a CDR3 (e.g., HCDR3). For example, the polypeptide comprises CDRs 1 and 2 (e.g., HCDR1 and 2) or CDRs 1-3 of a variable domain of an antibody (e.g., HCDRs1-3). In one example, the antibody binding site is provided by a single variable domain (e.g., a VH or VL domain). In another example, the binding site comprises a VH/VL pair, or two or more of such pairs.

As used herein, “OX40L antagonistic antibody” or “OX40L antagonist antibody” refers to an antibody or antigen-binding fragment thereof that is capable of inhibiting and/or neutralizing the biological signaling activity of OX40L, for example by blocking binding or substantially reducing binding of OX40 to OX40L.

As used herein, a “buffer” refers to a chemical agent that is able to absorb a certain quantity of acid or base without undergoing a strong variation in pH.

As used herein, the term “cell” is meant to refer to a cell that is in vitro, ex vivo, or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “dose” refers to a specified amount or quantity of a medication taken or recommended to be taken at a particular time. As used herein, it is typically expressed in mg of the antibody or fragment thereof. It may alternatively be expressed in terms of mg/kg, accounting for patient bodyweight. A “daily dose” refers to the total dosage amount administered to an individual in a single 24-hour day.

As used herein, the term “dosage” refers to the administering of a specific amount, number, and frequency of doses over a specified period of time. Dosage implies duration. A “dosage regimen” is a treatment plan for administering a drug over a period of time.

As used herein, the terms “improve,” “improving” or “improvement” or grammatical variations thereof used in relation to behaviors refer to the ability to achieve a measurable increase in performance in relation to tasks used to test these behaviors in a subject, including humans or non-human animals.

As used herein, “injection” refers to a means of administration and encompasses for example intravenous (IV) and subcutaneous injections. An IV injection may be referred to as an infusion. It is also used herein to refer to an instance of administration wherein that administration is by injection, for example in the phrase “one or more induction phase injections”. Each injection will involve administration of a dose of antibody or fragment thereof.

As used herein, “injection device” refers to a device that is designed for carrying out injections, an injection including the steps of temporarily fluidically coupling the injection device to a person's tissue, typically the subcutaneous tissue. An injection further includes administering an amount of aqueous, or liquid, drug into the tissue and decoupling or removing the injection device from the tissue. In some embodiments, an injection device can be an intravenous device or IV device, which is a type of injection device used when the target tissue is the blood within the circulatory system, e.g., the blood in a vein. A common, but non-limiting example of an injection device is a needle and syringe.

As used herein, “instructions” refers to a display of written, printed or graphic matter on the immediate container of an article, for example the written material displayed on a vial containing a pharmaceutically active agent, or details on the formulation and use of a product of interest included in a kit containing a formulation of interest. Instructions set forth the method of the treatment as contemplated to be administered or performed.

As used herein, the terms “isolated antibody” or “purified antibody” refer to an antibody that by virtue of its origin or source of derivation has one to four of the following: (1) is not associated with naturally associated components that accompany it in its native state, (2) is free or substantially free of other proteins from the same species, (3) is expressed by a cell from a different species, or (4) does not occur in nature. An isolated antibody is substantially free of other antibodies having different antigenic specificities.

As used herein, the term “formulation” as it relates to an antibody is meant to describe the antibody in combination with a pharmaceutically acceptable excipient comprising at least one buffer, at least one stabilizer, at least one surfactant, at least one chelating agent, and wherein the pH is as defined. As used herein, the term “formulation” may be used interchangeably with the term “composition.”

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example. As used herein, a “humanized” antibody refers to forms of non-human (e.g. murine) antibodies that are chimeric immunoglobulins, immunoglobulin chains, or fragments thereof (such as Fv, Fab, Fab′, F(ab′)2 or other antigen-binding subsequences of antibodies) that contain minimal sequence derived from non-human immunoglobulin. In certain exemplary embodiments, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance.

As used herein, the terms “level” and “levels” can be used interchangeably with the terms “concentration” and “concentrations.”

As use herein, the term “patient” or “subject” or “animal” or “host” refers to mammal.

The subject may be a human, but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

As used herein, the terms “peptide” or “polypeptide” are used interchangeably herein and refer to compounds consisting of from about 2 to about 90 amino acid residues, inclusive, wherein the amino group of one amino acid is linked to the carboxyl group of another amino acid by a peptide bond. A peptide can be, for example, derived or removed from a native protein by enzymatic or chemical cleavage, or can be prepared using conventional peptide synthesis techniques (e.g., solid phase synthesis) or molecular biology techniques (see Sambrook et al., MOLECULAR CLONING: LAB. MANUAL (Cold Spring Harbor Press, Cold Spring Harbor, NY, 1989)). A “peptide” can comprise any suitable L- and/or D-amino acid, for example, common a-amino acids (e.g., alanine, glycine, valine), non-a-amino acids (e.g., P-alanine, 4-aminobutyric acid, 6 aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and means for adding or removing protecting groups are known in the art. See, e.g., Green and Wuts, PROTECTING GROUPS IN ORGANIC SYNTHESIS (John Wiley and Sons, 1991). The functional groups of a peptide can also be derivatized (e.g., alkylated) using art-known methods.

As used herein, the term “pharmaceutical formulation” or “drug formulation” refers to a preparation which is in such form as to permit the biological activity of the active ingredients to be effective. “Pharmaceutical formulation” and the term “drug formulation” refer to a mixture or a structure in which different chemical substances, including the active drug, are combined to form a final medicinal product, such as a sterile product, a solution, a powder, an emulsion, a capsule, a tablet, a granule, a topical preparation, a non-conventional product such as semi-solid or sustained-release preparations, liquid, etc. Pharmaceutical formulation is prepared according to a specific procedure, a “formula.” The drug formed varies by the route of administration. As used herein, the term “formulation” as it relates to an antibody may refer to, for example, the antibody in combination with a pharmaceutically acceptable excipient comprising at least one buffer, at least one stabilizer, at least one surfactant, at least one chelating agent, and wherein the pH is as defined. Alternatively, the term “formulation” as it relates to an antibody may refer to, for example, the antibody in combination with a pharmaceutically acceptable excipient comprising at least one buffer, at least one stabilizer, at least one surfactant, and wherein the pH is as defined.

As used herein, the term “pharmaceutical formulation” is interchangeable with the term “pharmaceutical composition,” which further refers to the active agent in combination with a pharmaceutically acceptable carrier e.g., a carrier commonly used in the pharmaceutical industry. The phrase “pharmaceutically acceptable” is employed herein to refer to those compounds, materials, compositions or formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipients” (vehicles, additives) are those, which can safely be administered to a subject to provide an effective dose of the active ingredient employed. The term “excipient” or “carrier” as used herein refers to an inert substance, which is commonly used as a diluent, vehicle, preservative, binder or stabilizing agent for drugs. As used herein, the term “diluent” refers to a pharmaceutically acceptable (safe and non-toxic for administration to a human) solvent and is useful for the preparation of the aqueous formulations herein. Exemplary diluents include, but are not limited to, sterile water and bacteriostatic water for injection (BWFI).

As used herein, the term “pharmaceutically acceptable carrier” means a pharmaceutically acceptable material, composition, or formulation, or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, the term “pharmaceutically acceptable carrier” also includes any and all coatings, antibacterial and antifungal agents, and absorption delaying agents, and the like that are compatible with the activity of the compound useful within the present disclosure, and are physiologically acceptable to the subject. In certain situations, supplementary active compounds may also be incorporated into a pharmaceutical formulation. The “pharmaceutically acceptable carrier” may further include a pharmaceutically acceptable salt of the compound disclosed herein. Other additional ingredients that may be included in a pharmaceutical formulation are known in the art and described, for example, in Remington's Pharmaceutical Sciences (Genaro, Ed., Mack Publishing Co., 1985, Easton, PA), which is incorporated herein by reference.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are used. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the terms “portion”, “fragment,” “variant”, “derivative” and “analog”, when referring to a polypeptide of the present disclosure include any polypeptide that retains at least some biological activity referred to herein (e.g., antigen binding).

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment). For treatment to be effective a complete cure is not contemplated. The method can in certain aspects include cure as well.

As used herein, the term “mg/kg” refers to the dose of a substance administered to an individual in milligrams per kilogram of body weight of the individual.

As used herein, “packaging” refers to how the components are organized and/or restrained into a unit fit for distribution and/or use. Packaging can include, e.g., boxes, bags, syringes, ampoules, vials, tubes, clamshell packaging, barriers and/or containers to maintain sterility, labeling, etc.

As used herein, the term “recombinant antibody” is intended to include all antibodies that are prepared, expressed, created or isolated by recombinant means, for example antibodies expressed using a recombinant expression vector transfected into a host cell, antibodies isolated from a recombinant, combinatorial human antibody library, antibodies isolated from an animal (e.g., a mouse) that is transgenic for human immunoglobulin genes or antibodies prepared, such recombinant human antibodies can be subjected to in vitro mutagenesis.

As used herein, “compounding” or “sterile compound” involves preparing medication in an environment free from bacteria, viruses, or any other potentially infectious microorganisms.

Sterile compounding is used for preparations that will be administered either through an IV, injection, or directly into the eyes.

As used herein, the phrases “systemic administration,” “administered systemically,” “peripheral administration” and “administered peripherally” as used herein mean the administration of a compound, drug or other material other than directly into a target tissue (e.g., the nervous system), such that it enters the animal's system and, thus, is subject to metabolism and other like processes, for example, subcutaneous administration.

As used herein, the term “sequence identity,” “percent identity,” “percent homology,” or, for example, comprising a “sequence 80% identical to,” refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Calculations of sequence similarity or sequence identity between sequences (the terms are used interchangeably herein) can be performed as follows. To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, e.g. at least 40%, at least 50%, at least 60%, or at least 70%, at least 80%, at least 90%, 100% of the length of the reference sequence.

The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch, (1970, J. Mol. Biol. 48: 444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another exemplary embodiment, the percent identity between two nucleotide sequences is determined using the GAP program in the GCG software package, using an NWSgapdna.CMP matrix and a gap weight of 40, 50, 60, 70, or 80 and a length weight of 1, 2, 3, 4, 5, or 6. Another exemplary set of parameters includes a Blossum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller (1989, Cabios, 4: 11-17) which has been incorporated into the ALIGN program (version 2.0), using a PAM120 weight residue table, a gap length penalty of 12 and a gap penalty of 4.

For instance, the peptide sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to nucleic acid molecules of the disclosure. BLAST protein searches can be performed with the XBLAST program, score=50, wordlength=3 to obtain amino acid sequences homologous to protein molecules of the disclosure. To obtain gapped alignments for comparison purposes, Gapped BLAST can be utilized as described in Altschul et al. (Nucleic Acids Res. 25:3389-3402, 1997). When utilizing BLAST and Gapped BLAST programs, the default parameters of the respective programs (e.g., XBLAST and NBLAST) can be used.

Definitions of common terms in cell biology and molecular biology can be found in “The Merck Manual of Diagnosis and Therapy,” 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, standard procedures were used, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et al., ed., John Wiley and Sons, Inc.), Current Protocols in Cell Biology (CPCB) (Juan S. Bonifacino et al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various embodiments of the disclosure.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the claims set forth below.

### Pharmaceutical Formulations

As used herein, the expression “pharmaceutical formulation” means a combination of at least one active ingredient (e.g., a small molecule, macromolecule, compound, etc.) that can exert a biological effect in a human or non-human animal, and at least one inactive ingredient which, when combined with the active ingredient or one or more additional inactive ingredients, is suitable for therapeutic administration to a human or non-human animal. The term “formulation,” as used herein, means “pharmaceutical formulation” unless specifically indicated otherwise. “Pharmaceutically acceptable” excipients (vehicles, additives) are those which can reasonably be administered to a subject mammal to provide an effective dose of the active ingredient employed.

In various embodiments, pharmaceutical formulations according to the present disclosure are in aqueous form comprising an antibody or antigen binding fragment thereof that specifically binds to OX40L. More specifically, the present disclosure provides pharmaceutical formulations that comprise (a) a monoclonal antibody or antigen binding fragment thereof that specifically binds to OX40 ligand (OX40L mAb); (b) a stabilizer; (c) a surfactant; and (d) a buffer comprising histidine or a histidine salt. The pH of the aqueous pharmaceutical formulation may range from about 5.5 to about 6.3, e.g. from about 5.8 to about 6.2, or the pH is about 6.0.

The pharmaceutical formulations of the antibodies provided herein can be prepared by mixing the antibody, having the desired degree of purity, with one or more optional pharmaceutically acceptable carriers or excipients (Remington's Pharmaceutical Sciences 16th edition, Osol, A. Ed. (1980)). In exemplary embodiments, pharmaceutical formulations are provided in the form of aqueous solutions.

It has been demonstrated that the aqueous pharmaceutical formulations stably support the antibodies or antigen binding fragments thereof in a concentration as high as about 125 mg/mL. The disclosed aqueous pharmaceutical formulations are suitable for administering to a mammal subject by parenteral administration, including subcutaneous, intravenous, intramuscular, intraperitoneal, or intradermal injection.

Specific exemplary components and formulations included within the present disclosure are described in detail below.

### Antibodies or Fragments Thereof

According to various embodiments, the antibodies or fragments thereof comprised in the formulations disclosed herein may include, but are not limited to, synthetic antibodies, monoclonal antibodies, recombinantly produced antibodies, multispecific antibodies (including bi-specific antibodies), human antibodies, humanized antibodies, chimeric antibodies, intrabodies, single-chain Fvs (scFv) (e.g., including monospecific, bispecific, etc.), camelid antibodies, Fab fragments, F(ab′) fragments, disulfide-linked Fvs (sdFv), anti-idiotypic (anti-Id) antibodies, and epitope-binding fragments of any of the above that are able to bind to OX40L (OX40L) or to OX40.

The term “specifically binds,” or the like, means that an antibody or antigen-binding fragment thereof forms a complex with an antigen such as hOX40L that is relatively stable under physiologic conditions. Specific binding can be characterized by a dissociation constant of at least about 1×10⁻⁶ M or greater. Methods for determining whether two molecules specifically bind are well known in the art and include, for example, equilibrium dialysis, surface plasmon resonance, and the like. An isolated antibody that specifically binds hOX40L may, however, have cross-reactivity to other antigens, such as OX40L molecules from other species (orthologs). In the context of the present disclosure, multispecific (e.g., bispecific) antibodies that bind to human OX40L as well as one or more additional antigens are deemed to “specifically bind” human OX40L. Moreover, an isolated antibody may be substantially free of other cellular material or chemicals.

In particular, antibodies comprised in the formulations disclosed herein include immunoglobulin molecules and immunologically active portions of immunoglobulin molecules, i.e., molecules that contain an antigen binding site that specifically binds to a hOX40L antigen. The immunoglobulin molecules provided herein can be of any type (e.g., IgG, IgE, IgM, IgD, IgA and IgY), class (e.g., IgG1, IgG2, IgG3, IgG4, IgA1 and IgA2) or subclass of immunoglobulin molecule. In a specific embodiment, an antibody according to the present disclosure is an IgG antibody, e.g. an IgG1 or IgG4. In certain embodiments, the antibodies of the disclosure comprise a human gamma 4 constant region. In one embodiment, the antibody is human IgG4 and where its heavy chain constant region IgG4-PE comprises Leu235Glu and Ser228Pro Fc mutations.

Variants and derivatives of antibodies include antibody fragments that retain the ability to specifically bind to an epitope. Exemplary fragments include Fab fragments; Fab′ (an antibody fragment containing a single anti-binding domain comprising an Fab and an additional portion of the heavy chain through the hinge region); F(ab′)2 (two Fab′ molecules joined by interchain disulfide bonds in the hinge regions of the heavy chains; the Fab′ molecules may be directed toward the same or different epitopes); a bispecific Fab (a Fab molecule having two antigen binding domains, each of which may be directed to a different epitope); a single chain Fab chain comprising a variable region, also known as a scFv; a disulfide-linked Fv, or dsFv; a camelid VH (the variable, antigen-binding determinative region of a single heavy chain of an antibody in which some amino acids at the VH interface are those found in the heavy chain of naturally occurring camel antibodies); a bispecific scFv (a scFv or a dsFv molecule having two antigen-binding domains, each of which may be directed to a different epitope); a diabody (a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); and a triabody (a trimerized scFv, formed in a manner similar to a diabody, but in which three antigen-binding domains are created in a single complex; the three antigen binding domains may be directed towards the same or different epitopes). Derivatives of antibodies also include one or more CDR sequences of an antibody combining site. The CDR sequences may be linked together on a scaffold when two or more CDR sequences are present. In certain embodiments, the antibody comprises a single-chain Fv (“scFv”). scFvs are antibody fragments comprising the VH and VL domains of an antibody, wherein these domains are present in a single polypeptide chain. Generally, the scFv polypeptide further comprises a polypeptide linker between the VH and VL domains which enables the scFv to form the desired structure for antigen binding. For a review of scFvs see Pluckthun in The Pharmacology of Monoclonal Antibodies, vol. 113, Rosenburg and Moore eds. Springer-Verlag, New York, pp. 269-315 (1994).

In some embodiments, the antibodies comprised in the formulations disclosed herein may be from any animal origin including birds and mammals (e.g., human, murine, donkey, sheep, rabbit, goat, guinea pig, camel, horse, or chicken). In certain embodiments, the antibodies may be human or humanized monoclonal antibodies. As used herein, “human” antibodies include antibodies having the amino acid sequence of a human immunoglobulin and include antibodies isolated from human immunoglobulin libraries or from mice that express antibodies from human genes.

In some embodiments, the antibodies are fully human antibodies, such as fully human antibodies that specifically bind a hOX40L polypeptide, a hOX40L polypeptide fragment, or a hOX40L epitope. Such fully human antibodies would be advantageous over fully mouse (or other full or partial non-human species antibodies), humanized antibodies, or chimeric antibodies to minimize the development of unwanted or unneeded side effects, such as immune responses directed toward non-fully human antibodies (e.g., anti-hOX40L antibodies derived from other species) when administered to the subject.

In some embodiments, the antibodies may be monospecific, bispecific, trispecific or of greater multispecificity. Multispecific antibodies may be specific for different epitopes of a hOX40L polypeptide or may be specific for both a hOX40L polypeptide as well as for a heterologous epitope, such as a heterologous polypeptide or solid support material. In exemplary embodiments, the antibodies provided herein are monospecific for a given epitope of a hOX40L polypeptide and do not specifically bind to other epitopes.

In certain embodiments, an isolated antibody provided herein that specifically binds to a hOX40L epitope wherein the binding to the hOX40L epitope by the antibody is competitively blocked (e.g., in a dose-dependent manner) by an antibody or fragment of the disclosure. The antibody may or may not be a fully human antibody. In exemplary embodiments, the antibody is a fully human monoclonal anti-hOX40L antibody. In particularly exemplary embodiments, the antibody is a fully human, monoclonal, antagonist anti-hOX40L antibody. Exemplary competitive blocking tests that can be used are provided in the Examples herein.

In some embodiments, the antibody or fragment of the disclosure competes (e.g., in a dose-dependent manner) with OX40 Receptor (or a fusion protein thereof) for binding to cell surface-expressed hOX40L. In other embodiments, the antibody or fragment of the disclosure competes (e.g., in a dose-dependent manner) with OX40 receptor (or a fusion protein thereof) for binding to soluble hOX40L. In one embodiment, the antibody or fragment partially or completely inhibits binding of hOX40 to cell surface-expressed OX40L, such as hOX40L. In another embodiment, the antibody partially or completely inhibits binding of hOX40 to soluble hOX40L.

In some embodiments, the antibody or antigen binding fragment of the antibody are fully human, monoclonal antibodies, such as fully human, monoclonal antagonist antibodies, that specifically bind to hOX40L.

In some embodiments, the antibody or fragment binds to a hOX40L epitope that is a three-dimensional surface feature of a hOX40L polypeptide (e.g., in a trimeric form of a hOX40L polypeptide). A region of a hOX40L polypeptide contributing to an epitope may be contiguous amino acids of the polypeptide or the epitope may come together from two or more non-contiguous regions of the polypeptide A hOX40L epitope may be present in (a) the trimeric form (“a trimeric hOX40L epitope”) of hOX40L, (b) the monomeric form Ca monomeric hOX40L epitope”) of hOX40L, (c) both the trimeric and monomeric form of hOX40L, (d) the trimeric form, but not the monomeric form of hOX40L, or (e) the monomeric form, but not the trimeric form of hOX40L.

For example, in some embodiments, the epitope is only present or available for binding in the trimeric (native) form, but is not present or available for binding in the monomeric (denatured) form by an anti-hOX40L antibody. In other embodiments, the hOX40L epitope is linear feature of the hOX40L polypeptide (e.g., in a trimeric form or monomeric form of the hOX40L polypeptide). Antibodies provided herein may specifically bind to (a) an epitope of the monomeric form of hOX40L, (b) an epitope of the trimeric form of hOX40L, (c) an epitope of the monomeric but not the trimeric form of hOX40L, (d) an epitope of the trimeric but not the monomeric form of hOX40L, or (e) both the monomeric form and the trimeric form of hOX40L. In exemplary embodiments, the antibodies provided herein specifically bind to an epitope of the trimeric form of hOX40L but do not specifically bind to an epitope the monomeric form of hOX40L.

In some embodiments, the antibodies specifically bind to a hOX40L epitope, the antibodies comprising derivatives of the VH domains, VH CDRs, VL domains, and VL CDRs described herein that specifically bind to a hOX40L antigen. Some embodiments also provide antibodies comprising derivatives of antibodies specifically binding to a hOX40L epitope. Standard techniques known to those of skill in the art can be used to introduce mutations in the nucleotide sequence encoding a molecule, including, for example, site-directed mutagenesis and PCR-mediated mutagenesis which results in amino acid substitutions. In certain exemplary embodiments, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In another embodiment, the derivatives have conservative amino acid substitutions. In certain exemplary embodiments, the derivatives have conservative amino acid substitutions are made at one or more predicted non-essential amino acid residues. Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed, and the activity of the protein can be determined.

In another embodiment, an antibody that specifically binds to a hOX40L epitope comprises a variable domain amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to a variable domain amino acid sequence of the sequence listing.

In specific embodiments, the antibody is a fully human anti-human antibody, such as a fully human monoclonal antibody. Fully human antibodies may be produced by any method known in the art. Exemplary methods include immunization with a hOX40L antigen (any hOX40L polypeptide capable of eliciting an immune response, and optionally conjugated to a carrier) of transgenic animals (e.g., mice) that are capable of producing a repertoire of human antibodies in the absence of endogenous immunoglobulin production; see, e.g., Jakobovits et al., (1993) Proc. Natl. Acad. Sci., 90:2551; Jakobovits et al., (1993) Nature, 362:255 258 (1993); Bruggermann et al., (1993) Year in Immunol., 7:33. Other methods of producing fully human anti-hOX40L antibodies can be found in the Examples provided herein.

Alternatively, fully human antibodies may be generated through the in vitro screening of phage display antibody libraries; see e.g., Hoogenboom et al., J. Mol. Biol., 227:381 (1991); Marks et al., J. Mol. Biol., 222:581 (1991), incorporated herein by reference. Various antibody-containing phage display libraries have been described and may be readily prepared by one skilled in the art. Libraries may contain a diversity of human antibody sequences, such as human Fab, Fv, and scFv fragments, which may be screened against an appropriate target.

The disclosure encompasses the antibody or fragment conjugated to a therapeutic moiety (“immunoconjugate”), such as a cytotoxin, a chemotherapeutic drug, an immunosuppressant or a radioisotope. Cytotoxin agents include any agent that is detrimental to cells. Examples of suitable cytotoxin agents and chemotherapeutic agents for forming immunoconjugates are known in the art, see for example, WO 05/103081, which is incorporated by reference herein in its entirety.

In some embodiments, the antibodies and fragments may include antibodies and fragments that are chemically modified, i.e., by the covalent attachment of any type of molecule to the antibody. For example, but not by way of limitation, the antibody derivatives include antibodies that have been chemically modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formulation, metabolic synthesis of tunicamycin, etc. Additionally, the antibody may contain one or more non-classical amino acids.

In some embodiments, the antibodies that specifically bind to a hOX40L antigen comprise a framework region known to those of skill in the art (e.g., a human or non-human fragment). The framework region may, for example, be naturally occurring or consensus framework regions. In certain embodiments, the framework region of an antibody of the disclosure is human (see, e.g., Chothia et al., 1998, J. Mol. Biol. 278:457-479 for a listing of human framework regions, which is incorporated by reference herein in its entirety). See also Kabat et al. (1991) Sequences of Proteins of Immunological Interest (U.S. Department of Health and Human Services, Washington, D.C.) 5th ed.

In some embodiments, the antibodies that specifically bind to a hOX40L antigen comprise the amino acid sequence of one or more of the CDRs in the sequence listing (i.e. SEQ ID No:4, SEQ ID No:10, SEQ ID No:36, SEQ ID No:42, SEQ ID No:68, SEQ ID No:74, SEQ q ID No:96, or SEQ ID No:102; in particular, SEQ ID No:36 or SEQ ID No:42 for HCDR1; SEQ ID No:6, SEQ ID No:12, SEQ ID No:38, SEQ ID No:44, SEQ ID No:70, SEQ ID No:76, SEQ ID No:98, or SEQ ID No:104; in particular SEQ ID No:38 or SEQ ID No:44 for HCDR2; SEQ ID No:8, SEQ ID No:14, SEQ ID No:40, SEQ ID No:46, SEQ ID No:72, SEQ ID No:78, SEQ ID No:100, or SEQ ID No:106; in particular SEQ ID No:40 or SEQ ID No:46 for HCDR3; SEQ ID No:18, SEQ ID No:24, SEQ ID No:50, SEQ ID No:56, SEQ ID No:82, SEQ ID No:88, SEQ ID No:110, or SEQ ID No:116; in particular SEQ ID No:50 or SEQ ID No:56 for LCDR1; SEQ ID No:20, SEQ ID No:26, SEQ ID No:52, SEQ ID No:58, SEQ ID No:84, SEQ ID No:90, SEQ ID No:112, or SEQ ID No:118; in particular SEQ ID No:52 or SEQ ID No:58 for LCDR2; and SEQ ID No:22, SEQ ID No:28, SEQ ID No:54, SEQ ID No:60, SEQ ID No:86, SEQ ID No:92, SEQ ID No:114, or SEQ ID No:120, in particular SEQ ID No:54 or SEQ ID No:60 for LCDR3) and human framework regions with one or more amino acid substitutions at one, two, three or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In certain embodiments, antibodies that specifically bind to a hOX40L antigen comprising the human framework regions with one or more amino acid substitutions at one, two, three or more of the above-identified residues are antagonistic hOX40L antibodies.

In some embodiments, the antibodies that specifically bind to a hOX40L antigen comprise the amino acid sequence of the VH domain and/or VL domain in the sequence listing (i.e. SEQ ID No:2, SEQ ID No:34, SEQ ID No:66 or SEQ ID No:94, in particular SEQ ID No:34 for VH domains; SEQ ID No:16, SEQ ID No:48, SEQ ID No:80, or SEQ ID No:108, in particular SEQ ID No:48 for VL domains) but having mutations (e.g., one or more amino acid substitutions) in the framework regions. In certain embodiments, antibodies that specifically bind to a hOX40L antigen comprise the amino acid sequence of the VH domain and/or VL domain or an antigen-binding fragment thereof with one or more amino acid residue substitutions in the framework regions of the VH and/or VL domains.

In some embodiments, the antibodies that specifically bind to a hOX40L antigen comprise a heavy chain complementarity determining region (HCDR) 1 of SEQ ID NO: 42, an HCDR2 of SEQ ID NO:44, and an HCDR3 of SEQ ID NO:46; and a light chain complementarity determining region (LCDR) 1 of SEQ ID NO:56, an LCDR2 of SEQ ID NO:58, and an LCDR3 of SEQ ID NO:60.

In some embodiments, the antibodies that specifically bind to a hOX40L epitope comprise the amino acid sequence of the VH domain and/or VL domain in the sequence listing (i.e. SEQ ID No:2, SEQ ID No:34, SEQ ID No:66 or SEQ ID No:94, in particular SEQ ID No:34 for VH domains; SEQ ID No:16, SEQ ID No:48, SEQ ID No:80, or SEQ ID No:108, in particular SEQ ID No:48 for VL domains) but having mutations (e.g., one or more amino acid substitutions) in the framework regions. In certain embodiments, antibodies that specifically bind to a hOX40L antigen comprise the amino acid sequence of the VH domain and/or VL domain or an antigen-binding fragment thereof of an antibody disclosed in the Examples with one or more amino acid residue substitutions in the framework regions of the VH and/or VL domains.

In another embodiment, the antibody that specifically binds to a hOX40L epitope comprises a variable domain amino acid sequence that is at least 80%, at least 85%, at least 90%, at least 95%, or at least 99% identical to SEQ ID No:2, SEQ ID No:34, SEQ ID No:66 or SEQ ID No:94, in particular SEQ ID No:34 for VH domains; and at least 80%, at least 85%, at least 90%, at least 95%, or at least 99% identical to SEQ ID No:16, SEQ ID No:48, SEQ ID No:80, or SEQ ID No:108, in particular SEQ ID No:48 for VL domains.

In one embodiment, the antibody or a fragment thereof that specifically binds to an hOX40L antigen comprises the amino acid sequence of the VH domain and/or VL domain, where the VH comprises the amino acid sequence set forth in SEQ ID NO: 34 and the VL domain comprises the amino acid set forth in SEQ ID NO:48.

In one embodiment, the antibody or a fragment thereof that specifically binds to an hOX40L antigen is a fully human antibody which comprises a heavy chain and a light chain, where the heavy chain comprises a variable (VH) domain having at least 80%, at least 85%, at least 90%, at least 95%, or at least 99% identity to the amino acid sequence of SEQ ID NO: 34 and the light chain comprises a variable (VH) domain having at least 80%, at least 85%, at least 90%, at least 95%, or at least 99% identity to the amino acid sequence of SEQ ID NO: 48, as disclosed in U.S. Pat. No. 10,669,342 (WO2015/132580).

In one embodiment, the antibody is human IgG4 and where its heavy chain constant region IgG4-PE comprises Leu235Glu and Ser228Pro Fc mutations. In another embodiment, the heavy chain constant region is IgG4-PE.

In one embodiment, the antibody comprises a heavy chain having an amino acid sequence of SEQ ID NO:62 and a light chain having an amino acid sequence of SEQ ID NO:64.

In one embodiment, the antibody is amlitelimab or variants thereof.

In some embodiments, fusion proteins comprising an antibody provided herein that specifically binds to a hOX40L antigen and a heterologous polypeptide may be used. In some embodiments, the heterologous polypeptide to which the antibody is fused is useful for targeting the antibody to cells having cell surface-expressed hOX40L.

Optionally, the antibody or fragment specifically binds hOX40L with an affinity (apparent affinity, Kd) of less than 1 mM, 1000 nM to 100 nM, 100 nM to 10 nM, 10 nM to 1 nM, 1000 pM to 500 pM, 500 pM to 200 pM, less than 200 pM, 200 pM to 150 pM, 200 pM to 100 pM, 100 pM to 10 pM, 10 pM to 1 pM, e.g., in the range of 1 mM to 1 pM (e.g., 1 mM to 100 pM; 10 nM to 100 pM; 1 nM to 10 pM; or 100 pM to 1 pM) as determined by SPR, e.g., under SPR conditions disclosed herein).

In some embodiments, the antibody in the pharmaceutical formulations and/or formulated antibodies provided herein comprise one or more charge variants (also called species, or isoforms). Charge variants are typically described as main charge variant (or species), acidic variant (or species), and basic variant (or species). The main charge variant refers to the most prevalent charge variant in a given batch of antibody. Acidic variant has lower isoelectric point (pI) compared to the main charge variant, and basic variant has higher pH compared to the main charge variant. Charge variant can result from post-translational modifications to the amino acid sequence of the HC and/or LC. For example, increases in deamidation, glycation and sialylation, may cause a decrease in the pI of an antibody.

Charge variant of the antibodies provided herein can be separated and quantified using methods known in the art for separating polypeptides based on charge. For example, in some embodiments, a weak cation exchange column can be used in a high-performance liquid chromatography (HPLC) system with a phosphate/sodium chloride gradient buffer. In this system, following loading of the antibody onto the column, the acidic variant of the antibody would elute first, followed by the main charge variant and then the basic variant of the antibody. In another embodiment, capillary isoelectric focusing (cIEF) can be used. Capillary isoelectric focusing is a method that separates proteins by their isoelectric point (pI) values. In cIEF, the antibody sample migrates to its isoelectric points on a pH gradient within the capillary, thereby resolving the different charge variants along the length of the capillary. To identify and characterize the cIEF variants, charge variants can be isolated by strong cation exchange chromatography (SCX) and analyzed by cIEF. An image of the resolved charge variants in the capillary is taken using a charge coupled device camera using whole column detection and measuring absorbance at 280 nm. The charge variant distribution of the test sample is compared to a reference standard using cIEF to identify the charge variant of the antibody. In some embodiments, cIEF is used as a quality control measure to confirm the identity of the antibody during commercial production, and by determining the charge variant distribution of the antibody in comparison to a reference standard, e.g., a test antibody having a charge variant distribution pattern within a predefined distribution pattern or compared to the distribution pattern of a reference standard.

The amount of antibody, or antigen-binding fragment thereof, contained within the pharmaceutical formulations of the present disclosure may vary depending on the specific properties desired of the formulations, as well as the particular circumstances and purposes for which the formulations are intended to be used. In certain embodiments, the pharmaceutical formulations are aqueous formulations that contain the antibody in a concentration ranging from about 1 mg/mL to about 150 mg/ml, including all subranges therebetween, such as from about 20 mg/mL to about 140 mg/mL, e.g., 28±4.2 mg/mL to 138 mg/mL±20.7 mg/mL, or from 31±4.65 mg/mL to 125 mg/mL±18.75 mg/mL. In some embodiments, the antibody or antigen binding fragment thereof is present in a concentration of 28±4.2 mg/mL, 31 mg/mL±4.65 mg/mL, 125 mg/mL±18.75 mg/mL, or 138 mg/mL±20.7 mg/mL. In one embodiment, the antibody or antigen binding fragment thereof is present in a concentration of 28 mg/mL, 31 mg/mL, 125 mg/mL, or 138 mg/mL.

In certain embodiments, the pharmaceutical formulations are aqueous formulations that contain the antibody in a concentration ranging from about 75 mg/mL to about 138 mg/mL, such as 75 (±10%) mg/mL to 138 (±10%) mg/mL, from 85 (±10%) mg/mL to 138 (±10%) mg/mL, from 95 (±10%) mg/mL to 138 (±10%) mg/mL, from 105 (±10%) mg/mL to 138 (±10%) mg/mL, from 115 (±10%) mg/mL to 138 (±10%) mg/mL, from 125 (±10%) mg/mL to 138 (±10%) mg/mL, from 75 (±10%) mg/mL to 128 (±10%) mg/mL, from 75 (±10%) mg/mL to 118 (±10%) mg/mL, from 75 (±10%) mg/mL to 108 (±10%) mg/mL, from 75 (±10%) mg/mL to 98 (±10%) mg/mL, or from 75 (±10%) mg/mL to 88 (±10%) mg/mL. For example, the formulations of the present disclosure may comprise 75 (±10%) mg/mL, 85 (±10%) mg/mL, 95 (±10%) mg/mL, 105 (±10%) mg/mL, 115 (±10%) mg/mL, 125 (±10%) mg/mL, or 135 (±10%) mg/mL of an antibody or antigen-binding fragment thereof, that specifically binds to OX40L, such as hOX40L.

In one embodiment, the formulations of the present disclosure comprise about 125 mg/mL, such as 125 (±10%) mg/mL, of an antibody or antigen-binding fragment thereof that specifically binds to hOX40L. In one embodiment, the formulations of the present disclosure comprise about 125 mg/mL of amlitelimab or variants thereof. In one embodiment, the formulations of the present disclosure comprise about 75 mg/mL of amlitelimab or variants thereof.

### Excipients and pH

In various embodiments, the pharmaceutical formulations of the present disclosure further comprise one or more excipients such as a stabilizer, a surfactant, and a buffer comprising histidine or a histidine salt, which are non-therapeutic agents added to the formulation to provide a desired consistency, viscosity, or stabilizing effect to the formulations comprising an anti-hOX40L antibody described herein. In some embodiments, the pharmaceutical formulations provided herein comprise a formulated antibody comprising an antibody and one or more of the following excipients: sucrose, L-histidine, and polysorbate 80 (PS80).

The amount of each excipient may also vary depending on the specific properties desired of the formulations, as well as the particular circumstances and purposes for which the formulations are intended to be used. In some situations, concentration of each excipient may be chosen such that the pharmaceutical formulation can be diluted for administration by infusion.

### Surfactant

In certain embodiments, the surfactants that can be included in the formulations are non-ionic surfactants selected from polysorbates and/or polxamers. In certain embodiment, a surfactant included in the pharmaceutical formulations is polysorbate 80 (PS80).

The amount of the surfactant contained within the pharmaceutical formulations of the present disclosure may vary depending on the specific properties desired of the formulations, as well as the particular circumstances and purposes for which the formulations are intended to be used. In some embodiments, the surfactant is in an amount that stabilizes the antibody under conditions of rough handling or agitation, such as, e.g., vortexing. In some embodiments, what is meant by “stabilizes” is the prevention of the formation of more than 3% aggregated antibody of the total amount of antibody (on a molar basis) over the course of rough handling or agitation, such as, e.g., vortexing. In some embodiments, rough handling is vortexing a solution containing the antibody and the organic cosolvent for about 60 minutes or about 120 minutes.

In some embodiments, PS80 or PS20 is present in the pharmaceutical formulation at a concentration of about 0.005 (w/v) to about 0.1% (w/v), such as from 0.005% (w/v)±0.0015% to 0.1%±0.03% (w/v). For example, PS80 or PS20 may be present at a concentration of about 0.0085% (w/v), about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), or about 0.1% (w/v). In certain exemplary embodiments, the PS80 is present at a concentration of 0.04% (w/v)±0.006% (w/v) in the formulation. In certain exemplary embodiments, the PS80 is present at a concentration of 0.04% (w/v) in the formulation. In certain exemplary embodiments, the PS80 is present at a concentration of 0.06% (w/v)±0.009% (w/v) in the formulation. In certain exemplary embodiments, the PS80 is present at a concentration of 0.06% (w/v) in the formulation. In some embodiments, the PS80 is present at a concentration of 0.04% (w/v)±0.006% (w/v) in the formulation. In some embodiments, the PS80 is present at a concentration of 0.04% (w/v) in the formulation.

### Stabilizer

In some embodiments, the stabilizer in the pharmaceutical formulations of the present disclosure comprises mannitol and/or sucrose. In some embodiments, the stabilizer in the pharmaceutical formulations of the present disclosure is mannitol and/or sucrose. In certain embodiments, the stabilizer is sucrose. In some embodiments, sucrose is present within the pharmaceutical formulation in a concentration of about 200 mM to about 250 mM, such as 220 mM±33 mM to 250 mM±37.5 mM. In some embodiments, the pharmaceutical formulation comprises 220 mM sucrose.

In some embodiments, sucrose is present at a concentration that stabilizes the antibody under certain conditions. In some embodiments, such conditions may include thermal stress.

### Buffer or Buffer System

The pharmaceutical formulations of the present disclosure may also comprise a buffer or buffer system, which serves to maintain a stable pH and to help stabilize the antibody. The term “buffer” as used herein denotes a pharmaceutically acceptable buffer which maintains a stable pH or resists changes in pH of the solution. In exemplary embodiments, the buffer comprises histidine. In the context of this disclosure, “histidine buffer” or “buffer comprising histidine” is a buffer comprising the amino acid histidine. Examples of histidine buffers include histidine chloride, histidine acetate, histidine phosphate, and histidine sulfate. In an exemplary embodiment, the histidine buffer is prepared by dissolving L-histidine and L-histidine hydrochloride (e.g. as monohydrate) in a defined amount and ratio. In one embodiment, the histidine buffer is prepared by titrating L-histidine (free base, solid) with diluted hydrochloric acid. The term “histidine” is used interchangeably with “histidine buffer” throughout this disclosure.

In some embodiments, L-histidine or histidine hydrochloride is present. In some embodiments, the concentration of the L-histidine or histidine hydrochloride is about 10 mM, such as 10 mM±3 mM. In some embodiments, the concentration of the L-histidine or histidine hydrochloride is about 20 mM, such as 20 mM±3 mM.

### Chelator

In some embodiments, the pharmaceutical formulations of the present disclosure may comprise one or more chelators, e.g., a metal chelator. Metal chelators can protect the protein's chemical stability under metal exposure.

In certain embodiments, the pharmaceutical formulation further comprises a chelator selected from the group consisting of ethylenediaminetetraacetic acid (EDTA), diethylenetriamene pentaacetate (DTPA) and salts thereof, and any combinations thereof. In certain embodiments, the chelator is chosen from EDTA and/or DTPA.

In certain embodiments, the concentration of a chelator such as EDTA and DTPA is about 10 μM, such as 10 μM±1.5 μM.

### Additional Ingredients

In some embodiments, the pharmaceutical formulations of the present disclosure may comprise one or more additional excipients.

Yet in some embodiments, the pharmaceutical formulations do not require any additional excipients such as arginine that serve to maintain a reduced viscosity or to lower the viscosity of formulations disclosed here.

In some embodiments, the formulation does not comprise sodium chloride.

In some embodiments, a pharmaceutical formulation according to the present disclosure consists of, or consist essentially of an antibody disclosed herein, sucrose, polysorbate 80, and L-histidine or histidine hydrochloride, and EDTA, each ingredient being present in an amount described herein.

In some embodiments, a pharmaceutical formulation according to the present disclosure consists of, or consist essentially of an antibody disclosed herein, sucrose, polysorbate 80, and L-histidine or histidine hydrochloride, each ingredient being present in an amount described herein.

pH

pH of the pharmaceutical formulation is optimized to maintain stability of the antibody for example when the pharmaceutical formulation is in aqueous form. In some embodiments, the pH of the formulation has a pH of about 5.5 to about 6.3, such as 5.5±0.2 to 6.3±0.2. In some embodiments, the formulation has a pH of about 5.8 to 6.2. For example, the formulations of the present disclosure may have a pH of about 5.5, about 5.6, about 5.7, about 5.8, about 5.9; about 6.0, about 6.1, about 6.2, about 6.3, about 6.4, or about 6.5. In certain exemplary embodiments, the pH of the formulation is about 6.0±0.2. In one embodiment, the pH of the formulation is about 6.0 or 6.2.

### Stability and Other Attributes

It has been demonstrated that the excipients and the amounts thereof described herein provide desired consistency, viscosity, or stabilizing effects to the pharmaceutical formulation comprising an anti-hOX40L antibody or antigen binding fragment thereof in a concentration from about 75 mg/mL to about 138 mg/mL, such as from 75 mg/mL±11.25 mg/mL to 125 mg/mL±18.75 mg/mL or up to 138 mg/mL±20.7 mg/mL. The aqueous pharmaceutical formulations in various embodiments are typically free of particles or substantially free of particles.

In various embodiments, the pharmaceutical formulations of the present disclosure typically exhibit high levels of stability. The term “stable,”, as used herein in reference to the pharmaceutical formulations, means that the antibodies within the pharmaceutical formulations retain an acceptable degree of chemical structure or biological function after storage under defined conditions. A formulation may be stable even though the antibody contained therein does not maintain 100% of its chemical structure or biological function after storage for a defined amount of time. Under certain circumstances, maintenance of about 90%, about 95%, about 96%, about 97%, about 98% or about 99% of an antibody's structure or function after storage for a defined amount of time may be regarded as “stable.”

Stability can be measured, inter alia, by determining the percentage of native antibody that remains in the formulation after storage for a defined amount of time at a defined temperature. The percentage of native antibody can be determined by, inter alia, size exclusion chromatography (e.g., size exclusion high performance liquid chromatography [SE-HPLC]), such that “native” means non-aggregated and non-degraded. An “acceptable degree of stability,” as that phrase is used herein, means that at least 85% of the native form of the antibody can be detected in the formulation after storage for a defined amount of time at a given temperature.

In certain embodiments, at least about 85%, 90%, 91%, 92%, 93%, 94%, 95%, 96%, 97%, 98%, or 99% of the native form of the antibody can be detected by size exclusion chromatography in the formulation after storage for a defined amount of time. The defined amount of time after which stability is measured can be at least 14 days, at least 28 days, at least 1 month, at least 2 months, at least 3 months, at least 4 months, with a forecast of at least 1 year at about 5° C. The defined temperature at which the pharmaceutical formulation may be stored when assessing stability can be any temperature from about −80° C. to about 40° C., e.g., storage at about −80° C., about −65° C., about −30° C., about −20° C., about 0° C., about 4°−8° C., about 5° C., about 25° C., about 35° C., about 37° C., or about 40° C.

For example, a pharmaceutical formulation may also be deemed stable if after 28 days of storage at 40° C., greater than about 90%, 91%, 92%, 93%, 94%, 95%, 96%, 97% or 98% of native antibody may be detected by SE-HPLC. A pharmaceutical formulation may also be deemed stable if after three months of storage at −20° C., greater than about 96%, 97%, or 98% of native antibody is detected by SE-HPLC. A pharmaceutical formulation may also be deemed stable if after three months of storage at −30° C., greater than about 96%, 97% or 98% of native antibody is detected by SE-HPLC. A pharmaceutical formulation may also be deemed stable if after three months of storage at −80° C., greater than about 96%, 97% or 98% of native antibody is detected by SE-HPLC.

Stability can be measured, inter alia, by determining the percentage of antibody that forms in an aggregate within the formulation after storage for a defined amount of time at a defined temperature, wherein stability is inversely proportional to the percent aggregate that is formed. The percentage of aggregated antibody can be determined by, inter alia, size exclusion chromatography (e.g., size exclusion high performance liquid chromatography [SE-HPLC]). An “acceptable degree of stability,” as that phrase is used herein, means that at most 6% of the antibody is in an aggregated form detected in the formulation after storage for a defined amount of time at a given temperature. In certain embodiments an acceptable degree of stability means that at most about 6%, 5%, 4%, 3%, 2%, 1%, 0.5%, or 0.1% of the antibody can be detected in an aggregate in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The temperature at which the pharmaceutical formulation may be stored when assessing stability can be any temperature from about −80° C. to about 40° C., e.g., storage at about −80° C., about −65° C., about −30° C., about −20° C., about 0° C., about 4°−8° C., about 5° C., about 25° C., about 35° C., about 37° C. or about 40° C.

For example, a pharmaceutical formulation may be deemed stable if after six months of storage at 5° C., less than about 3%, 2%, 1%, 0.5%, or 0.1% of the antibody is detected in an aggregated form. A pharmaceutical formulation may also be deemed stable if after six months of storage at 25° C., less than 4%, 3%, 2%, 1%, 0.5%, or 0.1% of the antibody is detected in an aggregated form. A pharmaceutical formulation may also be deemed stable if after 28 days of storage at 40° C., less than 6%, 5%, 4%, 3%, 2%, 1%, 0.5%, or 0.1% of the antibody is detected in an aggregated form. A pharmaceutical formulation may also be deemed stable if after three months of storage at −20° C., −30° C., about −65° C., or −80° C. less than about 3%, 2%, 1%, 0.5%, or 0.1% of the antibody is detected in an aggregated form.

Stability can also be measured, inter alia, by determining the percentage of antibody that migrates in a more acidic fraction during ion exchange (“acidic form”) than in the main fraction of antibody (“main charge form”), wherein stability is inversely proportional to the fraction of antibody in the acidic form. While not wishing to be bound by theory, deamidation of the antibody may cause the antibody to become more negatively charged and thus more acidic relative to the non-deamidated antibody (see, e.g., Robinson, N., Protein Deamidation, PNAS, Apr. 16, 2002, 99(8):5283-5288). The percentage of “acidified” antibody can be determined by, inter alia, ion exchange chromatography (e.g., cation exchange high performance liquid chromatography [CEX-HPLC]), or by imaged capillary isoelectric focusing (iCIEF). An “acceptable degree of stability,” as that phrase is used herein, means that at most 49% of the antibody is in a more acidic form detected in the formulation after storage for a defined amount of time at a defined temperature. In certain embodiments an acceptable degree of stability means that at most about 45%, 40%, 35%, 30%, 25%, 20%, 15%, 10%, 5%, 4%, 3%, 2%, 1%, 0.5%, or 0.1% of the antibody can be detected in an acidic form in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The temperature at which the pharmaceutical formulation may be stored when assessing stability can be any temperature from about −80° C. to about 40° C., e.g., storage at about −80° C., about −65° C., about −30° C., about −20° C., about 0° C., about 4°−8° C., about 5° C., about 25° C., or about 40° C.

For example, a pharmaceutical formulation may be deemed stable if after three months of storage at −80° C., −30° C., or −20° C. less than about 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after six months of storage at 5° C., less than about 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after six months of storage at 25° C., less than about 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after 28 days of storage at 40° C., less than about 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody can be detected in a more acidic form.

Other methods may be used to assess the stability of the formulations of the present disclosure such as, e.g., differential scanning calorimetry (DSC) to determine thermal stability, controlled agitation to determine mechanical stability, and absorbance at about 350 nm or about 405 nm to determine solution turbidities. For example, a formulation of the present disclosure may be considered stable if, after 6 or more months of storage at about 5° C. to about 25° C., the change in OD₄₀₅ of the formulation is less than about 0.05 (e.g., 0.04, 0.03, 0.02, 0.01, or less) from the OD₄₀₅ of the formulation at time zero.

Measuring the biological activity or binding affinity of the antibody to its target may also be used to assess stability. For example, a formulation of the present disclosure may be regarded as stable if, after storage at e.g., 5° C., 25° C., 40° C., etc. for a defined amount of time (e.g., 1 to 12 months), the anti-hOX40L antibody contained within the formulation binds to hOX40L with an affinity that is at least 90%, 95%, or more of the binding affinity of the antibody prior to said storage. Binding affinity may be determined by e.g., ELISA or plasmon resonance. Biological activity may be determined by a hOX40L activity assay, such as e.g., contacting a cell that expresses hOX40L with the formulation comprising the anti-hOX40L antibody. The binding of the antibody to such a cell may be measured directly, such as e.g., via FACS analysis.

Alternatively, the downstream activity of the hOX40L system may be measured in the presence of the antibody and compared to the activity of the hOX40L system in the absence of antibody. In some embodiments, the hOX40L may be endogenous to the cell.

In one embodiment, at least 91% of the antibody in the aqueous pharmaceutical formulation has native conformation after 28 days at 40° C., measured by size exclusion chromatography (SE-HPLC). In one embodiment, at least 94% of the antibody in the aqueous pharmaceutical formulation has native conformation after 8 weeks at 25° C., measured by size exclusion chromatography. In one embodiment, at least 98% of the antibody in the aqueous pharmaceutical formulation has native conformation after 16 weeks at 5° C., measured by size exclusion chromatography. In one embodiment, at least 98% of the antibody in the aqueous pharmaceutical formulation has native conformation after 16 weeks at −65° C., measured by size exclusion chromatography. When measured by size exclusion chromatography, the aqueous pharmaceutical formulation comprises main peak species, high molecular weight (HMW) species, and low molecular weight (LMW) species. “Native conformation” as used herein refers to the main peak species measured by SE-HPLC.

In one embodiment, at least 55% of the antibody in the formulation disclosed herein is the main charge variant after 28 days at 40° C., measured by imaged capillary isoelectric focusing (iCIEF). In one embodiment, at least 65% of the antibody in the formulation disclosed herein is the main charge variant after 16 weeks at 25° C., measured by imaged capillary isoelectric focusing (iCIEF). In one embodiment, at least 70% of the antibody in the formulation disclosed herein is the main charge variant after 16 weeks at 5° C., measured by imaged capillary isoelectric focusing (iCIEF). In one embodiment, at least 70% of the antibody in the formulation disclosed herein is the main charge variant after 16 weeks at −65° C., measured by imaged capillary isoelectric focusing (iCIEF).

In some embodiments, the aqueous pharmaceutical formulation is stable upon freezing and thawing. In some embodiments, the aqueous pharmaceutical formulation is stable upon storage at −65° C., 5° C., or 25° C. for at least 16 weeks. In some embodiments, the aqueous pharmaceutical formulation is stable upon storage at 40° C. for at least 8 weeks. In some embodiments, the aqueous pharmaceutical formulation is stable upon storage at 5° C. for at least 1 year. In some embodiments, the aqueous pharmaceutical formulation is contained in a suitable container and maintains the stabilities described above.

### Pharmaceutical Unit Dosage Form

Unit dosage forms of formulated antibodies that specifically bind OX40L are provided. In various embodiments, a unit dosage form comprises the aqueous pharmaceutical formulation described herein and is suitable for parenteral administration to a mammalian subject. In some embodiments, the aqueous pharmaceutical formulation is suitable for intravenous, subcutaneous, or intramuscular administration. In some embodiments, the unit dosage form may be contained in a suitable container with a particular volume of an aqueous pharmaceutical formulation disclosed. In some embodiments, the unit dosage form can be in a container (e.g., a vial) such that a prescribed dosage of the antibody can be removed from the container for administration to a patient.

In some embodiments, a unit dosage form contained in a container may include about 2.4 mL of a disclosed formulation. In some embodiments, the extractable volume of the unit dosage form is about 2 mL of a disclosed formulation. In some embodiments, the extractable volume of the unit dosage form is about 1 mL of a disclosed formulation. In some embodiments, a unit dosage form is contained in a suitable container such as a glass vial with a stopper or a cap. In one embodiment, the suitable container is a syringe, where a unit dosage form of an aqueous pharmaceutical formulation disclosed herein is pre-filled within the syringe. For example, the syringe may be a pre-filled syringe, which may comprise a safety system, e.g. one with a needle guard such as the BD UltraSafe™ or BD UltraSafe Plus™ needle guard. In another example, the syringe may be a pre-filled pen or an autoinjector device. In some embodiments, a unit dosage form contained in the container (such as a vial) may comprise about 2.4 ml of a formulation disclosed herein, such that the unit dosage form comprises 180±27 mg to 331±49.7 mg of antibody. In some embodiments, a unit dosage form contained in the container include about 300±45 mg of the antibody disclosed herein. In some embodiments, a unit dosage form contained in the container (such as a pre-filled syringe, a pre-filled syringe with a safety system, a pre-filled pen or autoinjector device) may comprise about 1 mL of a formulation disclosed herein, such that the unit dosage form comprises about 125 mg of antibody. In some embodiments, a unit dosage form contained in the container (such as a pre-filled syringe, a pre-filled syringe with a safety system, a pre-filled pen or autoinjector device) may comprise about 2 mL of a formulation disclosed herein, such that the unit dosage form comprises about 250 mg of antibody.

In some embodiments, a unit dosage form of the formulated antibody is not lyophilized.

In some embodiments, the antibody, pharmaceutical formulation, and/or formulated antibody is in a glass vial fitted with an elastomeric closure.

### Packaged Drug Products and Kits

### Packaged Products

In one aspect, drug products are provided comprising the pharmaceutical formulations of the present disclosure within a container suitable for storage and/or administration of the pharmaceutical formulations described herein. For example, the pharmaceutical formulations may be contained within a sealed and sterilized plastic or glass container having a defined volume such as a vial, ampule, syringe, cartridge, or bottle. Different types of vials can be used to contain the formulations of the present disclosure including, e.g., clear and opaque (e.g., amber) glass or plastic vials. Likewise, any type of syringe can be used to contain or administer the pharmaceutical formulations of the present disclosure.

Specifically, the pharmaceutical formulations of the present disclosure may be contained within “normal tungsten” syringes or “low tungsten” syringes. As will be appreciated by persons of ordinary skill in the art, the process of making glass syringes generally involves the use of a hot tungsten rod which functions to pierce the glass thereby creating a hole from which liquids can be drawn and expelled from the syringe. This process results in the deposition of trace amounts of tungsten on the interior surface of the syringe. Subsequent washing and other processing steps can be used to reduce the amount of tungsten in the syringe. As used herein, the term “normal tungsten” means that the syringe contains greater than or equal to 500 parts per billion (ppb) of tungsten. The term “low tungsten” means that the syringe contains less than 500 ppb of tungsten.

For example, a low tungsten syringe, according to the present disclosure, can contain less than about 490, 480, 470, 460, 450, 440, 430, 420, 410, 390, 350, 300, 250, 200, 150, 100, 90, 80, 70, 60, 50, 40, 30, 20, 10 or fewer ppb of tungsten.

The rubber plungers used in syringes, and the rubber stoppers used to close the openings of vials, may be coated to prevent contamination of the medicinal contents of the syringe or vial, or to preserve their stability. Thus, pharmaceutical formulations of the present disclosure, according to certain embodiments, may be contained within a syringe that comprises a coated plunger, or within a vial that is sealed with a coated rubber stopper. For example, the plunger or stopper may be coated with a fluorocarbon film. Examples of coated stoppers or plungers suitable for use with vials and syringes containing the pharmaceutical formulations of the present disclosure are mentioned in, e.g., U.S. Pat. Nos. 4,997,423; 5,908,686; 6,286,699; 6,645,635; and 7,226,554, the contents of which are incorporated by reference herein in their entireties. Particular exemplary coated rubber stoppers and plungers that can be used in the context of the present disclosure are commercially available under the tradename “FLUROTEC®,” available from West Pharmaceutical Services, Inc. (Lionville, PA). FLUROTEC® is an example of a fluorocarbon coating used to minimize or prevent drug product from adhering to the rubber surfaces.

According to certain embodiments of the present disclosure, the pharmaceutical formulations may be contained within a low tungsten syringe that comprises a fluorocarbon-coated plunger. The pharmaceutical formulations can be administered to a patient by parenteral routes such as injection (e.g., subcutaneous, intravenous, intramuscular, intraperitoneal, etc.) or percutaneous, mucosal, nasal, pulmonary or oral administration. Numerous reusable pens or autoinjector delivery devices can be used to subcutaneously deliver the pharmaceutical formulations of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany). Examples of disposable pen or autoinjector delivery devices having applications in subcutaneous delivery of a pharmaceutical formulation of the present disclosure include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN® (Dey, L. P.), the HUMIRA™ Pen (Abbott Labs, Abbott Park, IL), the MOLLY@ Autoinjector (SHL Medical AG, Zug, Switzerland), the YPSOMATE Autoinjector (Ypsomed AG, Burgdorf, Switzerland), the BD PHYSIOJECT™ Autoinject or (Becton, Dickinson and Company), and the REFLEX Autoinjector (Sanofi-Aventis).

The use of a microinfusor to deliver the pharmaceutical formulations of the present disclosure is also contemplated herein. As used herein, the term “microinfusor” means a subcutaneous delivery device designed to slowly administer large volumes (e.g., up to about 2.5 mL or more) of a therapeutic formulation over a prolonged period of time (e.g., about 10, 15, 20, 25, 30 or more minutes). See, e.g., U.S. Pat. Nos. 6,629,949; 6,659,982; and Meehan et al., J. Controlled Release 46:107-1 16 (1996). Microinfusors are particularly useful for the delivery of large doses of therapeutic proteins contained within high concentration (e.g., about 100, 125, 150, 175, 200 or more mg/mL) or viscous solutions.

Non-limiting exemplary embodiments of suitable container and packaged drug comprising an aqueous formulation disclosed here are further listed below:
A glass vial containing an aqueous formulation comprising an anti-OX40L antibody, or antigen-binding fragment thereof.
A drug delivery device containing an aqueous formulation comprising an anti-OX40L antibody, or antigen-binding fragment thereof.
A prefilled syringe containing an aqueous formulation comprising an anti-OX40L antibody, or antigen-binding fragment thereof. The prefilled syringe may have a safety system, for example, a passive needle guard (e.g. the BD UltraSafe™ or BD UltraSafe Plus™ needle guard). The prefilled syringe may contain an aqueous formulation comprising an anti-OX40L antibody, or antigen-binding fragment thereof in a volume of up to 1 mL or up to 2 mL. In one embodiment, the pre-filled syringe comprises about 125 mg of amlitelimab in a 1 mL solution (125 mg/mL±18.75 mg/mL). In one embodiment, the pre-filled syringe comprises about 250 mg of amlitelimab in a 2 mL solution (125 mg/mL±18.75 mg/mL).
A microinfusor containing an aqueous formulation comprising an anti-OX40L antibody, or antigen-binding fragment thereof.
A pen delivery device containing an aqueous formulation comprising an anti-OX40L antibody, or antigen-binding fragment thereof. The pen delivery device may be a reusable pen delivery device. The pen delivery device may be a disposable pen delivery device. The pen delivery device may contain an aqueous formulation comprising an anti-OX40L antibody, or antigen-binding fragment thereof in a volume of up to 1 mL or up to 2 mL. In one embodiment, the pen delivery device comprises 125 mg of amlitelimab in a 1 mL solution (125 mg/mL±18.75 mg/mL). In one embodiment, the pen delivery device comprises 250 mg of amlitelimab in a 2 mL solution (125 mg/mL±18.75 mg/mL).
An autoinjector delivery device containing an aqueous formulation comprising an anti-OX40L antibody, or antigen-binding fragment thereof. The autoinjector delivery device may contain an aqueous formulation comprising an anti-OX40L antibody, or antigen-binding fragment thereof in a volume of up to 1 mL or up to 2 mL. In one embodiment, the autoinjector delivery device comprises 125 mg of amlitelimab in a 1 mL solution (125 mg/mL±18.75 mg/mL). In one embodiment, the autoinjector delivery device comprises 250 mg of amlitelimab in a 2 mL solution (125 mg/mL±18.75 mg/mL).

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

An example sleeve-triggered autoinjector with manual needle insertion is described in International Publication WO2015/004052. Example audible end-of-dose feedback mechanisms are described in International Publications WO2016/193346 and WO2016/193348. An example needle-safety mechanism after using an autoinjector is described in International Publication WO2016/193352. An example needle sheath remover mechanism for a syringe autoinjector is described in International Publication WO2016/193353. An example support mechanism for supporting an axial position of a syringe is described in International Publication WO2016/193355.

In embodiments further provide sealed containers comprising the aqueous pharmaceutical formulations disclosed herein. In some embodiments, the sealed container may be a vial, a syringe, a microinfusor, a pen delivery device, or an autoinjector containing an aqueous formulation disclosed herein in a volume of up to 1 mL, up to 2 mL or up to 2.25 mL. In some embodiments, a glass injection vial sealed with a rubber stopper or a cap. In one embodiment, the container is a single or multi-chambered syringes.

In one embodiment, provided is a pre-filled syringe containing an aqueous pharmaceutical formulation comprising 125 mg/mL±18.75 mg/mL to 138 mg/mL±20.7 mg/mL of the antibody or antigen binding fragment thereof; 20±3 mM L-histidine or histidine hydrochloride; 220 mM±33 mM sucrose, and 0.04% (w/v)±0.006% (w/v) polysorbate 80, with a pH of about 5.8 to about 6.2. The aqueous pharmaceutical formulation may be in a volume of approximately 1 mL±0.15 ml to 2.4 ml±0.36 ml (e.g. 2 mL). In one embodiment, the syringe is a 1 mL long glass syringe filled with a 27-gauge thin wall needle, a fluorocarbon coated rubber plunger and a rubber needle shield. In one embodiment, the syringe is an OMPI 1 mL long glass syringe fitted with a 27-gauge needle, a FM27 rubber needle shield, and a FLUROTEC® coated 4023/50 rubber plunger.

In one embodiment, provided is a pre-filled syringe containing an aqueous pharmaceutical formulation comprising about 62.5 mg/mL of the antibody or antigen binding fragment thereof (e.g. amlitelimab); 10 mM±1.5 mM or about 10 mM L-histidine or histidine hydrochloride; 220 mM±33 mM or about 220 mM sucrose; about 10 μM EDTA; about 0.06% (w/v) polysorbate 80, and water, with a pH of about 5.8 to about 6.2, preferably about 6. the pre-filled syringe may comprise about 125 mg of the antibody or antigen binding fragment thereof (e.g. amlitelimab) in a 2 mL solution (62.5 mg/mL). Thus, it is appreciated that the invention provides a pre-filled syringe containing a 2 mL volume of an aqueous pharmaceutical formulation comprising about 62.5 mg/mL of the antibody or antigen binding fragment thereof (e.g. amlitelimab); about 10 mM L-histidine or histidine hydrochloride; about 220 mM sucrose; about 10 μM EDTA; about 0.06% (w/v) polysorbate 80, and water, with a pH of about 5.8 to about 6.2, preferably about 6.

In one embodiment, provided is a pre-filled syringe containing an aqueous pharmaceutical formulation comprising 125 mg/mL±18.75 mg/mL or about 125 mg/mL of the antibody or antigen binding fragment thereof (e.g. amlitelimab); 10 mM±1.5 mM or about 10 mM L-histidine or histidine hydrochloride; 220 mM±33 mM or about 220 mM sucrose; about 10 μM EDTA; about 0.06% (w/v) polysorbate 80, and water, with a pH of about 5.8 to about 6.2, preferably about 6. The pre-filled syringe may comprise about 250 mg of the antibody or antigen binding fragment thereof (e.g.amlitelimab) in a 2 mL solution (125 mg/mL). Thus, it is appreciated that the invention provides a pre-filled syringe containing a 2 mL volume of an aqueous pharmaceutical formulation comprising about 125 mg/mL of the antibody or antigen binding fragment thereof (e.g. amlitelimab); about 10 mM L-histidine or histidine hydrochloride; about 220 mM sucrose; about 10 μM EDTA; about 0.06% (w/v) polysorbate 80, and water, with a pH of about 5.8 to about 6.2, preferably about 6.

In one embodiment, provided is a pre-filled syringe, comprising a safety system such as a passive needle guard (e.g. the BD UltraSafe™ or BD UltraSafe Plus™ needle guard), containing an aqueous pharmaceutical formulation comprising 125 mg/mL±18.75 mg/mL or about 125 mg/mL of the antibody or antigen binding fragment thereof (e.g. amlitelimab); 10 mM±1.5 mM or about 10 mM L-histidine or histidine hydrochloride; 220 mM±33 mM or about 220 mM sucrose; about 0.06% (w/v) polysorbate 80; about 10 μM EDTA, and water; with the pH of the aqueous pharmaceutical formulation being about 5.8 to about 6.2. The aqueous pharmaceutical formulation may be in a volume of approximately 1 mL±0.15 ml to 2.4 ml±0.36 ml. In one embodiment, the syringe is a 1 mL long glass syringe filled with a 27-gauge thin wall needle, a fluorocarbon coated rubber plunger and a passive needle guard. In one embodiment, the syringe is an OMPI 1 mL long glass syringe fitted with a 27-gauge needle, a FM27 rubber needle shield, and a FLUROTEC® coated 4023/50 rubber plunger. In one embodiment, the pre-filled syringe comprises 125 mg or about 125 mg of amlitelimab in a 1 mL solution (125 mg/mL±18.75 mg/mL). Thus, it is appreciated that the invention provides a pre-filled syringe comprising a safety system that contains a 1 mL volume of an aqueous pharmaceutical formulation comprising about 125 mg/mL of the antibody or antigen binding fragment thereof (e.g. amlitelimab); about 10 mM L-histidine or histidine hydrochloride; about 220 mM sucrose; about 10 μM EDTA; about 0.06% (w/v) polysorbate 80, and water, with a pH of about 5.8 to about 6.2. In another embodiment, the pre-filled syringe comprises 250 mg or about 250 mg of amlitelimab in a 2 mL solution (125 mg/mL±18.75 mg/mL). Thus, it is appreciated that the invention provides a pre-filled syringe comprising a safety system that contains a 2 mL volume of an aqueous pharmaceutical formulation comprising about 125 mg/mL of the antibody or antigen binding fragment thereof (e.g. amlitelimab); about 10 mM L-histidine or histidine hydrochloride; about 220 mM sucrose; about 10 μM EDTA; about 0.06% (w/v) polysorbate 80, and water, with a pH of about 5.8 to about 6.2.

In one embodiment, provided is a pre-filled pen or autoinjector device containing an aqueous pharmaceutical formulation comprising 125 mg/mL±18.75 mg/mL or about 125 mg of the antibody or antigen binding fragment thereof (e.g. amlitelimab); 10 mM±1.5 mM or about 10 mM L-histidine or histidine hydrochloride; 220 mM±33 mM or about 220 mM sucrose; about 0.06% (w/v) polysorbate 80; about 10 μM EDTA, and water; with the pH of the aqueous pharmaceutical formulation being about 5.8 to about 6.2. The aqueous pharmaceutical formulation may be in a volume of approximately 1 mL±0.15 ml to 2.4 ml±0.36 ml. In one embodiment, the pre-filled pen device comprises 125 mg or about 125 mg of amlitelimab in a 1 mL solution (125 mg/mL±18.75 mg/mL). Thus, it is appreciated that the invention provides a pre-filled pen or autoinjector device that contains a 1 mL volume of an aqueous pharmaceutical formulation comprising about 125 mg/mL of the antibody or antigen binding fragment thereof (e.g. amlitelimab); about 10 mM L-histidine or histidine hydrochloride; about 220 mM sucrose; about 10 μM EDTA; about 0.06% (w/v) polysorbate 80, and water, with a pH of about 5.8 to about 6.2. In another embodiment, the pre-filled pen device comprises 250 mg or about 250 mg of amlitelimab in a 2 mL solution (125 mg/mL±18.75 mg/mL). Thus, it is appreciated that the invention provides a pre-filled pen or autoinjector device that contains a 2 mL volume of an aqueous pharmaceutical formulation comprising about 125 mg/mL of the antibody or antigen binding fragment thereof (e.g. amlitelimab); about 10 mM L-histidine or histidine hydrochloride; about 220 mM sucrose; about 10 μM EDTA; about 0.06% (w/v) polysorbate 80, and water, with a pH of about 5.8 to about 6.2.

In some embodiments, a drug product which comprises an aqueous pharmaceutical formulation contained in a container, e.g., a pre-filled syringe or a glass vial, has the stability attributes as described above.

### Kits

The present disclosure also provides kits comprising the pharmaceutical formulation disclosed herein.

In some embodiments, a kit comprises a container such as a glass vial, drug delivery device, prefilled syringe, microinfusor, pen delivery device or autoinjector described above, comprising an aqueous pharmaceutical formulation disclosed herein.

In some embodiments, a kit comprises a sealed container comprising an aqueous pharmaceutical formulation described herein and at least one separate injection device for delivery the aqueous pharmaceutical formulation to a mammalian subject in need thereof.

In some embodiments, the injection device is a syringe, a microinfusor, a pen delivery device, or an autoinjector. In some embodiments, the injection device is a single or multi-chambered syringe.

In certain exemplary embodiments, the kits of the present disclosure further comprise a control antibody which does not react with the hOX40L antigen. In a specific embodiment, the kits of the present disclosure may contain a substantially isolated hOX40L antigen as a control.

In some embodiments, a kit disclosed herein may further comprise a label or instructions comprise a marketing authorization number (e.g., an FDA or EMA authorization number).

In another specific embodiment, the kits of the present disclosure contain a means for detecting the binding of a modified antibody to a hOX40L.

In an example, provided is a kit comprising an aqueous formulation comprising the anti-OX40L antibody, or antigen-binding fragment thereof, packaging and instructions for use in treating Atopic Dermatitis.

In one embodiment, a kit comprises an antibody of the disclosure, e.g., a purified antibody, in one or more containers and an antigen (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate). In specific embodiments, the kit may include a recombinantly produced or chemically synthesized hOX40L antigen. The hOX40L antigen provided in the kit may also be attached to a solid support. In a more specific embodiment the detecting means of the above-described kit includes a solid support to which hOX40L antigen is attached. Such a kit may also include a non-attached reporter-labelled anti-human antibody. In this embodiment, binding of the antibody to the hOX40L antigen can be detected by binding of the said reporter-labelled antibody.

### Preparation Process

Embodiments of the present disclosure provide processes for preparing pharmaceutical formulations and unit dosage forms described herein.

In some embodiments, an antibody specifically binding to OX40 ligand (OX40L) is expressed in a suitable cell culture. The antibody in the cell culture is typically subjected to at least one of a chromatography step and at least one ultrafiltration step, thereby producing a purified antibody solution. The purified antibody solution may be adjusted to have a particular desired concentration of the antibody and to add particular excipients and adjust pH.

In one embodiment, a pharmaceutical formulation comprising the formulated anit-OX40L antibody according to the disclosed formulations may be prepared by 1) buffer exchange to achieve target buffer concentration and pH followed by 2) the up concentration above the target concentration.

In some embodiments, the concentration of the antibody is adjusted to a desired concentration ranging from about 28 mg/mL to about 138 mg/mL, EDTA, sucrose, L-histidine or histidine hydrochloride, and polysorbate 80 are added, and the pH is adjusted to be about 5.5 to about 6.5. In one embodiment, the concentration of the antibody is adjusted to about 28 mg/mL.

In one embodiment, the concentration of the antibody is adjusted to about 31 mg/mL to about 125 mg/mL mg/mL. In one embodiment, the concentration of the antibody is adjusted to about 31 mg/mL.

In some embodiments, the concentration of the antibody is adjusted to a concentration ranging from about 75 mg/mL to about 138 mg/mL, sucrose, L-histidine or histidine hydrochloride, and polysorbate 80 are added, and the pH is adjusted to be about 5.5 to about 6.5. In one embodiment, the concentration of the antibody is adjusted to 75±11.25 mg/mL. In another embodiment, the concentration of the antibody is adjusted to 138±20.7 mg/mL. In yet another embodiment, the concentration of the antibody is adjusted to 125±18.75 mg/mL.

In some embodiments, the pH of the formulation is adjusted to about 5.5 to about 6.5, such as 5.5±0.2 to 6.5±0.2. In some embodiments, the pH is adjusted to about 6, such as 6.0±0.2. In some embodiments, the pH is adjusted to 6.0. In some embodiments, the pH is adjusted to 6.2.

In some embodiments, the sucrose is at a concentration of about 220 mM, such as 220 mM±33 mM; the EDTA is at a concentration of about 10 μM, such as 10 μM±1.5 μM; the L-histidine or histidine hydrochloride is at a concentration of about 10 mM, such as 10 mM±1.5 mM; and the polysorbate 80 is at a concentration of about 0.04% v/v, such as 0.04% (w/v)±0.006% (w/v), or is at a concentration of about 0.06% v/v, such as 0.06% (w/v)±0.009% (w/v).

In some embodiments, the sucrose is at a concentration of about 220 mM, such as 220 mM±33 mM, the L-histidine or histidine hydrochloride is at a concentration of about 20 mM, such as 20 mM±3 mM; and the polysorbate 80 is at a concentration of about 0.04% v/v, such as 0.04% (w/v)±0.006% (w/v).

A pharmaceutical formulation disclosed herein may be prepared from a bulk purified drug substance (BPDS) of amlitelimab in a solution containing amlitelimab at a low concentration such as approximately 10.1 g/L, in a buffer, such as a buffer with 25 mM L-Histidine/L-Histidine hydrochloride at pH 6.2. Bulk purified drug substance in the solution may be stored at ≤65° C. from the date of manufacture to the date that the material is aliquoted, compounded, and transferred to another appropriate storage condition.

In some embodiments, the formulated antibody is not freeze-dried, and the pharmaceutical formulation is not in lyophilized form.

In some embodiments, the formulated antibody is freeze-dried, and the pharmaceutical formulation is in lyophilized form.

The protein concentration, pH value, and density may be determined upon processing of the BPDS and utilized for a required calculation of a batch formulation by using standard dilution procedure. The formulations may be compounded by spiking with excipient stock solutions to the target formulation of the bulk drug product. The protein concentration, osmolality, and pH of the finally compounded solutions can be determined and confirmed.

In some embodiments, prepared formulation solutions may be filtered using a 0.22 μm polyvinylidene fluoride (PVDF) membrane filter.

In some embodiments, formulation solutions containing the antibodies prepared according to the processes described herein may be used as primary packaging materials and filled in a proper container to produce packaged drug products. In certain embodiments, the packaging materials may be filled, observing aseptic technique, in a vial, e.g., a glass vial. In some embodiment, the container is a 6R/20 mm glass type I vial, where the formulated solution is filled with a target fill volume of 2.4 mL, and the vial is then stoppered with 20 mm bromobutyl rubber stoppers (injection and freeze-drying stoppers) and sealed with 20 mm aluminum flip-off seals.

In some other embodiments, the container is a syringe. In some embodiments, the syringe is a low-tungsten glass syringe. In one embodiment, the syringe is a NUOVA OMPI 1 mL long glass syringe equipped with a 27-G thin wall needle, an FLUROTEC-coated 4023/50 rubber stopper, and a FM 27 rubber tip cap.

### Use of the Pharmaceutical Formulations

The pharmaceutical formulations and packaged products of the present disclosure are suitable for administration to a mammal (human or non-human) subject by parenteral routes, such as subcutaneous, intravenous, intramuscular, intraperitoneal, or intradermal injection, for treating, preventing, or ameliorating any disease or disorder associated with OX40L activity, including diseases or disorders mediated by OX40L. Exemplary, non-limiting diseases and disorders that can be treated or prevented by the administration of the pharmaceutical formulations of the present disclosure include autoimmune disorders, inflammatory diseases or conditions, transplant rejection, etc. In some embodiments, the pharmaceutical formulations and packaged products of the present disclosure are suitable for treating a disease such as asthma or atopic dermatitis.

### Exemplary Embodiments

TABLE 1a
Formulations

| Ingredients | FDS1 | FDS1.1 | FDS1.2 |
|---|---|---|---|
| Antibody | 31 mg/mL- | 62.5 mg/mL | 125 mg/mL |
| (amlitelimab) | 125 mg/mL | | |
| Histidine/histidine | 10 mM | 10 mM | 10 mM |
| hydrochloride | | | |
| Sucrose | 220 mM | 220 mM | 220 mM |
| Polysorbate 80 | 0.06% (w/v) | 0.06% (w/v) | 0.06% (w/v) |
| Chelator | 10 μM EDTA or | 10 μM EDTA | 10 μM EDTA |
| | 10 μM DTPA | | |
| PH | 6.0 | 6.0 | 6.0 |
| Water | QS | QS | QS |

In addition to their good properties in term of stability and quality, the above exemplified formulations FDS1.1 and FDS1.2 are safe and well tolerated in humans.

TABLE 1b
Formulations

| Ingredients | FDS1a | FDS2a | FDS3a |
|---|---|---|---|
| Antibody | 125 mg/mL | 138 mg/mL | 75 mg/mL |
| (amlitelimab) | | | |
| Histidine/histidine | 20 mM | 20 mM | 20 mM |
| hydrochloride | | | |
| Sucrose | 220 mM | 220 mM | 220 mM |
| Polysorbate 80 | 0.04% (w/v) | 0.04% (w/v) | 0.04% (w/v) |
| PH | 6.0 | 6.0 | 6.0 |
| Water | QS | QS | QS |

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the formulations, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure. Any part of this disclosure may be read in combination with any other part of the disclosure, unless otherwise apparent from the context.

### EXAMPLES

The following examples further illustrate aspects of the present disclosure. However, they are in no way a limitation of the teachings of the present disclosure as set forth. It should be understood that these Examples are given by way of illustration only. From the above discussion and these Examples, one of ordinary skill in the art can ascertain the essential characteristics of embodiments of the present disclosure. Without departing from the spirit and scope thereof, one skilled in the art can make various changes and modifications of the disclosure to adapt it to various usages and conditions. All publications, including patents and non-patent literature, referred to in this specification are expressly incorporated by reference herein.

### Abbreviations

ArgHCl: Arginine hydrochloride
BDP: Bulk Drug Product
BPDS: Bulk Purified Drug Substance
CE-SDS: Capillary Electrophoresis Sodium Dodecyl Sulfate
CAD: Charged aerosol detector
cIEF: Capillary isoelectric focusing
CR: comparable to reference
DLS: Dynamic Light Scattering
DP: Drug product
DS: Drug substance
DSC: Differential scanning calorimetry
DTPA: Diethylenetriaminepentaacetic acid
EDTA: Ethylenediaminetetraacetic acid
ELISA: Enzyme-linked immunosorbent assay
Eur. Ph.: European Pharmacopeia
FCC: Food Chemical Codex
FDS: Formulated drug substance
FIH: First in human
F/T: Freeze/Thaw
GMP: Good Manufacturing Practices
HDPE: High-Density Polyethylene
HisHCl: Histidine hydrochloride
iCIEF Imaged Capillary Isoelectric Focusing
iLAB LES Laboratory Execution System
HIAC: High accuracy liquid particle counter
HMWS: High molecular weight species
LC-MS: Liquid chromatography mass spectrometry
LIMS Laboratory Information and Management System
LMWS: Low molecular weight species
<LoQ: Less than limit of quantitation for the method
NaCl: Sodium chloride
N/A: Not applicable
NBE: No blow back
NF: National Formulary
NT: not tested
NTU: Nephelometric turbidity units
OD: Optical density
PFVP: Practically free from visible particles
pI: Isoelectric point
PS80: Polysorbate 80
PVDF: Polyvinylidene Fluoride
RP-HPLC: Reversed Phase-High Performance Liquid Chromatography
SC: Subcutaneous
SEC: Size-exclusion chromatography
SE-HPLC: Size Exclusion High Performance Liquid Chromatography
SL: Standard Line
SOP: Standard Operating Procedure
Tbd: not available yet
TM: Test Method
TPP: Technical Pilot Plant
UF/DF: Ultrafiltration/diafiltration
USP: United States Pharmacopeia
PFS: Pre-filled syringe

In the following examples, amlitelimab (also known as SAR 445229 and KY1005) was used exemplarily as the antibody specifically binding hOX40L. Amlitelimab is a fully human IgG4 Kappa monoclonal antibody having a “IgG4-PE” constant region with Leu235Glu and Ser228Pro Fc mutations. Amlitelimab comprises a VH domain comprising an amino acid sequence set forth in SEQ ID NO: 34 and a VL domain comprising an amino acid sequence set forth in SEQ ID NO: 48.

### Example 1—Development of Optimized Stable Anti-hOX40L Antibody Formulations Objectives

Amlitelimab (also known as SAR 445229 or KY1005) is a human anti-OX40L subclass G4PE kappa mAb that binds OX40L to block interaction with its receptor OX40, a key regulator of the immune system. Amlitelimab is designed to rebalance the immune system by blocking irregular activation and proliferation of pro-inflammatory effector T-cells and promoting expansion of anti-inflammatory regulatory T-cells, without broad suppression of the immune system. Amlitelimab is currently in clinical trial as a therapy for atopic dermatitis.

It is desired that a new stable formulation can be properly used for any dose, such as from about 62.5 mg-550 mg, such as a dose of 62.5 mg, or a dose of 125 mg or a dose of 250 mg or a dose of 500 mg. It is also desired to be presented as a drug product with a shelf life of 24 months.

In this example, a set of comprehensive studies were performed to develop an optimized anti-hOX40L formulation. The studies assessed the stability, feasibility, and liabilities of the antibody formulations. The impacts of buffer concentrations and pH, excipient species, polysorbate 80 (PS80) concentrations, and metal chelators were all critically assessed for optimized development of an aqueous Amlitelimab drug product (DP) and bulk formulated drug substance (FDS).

The preliminary quality target product profile for formulations and products to be developed is shown in Table 1-1.

TABLE 1-1
Preliminary Quality target product profile (QTPP)

| Product attribute Target | Target |
|---|---|
| Drug delivery requirements | Subcutaneous (SC) injection |
| Dosage form | Solution for injection |
| Primary packaging | BD Neopak 2.25 mL 27G 1/2 |
| | 5B STW RNS FM30 HS syringe |
| | closed with West 1-3 mL Novapure |
| | 4023/50 Gray plunger stopper |
| Strength | 62.5 mg/PFS; 125 mg/PFS; |
| | 250 mg/PFS |
| Protein concentration | 31.25 mg/mL; 62.5 mg/mL; |
| | 125 mg/mL |
| Nominal volume | 2 mL |
| Storage condition | 5 ± 3° C. |
| Targeted shelf life ≥24 months | ≥24 months |

### Materials and Methods

### Drug Substance

Amlitelimab formulated drug substance (FDS) was prepared. The FDS concentration was generally prepared at >138 mg/mL. For these studies in this example, FDS was formulated to the target protein and excipient concentrations, and 0.22 μm filtered under laminar flow before aseptic filling.

### Excipients

All excipients used during formulation development studies were United States Pharmacopeia (USP) and European Pharmacopeia (Eur. Ph.) grade raw materials.

### Inspection for Visible Particles

Visible particles were analyzed, by one or two analysts, under a visual inspection unit (Seidenader Maschinenbau GmbH). DP containers were cleaned with lens paper before inspection to remove dust and fingerprints from the outer surface.

### PH Measurements

The pH of buffers and formulated mAb solutions were measured using a Thermo-Scientific pH probe and meter. The results were considered comparable when the difference between repeated measurements was within 0.1 pH units.

### Osmolality Measurements

Osmolality measurements were performed on 20 μL samples (n=2-3) using a freezing point depression osmometer (Advanced Instruments, OsmoPRO). Osmolality standards were run before and after sample analysis to ensure measurement accuracy.

### Protein Concentration Measurements

Total protein concentration was determined by measuring the UV absorbance at 280 nm on microfluidic chips on a Big Lunatic system from Unchained Labs. Measurements were performed in duplicate on 2 to 5 μL of sample.

### Turbidity Analysis

Sample turbidity was quantified by measuring optical density (OD) from 340 nm to 360 nm on a Synergy neo2 multi-mode reader from BioTek. For each sample, 200 μL was loaded onto a UV-Vis transparent 96 well plate. The OD was determined as the average of absorbance values at 340 nm, 345 nm, 350 nm, 355 nm, 360 nm.

### SEC-HPLC for HMWS

Aggregation analysis was performed by size exclusion chromatography (SEC). Samples were run on a 1260 series HPLC (Agilent, Santa Clara, CA) equipped with a TSK-GEL G3000SWXL (Tosoh Bioscience, Tokyo, Japan) analytical column and matching guard column. The mobile phase used was 40 mM phosphate and 150 mM sodium chloride, pH 7.2 with a flowrate of 0.75 mL/min for 25 minutes. The protein concentration upon injection was 5 mg/mL. Two injections were performed for each sample. Detection was carried out by UV absorbance at 280 nm and the chromatographic peaks were integrated to determine the relative percentage of each eluted species.

### HIAC for Subvisible Particles

Subvisible particles were measured by light obscuration on a Beckman Coulter high accuracy particle counter (HIAC) Model 9703⁺. The system was flushed with MilliQ water until particle counts were ≤1 particle/mL at 10 μm. 2 μm, 10 μm and 25 μm standards were measured to ensure accurate particle concentrations followed by extensive washing to remove any background. Using a 0.8 mL method, samples were measured with four separate injections of 0.2 mL. The first sample measurement was disregarded and the following three were averaged.

### PS80 Quantification by HPLC/CAD

PS80 was analyzed by HPLC-CAD. Samples were run on a 1260 series HPLC (Agilent, Santa Clara, CA) equipped with a Water Oasis MAX column and a Charged Aerosol Detector. The mobile phase used was 2% Formic acid in water and 2% Formic acid in isopropyl alcohol with a flow rate of 1 mL/min for 8 minutes. Three injections were performed for each sample. Detection was carried out by CAD and the chromatographic peaks were integrated to determine the area under the curve which was compared with the calibration curve to give the PS80 concentrations.

### Capillary Gel Electrophoresis

Fragmentation analysis was performed by non-reducing capillary gel electrophoresis (NR-CE). Samples were run on a LabChip GXII instrument (PerkinElmer, Waltham, MA), using the supplier's Protein Clear HR chip, predeveloped method, and reagent kit. The electropherogram peaks were integrated to determine the percentage of monomer and low molecular weight species (LMWS) and the percentage of monomer is reported as purity percent and LMWS reported as fragmentation percent.

### PTM Quantification by LC-MS

Protein sample solutions and reference standards were diluted to a concentration of 0.46 mg/mL using 6 M guanidine HCl, 250 mM Tris pH 8.5 (reduction/alkylation Buffer; RAB). Initial concentrations were entered, and the dilution was calculated by the Hamilton software using the following formula:

$$Sample volume ( μL ) = 60 μL / Sample concentration ( mg / mL )$$

$$RAB volume ( μL ) = 130 μL - Sample volume ( μL )$$

Trypsin/Lys-C digestion of samples was performed using 25 mM Tris, 20 mM methionine, pH 7.1 digest buffer. For each sample, 25 μL of the digest was injected onto a C18 column for LC-MS analysis using a trifluoroacetic acid buffer system. Samples were analyzed using the Q Exactive Full MS method.

Generated LC-MS data were processed using Chromeleon 7.2.10 software for relative quantitation of modifications including Met/Trp oxidation, deamidation, Asp isomerization, and HC C-terminal modification, as well as for glycan and charge analysis.

cIEF for Charge Variants

The protein charge heterogeneity was measured by capillary isoelectric focusing (cIEF), by Fluorescence detection mode with a 10 second exposure, using a Protein Simple Maurice instrument, The samples and the standard solutions were first diluted to 5 mg/mL in water, followed by dilution to 0.5 mg/mL with water. Onboard mixing was used to mix samples with MasterMix before analyses. The results were considered comparable when the difference was equal or less than 10%.

### Buffer/pH Screening

Study was conducted to evaluate the effects of buffer identity and pH on the physical and chemical stability of amlitelimab aqueous formulations at low and high concentrations in PFS during refrigerated storage, accelerated storage, and stressed storage conditions. The study was also conducted to monitor the stability of high concentration aqueous formulations in vials stored at frozen storage temperatures. Histidine buffer systems were chosen for this study at pH values 5.5 to pH 6.3.

### Sample Conditions

To bracket amlitelimab DP concentrations from 31-125 mg/mL, 28 mg/mL and 138 mg/mL of amlitelimab were selected which includes 10% variability in the concentrations. Formulations comprising 10 mM or 20 mM Histidine buffer at pH ranging from 5.5 to 6.3, along with 220 mM sucrose, 0.04% PS80, and optionally, 10 μM EDTA, as shown in Table 1-2 below, were prepared, stored in OMPI syringes, and analyzed as detailed below.

TABLE 1-2
Formulation conditions and sample references

| Syringe | amlitelimab | Buffer | | | % | μM | |
|---|---|---|---|---|---|---|---|
| Ref # | (Conc mg/ml) | (Histidine) | pH | Sucrose | PS80 | EDTA | Syringe |
| 1_s | 28 | 10 mM | 6.0 | 220 mM | 0.04 | 10 | OMPI |
| 2_s | 28 | 10 mM | 5.5 | 220 mM | 0.04 | 10 | OMPI |
| 3_s | 28 | 10 mM | 5.8 | 220 mM | 0.04 | 10 | OMPI |
| 4_s | 28 | 10 mM | 6.3 | 220 mM | 0.04 | 10 | OMPI |
| 5_s | 28 | 20 mM | 6.0 | 220 mM | 0.04 | 10 | OMPI |
| 6_s | 28 | 20 mM | 6.0 | 220 mM | 0.04 | — | OMPI |
| 11_s | 138 | 10 mM | 6.0 | 220 mM | 0.04 | 10 | OMPI |
| 12_s | 138 | 10 mM | 5.5 | 220 mM | 0.04 | 10 | OMPI |
| 13_s | 138 | 10 mM | 5.8 | 220 mM | 0.04 | 10 | OMPI |
| 14_s | 138 | 10 mM | 6.3 | 220 mM | 0.04 | 10 | OMPI |
| 15_s | 138 | 20 mM | 6.0 | 220 mM | 0.04 | 10 | OMPI |
| 16_s | 138 | 20 mM | 6.0 | 220 mM | 0.04 | — | OMPI |

### Visual Inspection

It was observed that all formulations containing 28 mg/mL in syringe Ref #1_s to 6_s, and all formulations containing 138 mg/mL of amlitelimab in syringe Ref #11_s to 16_s remained visually clear and colorless after 12 weeks storage in the OMPI syringes at 5° C., 25° C., and 40° C.

### Aggregation by SEC-HPLC

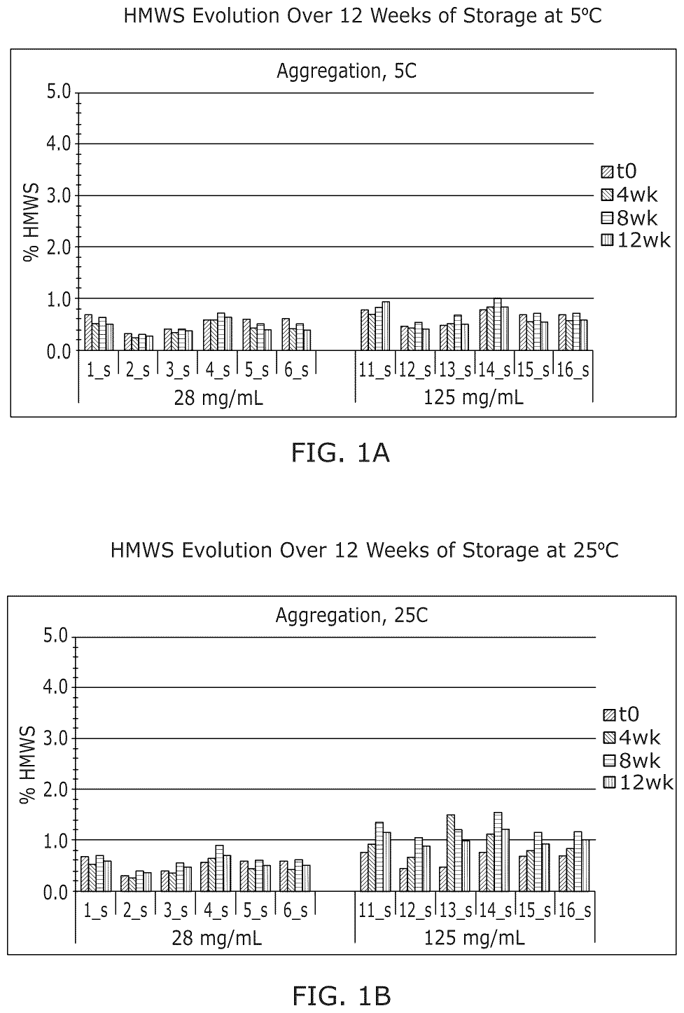

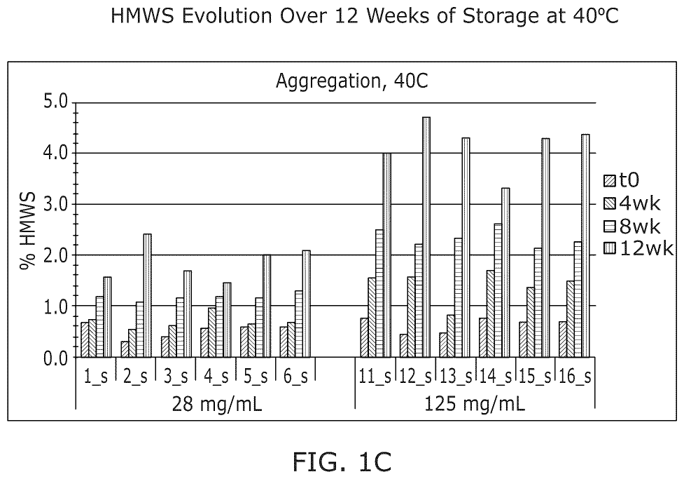

Aggregation of the formulations contained in Syringe Ref #1_s to 6_s and 11_s to 16_s was evaluated by SEC-HPLC. FIGS. 1A, 1B, and 1C graphically depict HMWS evolution of the formulations contained in OMPI syringes upon storage over 12 weeks at 5° C., 25° C., and 40° C., respectively. As shown in FIGS. 1A, 1B, and 1C, none of the example formulations studied demonstrated a substantial increase in HMWS % after 12 weeks of storage at 5° C. and 25° C. though there was an increase in HMWS % in all formulations stored at 40° C. In general, samples at higher protein concentrations showed a propensity to aggregation than those at lower protein concentrations. There are some minor differences in the propensities of HMWS formation between different formulations, though overall, samples in all formulations have less than 1% HMWS after 12 weeks at 5° C., less than 2% HMWS after 12 weeks at 25° C., and less than 5% HMWS after 12 weeks 40° C.

### Subvisible Particles by HIAC

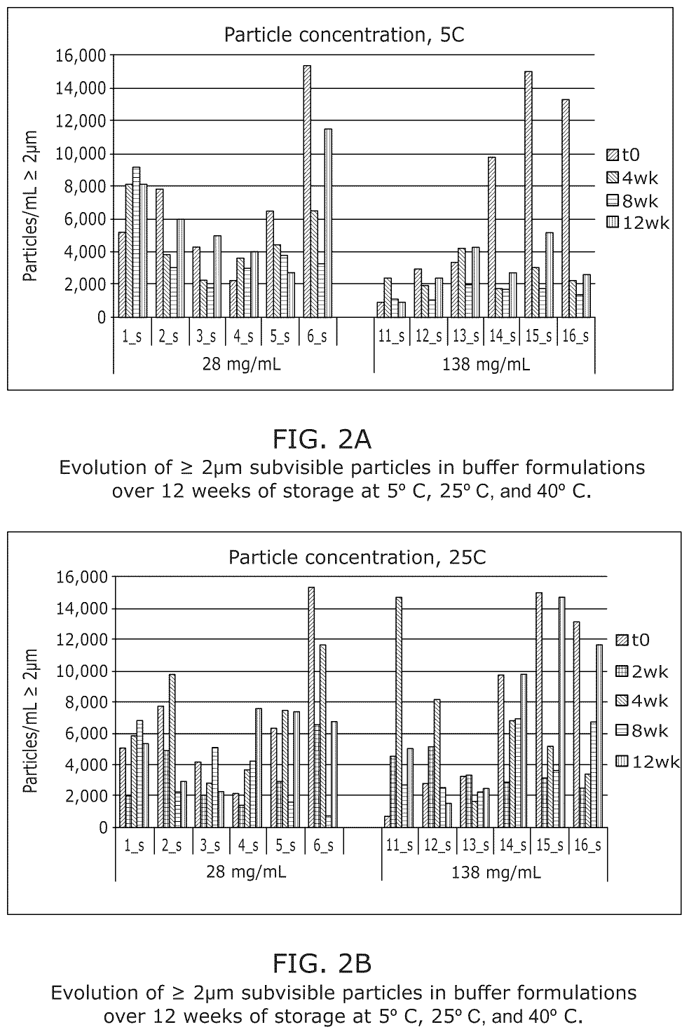

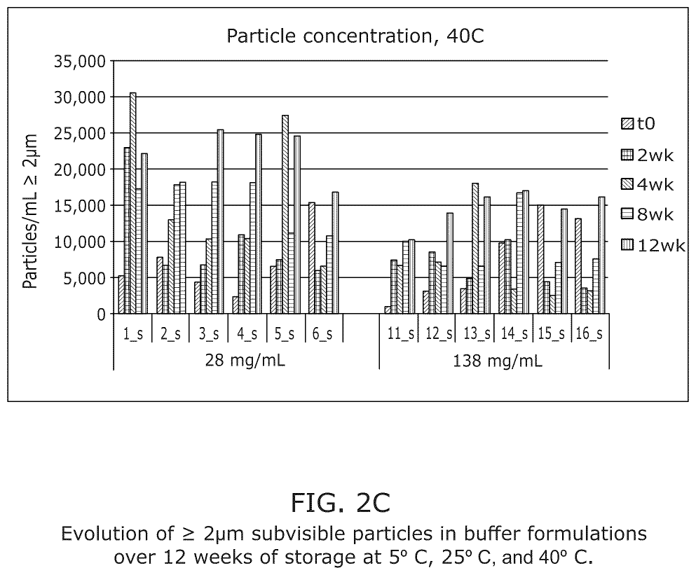

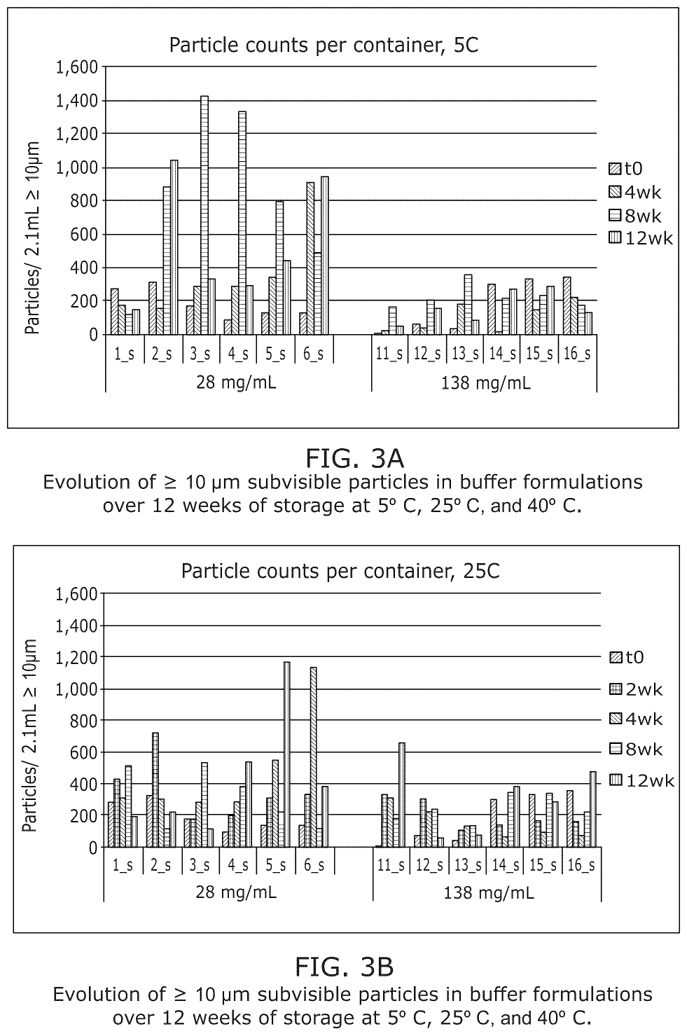

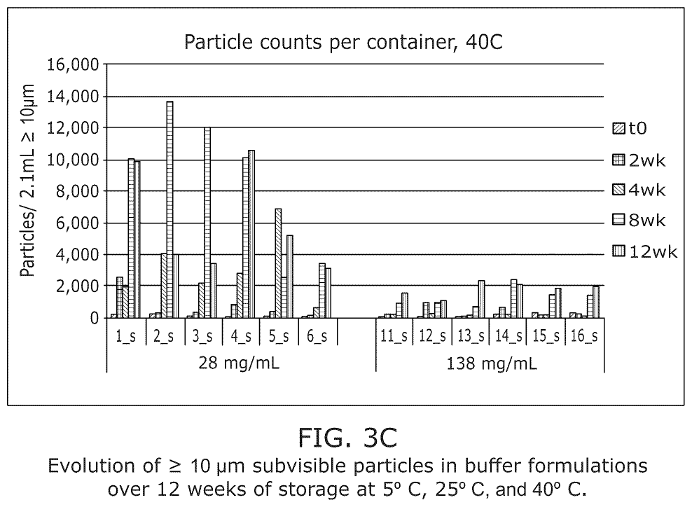

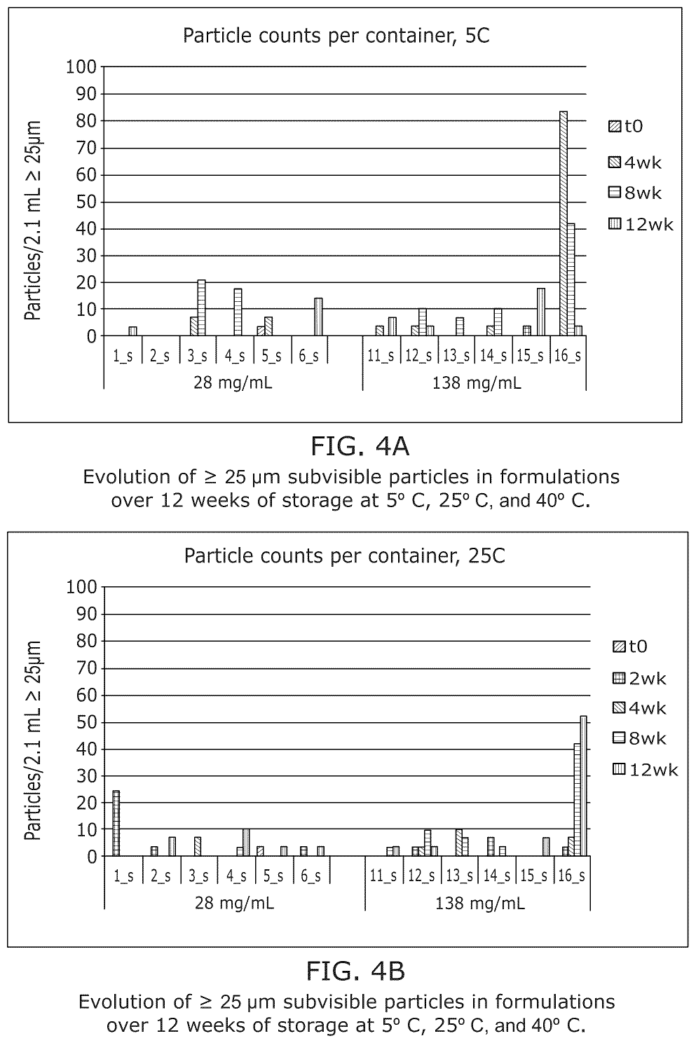

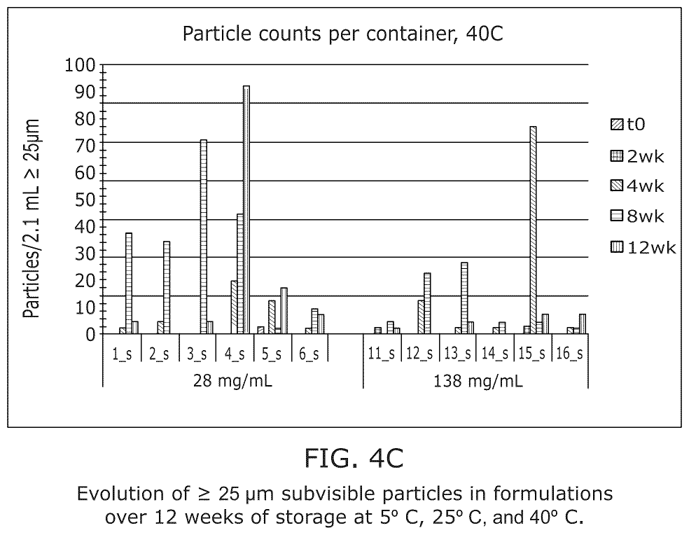

Subvisible particles in example formulations contained in Syringe Ref #1_s to 6_s and 11_s to 16_s were evaluated by HIAC. FIGS. 2A, 2B, 2C graphically depict the evolution of ≥2 μm subvisible particles in the example formulations contained in OMPI syringes upon storage over 12 weeks at 5° C. (FIG. 3A), 25° C. (FIG. 2B), and 40° C. (FIG. 2C). FIGS. 3A, 3B, and 3C graphically depict the evolution of ≥10 μm subvisible particles in the example formulations upon storage over 12 weeks at 5° C. (FIG. 3A), 25° C. (FIG. 3B), and 40° C. (FIG. 3C). FIGS. 4A, 4B, and 4C graphically depict the evolution of ≥25 μm subvisible particles in the example formulations contained in the OMPI syringes upon storage over 12 weeks at 5° C. (FIG. 4A), 25° C. (FIG. 4B), and 40° C. (FIG. 4C).

As shown in FIGS. 2-4, subvisible particle formation, at all particle sizes, was relatively prominent at the stressed temperature of 40° C. compared to 5° C. and 25° C. At 40° C. all formulations showed a general increase in particles over 12 weeks with the lower protein-concentration samples showing relatively larger particle generation than the high protein-concentration samples.

### Turbidity Analysis

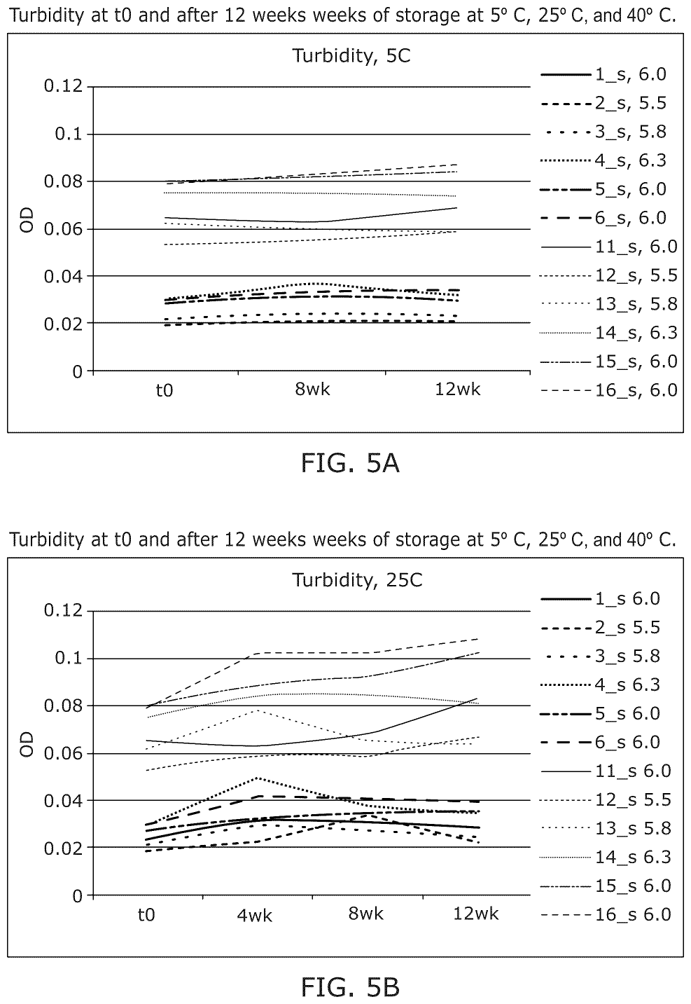

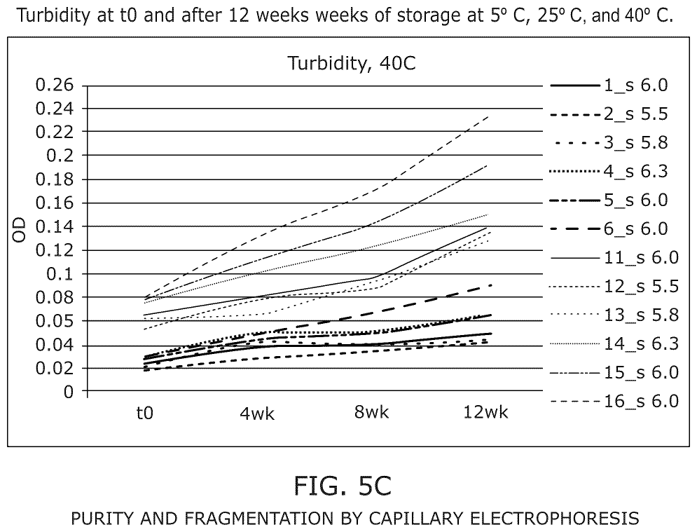

Plate-based UV measurements were made to track turbidity and opalescence during storage for all the formulation samples in syringe ref #1_s to 6_s and in syringe ref #11_s to 16_s. The results of turbidity measured for all formulation samples after 12 weeks of storage at 5° C., 25° C., and 40° C. are graphically illustrated in FIGS. 5A, 5B, and 5C, where FIG. 5A depicts the turbidity results for the formulation samples upon the storage over 12 weeks at 5° C., FIG. 5B depicts the turbidity results for the formulation samples upon the storage over 12 weeks at 25° C., and FIG. 5C depicts the turbidity results for the formulation samples upon the storage over 12 weeks at 40° C.

It was found that protein concentration and storage temperature influenced turbidity. With respect to formulation, similar trends were observed for samples with low and high protein concentrations.

It was surprisingly found that formulations containing 20 mM histidine tended to have higher turbidity to start and had a higher propensity to increase compared to formulations with 10 mM histidine, especially under stressed condition at 40° C.

### Purity and Fragmentation by Capillary Electrophoresis

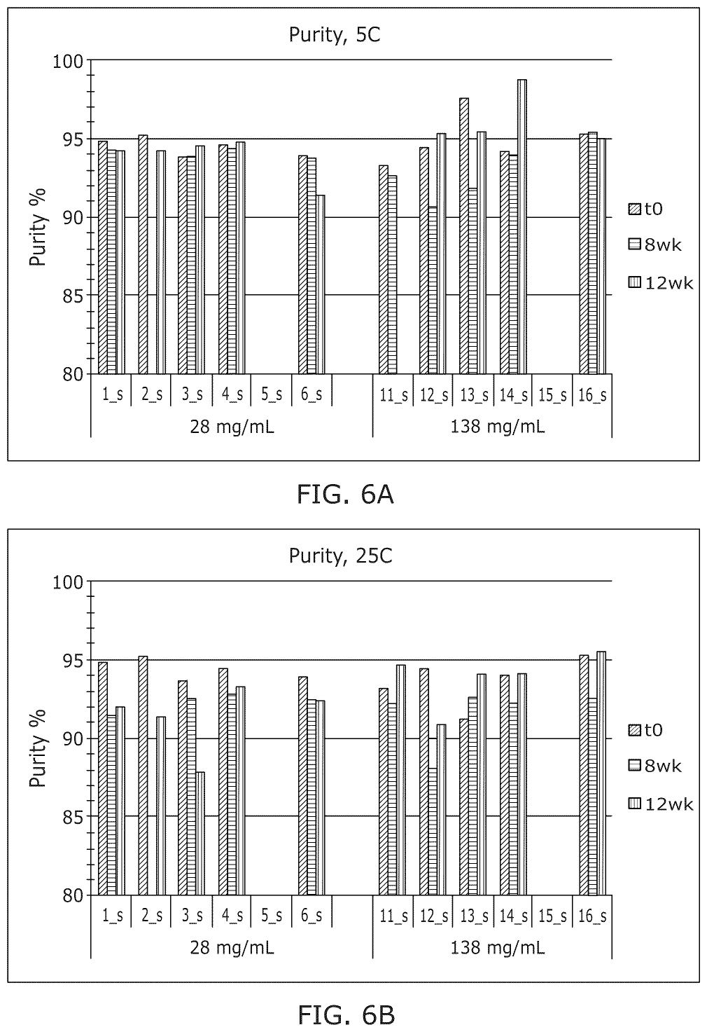

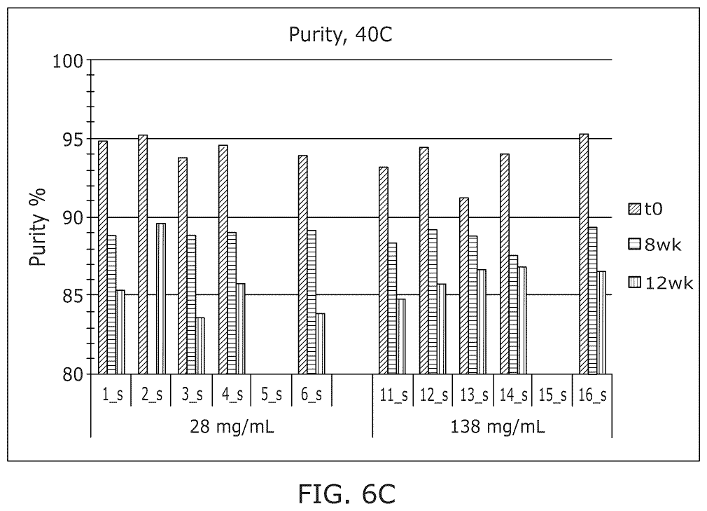

Purity was measured by capillary electrophoresis, for most samples, monitoring the fractional content of the monomer species in the formulation samples over 12 weeks of storage at 5° C., 25° C., and 40° C. The results were shown in FIGS. 6A, 6B, and 6C, where FIG. 6A illustrates the results for the storage at 5° C., FIG. 6B illustrates the results for the storage at 25° C., and FIG. 6C illustrates the results for the storage at 40° C. As can be seen from FIGS. 6A, 6B, and 6C, most of the samples remained >90% purity at 12 weeks at both 5° C. and 25° C. At 40° C., fragmentation was common, with slightly larger increases in the lower concentration samples. It was found that increases in LMWS % were comparable between low- and high-concentration samples.

### PS80 Quantification

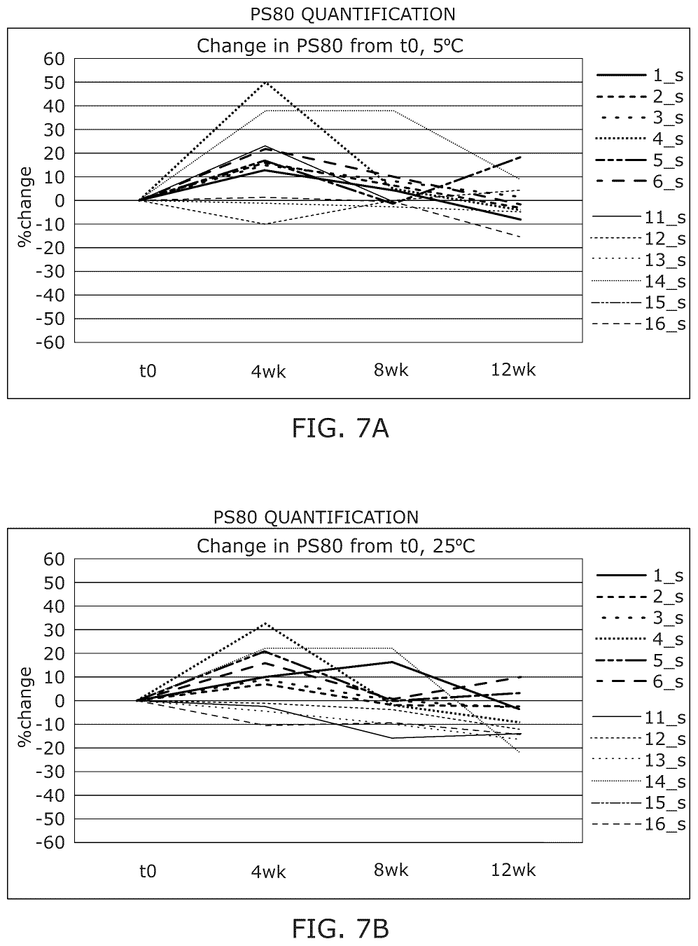

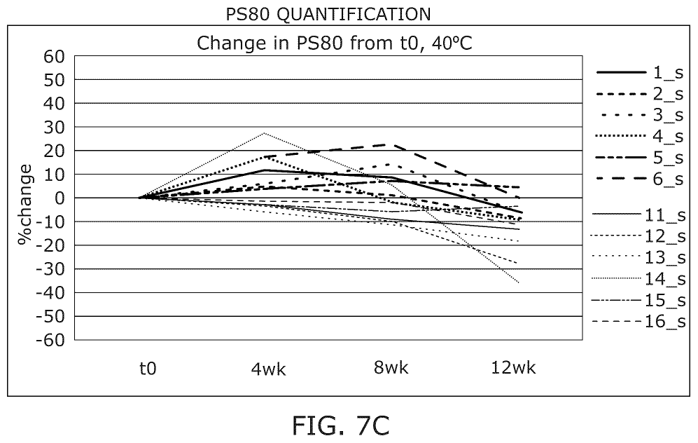

PS80 quantification of the example formulations stored in Syringe Ref #1_s to 6_s and 11_s to 16_s over 12 weeks at 5° C., 25° C., and 40° C. for were analyzed. The results are graphically depicted in FIGS. 7A, 7B, and 7C, where FIG. 7A illustrates the PS80 quantification of the example formulations stored in the syringes over 12 weeks of storage at 5° C., FIG. 7B illustrates PS80 quantification of formulations over 12 weeks of storage at 25° C., and FIG. 7C illustrates PS80 quantification of formulations over 12 weeks of storage at 40° C. The method variability for PS80 analysis by CAD was about 25% (±100 ppm PS80) in this study. At 5° C., 25° C. and 40° C., no trend was observed for change in PS80 over time, and the percent change was within the variability of the analysis. 10 mM histidine pH 6.3 at both low and high protein concentrations (Syringe Ref #4_s and 14_s, respectively) showed an increase in percent change at 4 weeks and 8 weeks but the PS80 concentration was within the variability for 12 weeks. There was no significant decrease in PS80 in these formulations after 12 weeks at all temperatures.

### Charge Variants

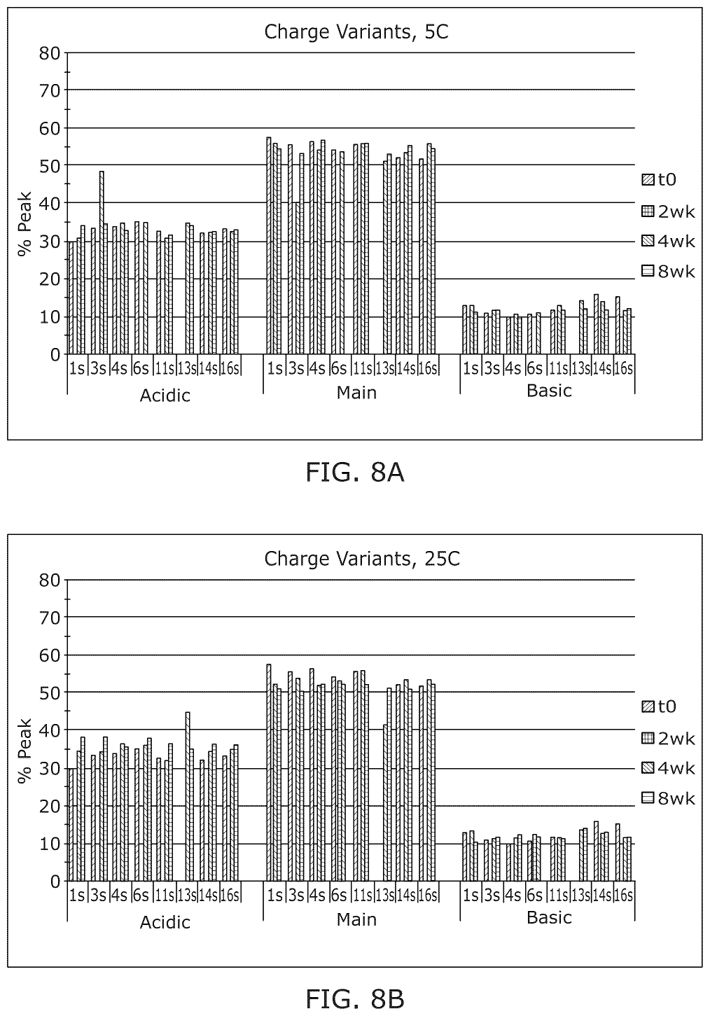

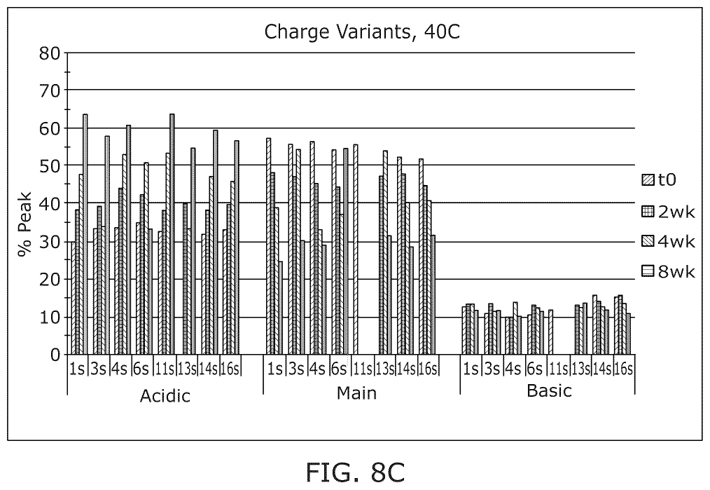

Charge variants were analyzed by capillary isoelectric focusing (cIEF) for the example formulations stored in Syringe Ref #1_s to 6_s and 11_s to 16_s over 8 weeks at 5° C., 25° C., and 40° C. The results are graphically depicted in FIGS. 8A, 8B, and 8C, where FIG. 8A depicts the charge variants of the formulations over 8 weeks of storage at 5° C., FIG. 8B depicts the charge variants of the formulations over 8 weeks of storage at 25° C., and FIG. 8C depicts the charge variants of the formulations over 8 weeks of storage at 40° C. The results show that all formulations had comparable variants for each timepoint. At 40° C. most formulations tested had an increase in acidic species and a decrease in the main peak, over 8 weeks, while the basic species remained constant.

### Peptide Mapping

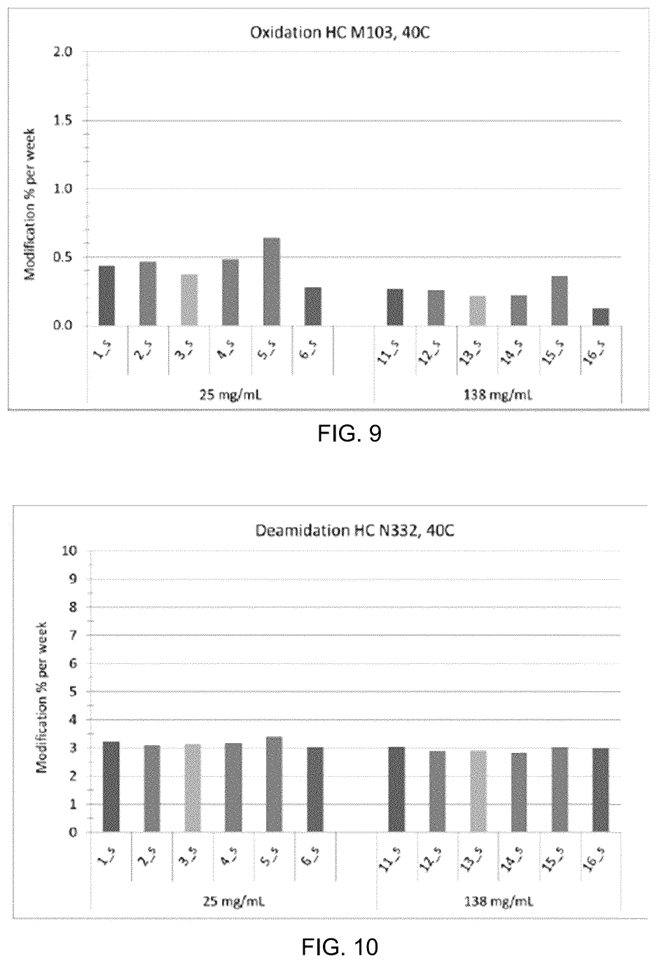

Modification of the antibody in the formulations was measured by peptide mapping after 3-month storage at 40° C. The percent change per week was calculated for deamidation at HC N332 and oxidation at HC M103 of the antibody. The results are graphically depicted in FIG. 9 and FIG. 10, where FIG. 9 depicts the deamidation at HC N332 and FIG. 10 depicts the oxidation at HC M103. It was observed that there was no significant difference, across formulations or protein concentrations, of change per week in deamidation. The oxidation at HC M103 in the antibody in formulations with 25 mg/mL protein had slightly higher oxidation percent per week compared to the formulations with higher protein concentration of 138 mg/mL.

pH

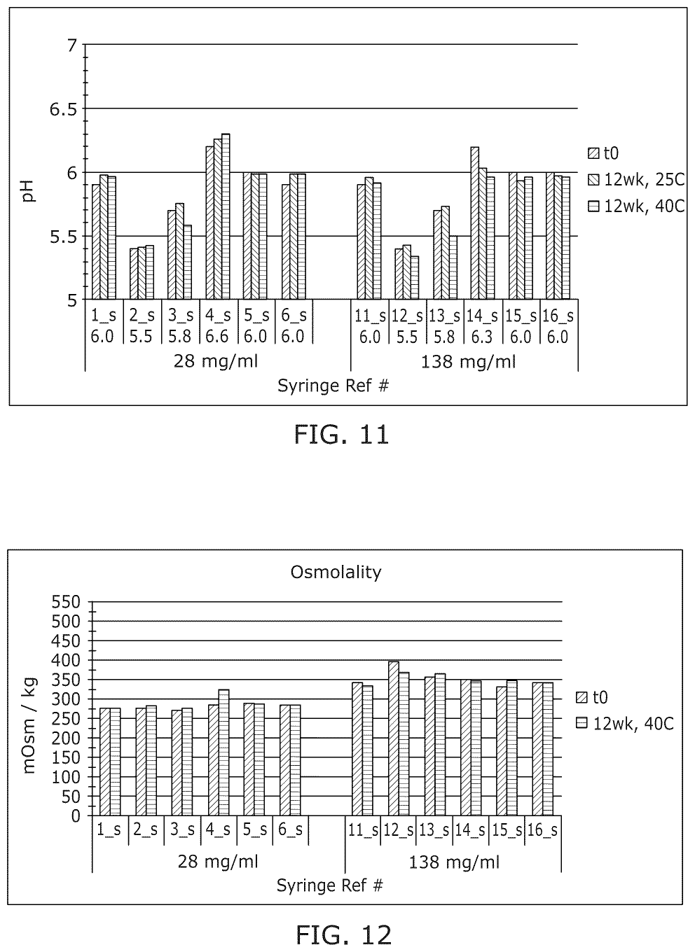

The pH values of the formulations were measured at t0 and after 12 weeks of storage at 25° C. and 40° C. As shown in FIG. 11, there were no significant changes in pH for any formulations at any time points even at accelerated or stressed storage conditions (25° C. and 40° C.) for 28 mg/mL or 138 mg/mL.

### Osmolality

Osmolality of the formulations were measured at t0 and over 12 weeks of storage at 40° C. As shown in FIG. 12, there were no significant changes in osmolality for any formulations up to 12 weeks under stressed condition at 40° C., at 28 mg/mL or 138 mg/mL.

### Buffer Influence on Frozen Stability

Buffer/pH influence on mAb frozen stabilities after storage were evaluated in 6R Schott vials to solutions in Vial Ref Nos 11_v to 16_v, which comprise 138 mg/mL of mAb in buffers with 10 mM or 20 mM histidine, and varied in pH from 5.5 to 6.3. Frozen stabilities from t0, after storage for up to 12 weeks at −30° C. and −80° C. were analyzed for HMWS %, turbidity, pH, concentration, subvisible particles (≥2 μm, ≥10 μM, ≥25 μm).

Obtained results show that frozen stability data was comparable for samples among different formulations stored at −30° C. and −80° C. No significant changes in aggregation, turbidity, pH, concentration, and particle formation were observed for up to 12 weeks.

### Stabilizer Study

Stabilizer study was conducted to assess the effects of different stabilizers, or bulking agents, on the physical and chemical stability of amlitelimab aqueous formulations at low and high concentrations in PFS during refrigerated storage, accelerated storage, and stressed storage conditions. The study also monitored the stability of high concentration aqueous formulations in vials stored at frozen storage temperatures.

### Sample Conditions

Aqueous formulations comprising 28 mg/mL or 138 mg/mL of amlitelimab, with 10% variability in the concentration, were prepared according to the ingredients and concentrations of the ingredients listed in Table 1-3. Such prepared aqueous formulations were filled in OMPI syringes.

TABLE 1-3
Formulation conditions and sample references

| Syringe | amlitelimab | Buffer | | Sucrose/ | | | |
|---|---|---|---|---|---|---|---|
| Ref # | (Conc mg/ml) | (Histidine) | pH | Trehalose | Stabilizer | % PS80 | μM EDTA |
| 1_s | 28 | 10 mM | 6.0 | 220 mM | — | 0.04 | 10 |
| | | | | sucrose | | | |
| 7_s | 28 | 10 mM | 6.0 | 220 mM | — | 0.04 | 10 |
| | | | | trehalose | | | |
| 8_s | 28 | 10 mM | 6.0 | 120 mM | 50 mM | 0.04 | 10 |
| | | | | sucrose | Arg-HCl | | |
| 11_s | 138 | 10 mM | 6.0 | 220 mM | | 0.04 | 10 |
| | | | | sucrose | | | |
| 17 s | 138 | 10 mM | 6.0 | 220 mM | | 0.04 | 10 |
| | | | | trehalose | | | |
| 18_s | 138 | 10 mM | 6.0 | 120 mM | 50 mM | 0.04 | 10 |
| | | | | sucrose | Arg-HCl | | |

### Visual Inspection

It was observed that all formulations containing 28 mg/mL in syringe ref #1_s, 7_s, and 8_s, and all formulations containing 138 mg/mL of amlitelimab in syringe ref #11_s, 17_x, and 18_s remained visually clear and colorless after 12 weeks at 5° C., 25° C., and 40° C.

### Aggregation by SEC-HPLC

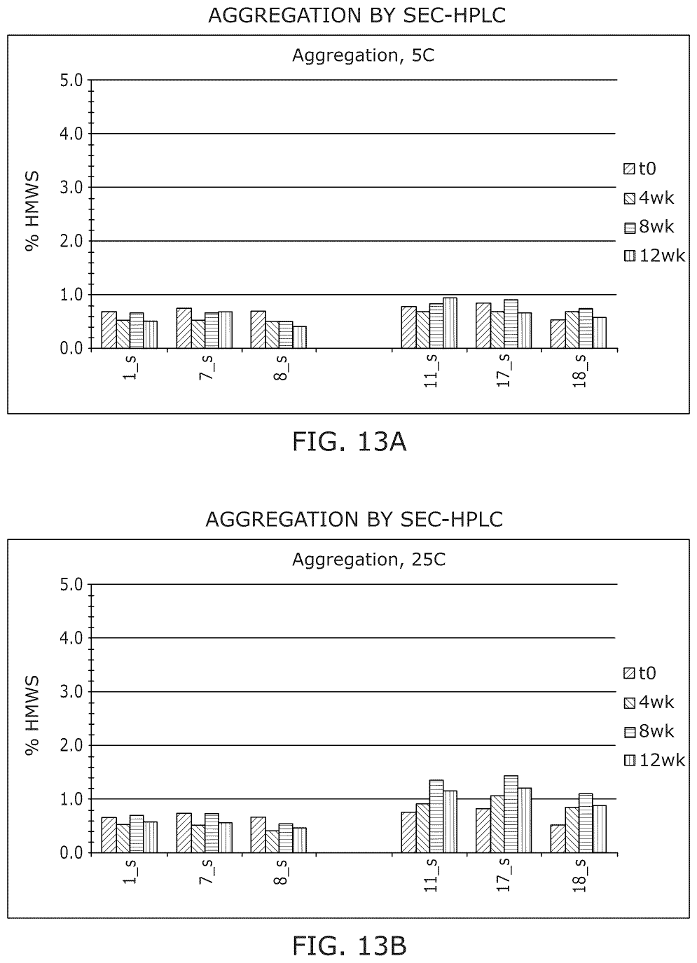

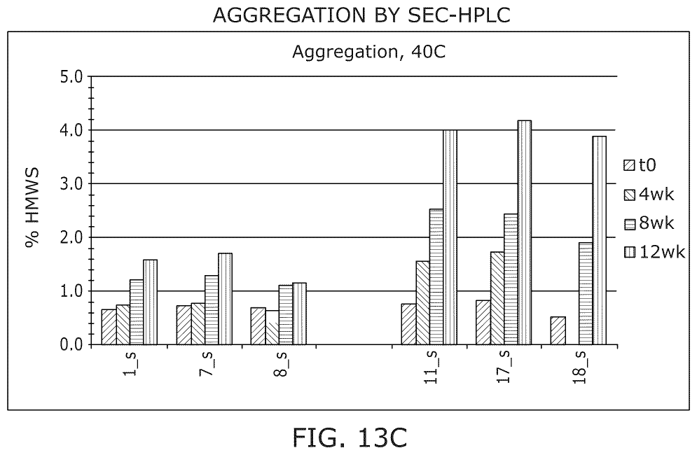

Aggregation of the formulations in Syringe Ref #1_s, 7_s, 8_s, 11_s, 17_x, and 18_s was evaluated by SEC-HPLC. FIGS. 13A, 13B, and 13C graphically depict HMWS evolution of the formulations contained in these syringes over 12 weeks of storage at 5° C. (FIG. 13A), 25° C. (FIG. 13B), and 40° C. (FIG. 13C). The results show that there was a general increase of HMWS % over 12 weeks of storage at 40° C., especially for high concentration samples, but there were no significant differences across the formulations.

### Subvisible Particles by HIAC

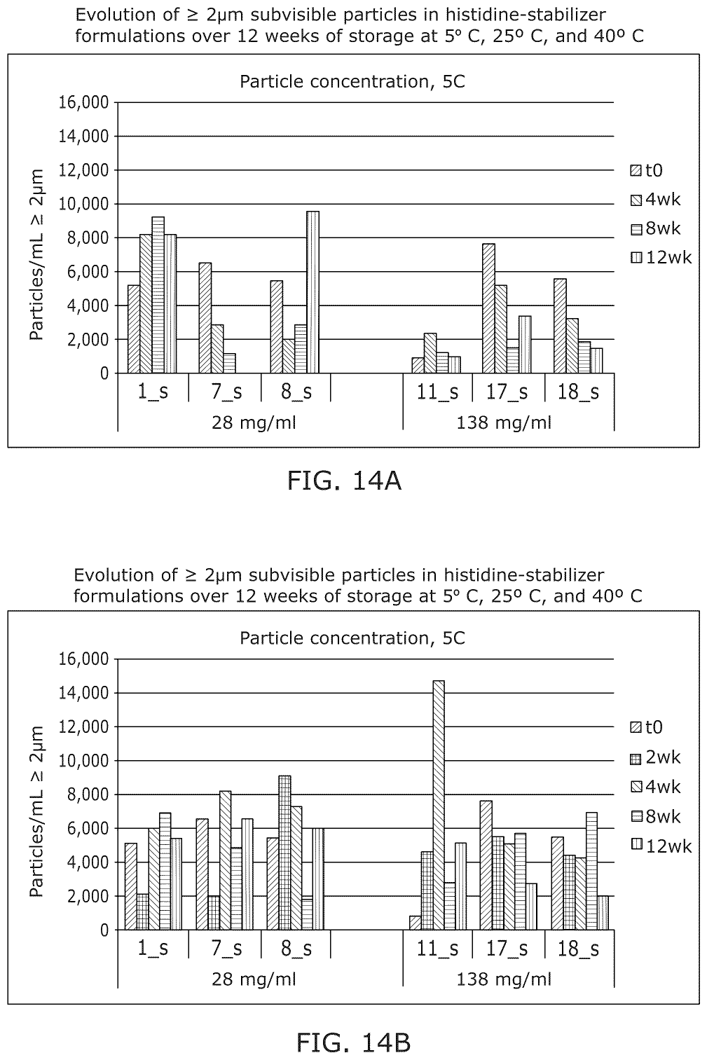

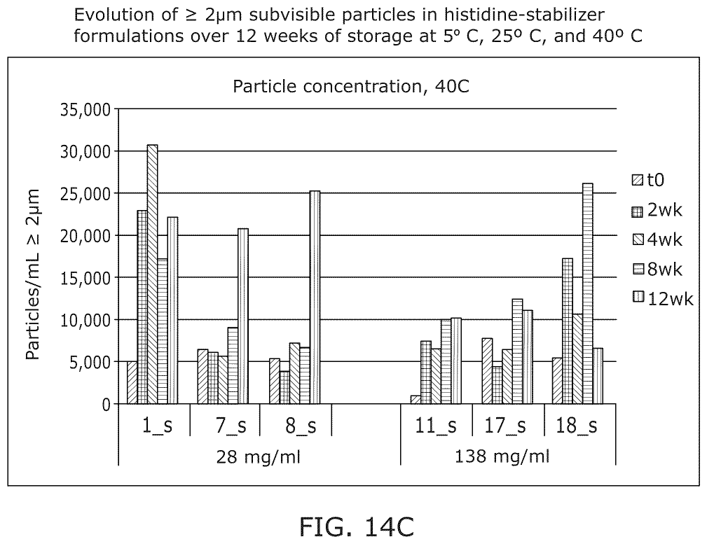

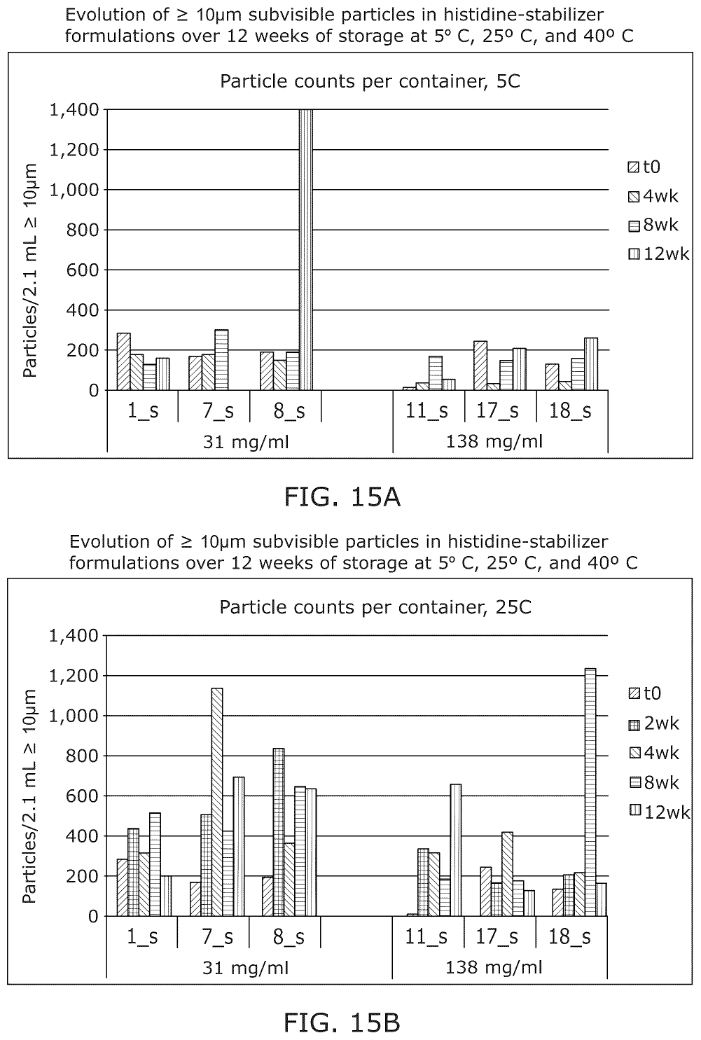

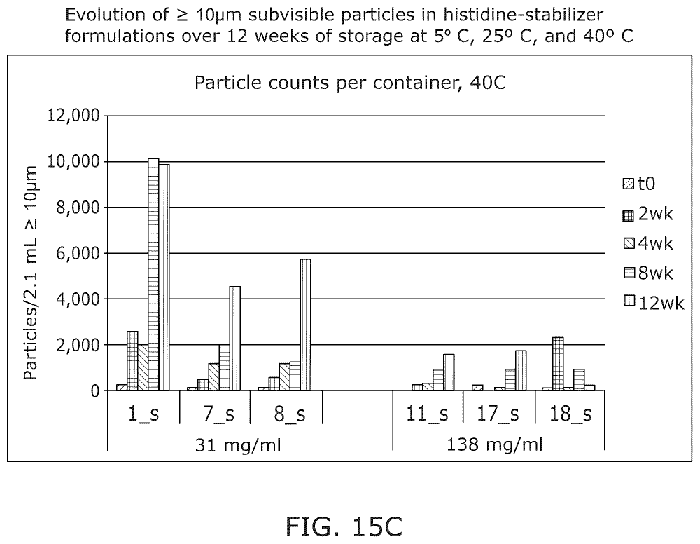

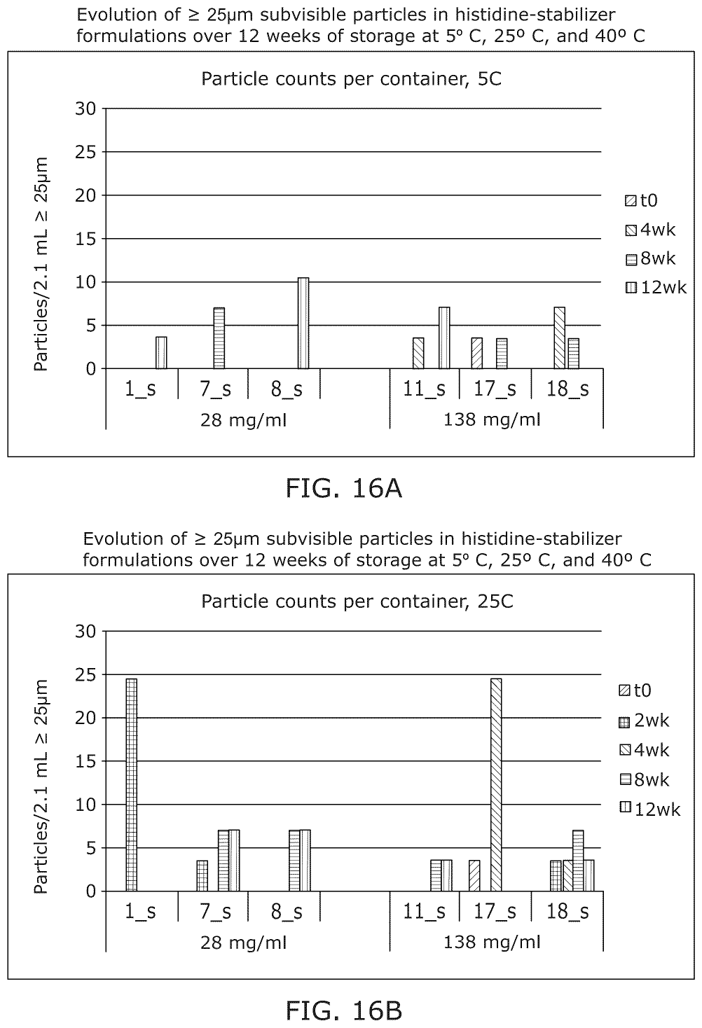

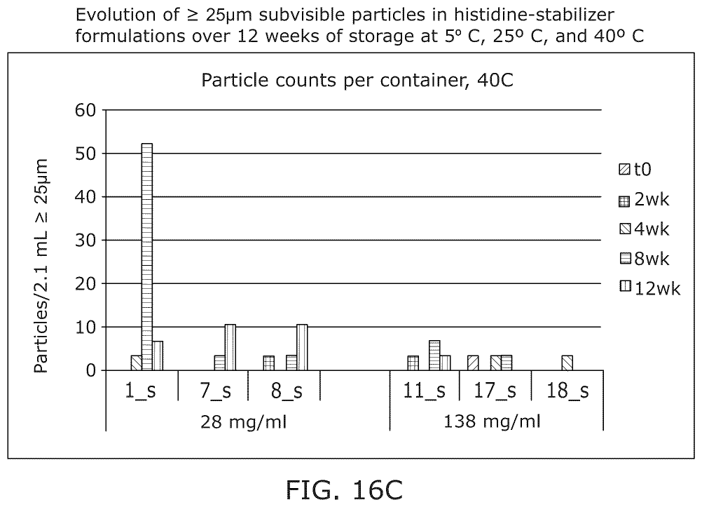

Subvisible particles in these example formulations in Syringe Ref #s, 7_s, 8_s, 11_s, 17_x, and 18_s were evaluated by HIAC. FIGS. 14A, 14B, and 14C graphically illustrate the evolution of ≥2 μm subvisible particles in example formulations over 12 weeks of storage at 5° C. (FIG. 14A), 25° C. (FIG. 14B), and 40° C. (FIG. 14C). FIGS. 15A, 15B, and 15C graphically depict the evolution of ≥10 μm subvisible particles in the example formulations over 12 weeks of storage at 5° C., 25° C., and 40° C., respectively. FIGS. 16A, 16B, and 16C graphically depict the evolution of ≥25 μm subvisible particles in example formulations over 12 weeks of storage at 5° C., 25° C., and 40° C., respectively.

It was observed that subvisible particle formation, at all particle sizes, was relatively prominent at the stressed temperature of 40° C. compared to 5° C. and 25° C. At 40° C. all formulations showed a general increase in particles over 12 weeks with the lower protein-concentration samples showing relatively larger particle generation than the high protein-concentration samples.

### Turbidity Analysis

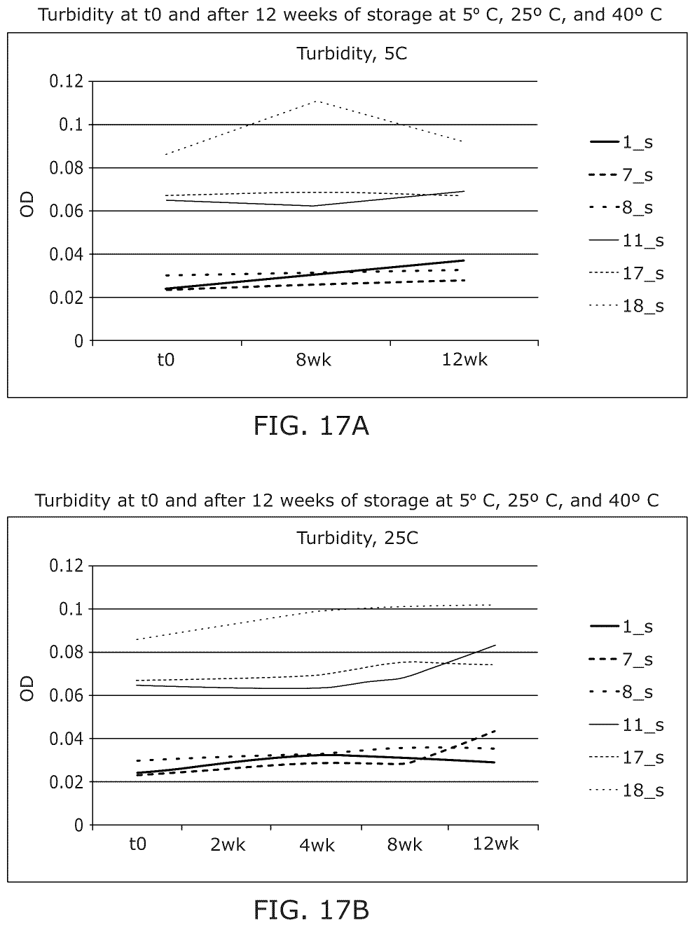

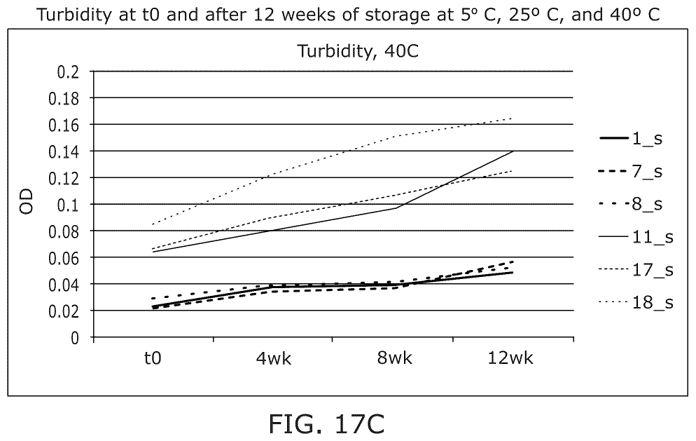

Turbidity analysis was performed to sample formulations stored in the Syringe Ref #s, 7_s, 8_s, 11_s, 17_x, and 18_s at t0 and after 2 weeks, 4 weeks, 8 weeks, and/or 12 weeks of storage at 5° C., 25° C., and 40° C. The results are shown in FIGS. 17A, 17B, and 17C, which respectively depict the results for storage at 5° C., 25° C., and 40° C.

The results show that for all stabilizer formulations, turbidity values were dependent on protein concentration and storage temperature. With respect to formulation trends the high protein concentration sample containing 120 mM sucrose and 50 mM arginine hydrochloride had the highest turbidity of the samples tested. All low protein-concentration formulations were comparable across all temperatures.

### PS80 Quantification

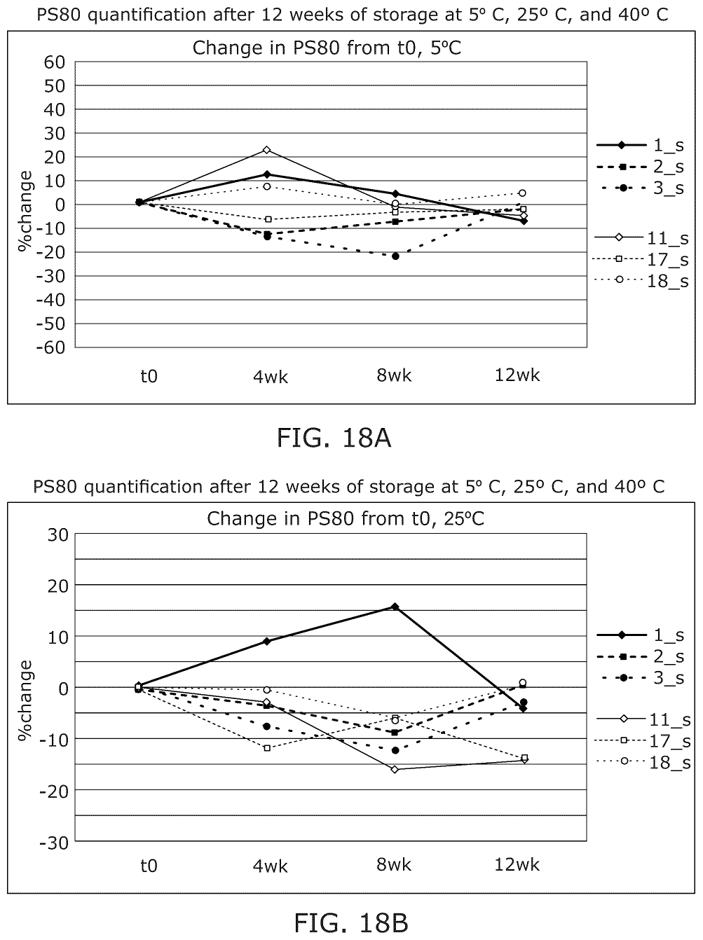

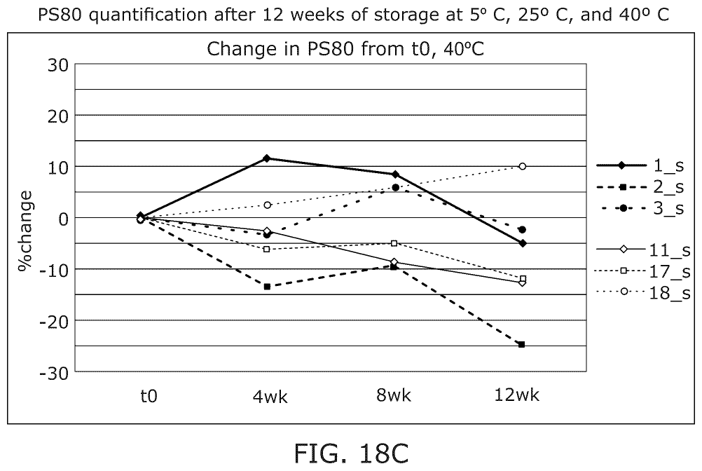

PS80 quantification of the formulations stored in the syringes at 5° C., 25° C., and 40° C. at T0 and after 12 weeks were analyzed. In this study, the method variability for PS80 analysis by CAD was about 25% (±100 ppm PS80). The results are graphically depicted in FIGS. 18A, 18B, and 18C. At 5° C., 25° C. and 40° C., no trend was observed for change in PS80 over time, and the percent change was within the variability of the analysis. There was no significant decrease in PS80 in these formulations after 12 weeks at all temperatures.

### Peptide Mapping

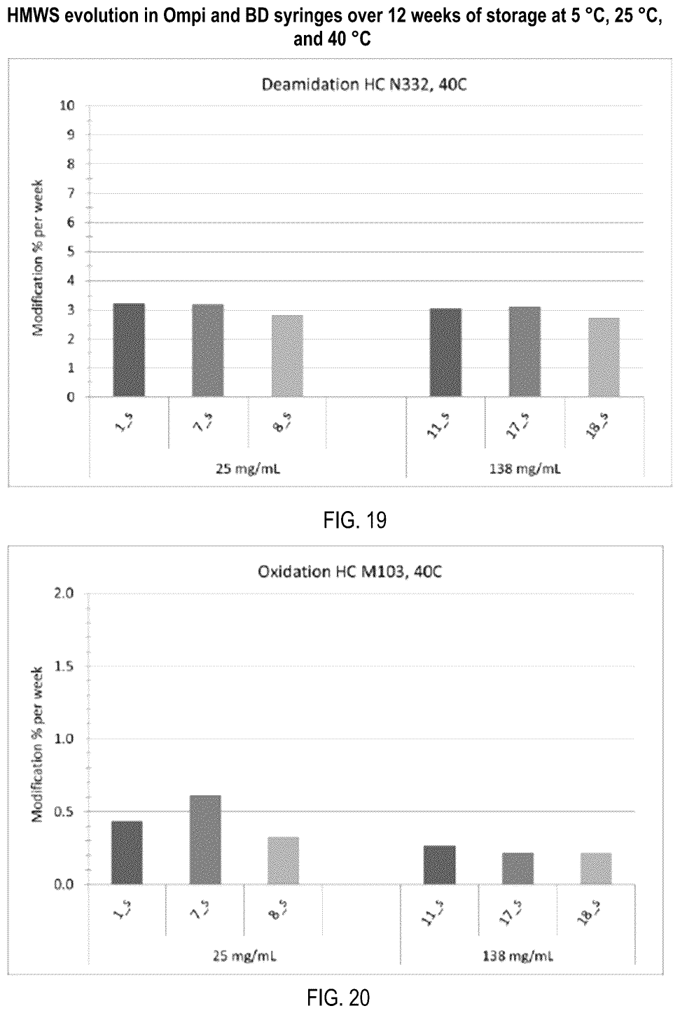

Modification was measured by peptide mapping after 3-month storage at 40° C. The percent change per week was calculated for deamidation of the antibody at HC N332 and oxidation at HC M103. The results are graphically depicted in FIG. 19 and FIG. 20, where FIG. 19 depicts the deamidation at HC N332 and FIG. 20 depicts the oxidation of the antibody at HC M103. The results showed that there was no significant difference, across formulations or protein concentrations, of change per week in deamidation.

### Influence of Stabilizers on Frozen Stability

Influence of stabilizers on frozen stability were evaluated in 6R Schott vials to solutions in Vial Ref Nos 11_v. 17_v, and 18_v, which comprise 138 mg/mL of mAb. Frozen stabilities from t0, after storage for up to 12 weeks at −30° C. and −80° C. were analyzed for HMWS %, turbidity, pH, concentration, subvisible particles (≥2 μm, ≥10 μM, ≥25 μm).

The results showed that frozen stability data was comparable for stabilizer samples stored at −30° C. and −80° C. No significant changes in aggregation, turbidity, pH, concentration, and particle formation have been observed for up to 12 weeks.

### Syringe Comparability Study

Study was performed to evaluate the effects of OMPI EZ fill syringes and BD Neopak syringes on the physical and chemical stability of amlitelimab aqueous formulations at low and high concentrations during refrigerated storage, accelerated storage, and stressed storage conditions.

### Sample Conditions

Pre-filled OMPI or BD syringes comprising aqueous formulations comprising 28 mg/mL or 138 mg/mL of amlitelimab, with 10% variability in the concentration, were prepared according to the ingredients and concentrations of the ingredients listed in Table 1-4.

TABLE 1-4
Formulation conditions and sample references

| Syringe | amlitelimab | Buffer | | | | % | μM | |
|---|---|---|---|---|---|---|---|---|
| Ref # | (Conc mg/ml) | (Histidine) | pH | Sucrose | Stabilizer | PS80 | EDTA | Syringe |
| 1_s_Omp | 28 | 10 mM | 6.0 | 220 mM | — | 0.04 | 10 | OMPI |
| 9_s_BD | 28 | 10 mM | 6.0 | 220 mM | — | 0.04 | 10 | BD |
| 10_s_BD | 138 | 10 mM | 6.0 | 220 mM | — | 0.04 | 10 | BD |
| 11_s_Omp | 138 | 10 mM | 6.0 | 220 mM | — | 0.04 | 10 | OMPI |

### Visual Inspection

It was observed that all formulations containing 28 mg/mL and all formulations containing 138 mg/mL of amlitelimab in both OMPI and BD syringes remained visually clear and colorless after 12 weeks at 5° C., 25° C., and 40° C. There were no visual differences between OMPI and BD syringes.

### Aggregation by SEC-HPLC

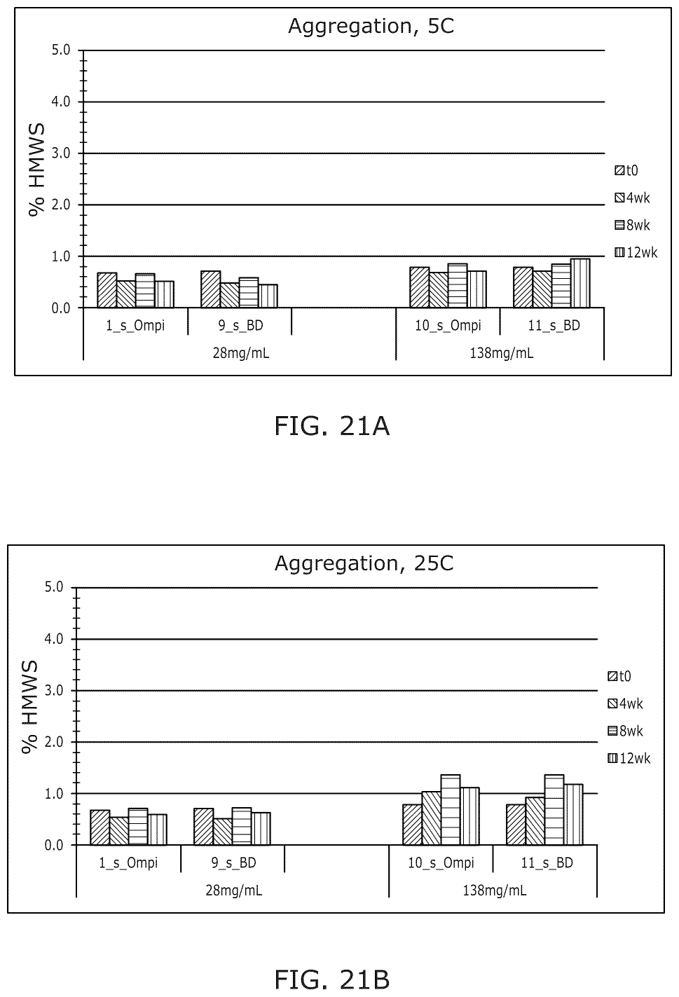

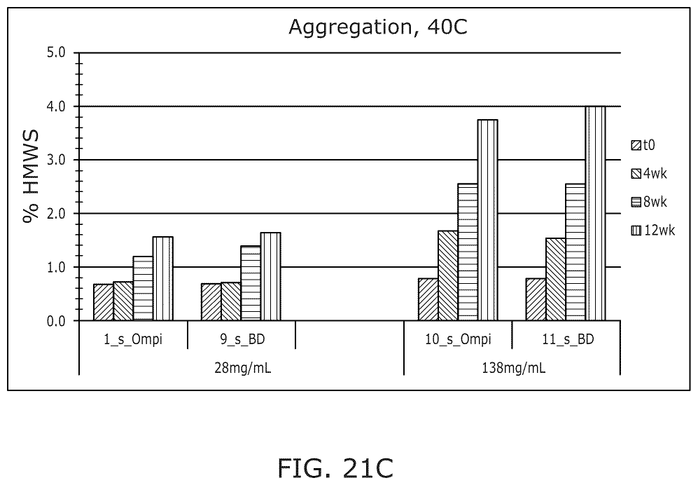

Aggregation of the formulations in both OMPI and BD Syringes was evaluated by SEC-HPLC. FIGS. 21A, 21B, and 21C graphically depict HMWS evolution of the formulations in the syringes 1_s_Omp, 9_s_BD, 10_s_BD, and 11_s_Omp over 12 weeks of storage at 5° C. (FIG. 21A), 25° C. (FIG. 21B), and 40° C. (FIG. 21C). The results show that HMWS levels of each formulation in an OMPI or BD syringe were nearly unchanged up to 12 weeks of storage at 5° C. and 25° C., with a moderate increase over 12 weeks of storage at 40° C. There was no significant difference in HMWS between samples stored in OMPI and BD syringes.

### Subvisible Particles by HIAC

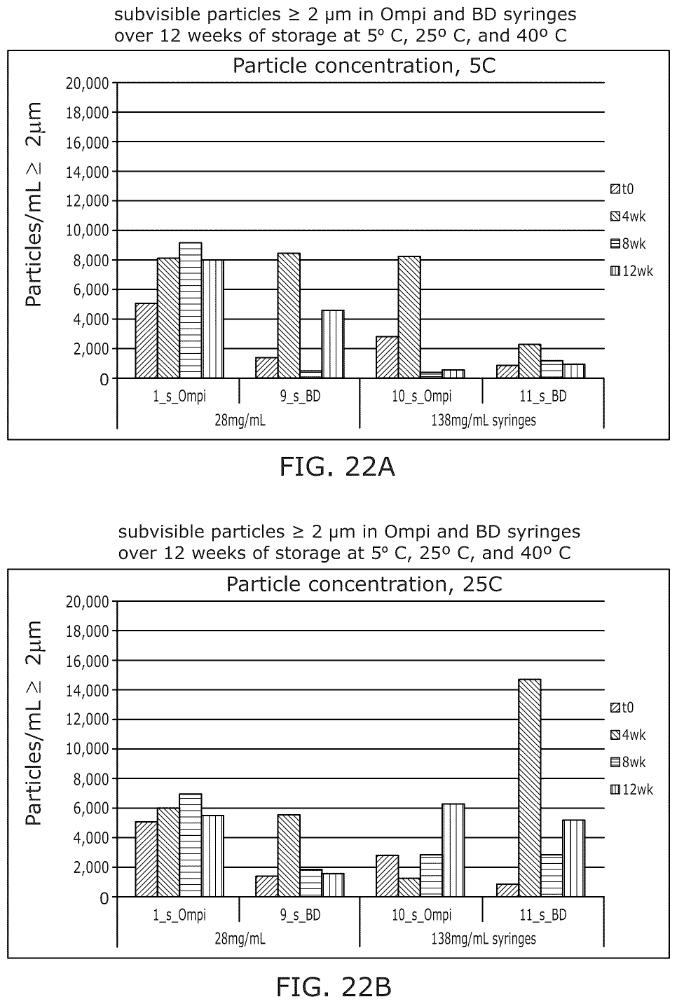

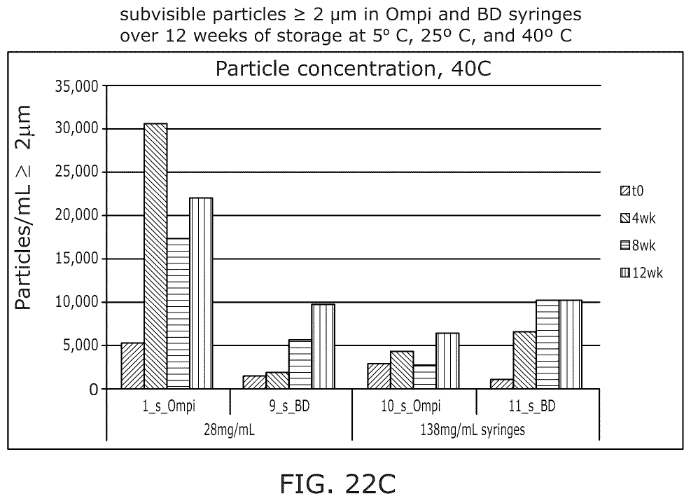

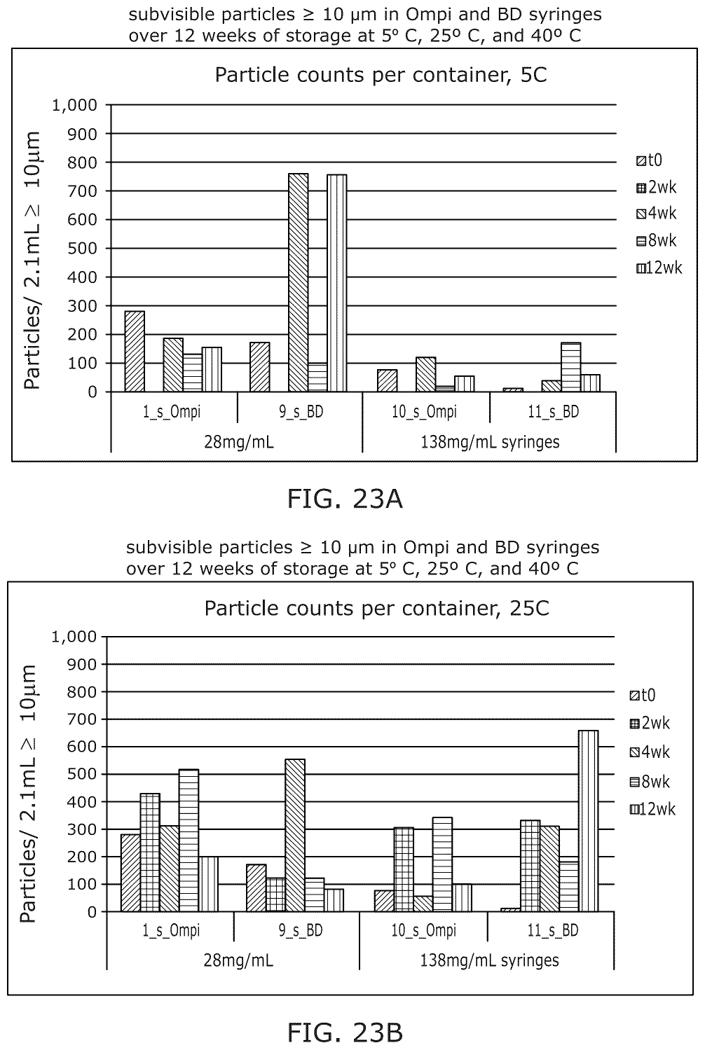

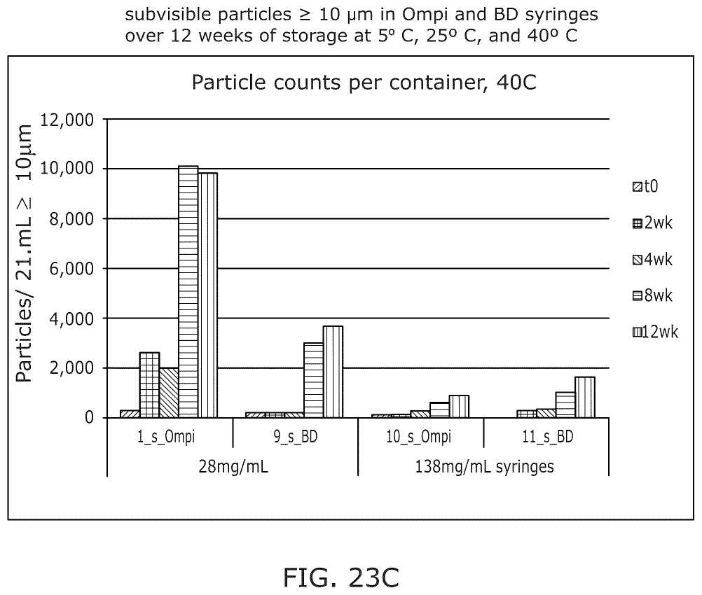

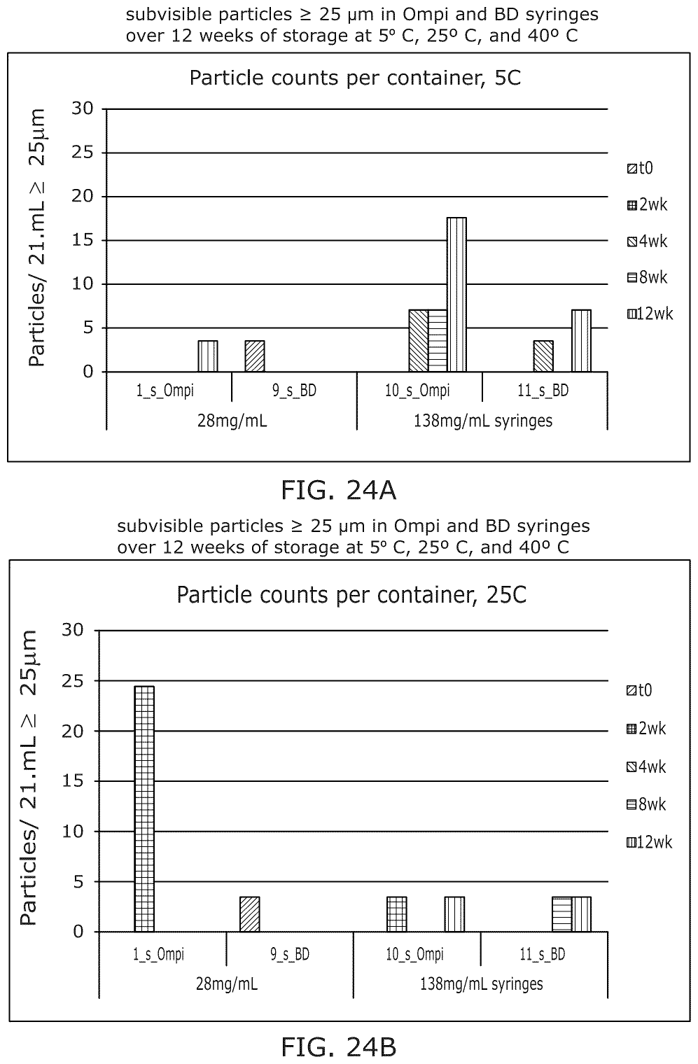

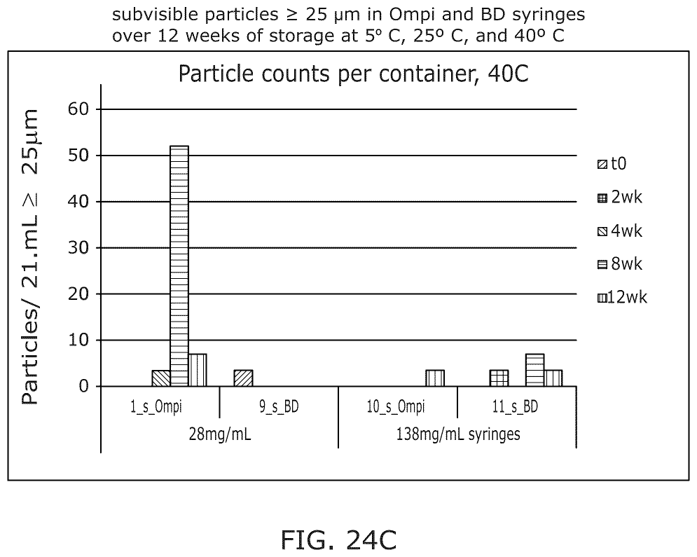

Subvisible particles in these example formulations in both OMPI and BD syringes were evaluated by HIAC. FIGS. 22A, 22B, and 22C graphically depict the concentration of subvisible particles of ≥2 μm in OMPI and BD syringes over 12 weeks of storage at 5° C., 25° C., and 40° C., respectively. FIGS. 23A, 23B, and 23C graphically depict the concentration of subvisible particles of ≥10 μm subvisible particles in example formulations over 12 weeks of storage at 5° C., 25° C., and 40° C., respectively. FIGS. 24A, 24B, and 24C graphically depict the concentration of subvisible particles of ≥25 μm subvisible particles in example formulations over 12 weeks of storage at 5° C., 25° C., and 40° C., respectively.

The results show that at 5° C. and 25° C., the counts of subvisible particles at all particle sizes were elevated in OMPI as compared to BD syringes for both protein concentrations. Only under stressed conditions of 40° C. were excessive levels observed in low-concentration samples in OMPI syringes.

### Turbidity Analysis

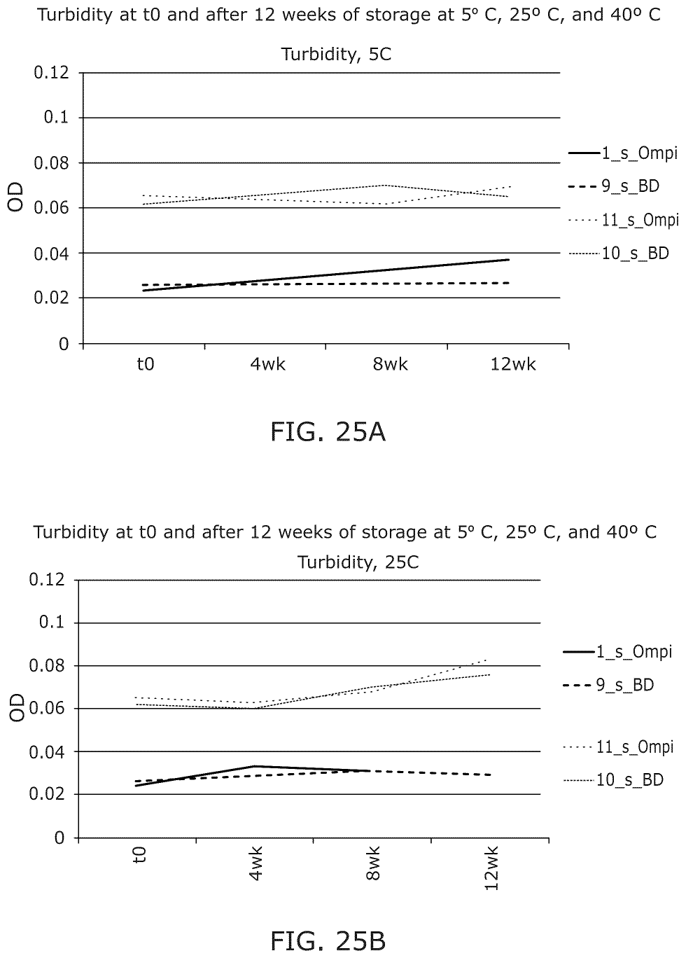

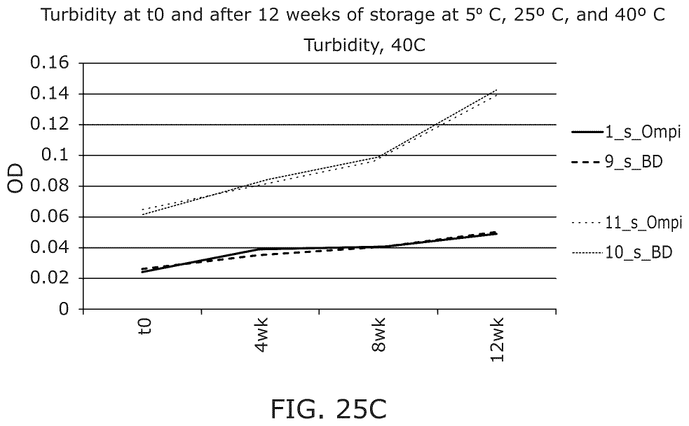

Turbidity analysis was performed to sample formulations at t0 and after 12 weeks of storage in the syringes at 5° C., 25° C., and 40° C., where the results are respectively graphically depicted in FIGS. 25A, 25B, and 25C. As shown in FIGS. 25A, 25B, and 25C, high protein-concentration samples exhibited higher OD values relative to low protein-concentration samples, with comparable levels for formulations in both syringe types.

### PS80 Quantification

PS80 quantification of the formulations stored in the syringes at 5° C., 25° C., and 40° C. for over 12 weeks were analyzed. In this study, the method variability for PS80 analysis by CAD was about 25% (±100 ppm PS80). At 5° C., 25° C. and 40° C., no trend was observed for change in PS80 over time, and the percent change was within the variability of the analysis. There was no significant decrease in PS80 in these formulations after 12 weeks at all temperatures.

### Charge Variants

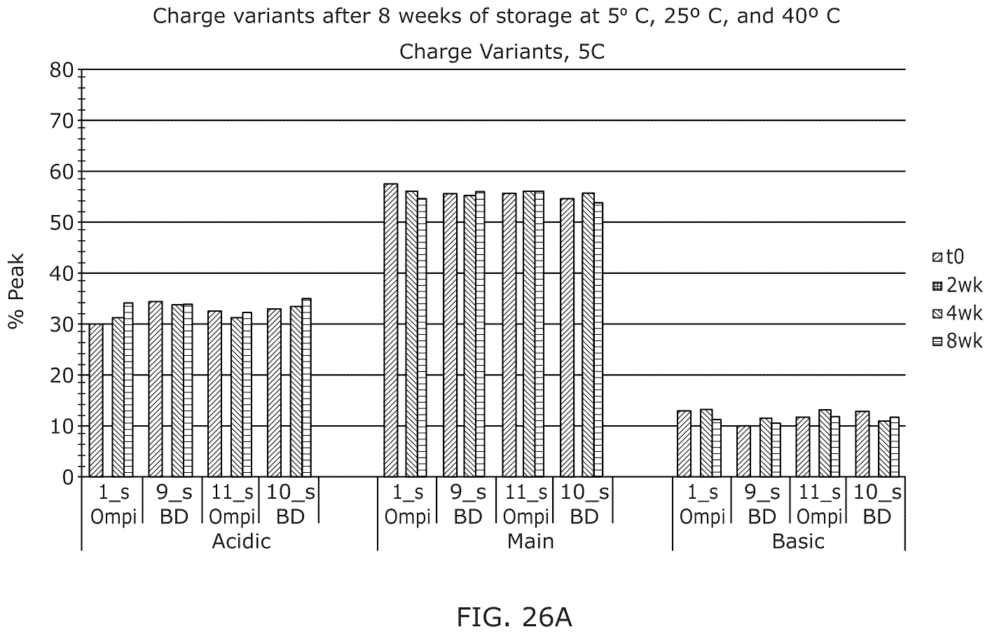

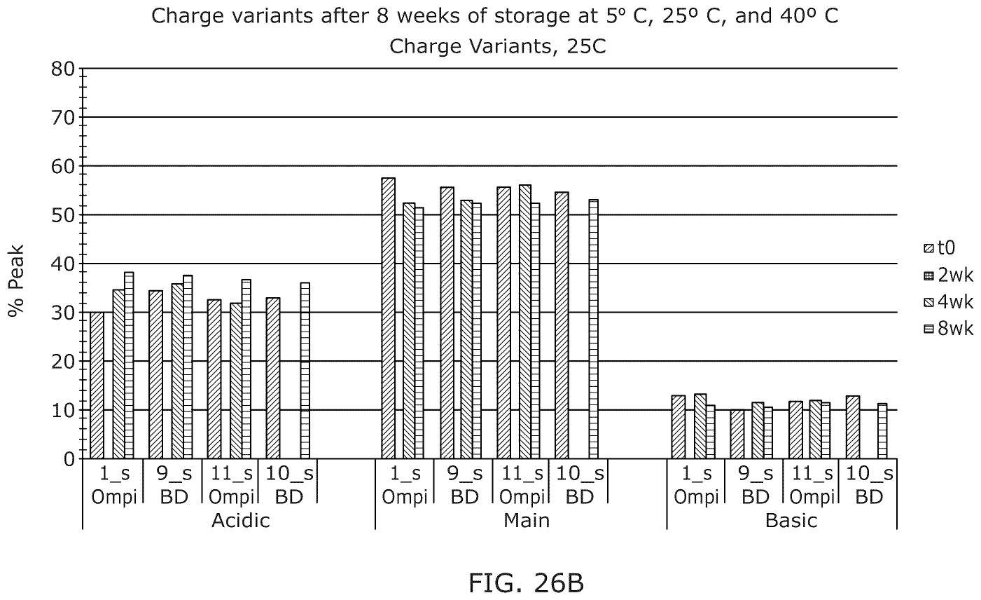

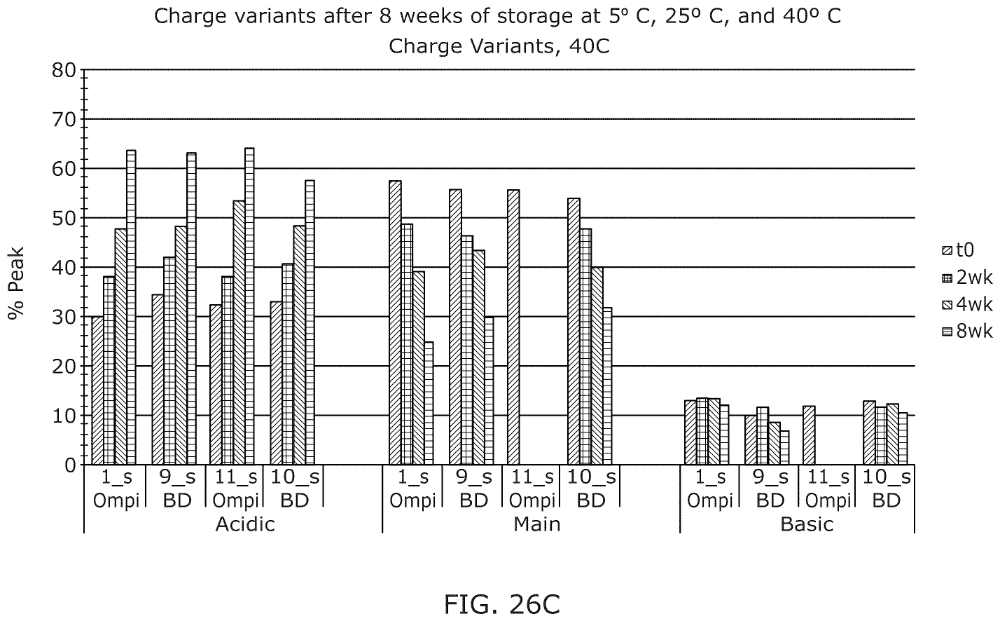

Charge variants were analyzed for the formulations in both OMPI and BD syringes stored over 8 weeks at 5° C., 25° C., and 40° C. The results are respectively graphically depicted in FIGS. 26A, 26B, and 26C. The results show that at 5° C. and 25° C. (FIG. 26A and FIG. 26B) the formulations in both syringes had comparable variants for each timepoint. At 40° C. (FIG. 26C), OMPI and BD syringes at both protein concentrations, had an increase in acidic species and a decrease in the main peak, over 8 weeks, while the basic species remained relatively constant.

### Peptide Mapping

Modification was measured by peptide mapping after 3-month storage at 40° C. The percent change per week was calculated for deamidation of the antibody in the example formulations at HC N332 and oxidation of the antibody in the example formulations at HC M103. No significant difference, across formulations or protein concentrations, of change per week in deamidation was observed (all were about 3% modification/per week). No significant difference was observed across formulations with the same concentration of the antibody in OMPI or BD syringes, while higher percentage of oxidation at HC M103 was observed in the formulations with the lower concentration of antibody (i.e., 28 mg/mL), compared to the formulations with 138 mg/mL antibody. The HC M103 oxidation % per week was about 0.35 to 0.43 in the formulations with 28 mg/mL of antibody, and about 0.23-0.26 in the formulations with 138 mg/mL antibody).

### Chelator Justification Study

This study was performed to examine the impact of transition metals, which are sometimes leached into the DS during manufacturing, on the chemical and physical stability of the protein. There is a low risk of transition metal contamination in the DS and DP during the current manufacturing processes for amlitelimab. If there is contamination, however, these transition metals can lead to chemical instability and aggregation in the liquid solution. Metal chelators can protect the protein's chemical stability under metal exposure. EDTA and DTPA were evaluated for their ability to protect the protein during a set of worst-case experiments.

### Study Design

TABLE 1-5
Formulation conditions and sample references

| Ref | | Base | EDTA | DTPA | Fe | Cu | W |
|---|---|---|---|---|---|---|---|
| No. | | formulation | (μM) | (μM) | (ppb) | (ppb) | (ppb) |
| C_1 | No | 125 mg/mL | — | — | — | — | — |
| | chelator | protein, 10 | | | | | |
| C_2 | No | mM Histidine, | — | — | 50 | 50 | 50 |
| | chelator + | 220 mM | | | | | |
| | ions | sucrose, 0.04% | | | | | |
| C_3 | 10 μM | PS80 | 10 | — | — | — | — |
| | EDTA | | | | | | |
| C_4 | 10 μM | | 10 | — | 50 | 50 | 50 |
| | EDTA + | | | | | | |
| | ions | | | | | | |
| C_5 | 10 μM | | — | 10 | — | — | — |
| | DTPA | | | | | | |
| C_6 | 10 μM | | — | 10 | 50 | 50 | 50 |
| | DTPA + | | | | | | |
| | ions | | | | | | |

* Fe: iron;
Cu: copper;
W: Tungsten.

### Visual Inspection

No significant visual difference in turbidity or color was observed, comparing formulations over time, for all three temperatures.

### Aggregation by SEC-HPLC

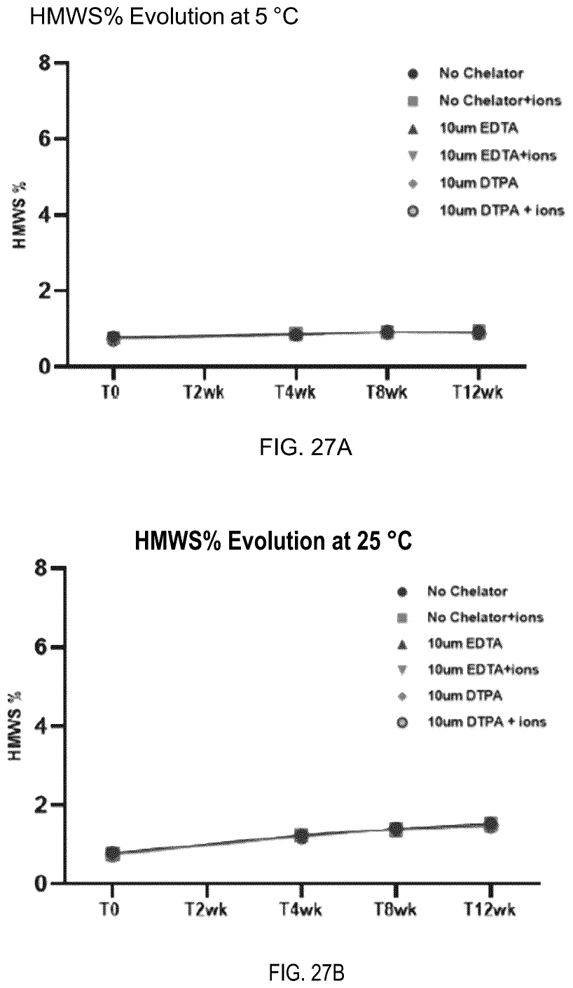

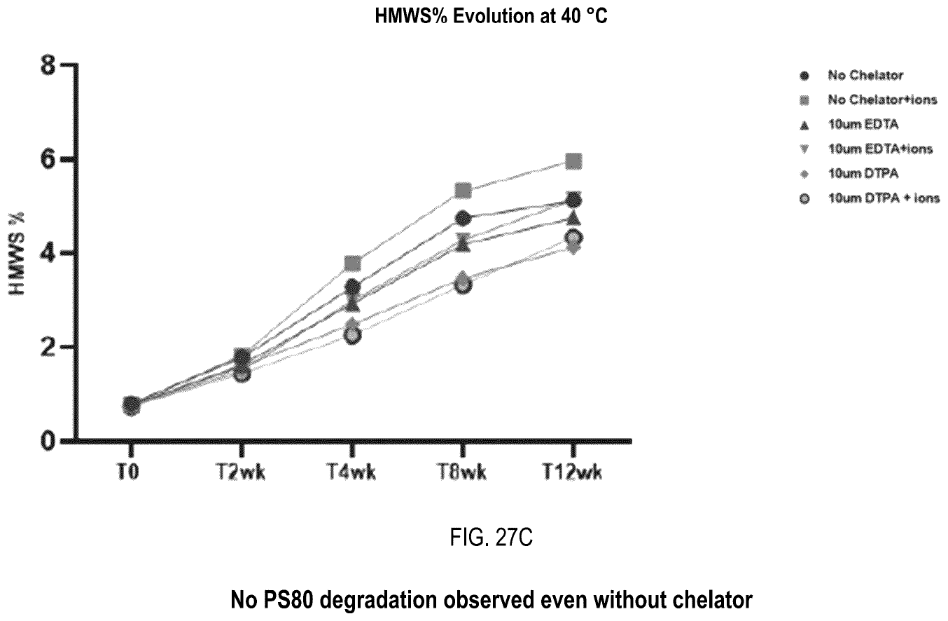

Aggregation of the formulations was evaluated by analyzing evolution of the sample formulations in the syringes with Ref No. C-1 to C_6 over 12 weeks of storage at 5° C., 25° C., and 40° C. via SEC-HPLC. FIGS. 27A, 27B, and 27C graphically depicts the evolution of HMWS % at 5° C. (FIG. 27A), 25° C. (FIG. 27B), and 40° C. (FIG. 27C).

It was found that at 5° C. and 25° C. (FIGS. 27A and 27B), no significant differences in HMWS % among the formulations C-1 to C_6 for up to 12 weeks, while a minor increase over time of HMWS % at 25° C. was shown in all of the formulations. As can be seen in FIG. 27C, there was a general increase of HMWS over 12 weeks of storage at 40° C., where the greatest increase of HMWS was observed for formulations without chelator and with ions at T12 wk. 10 μM DTPA formulations show marginally lower HMWS at all timepoints when compared with 10 μM EDTA formulations. In the presence of chelator (EDTA or DTPA), ions did not significantly impact aggregation. FIG. 27C further depicts the comparison of the changes of HMWS in the sample formulations over 2 months of storage at 40° C. As shown in FIG. 27C, during the storage at 40° C., about 1-2% decrease in HMWS % is observed when a chelator is present in the formulation.

### PS80 Quantification

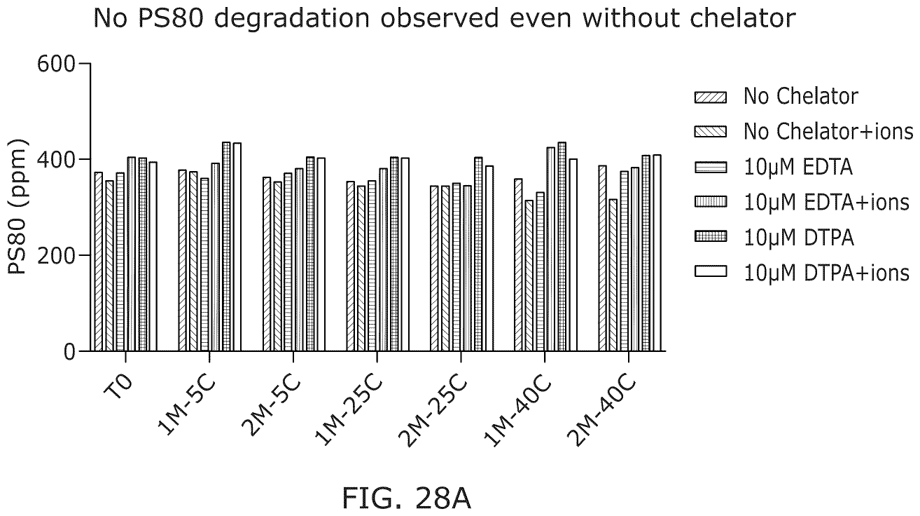

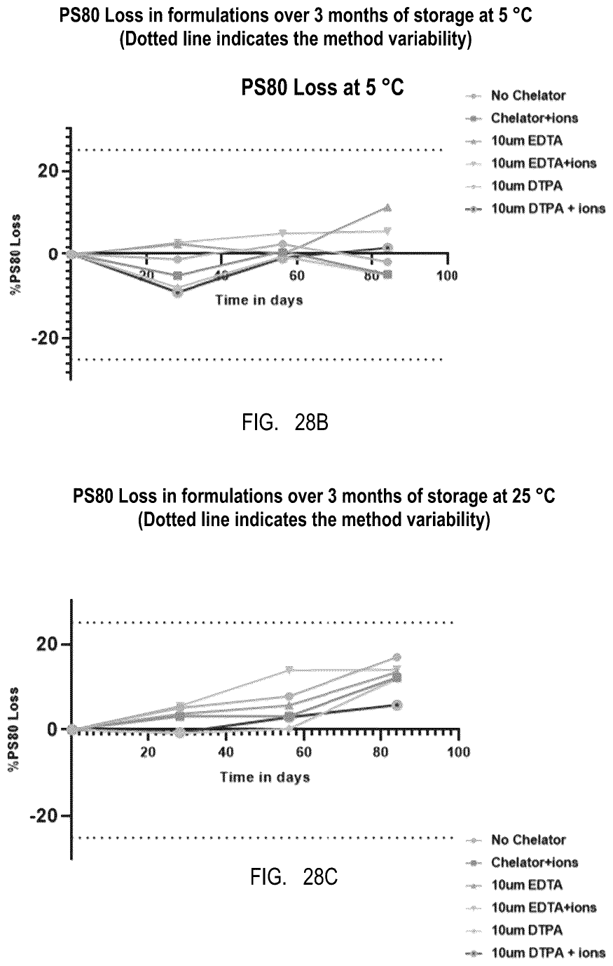

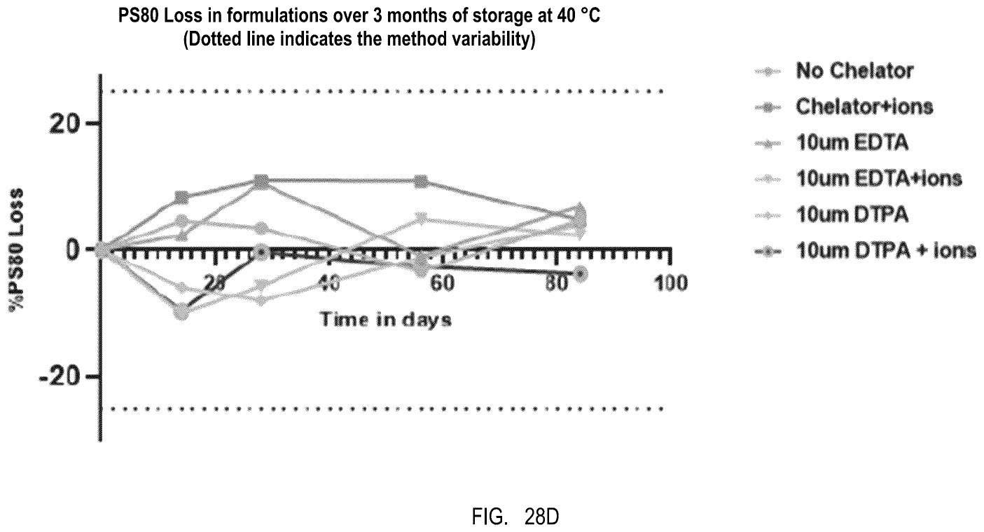

PS80 quantification of the formulations of Ref. Nos. C-1 to C_6 were analyzed. In this study, the method variability for PS80 analysis by CAD is about 25% (±100 ppm PS80). Obtained results are illustrated in FIGS. 28A, 28B, 28C, and 28D, where FIG. 28A depicts PS80 (ppm) in the formulations over 2-month of storage at 5° C., 25° C., and 40° C., and FIGS. 28B, 28C, and 28D depict the change of PS80 in the formulations of Ref. Nos. C_1 to C_6 over 3 months of storage at 5° C. (FIG. 28B), 25° C. (FIG. 28C), and 40° C. (FIG. 28D) (Dotted line indicates the method variability).

As can be seen in FIG. 28 A, there was no significant decrease in PS80 in these formulations after 2 months of storage at all temperatures. As can be seen in FIGS. 28B and 28D, at 5° C. and 40° C., no trend was observed for changes in PS80 over time, and the percent change was within the variability of the analysis. At 25° C. (FIG. 28C), there appeared to be a gentle increased trend over time but the changes are at levels within the method variability. The highest PS80 loss was observed in formulations without chelator, and the lowest reduction in PS80 was observed in formulations with 10 μM DTPA with ions. It was observed that there was no significant decrease in PS80 in these formulations after T12 wk at all temperatures.

### Peptide Mapping

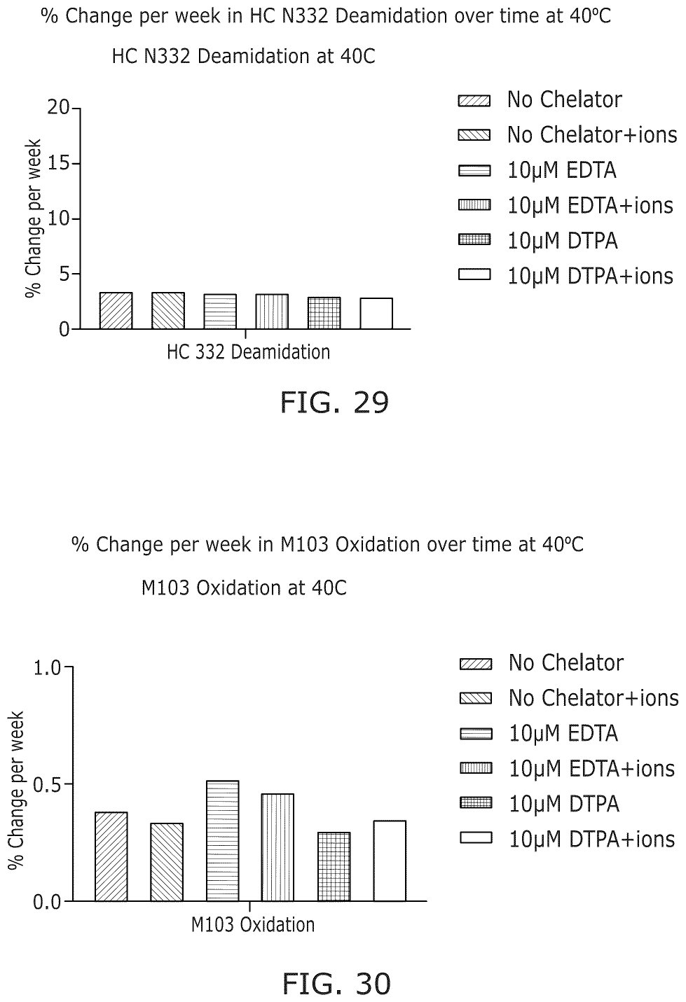

M103 Oxidation and HC N332 deamidation was monitored by peptide mapping for all formulations stored in the syringes for up to 8 weeks at 40° C. The percent change per week was calculated for deamidation at HC N332 (shown in FIG. 29) and oxidation at HC M103 (shown in FIG. 30). The results showed that there was no significant difference across these formulations, whether or not a chelator is present or not. The HC N332 deamidation measurements for the antibody in all example formulations were about 2.5 to about 3.5, and the HC M103 oxidation was about 0.25 to about 0.5 in all example formulations. There was minimal impact of chelators on deamination and oxidation.

### Charge Variants

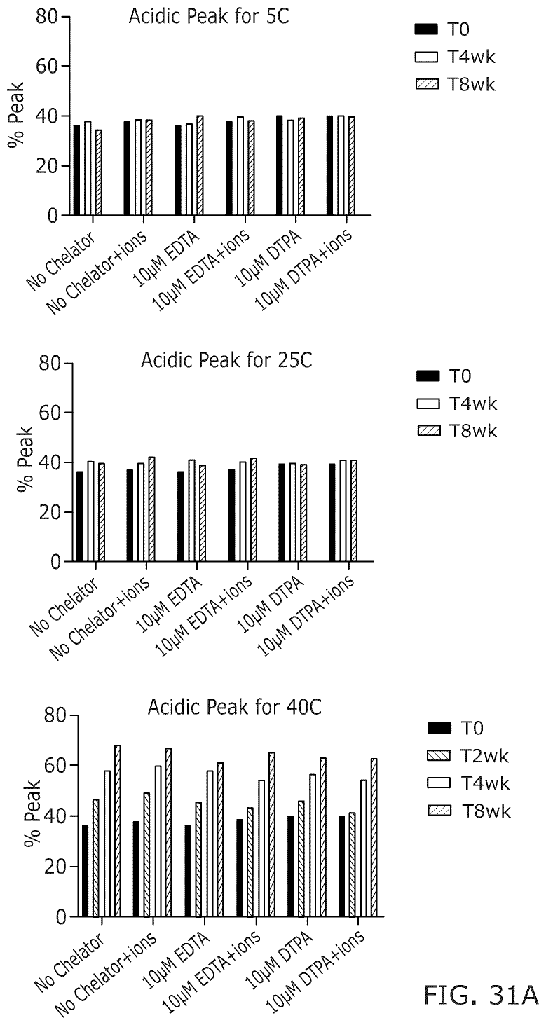

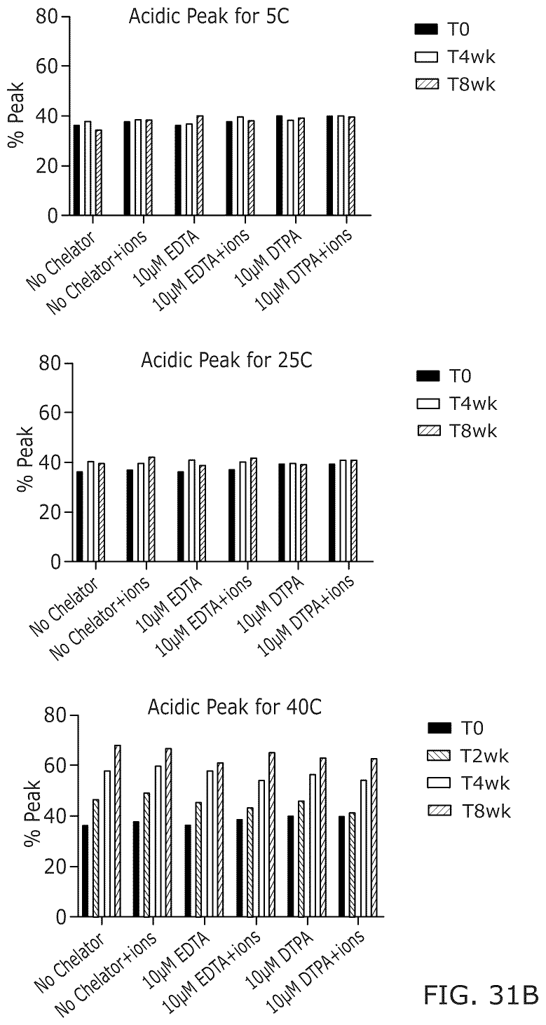

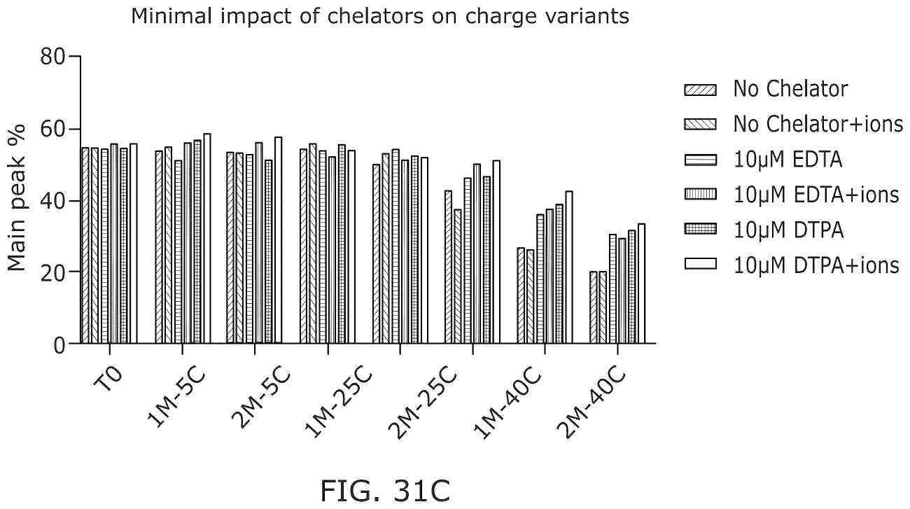

Charge variants were analyzed at T0, T2 wk (two weeks), T4 wk (4 weeks), and T8 wk (8 weeks) by CIEF for formulations of Ref. Nos. C-1 to C_6 stored over 8 weeks at 5° C., 25° C., and 40° C. The results are illustrated in FIGS. 31A, 31B, and 31 C, where acidic peak is illustrated in FIG. 31A, the basic peak is illustrated in FIG. 31B, and the main peak is illustrated in FIG. 31C. The results show that all formulations had comparable variants for each timepoint tested. At 40° C., most formulations tested had an increase in acidic species and a decrease in the main peak, over 8 weeks, while the basic species remained constant. It is observed that the impact of chelators on charge variants is minimal.

Overall, based on this study, 10 μM EDTA or DTPA is recommended to be included in the formulation to provide mitigation against potential impurity excursions.

### Polysorbate 80 Justification

This study was performed to determine the optimal concentrations of PS80 needed to stabilize amlitelimab aqueous formulations upon agitation-induced stress.

Formulations F1-1 to F1-12 varying in PS 80 content as listed in Table 1-6 were prepared. Various attributes of these formulations were analyzed at room temperature. The study comprised three arms as follows. Wrist-action: Samples were put on a shaker for 1 h, 3 h and 6 h. Orbital-shaking: Samples were put on orbital shaker with a speed of 300 rpm for 1 h, 6 h and 3 days. Wrist-action+silicone oil: Samples spiked with silicone oil were put on a wrist-action shaker for 1 h, 3 h and 6 h. Schott 6R vials were used for this study.

TABLE 1-6
Formulation conditions and sample references

| Formulation | % | protein | | Agitation |
|---|---|---|---|---|
| Number | PS80 | conc. | Buffer | method/Timepoint |
| F1-1 | 0.0 | 31 mg/mL | 10 mM Histidine, | Wrist action/T0, |
| F1-2 | 0.02 | | 220 mM Sucrose, | T1 hr, T3 hr, T6 hr |
| F1-3 | 0.04 | | 10 μM EDTA, | *Orbital Shaking at |
| F1-4 | 0.06 | | pH 6.0 | 300 rpm/T0, T1 hr, |
| F1-5 | 0.08 | | | T6 hr, t3 day |
| F1-6 | 0.1 | | | *Wrist action + |
| F1-7 | 0.14 | | | Silicon oil/T0, |
| F1-8 | 0.005 | 125 mg/mL | | T1 hr, T3 hr, T6 hr |
| F1-9 | 0.025 | | | |
| F1-10 | 0.045 | | | |
| F1-11 | 0.065 | | | |
| F1-12 | 0.105 | | | |

*Orbital-shaking and Wrist-action + silicone oil was not performed for F1-1 and F1-2 due to operational reasons.

### Wrist-Action Study

### SEC-HPLC for HMWS

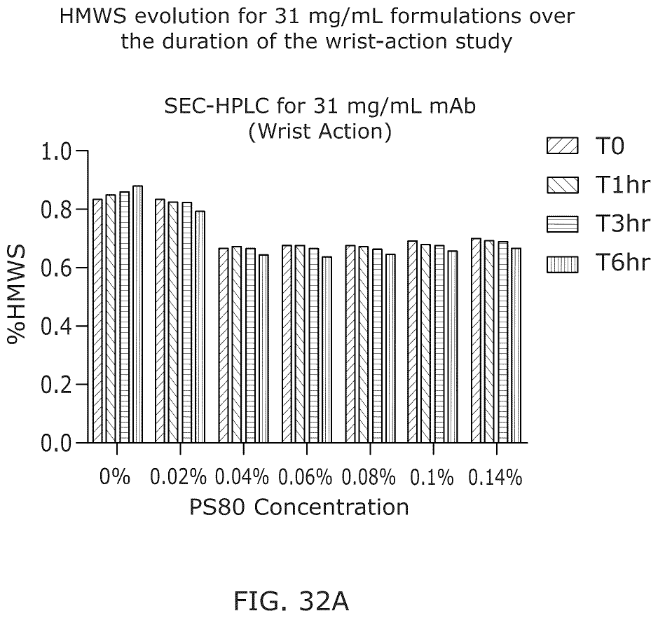

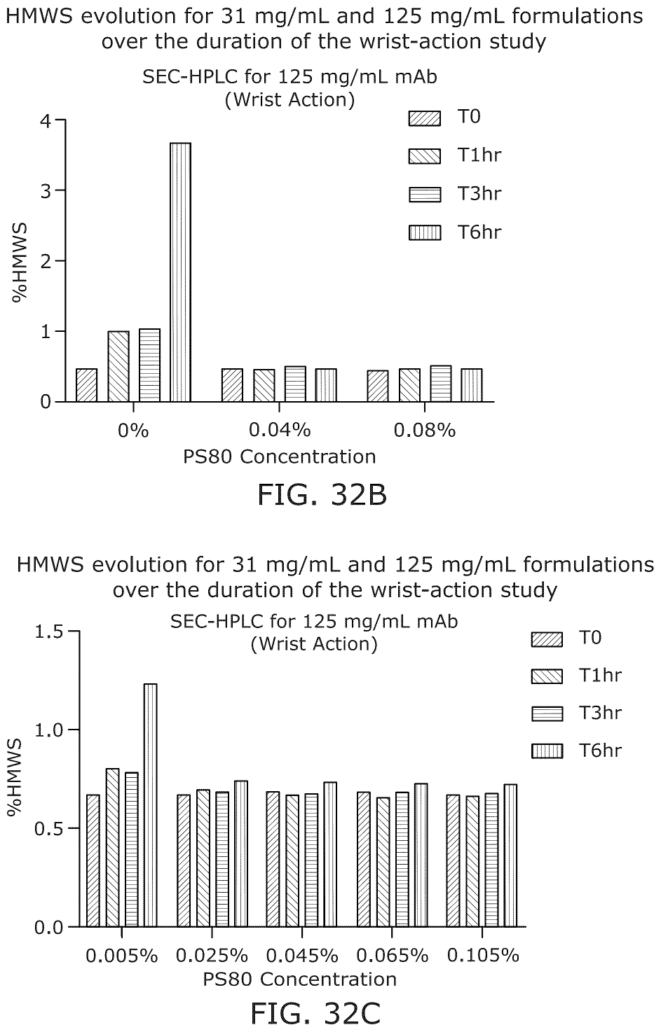

All formulations subjected to wrist-action were analyzed by SEC-HPLC for HMWS evolution at timepoints of T0, T1 hr, T3 hr, and T6 hr. FIGS. 32 A, 32B, and 32C are graphs depicting the measurements of HMWS evolution for formulations comprising 31 mg/mL and 125 mg/mL antibody over the duration of the wrist-action study.

The results shown that generally, HMW % increases with decrease of PS80% during the wrist-action. However, no substantial increase in HMWS during agitation was observed for the 31 mg/mL concentration formulations with all PS80 concentrations. HMWS levels were marginally higher for 31 mg/mL formulations with 0% and 0.02% PS80 as compared to the 31 mg/mL formulations with PS80 0.04% to 0.14%.

There was a noticeable increase in HMWS during the agitation for the 125 mg/mL formulation at 0.005% PS80 concentration, which was not observed in the rest of 125 mg/ml formulations. There was minimal change on HMW when PS80 is present in a concentration of at least 0.02%. As such, a minimum of 0.02% of PS80 concentration appears necessary to protect against aggregation.

### Turbidity Analysis

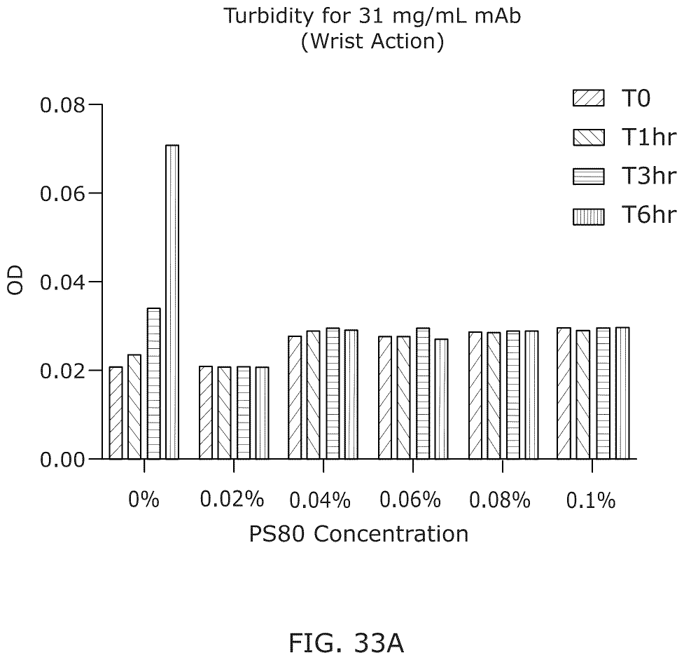

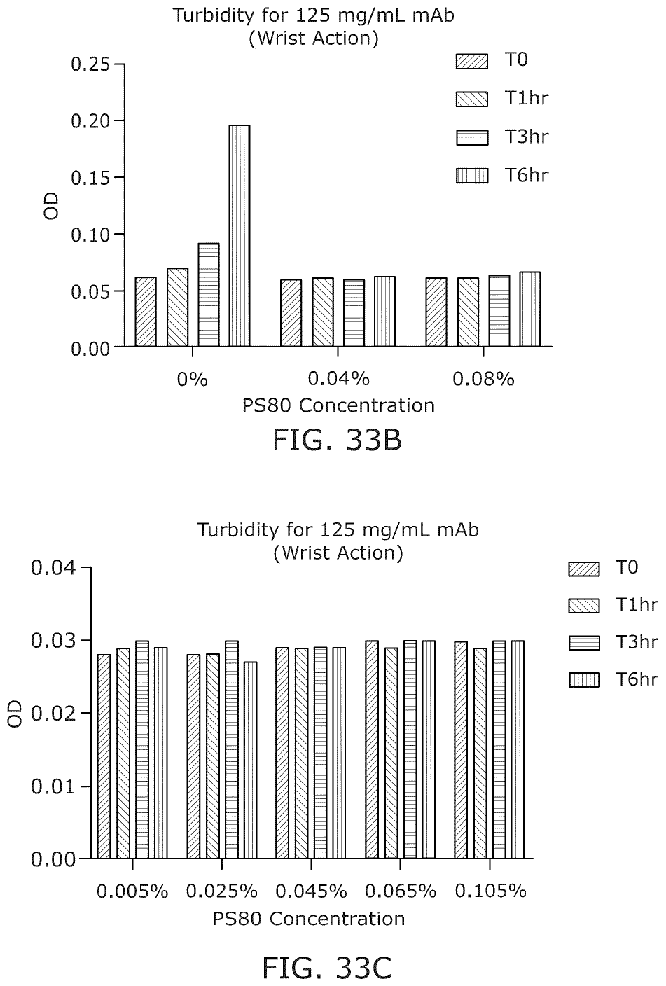

Turbidity analysis was performed to example formulations with 31 mg/mL and 125 mg/mL antibody at timepoints of T0, T1 hr, T3 hr, and T6 hr duration of the wrist-action study. Obtained results were illustrated in FIGS. 33 A, 33B, and 33C.

As can be seen from FIGS. 33 A, 33B, and 33C, turbidity increased significantly for the 31 mg/mL formulation in the absence of PS80. There was no significant increase in turbidity for the 31 mg/mL formulations with PS80 concentrations from 0.02% to 0.14%. There was no significant increase in turbidity for the 125 mg/mL concentration formulations for all PS80 concentrations. According to the turbidity data for the wrist-action study, a minimum of 0.02% of PS80 concentration is necessary to minimize the increase in turbidity when exposed to agitation.

### HIAC for Subvisible Particles

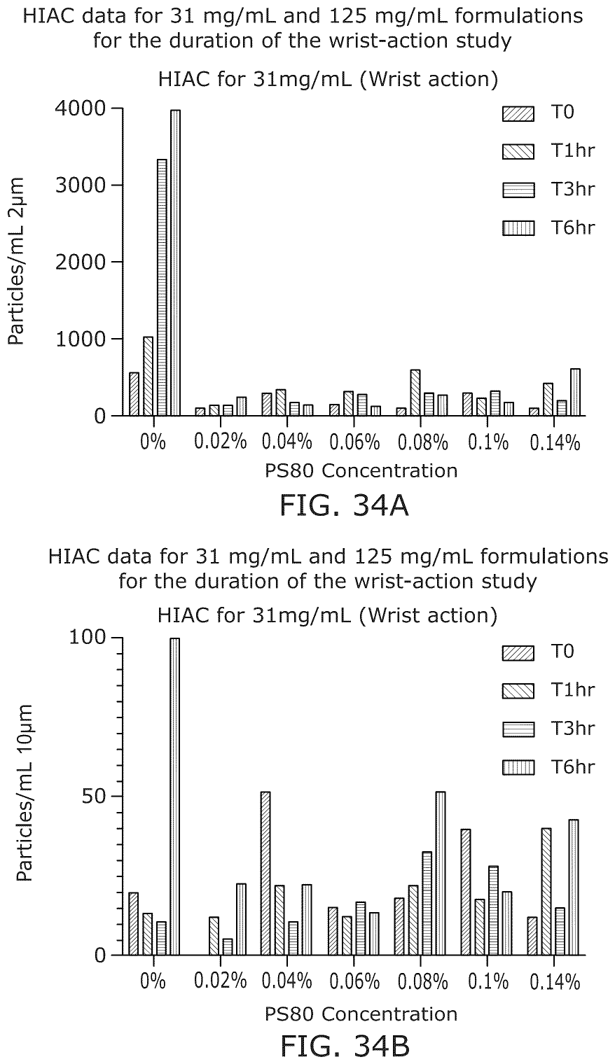

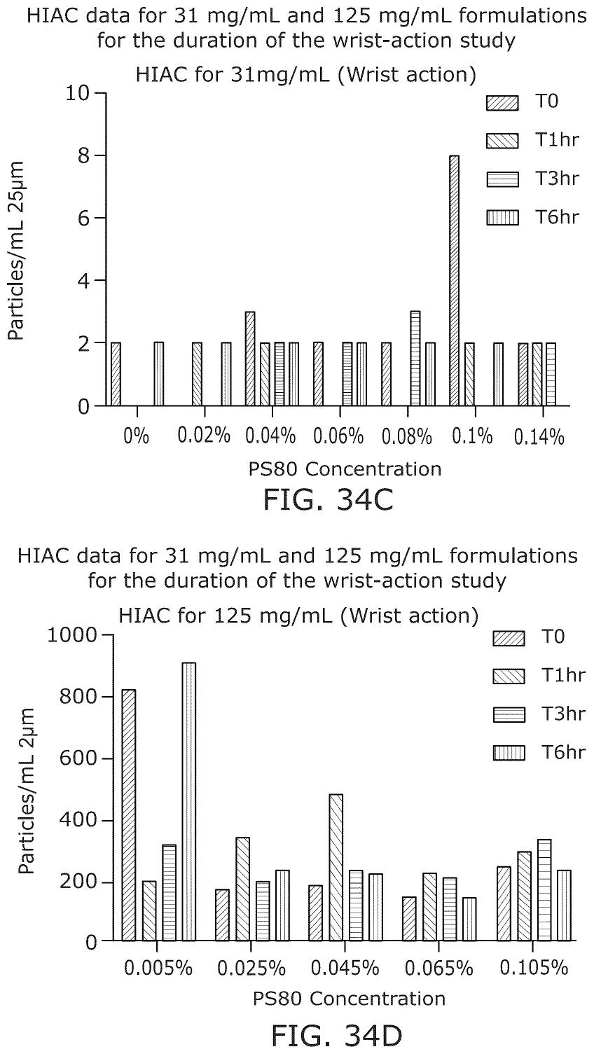

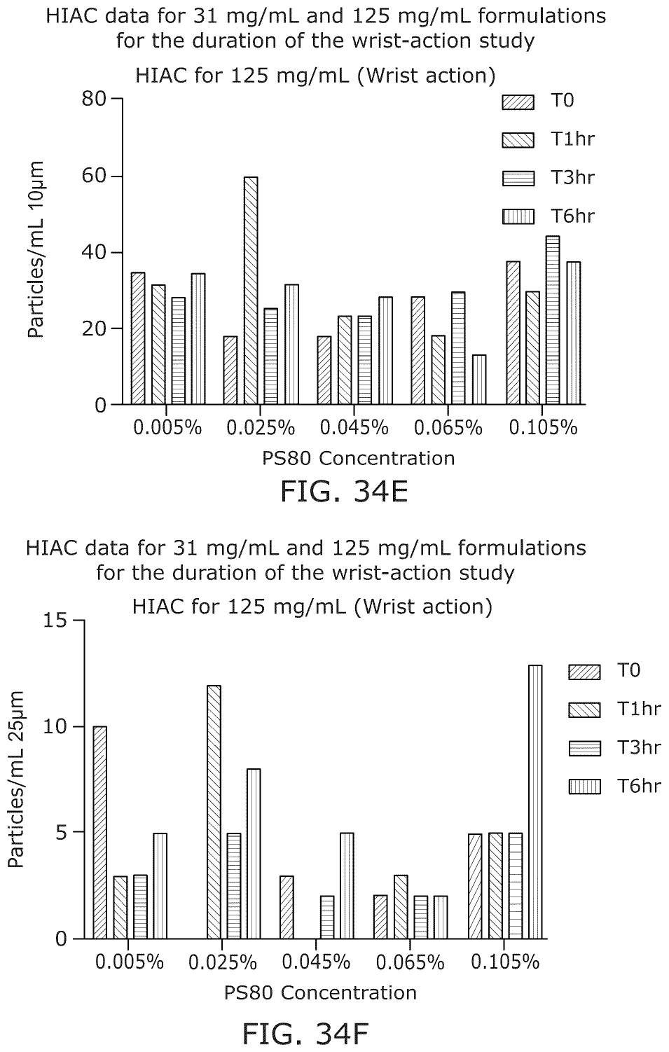

Subvisible particles of at least 2 μm, at least 10 μm, or at least 25 μm in the example formulations were measured by HIAC. Obtained results are shown in FIGS. 34A-34F, which graphically depict HIAC data for 31 mg/mL and 125 mg/mL formulations for the duration of the wrist-action study.

As shown in FIGS. 34A-34F, there was an obvious increase in the ≥2 μm particles for the 31 mg/mL formulation in the absence of PS80 over time when the formulation was subjected to wrist-action shaking. This was also seen in the ≥2 μm particles sizes in the 125 mg/mL formulations with 0.005% PS80.

### Orbital Shaking Study

### SEC-HPLC for HMWS

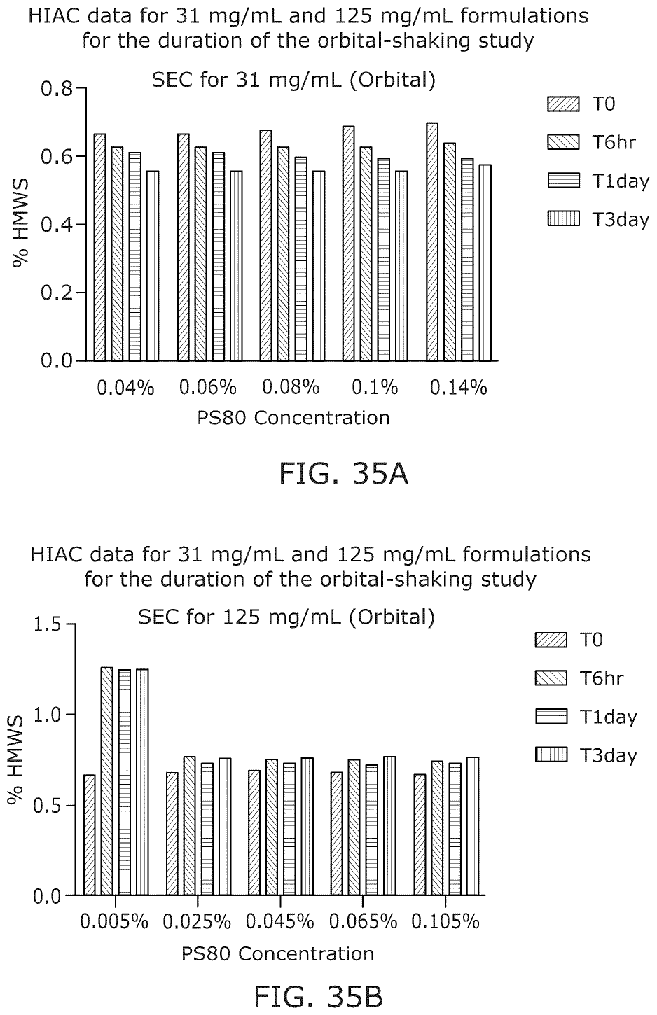

All formulations subjected to orbital shaking were analyzed by SEC-HPLC for HMWS evolution at timepoints of T0, T1 hr, T3 hr, and T6 hr. FIGS. 35A and 35B are graphs depicting the results of HMWS evolution for 31 mg/mL and 125 mg/mL formulations over the duration of the orbital shaking study.

As can be seen from FIGS. 35A and 35B, there was no significant increase in HMWS over time for the 31 mg/mL concentration formulations for all PS80 concentrations, when subjected to orbital shaking. A noticeable increase in HMWS, from T0, for the 125 mg/mL formulation at 0.005% PS80 concentration was observed, while the rest of the 125 mg/mL formulations had relatively constant HMWS % during the shaking.

### Turbidity Analysis

Turbidity analysis was performed to example formulations subjected to orbital shaking at timepoints of T0, T1 hr, T3 hr, and T6 hr. Obtained results show that there was no significant increase in turbidity for the 31 mg/mL formulations with PS80 concentrations from 0.04% to 0.14%. There was a significant increase in turbidity at all three timepoints when compared with T0, for the 125 mg/mL concentration formulations with all PS80 concentrations, which may be due to instrumental issues.

### HIAC for Subvisible Particles

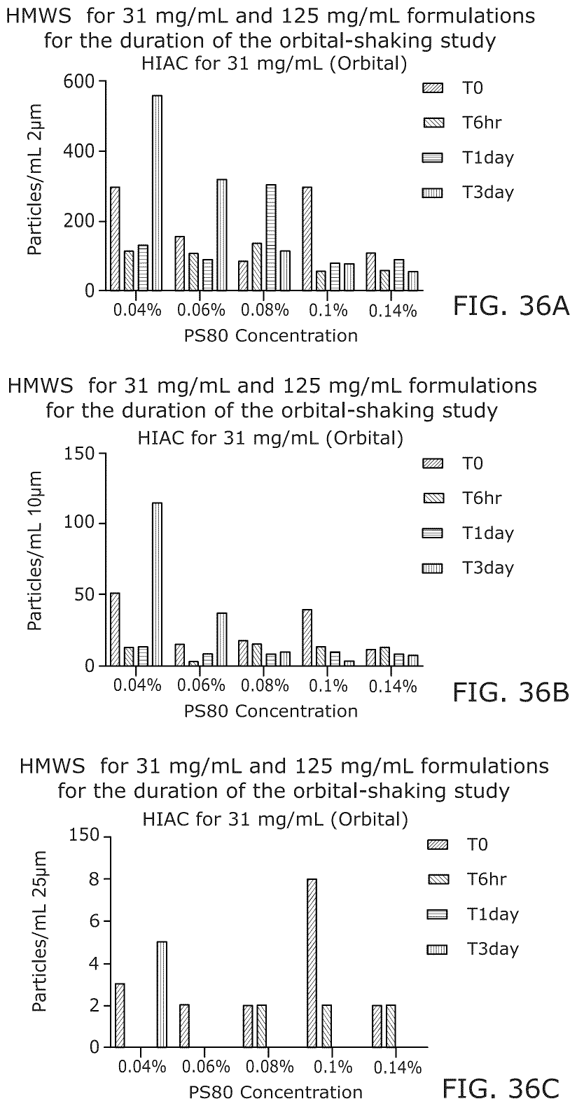

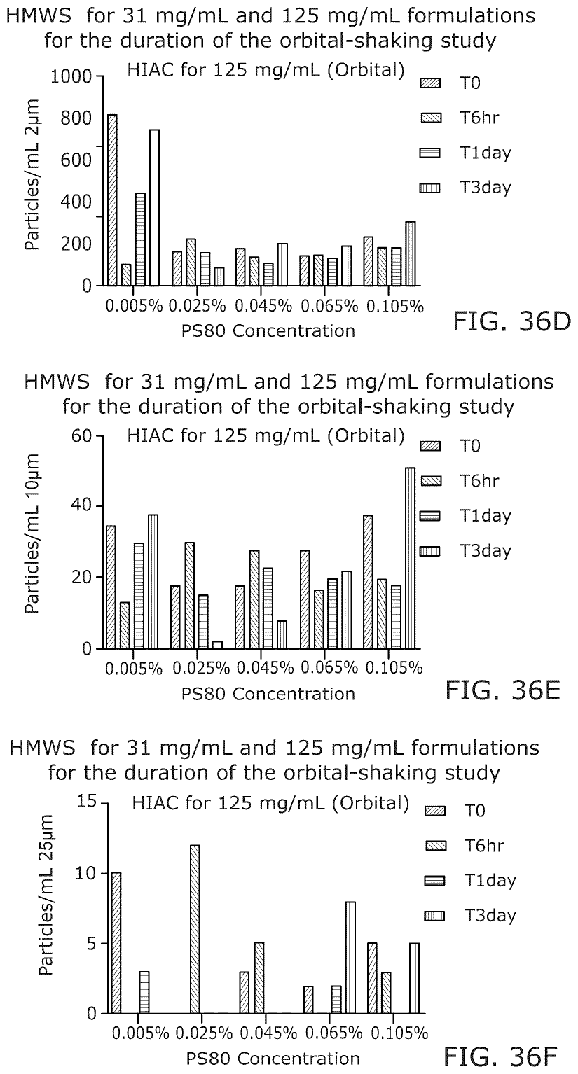

Subvisible particles of at least 2 μm, at least 10 μm, or at least 25 μm in the example formulations subjected to orbital shaking were measured by HIAC. FIGS. 36A, 36B, 36C, 36D, 36E, and 36F are graphs depicting HIAC data for 31 mg/mL and 125 mg/mL formulations for the duration of the orbital shaking.

Obtained results show that for 125 mg/mL formulation with 0.005% PS80, relatively higher particle counts were observed for particles ≥2 μm but not for particles at other sizes. Formulation at 31 mg/mL with 0.04% PS80 had marginally higher levels for particles ≥2 μm and ≥10 μm, compared to other formulations at the same protein concentration.

### Wrist-Action Stress+Silicone Oil

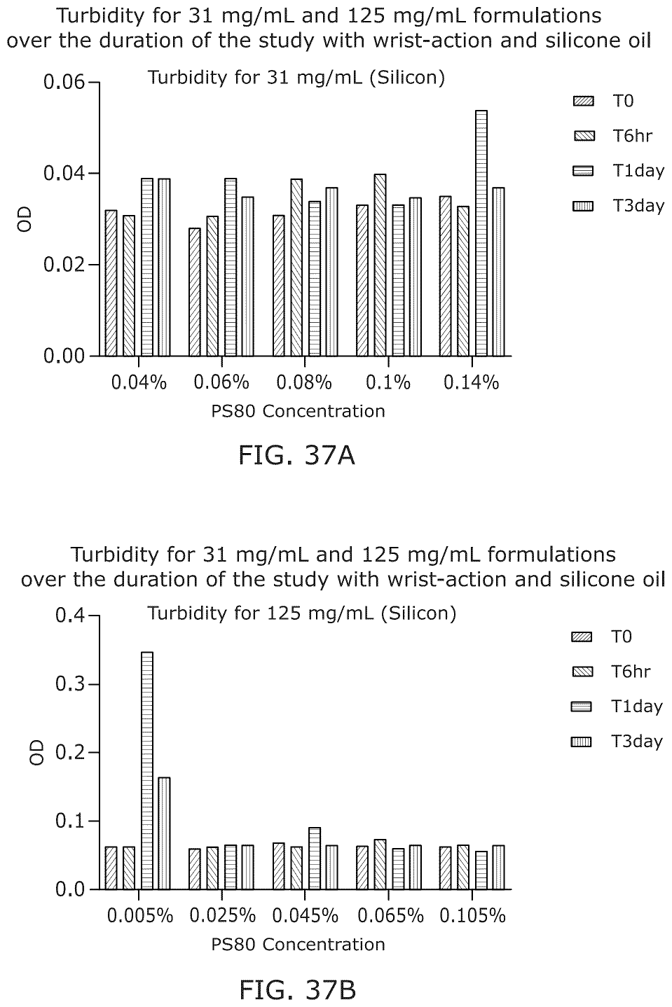

Turbidity analysis was performed to example formulations subjected to wrist-action stress plus silicone oil at timepoints of T0, T1 hr, T3 hr, and T6 hr. Obtained results are illustrated in FIGS. 37A and 37B, which are graphs depicting turbidity data for 31 mg/mL and 125 mg/mL formulations over the duration of the study.

No significant increase in turbidity was observed for 31 mg/mL formulations with varying PS80 concentrations while subjecting to wrist-action and silicone oil. There was a drastic increase in turbidity for the 125 mg/mL concentration formulations for PS80 concentration 0.005%. No significant increase in turbidity was observed for the 125 mg/mL concentration formulations with 0.025% to 0.105% PS80.

### CONCLUSION

The goals of this formulation development activity were to identify the preferred concentration of PS80 and the type and concentration of the chelator to be used. Additionally, to perform a pH/buffer screen and demonstrate DP stability bracketing the target pH, to assess the feasibility and liabilities associated with switching to a PFS, and to demonstrate good DP robustness against physicochemical stresses.

The PS80 concentration was evaluated by subjecting formulations containing different PS80 concentrations to mechanical stresses. 0.06% (w/v) PS80 was identified as the surfactant concentration to mitigate potential impact of PS80 loss on quality attributes.

Metal chelators were added to the formulation to provide protection from potential metal-induced protein and excipient degradation. 10 μM EDTA and 10 μM DTPA provided comparable levels of protection. As a result, EDTA was ultimately selected.

Stability of amlitelimab was evaluated in histidine buffers. DP stability studies in vials and PFS were performed between pH 5.5-6.3 under refrigerated, ambient, accelerated, and frozen conditions and demonstrated a good product profile. 10 mM histidine buffer with a target pH 6.0 was selected as buffer of choice.

Sucrose is a well-documented excipient used in protein formulations as a stabilizer/cryoprotectant. 220 mM sucrose is about 7.5% which represents a ratio suitable to protect both the DP and FDS concentrations of 31-125 mg/mL.

Further, stability of amlitelimab in both BD and OMPI PFS's were compared and demonstrated similar, except less subvisible particle formations in BD PFS. DP stability studies in vials and PFS were performed between pH 5.5-6.3 under refrigerated, ambient, accelerated, and frozen conditions and demonstrated a good product profile.

The results from these stability studies overall demonstrate the robustness of the target formulation to stabilize frozen FDS as well as an aqueous DP in PFS under a variety of the storage and other important stress conditions. Thus, the final target amlitelimab aqueous formulation derived from the results of this work was 10 mM histidine, 220 mM sucrose, 10 μM EDTA, 0.06% PS80, at pH 6.0+0.2, and is optimal for stabilizing the 31-125 mg/mL DP as well as the 125 mg/mL FDS.

### Example Drug Products

Example amlitelimab drug products (DP) were developed from this study. The amlitelimab DPs were composed of amlitelimab, L-histidine, L-histidine hydrochloride monohydrate, sucrose, EDTA, and super-refined polysorbate 80. The excipients in the amlitelimab formulations are known to be well-tolerated following parenteral administration and are water soluble. The DP formulations and example container are summarized in Table 1-7 below.

TABLE 1-7
Drug products (DP) for amlitelimab

| | Contents/ | | | |
|---|---|---|---|---|
| Ingredients/Items | PFS | DP1-1 | DP1-2 | DP1-3 |
| amlitelimab | mg | 62.15 | 125 | 250 |
| L-Histidine | mg | 1.49 | 1.49 | 1.49 |
| L-Histidine | mg | 2.18 | 2.18 | 2.18 |
| hydrochloride | | | | |
| monohydrate | | | | |
| sucrose | mg | 150.62 | 150.62 | 150.62 |
| EDTA disodium salt | mg | 0.0074 | 0.0074 | 0.0074 |
| dihydrate | | | | |
| Super refined | mg | 1.2 | 1.2 | 1.2 |
| Polysorbate 80 | | | | |
| Water | | | | |

| | | |
|---|---|---|
| Container | glass | BD Neopak 2.25 mL, |
| | syringe | 27G ½ 5B STW RNS |
| | | FM30 HS syringe |
| Closure | rubber | West 1-3 mL, Novapure |
| | stopper | 4023/50, Gray plunger stopper |

### Example 2—Product Quality of Formulations

Formulations F2-1, F2-2, and F2-3 and containers comprising these formulations were prepared as listed in Table 2-1. The formulation of F2-1 (also called “Cycle 1” formulation herein) distinguishes from the formulations of F2-2 (also called “Cycle 2 ESB0.A_F1” herein) and F2-3 (also called “Cycle 2” ESB0.B-F2” herein) that were developed from the Cycle 2 study described in Example 1. These formulations were aseptically filled into a 6 ml injection vial (6R) and sealed with FLUROTEC coated stoppers and crimped with a cap, or aseptically filled into a BD Neopak with 2.25 mL syringe closed with West 1-3 mL Novapure 4023/50 Gray plunger stopper.

TABLE 2-1
Example Formulations

| | | Primary |
|---|---|---|
| Product | Formulation | Container |
| F2-1 | 125-138 mg/mL amlitelimab | 6R vial |
| (Cycle 1) | 20 mM L-histidine/histidine hydrochloride | |
| | 220 mM sucrose, | |
| | 0.04% (w/v) polysorbate 80, | |
| | pH 6.0 | |
| F2-2 | 125-138 mg/mL amlitelimab | 6R vial |
| (Cycle 2 | 20 mM L-histidine/histidine hydrochloride | |
| ESB0.A_F1′) | 220 mM sucrose, | |
| | 0.04% (w/v) polysorbate 80, | |
| | pH 5.8 | |
| F2-3 | 125-138 mg/mL amlitelimab | BD 2.25 mL |
| (Cycle 2 | 10 mM L-histidine/histidine hydrochloride | PFS |
| ESB0.B_F2′) | 220 mM sucrose, | |
| | 0.04% (w/v) polysorbate 80, | |
| | 10 μM EDTA, | |
| | pH 6.0 | |

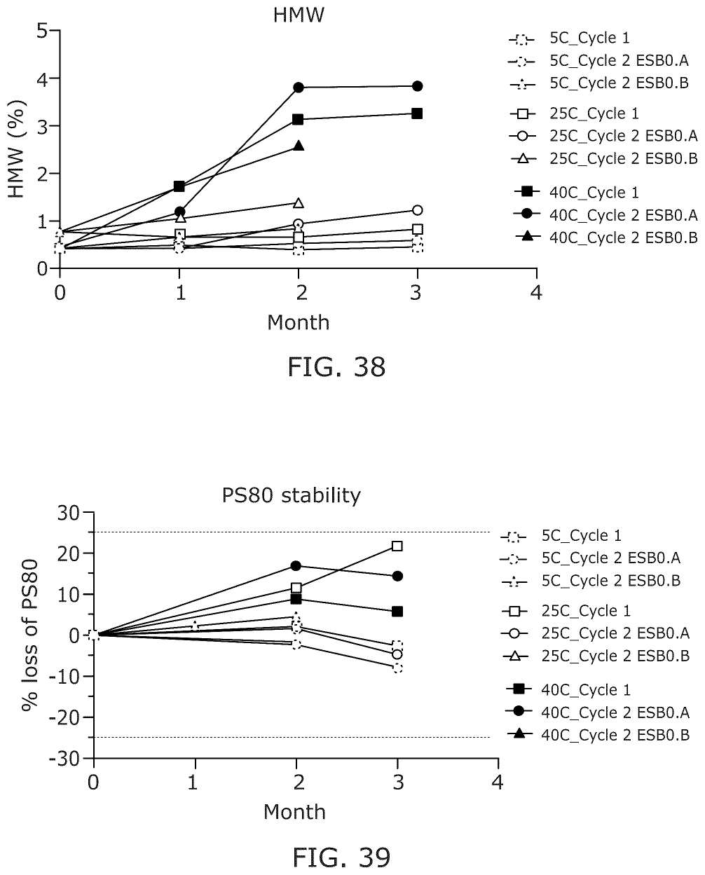

The formulations F2-1, F2-2, and F2-3 were stored in the containers over a period of up to 3 months at 5° C., 25° C., and 40° C. HMWS, PS80 degradation kinetics, deamidation, oxidation, charge variants of the formulations were assessed at timepoints of T0, 1 month storage (1M), 2-month storage (2M), and 3-month storage (3M), according to the methods detailed in Example 1. Obtained results are illustrated in FIGS. 38-42.

FIG. 38 is a graph depicting the evolution of HMWS analyzed by SEC-HPLC for the formulations F2-1, F2-2, and F2-3 over the 3 months of storage at 5° C., 25° C., and 40° C. As can be seen in FIG. 38, the HMWS in the formulations F2-1, F2-2, and F2-3 stored at a specific temperature such as 5° C., 25° C., or 40° C. were comparable, with the highest HMW % for all the formulations after storage at 40° C. for over 2 months.

FIG. 39 is a graph depicting the PS80 loss of the formulations F2-1, F2-2, and F2-3 over 3 months of storage at 5° C., 25° C., and 40° C., analyzed by CAD according to the method described in Example 1. The dotted line in FIG. 39 indicates the method variability. The results demonstrate that PS80 in all formulations are stable. The change of PS80 over the 3 months of storage at 5° C., 25° C., and 40° C. was within the variability of the analysis (below the dotted line). The changes of PS80 in the three formulations stored at a particular temperature were comparable, while the highest loss of PS80 was observed in F2-1 (Cycle 1) after 3 months storage at 25° C.

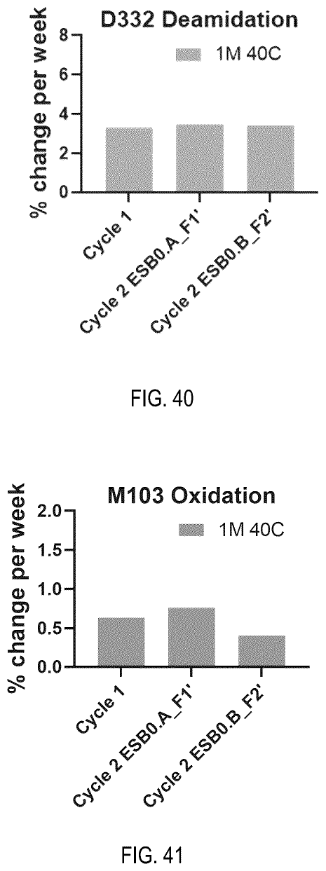

FIG. 40 is a graph depicting the percent change per week of deamidation at HC D332 of the antibody for formulations F2-1, F2-2, and F2-3 over 1 month of storage at 40° C., measured by peptide mapping. As can be seen from FIG. 40, there was no significant difference between the percent changes of D332 deamidation in all three formulations measured.

FIG. 41 is a graph depicting the percent change per week of oxidation at HC M103 of the antibody, measured by peptide mapping, for F2-1, F2-2, and F2-3 over 1 month of storage at 40° C. As can be seen from FIG. 41, there was no significant difference between the percent changes of M103 Oxidation in all three formulations measured.

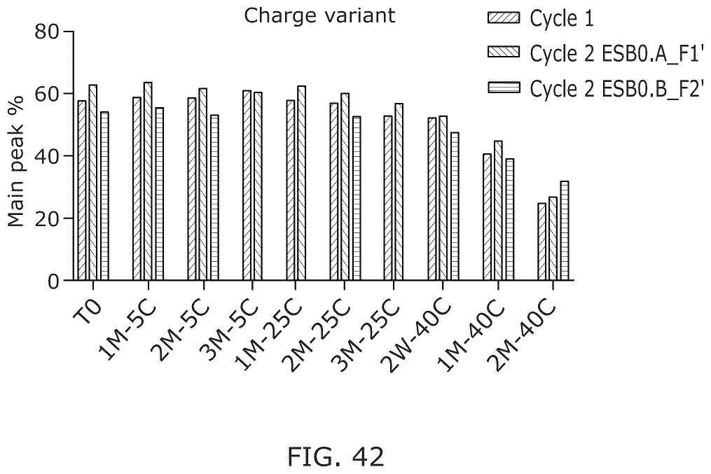

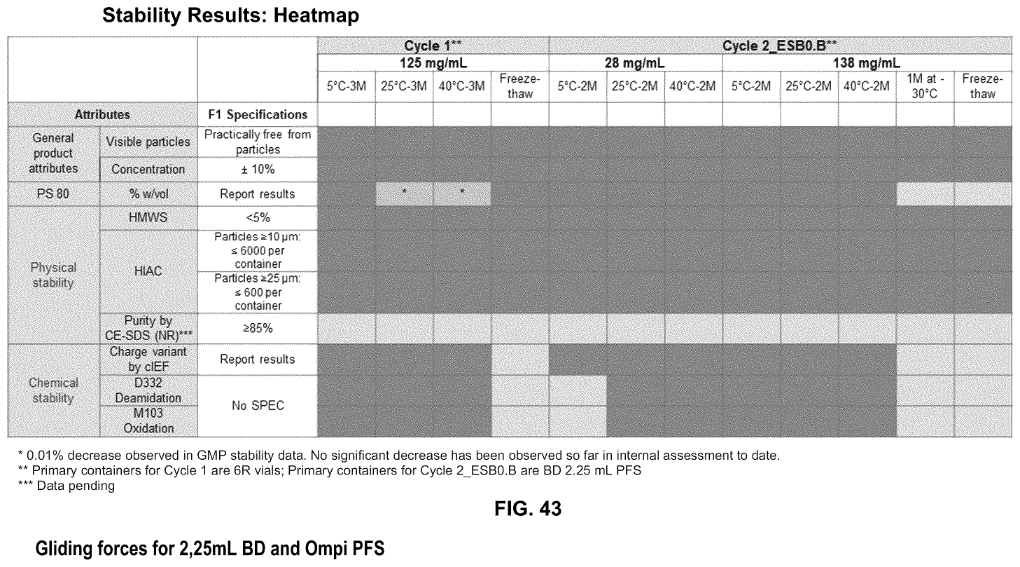

FIG. 42 is a graph depicting the charge variants measured by capillary isoelectric focusing (cIEF) for formulations F2-1, F2-2, and F2-3 over two months of storage at 5° C., 25° C., and 40° C. As can been seen in FIG. 43, the percent of main peak was stable in all three formulations stored at 5° C. and 25° C. for up to 3 months. A slight decrease of the main peak started when the formulations were stored at 40° C. for 2 weeks, and the percent of main peak remained decrease over the longer time of storage at 40° C., with the lowest amount of main peak in formulations stored at 40° C. for 2 months. There were no significant differences of the main peak between the formulations stored over the time at the same temperature.

In sum, there was no major differences in product quality, including physical or chemical properties or characteristics, between formulations F6-1, F6-2, and F6-3.

### Example 3-28 mg/mL and 138 mg/mL Formulations are Stable

Formulations F3-1, F3-2, and F3-3 were prepared according to Table 3-1. The formulation of F3-1 (also called “Cycle 1” formulation herein) was developed from an alternative study for developing formulations for Phase I and Phase II clinic trial studies (Cycle 1 study). The formulations of F3-2 (also called “Cycle 2 ESB0.B_1” herein) and F3-3 (also called “Cycle 2 ESB0.B_2” herein) were developed from the study described in Example 1. These formulations were aseptically filled into a 6 mL injection vial (6R) and sealed with FLUROTEC® coated stoppers and crimped with a cap, or aseptically filled into a BD Neopak with 2.25 mL syringe closed with West 1-3 mL NOVAPURE® 4023/50 Gray plunger stopper.

TABLE 3-1
Example Formulations

| Product | Formulation | Container |
|---|---|---|
| F3-1 | 125 mg/mL amlitelimab | 6R vial |
| (Cycle 1) | 20 mM L-histidine/histidine hydrochloride | |
| | 220 mM sucrose, | |
| | 0.04% (w/v) polysorbate 80, | |
| | pH 6.0 | |
| F3-2 | 28 mg/mL amlitelimab | 6R vial |
| (Cycle 2 | 10 mM L-histidine/histidine hydrochloride | |
| ESB0.B_1) | 220 mM sucrose, | |
| | 0.04% (w/v) polysorbate 80, | |
| | 10 μM EDTA, | |
| | pH 5.8 | |
| F3-3 | 125-138 mg/mL amlitelimab | BD 2.25 mL |
| (Cycle 2 | 10 mM L-histidine/histidine hydrochloride | PFS |
| ESB0.B_2) | 220 mM sucrose, | |
| | 0.04% (w/v) polysorbate 80, | |
| | 10 μM EDTA, | |
| | pH 6.0 | |

The formulations F3-1, F3-2, and F3-3 were stored in the containers over a period of up to 3 months at 5° C., 25° C., and 40° C. General product attributes such as visible particles and concentration, PS80 degradation kinetics, physical stability measured by HMWS, HIAC, and purity by CE-SDA (NR), and chemical stability measured by charge variant by cIEF, deamidation and oxidation were assessed over 3 months of storage at 5° C., 25° C., and 40° C. Obtained results are illustrated in table 3-2 shown in FIG. 43.

The results show that the formulations F3-2 and F3-3 were stable over at least 2 months of storage at 5° C., 25° C., and 40° C. The results demonstrate that the formulation comprising the excipients in particular concentrations as developed in the study described in Example 1 was suitable for use with the antibody over a broad range of concentrations, from low to high. The formulations developed in Example 1 are suitable for making drug products with low to high concentrations of anti-hOX40L antibody for clinical trial or commercial purposes.

### Example 4—125 mg/mL PFS Contained in BD and OMPI are Stable

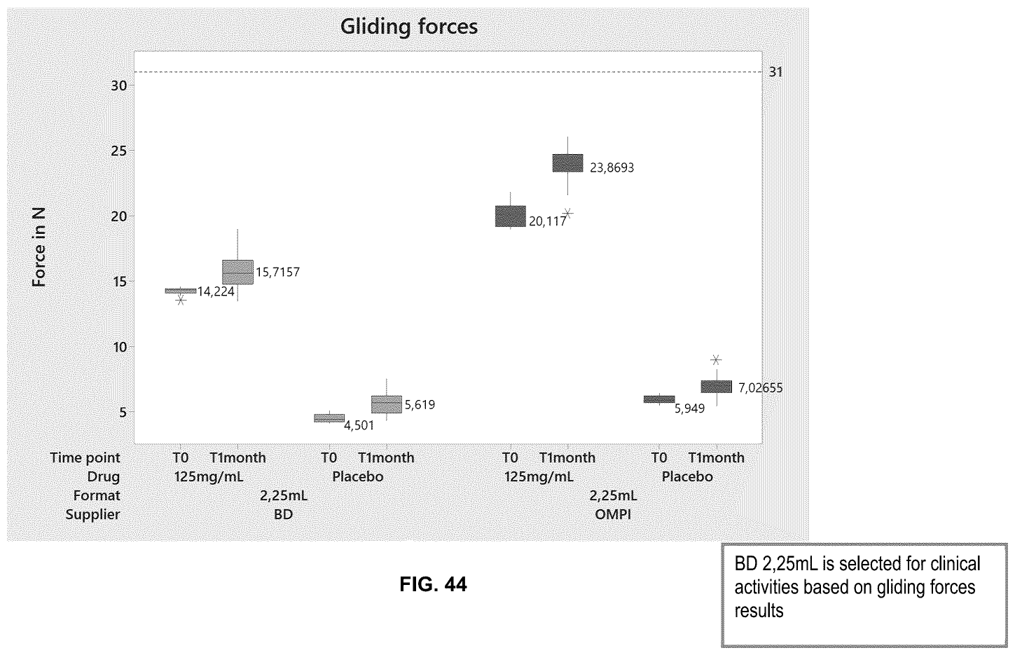

In this example, amlitelimab was formulated in a solution including 10 mM His, 220 mM sucrose, 0.04% PS80, and 10 μM EDTA in water, with pH 6.0. The formulation was filled in 2.25 mL BD and OMPI syringes, respectively. The products were stored at 40° C. for at least 1 month. After 1 month of storage at 40° C., gliding forces for the products were evaluated. Obtained results are shown in FIG. 44. The gliding forces results for 2.25 mL BD and OMPI PFS indicated that the products would be stable over at least one year of storage at 5° C.

### Example 5 Rheological Characterization and Viscosity Screening for Developing Anti-hOX40L Antibody Formulations

For rheological characterization, the DS of amlitelimab was up-concentrated by centrifugation in 25 mM His/HisHCl buffer at pH 6.2 to maximum possible concentration (around 263 mg/ml). The concentration value obtained for the product at the start of the study was utilized for further dilution calculations required for the study.

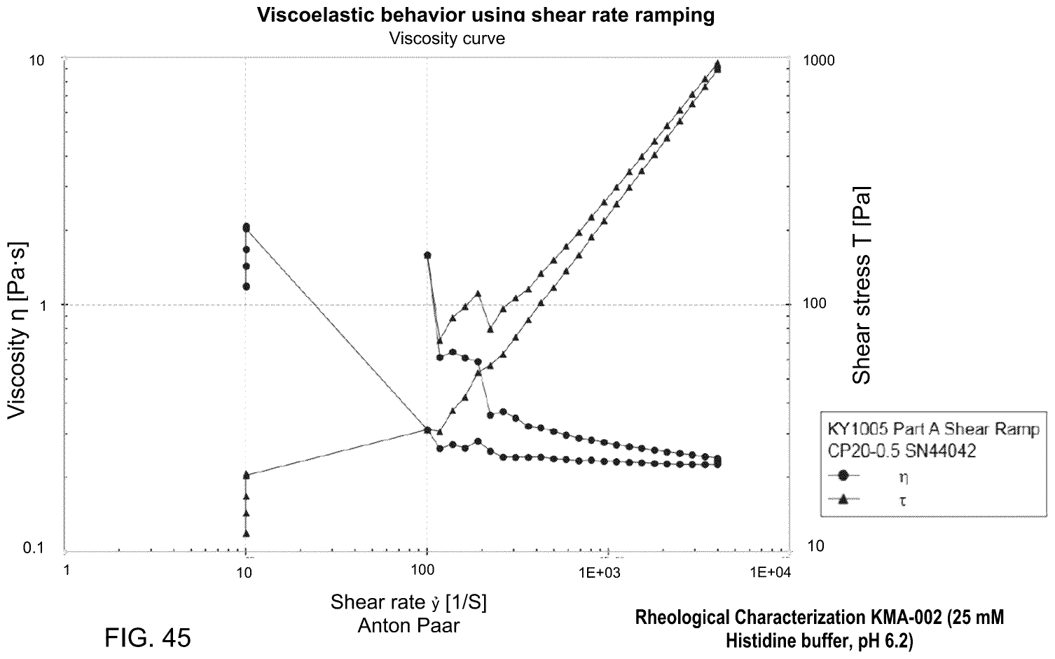

Viscosity measurements were performed using an Anton Paar Rheometer (Graz, Austria). Viscoelastic behavior was studied at high concentration of 263 mg/ml of the antibody in 25 mM histidine/histidine hydrochloride buffer (HisHCl) at pH 6.2 by shear rate ramping from 0-4000 s⁻¹ at 25° C. as shown in FIG. 45. Amlitelimab showed shear thinning with increasing shear rate, and 2000 s⁻¹ was chosen as the shear rate for all future studies based on the shear thinning profile.

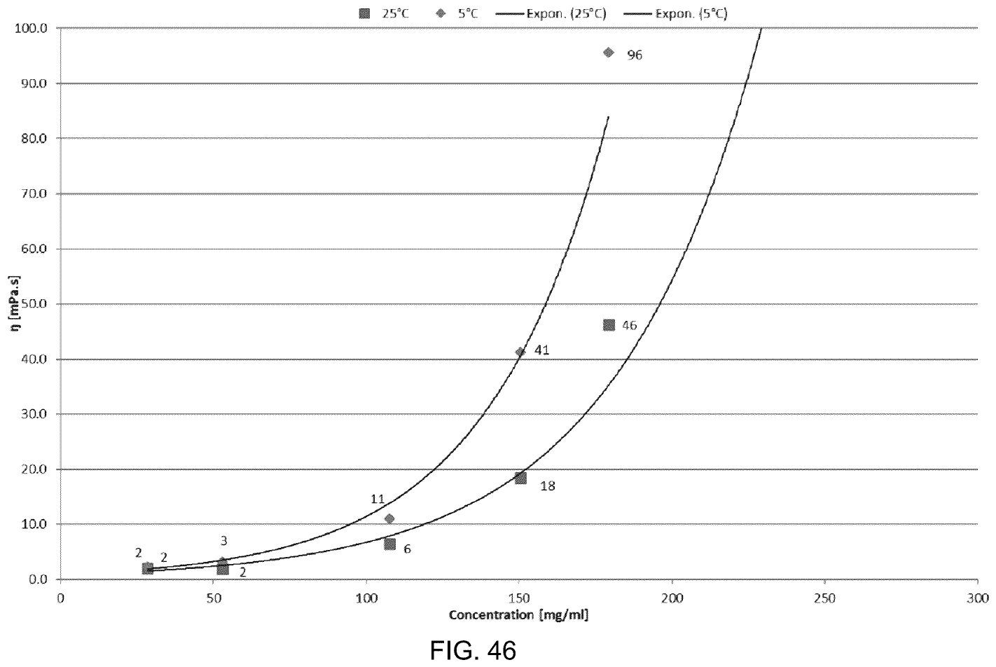

Serial dilutions to five lower concentrations were performed and verified by UV-absorbance measurements (A280) using an Agilent Cary 60 spectrophotometer (Agilent Technologies, Santa Clara, CA, USA) with variable path length extension (Repligen CTech™ SoloVPE®, Waltham, MA, USA). The viscosity of each concentration in 25 mM histidine/histidine hydrochloride buffer (HisHCl) at pH 6.2 was measured at a shear rate of 2000 s⁻¹ at 25° C. and 5° C. Measurements were performed in duplicate at 25° C. The resulting concentration-dependent viscosity profile of amlitelimab showed exponential increase in viscosity around 150 mg/ml concentration at 25° C., while at 5° C. exponential increase was observed after around 100 mg/ml concentration (FIG. 46). Obtained concentration and viscosity measurements are further detailed in Table 1a-1 below.

TABLE 1a-1
Results of Concentration and Viscosity Measurements

| Target | Actual | Viscosity at | Average |
|---|---|---|---|
| Concentration | Concentration | +5° C. | viscosity at |
| (mg/ml) | (mg/ml) | (mPa s) | +25° C. (mPa s) |
| Max. | 263 | 668 | 192 |
| 180 | 179.2 | 96 | 46 |
| 150 | 150.5 | 41 | 19 |
| 100 | 107.7 | 11 | 6 |
| 50 | 52.9 | 3 | 2 |
| 25 | 28.4 | 2 | 2 |

Viscosity data at 25° C. suggests processing and up-concentration can be done up to 150 mg/ml at room temperature under the standard processing conditions. The target concentration for Part B was set at 180 mg/mi.

For the viscosity lowering study, DS of amlitelimab was subjected to buffer exchange in 20 mM His/HisHCl buffer pH 6.0. After the buffer exchange, up-concentration was performed to approximately 230 mg/ml followed by the pH adjustment. Protein concentration and pH was monitored during the processing. Stock solutions of different excipients were prepared to achieve target formulations.

As shown in Table 1a-2, 12 formulation conditions with different excipients and pH were formulated for assessment of viscosity. Prior to the viscosity measurements, all formulations were stored at 5° C. for at least one day to evaluate signs of visible phase transition. Viscosity of different formulations was measured at shear rate of 2000 s⁻¹ at 25° C. Measurements were performed in duplicate.

TABLE 1a-2
Result Summary for Viscosity and Phase Separation Lowering Study

| | | | | Average | |
|---|---|---|---|---|---|
| | | | | viscosity | Measured |
| Formulation | Buffer | | | at +25° C. | Concentration |
| No. | (HisHCl) | pH | Excipients | (mPa · s) | (mg/ml) |
| F1a-1 | 20 mM | 6.0 | — | 36 | — |
| F1a-2 | 20 mM | 6.0 | 220 mM | 44 | — |
| | | | Sucrose | | |
| F1a-3 | 20 mM | 6.0 | 150 mM | 17 | 186 |
| | | | NaCl | | |
| F1a-4 | 20 mM | 6.0 | 50 mM | 19 | — |
| | | | NaCl | | |
| F1a-5 | 20 mM | 6.0 | 150 mM | 14 | — |
| | | | Arginine | | |
| | | | HCl | | |
| F1a-6 | 20 mM | 6.0 | 50 mM | 19 | — |
| | | | Arginine | | |
| | | | HCl | | |
| F1a-7 | 20 mM | 6.0 | — | 34 | — |
| F1a-8 | 20 mM | 5.0 | — | 33 * | 186 |
| F1a-9 | 20 mM | 5.5 | — | 43 * | 184 |
| F1a-10 | 20 mM | 6.5 | — | 11 | — |
| F1a-11 | 20 mM | 6.5 | 150 mM | 5 | 125 |
| | | | NaCl | | |
| F1a-12 | 20 mM | 6.5 | 150 mM | 5 | — |
| | | | Arginine | | |
| | | | HCl | | |

*Viscosity measured at 5° C.

The performed rheological characterization and viscosity lowering studies showed the following,
Amlitelimab processing and up-concentration in 20 mM HisHCl, at different pH values was successfully performed.
Up-concentration processing was faster at pH 5.0 and pH 5.5.
Slower up-concentration in 20 mM HisHCl at pH 6.5 observed.
Target concentration of 180 mg/ml was successfully achieved in 20 mM HisHCl at pH 5.0, pH 5.5, and pH 6.0 by centrifugation.
Maximum concentration after compounding was 125 mg/mL for formulations in 20 mM HisHCl at pH 6.5.
Formulations containing 150 mM arginine-HCl showed major lowering in viscosity as compared to histidine only formulation (around −60% of histidine only formulation, depending on pH).
Also, formulations containing 150 mM arginine-HCl were more effective in lowering viscosity as compared to formulations containing 50 mM Arginine-HCl.
Formulations containing 50 mM or 150 mM sodium chloride showed comparable lowering in viscosity as compared to histidine only formulation (around −50% compared to histidine only formulation at pH 6.0).
Formulations containing 150 mM arginine —HCl showed slightly superior lowering in viscosity as compared formulations with 150 mM sodium chloride.
Lowest viscosity measured for formulation containing 20 mM HisHCl and 150 mM arginine-HCl.
Sucrose increased viscosity as compared to histidine only formulation (around +20% compared to histidine only formulation).

Based on the obtained results, it has been shown that arginine hydrochloride and sodium chloride effectively reduced the viscosity of amlitelimab. Viscosity around 15-25 mPas at a target concentration of 125 mg/ml is expected for formulations without viscosity reducing agents depending on pH, temperature and formulation. In the presence of arginine hydrochloride or sodium chloride, a reduction of viscosity to ≤15 mPas can be expected. A target formulation concentration of 125 mg/mL at pH of 6.0 is suggested for s.c. formulation development.

### Example 6—Development of Anti-OX40L Antibody Formulations

The aim of the study was to screen pH, buffer, excipient, and surfactant concentration under specific stress conditions to develop formulations with high concentration drug product (DP) in an aqueous form, i.e., a liquid form, for subcutaneous (SC) administration of amlitelimab.

Based on the study data from Example 5, different antibody aqueous formulations of Anti-OX40L at a concentration of 125 mg/mL were prepared and investigated. An additional formulation at comparatively lower concentration of 75 mg/mL without viscosity-reducing excipients was also investigated. Aqueous formulations are listed in Table 2a-1.

TABLE 2a-1
Aqueous Formulations under Evaluation

| | | | | Tonicifier/ | Tonicifier/ | | |
|---|---|---|---|---|---|---|---|
| Formulation | Protein | Buffer | | Bulk agent | Bulk agent | | Fill |
| code | conc. | (His/HCl) | pH | 1 | 2 | Surfactant | Volume ¹ |
| F2a-1 | 125 | 20 mM | 6.0 | 220 mM | — | 0.04% w/v | 2.4 mL |
| | mg/mL | | | Sucrose | | PS80 | |
| F2a-2 | 125 | 20 mM | 6.0 | 90 mM | 50 mM | 0.04% w/v | 2.4 mL |
| | mg/mL | | | Sucrose | Sodium | PS20 | |
| | | | | | chloride | | |
| F2a-3 | 125 | 20 mM | 6.0 | 90 mM | 50 mM | 0.04% w/v | 2.4 mL |
| | mg/mL | | | Sucrose | Sodium | PS20 | |
| | | | | | chloride | | |
| F2a-4 | 75 | 20 mM | 6.0 | 220 mM | — | 0.02% w/v | 2.4 mL |
| | mg/mL | | | Sucrose | | PS80 | |

¹ filling in 6R vials

The formulations listed in Table 2a-1 were prepared by 1) buffer exchange to achieve target buffer concentration and pH followed by 2) the up-concentration above the target concentration.

The protein concentration, pH value, and density were determined upon processing of the BPDS and utilized for the required calculation of each batch formulation by using standard dilution procedure. The formulations were compounded by spiking with excipient stock solutions to the target formulation of the bulk drug product. The protein concentration, osmolality, and pH of the finally compounded solutions were determined and confirmed. All bulk drug product (BDP) formulation solutions were filtered using a 0.22 μm Polyvinylidene Fluoride (PVDF) membrane filter.

Each formulation was filled manually, observing aseptic technique, into 6R/20 mm glass type I vials stoppered with 20 mm bromobutyl rubber stoppers and sealed with 20 mm aluminum flip-off seals.

Prepared formulations were then subjected for stability studies by being placed under thermal stress (−65° C. or below, +5° C.±3° C., +25° C.±2° C., and +40° C.±2° C. for up to 8 weeks for all formulations and for up to 16 weeks for F2a-1 and F2a-4) and mechanical stress conditions (freeze/thaw and agitation). Each formulation sample was tested according to the test methods listed in Table 2a-2.

TABLE 2a-2
Analytical testing plan per formulation

| | | Stability | |
|---|---|---|---|
| Nr | Test Method | timepoint | Instrument |
| 1 | Color measurement using | All | Lico 690, (Hach Lange GmbH, |
| | colorimeter | | Dusseldorf, Germany) |
| 2 | Clarity & opalescence of a | All | TL23 Series Laboratory Turbidimeter |
| | solution (turbidimetry) | | (Hach Lange GmbH, Dusseldorf, |
| | | | Germany) |
| 3 | Determination of pH | All | pH meter 780, (Metrohm, Herisau, |
| | | | Switzerland) |
| 4 | Determination of Polysorbate | All | Waters alliance 2695 HPLC system, |
| | 20/80 Content | | (Waters, Milford, MA, USA) equipped |
| | | | with a Knitted reaction coil 1 mL |
| 5 | Osmolality by Freezing Point | All | Advanced Micro-Osmometer Model |
| | Depression | | OsmoPRO ™, (Advanced Instruments, |
| | | | Norwood, MA, USA) |
| 6 | Protein concentration | All | CTech ™ Solo VPE ®, (Repligen, |
| | determination by SoloVPE | | Waltham, MA, USA) |
| 7 | Purity by Size exclusion | All | Waters alliance 2695 HPLC system, |
| | chromatography (SE-HPLC) | | (Waters, Milford, MA, USA) |
| 8 | Purity by Chip-based CE-SDS | All | LabChip GXII instrument |
| | (non-reducing and reducing | | (PerkinElmer, Waltham, MA, USA), |
| | conditions) | | |
| 9 | Purity by Imaged Capillary | All | ICE3, (ProteinSimple, San Jose, CA, |
| | Isoelectric Focusing (icIEF) | | USA) |
| 10 | Purity by reverse phase-HPLC | All | Agilent HPLC equipment (Agilent |
| | (RP-HPLC) (reduced and non- | | Technologies, Santa Clara, CA, USA) |
| | reduced condition) | | |
| 11 | Sub-visible particles by Light | All | HIAC 9703+ Pharmaceutical Particle |
| | Obscuration (LO), low volume | | Counter, (Beckman Coulter, Brea, |
| | method | | California, USA) |
| 12 | Visible particles | All | Apollo II Liquid Viewer, (Adelphi |
| | | | Manufacturing, Haywards Heath, UK) |

The pH, protein concentration, and osmolality values obtained for the compounded solutions for the aqueous formulations (F2a-1, F2a-2, F2a-3, and F2a-4) after compounding and filtration were at target as shown in Table 2a-3.

TABLE 2a-3
Results of Characterization of Formulations After Compounding

| | Formulations | | | |
|---|---|---|---|---|
| Test | F2a-1 | F2a-2 | F2a-3 | F2a-4 |
| pH ^{a} | 5.9 | 6.0 | 6.0 | 5.9 |
| Osmolality by freezing point | 367 | 337 | 298 | 327 |
| [mOsm/Kg] ^{a} | | | | |
| Protein concentration ^{a} | 120.5 | 125.2 | 124.4 | 76.3 |
| [mg/mL] | | | | |

^{a} T0 results.

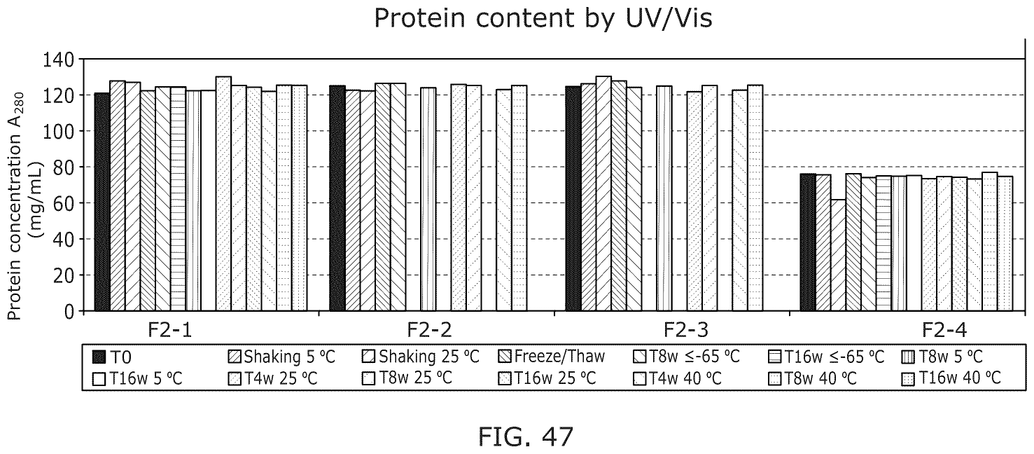

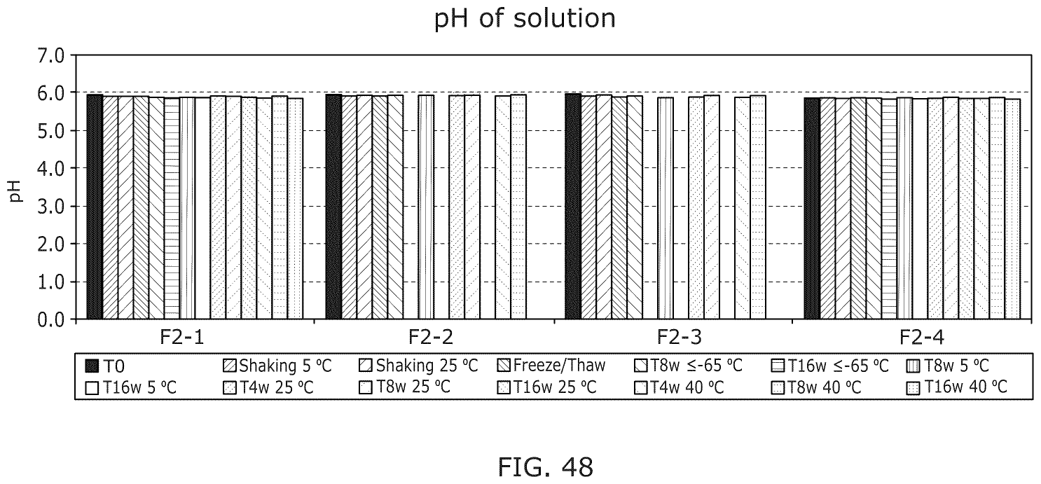

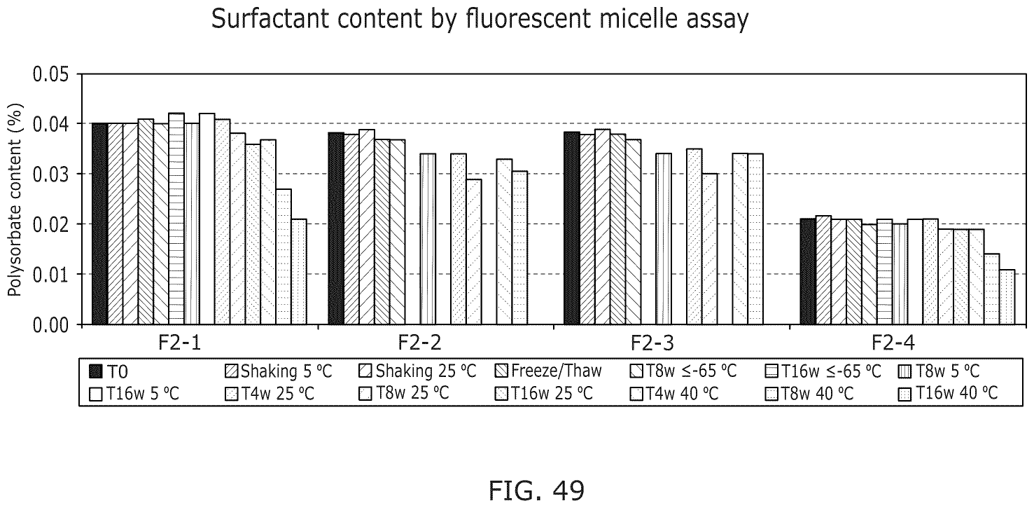

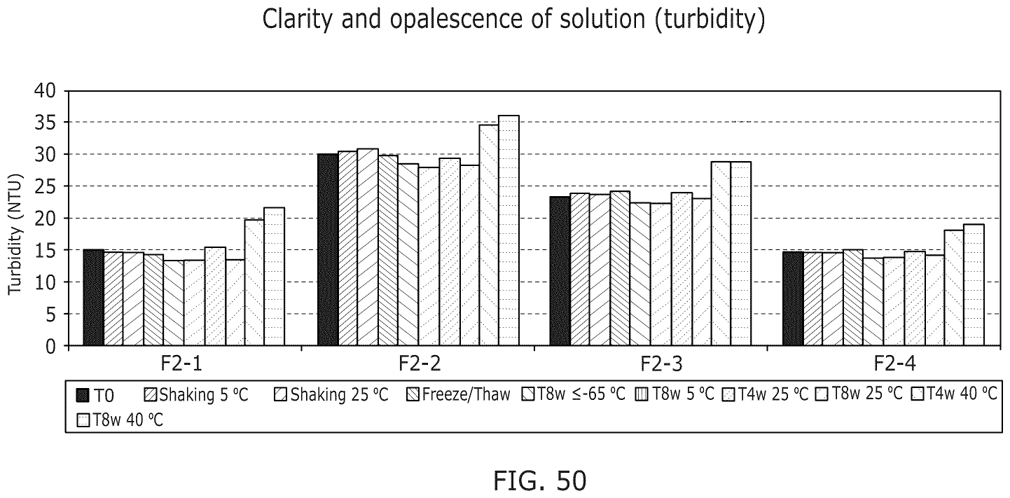

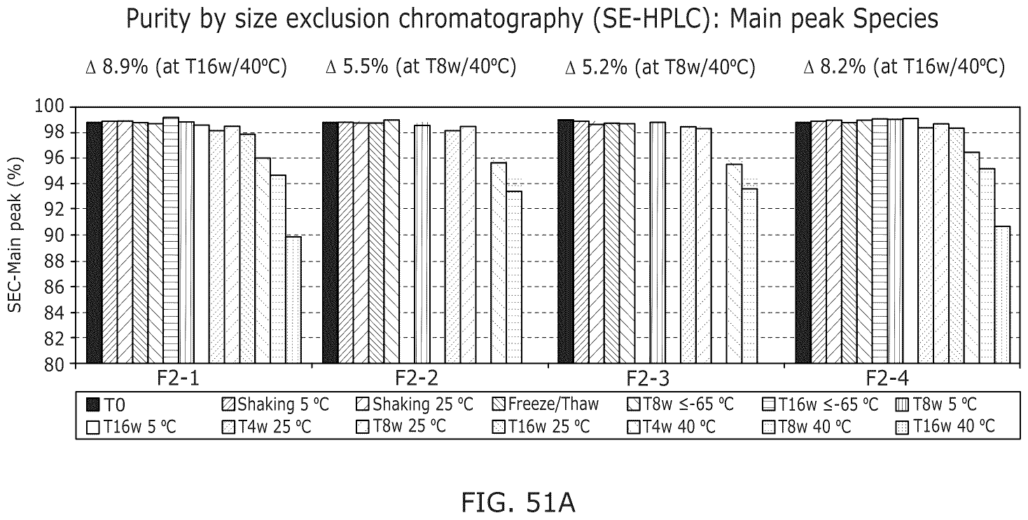

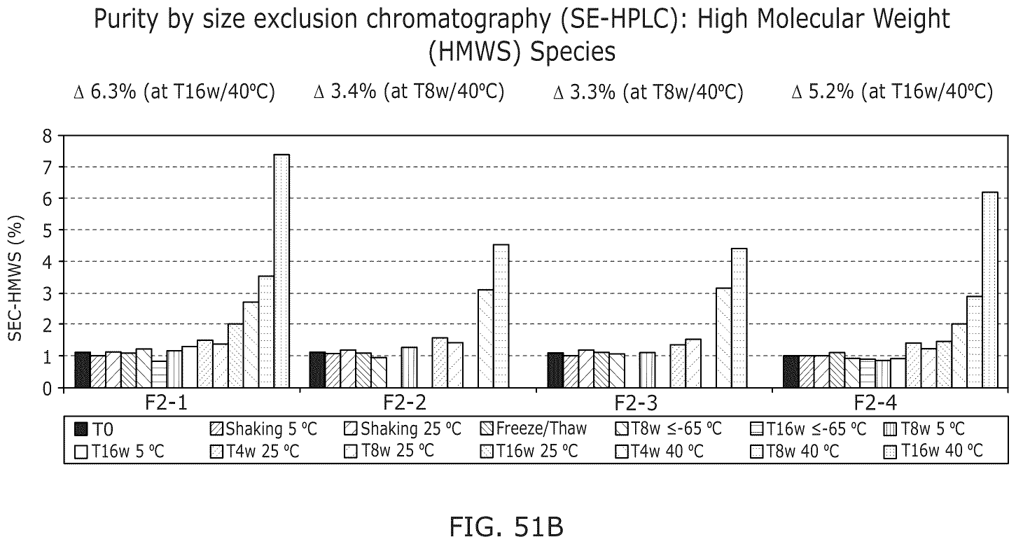

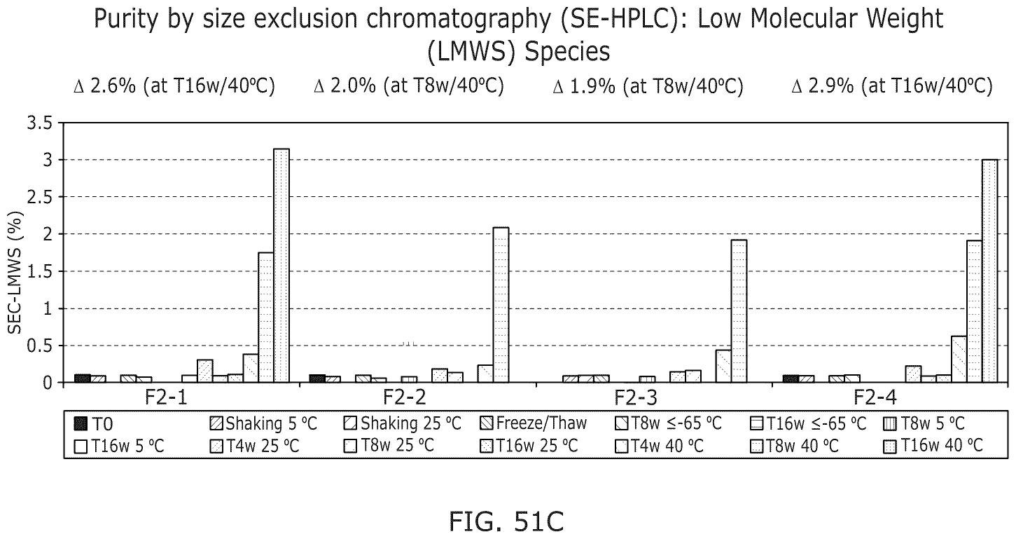

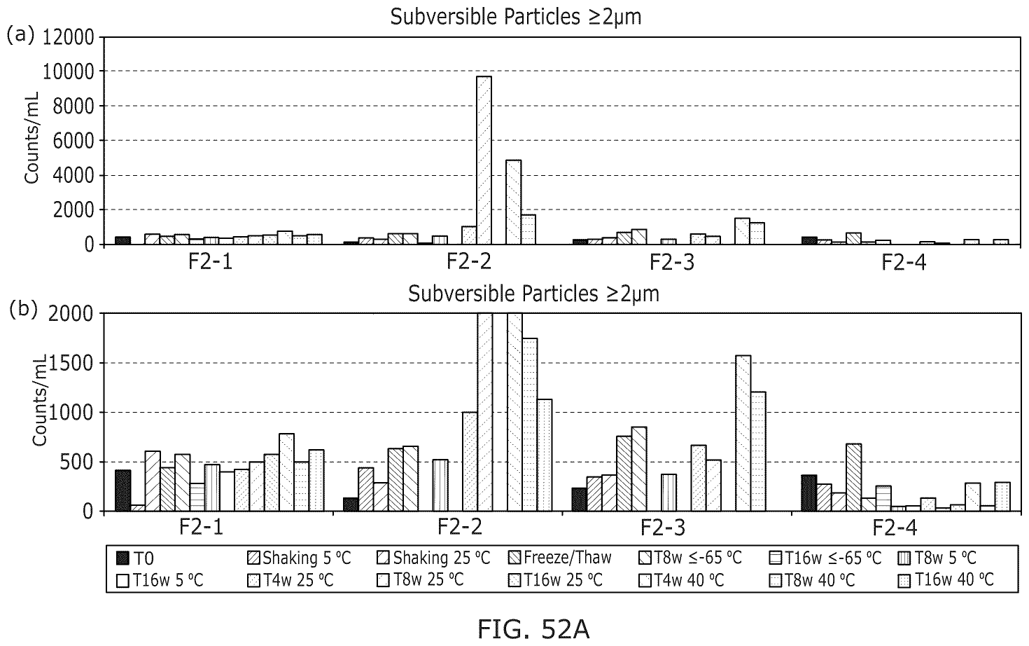

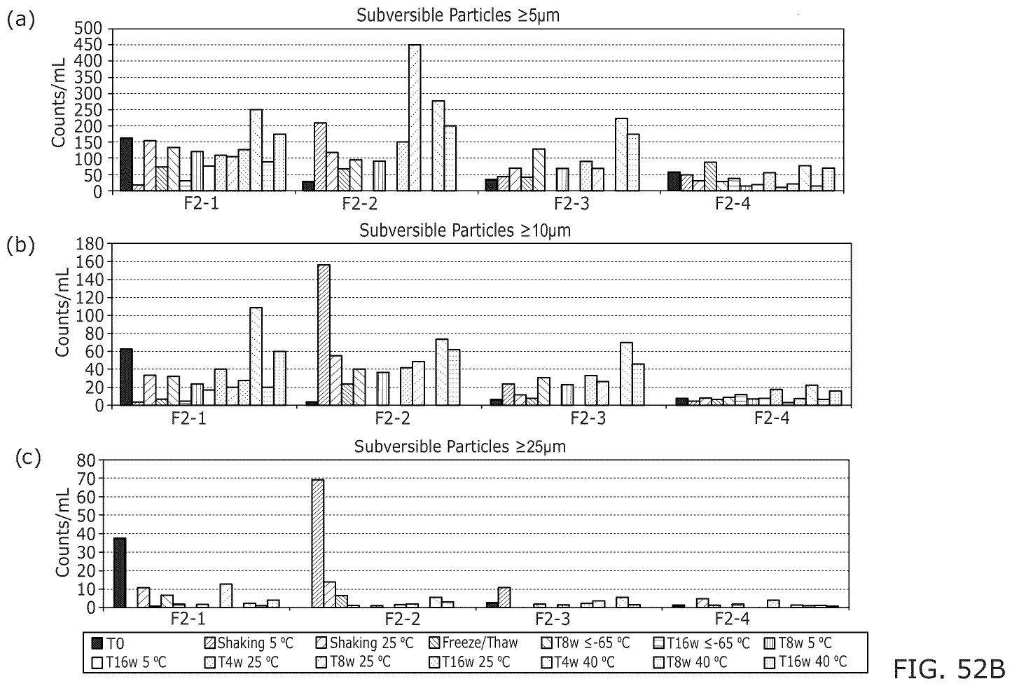

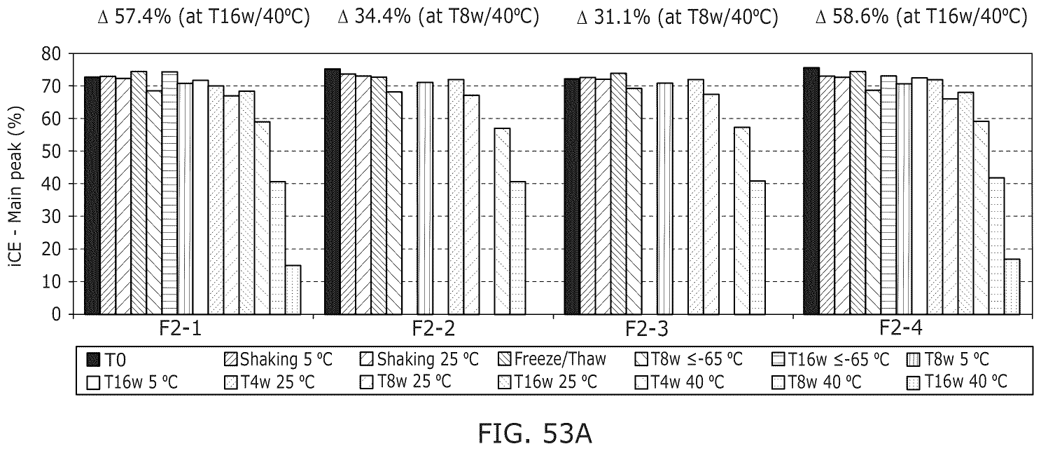

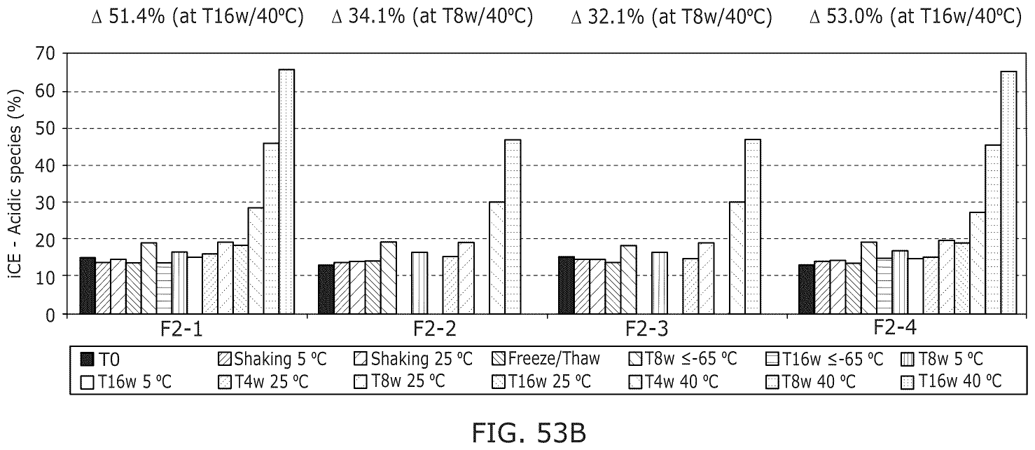

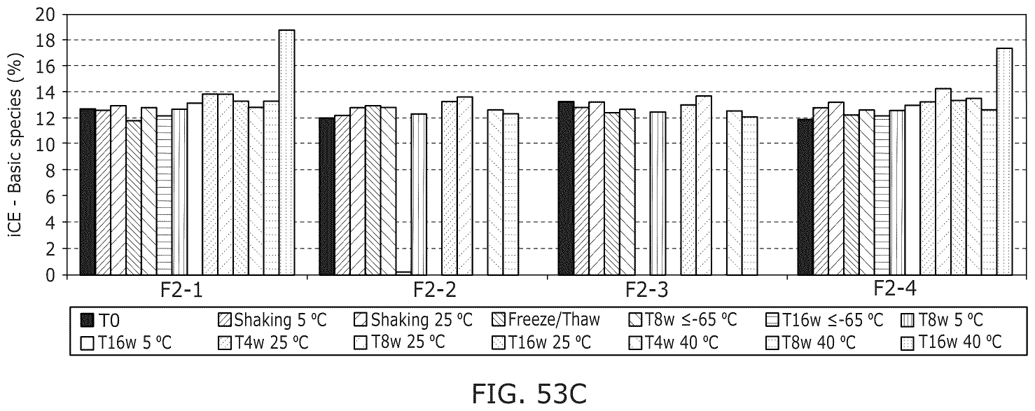

Data from stability studies at initial timepoint T0 and timepoints after being subjected to thermal stress, and data after freeze-thaw stress (−65° C. to 25° C.) or shaking stress at ambient temperature and at cool temperature were graphically depicted in FIGS. 47-53C. FIG. 47 graphically depicts the protein contents of the formulation samples measured by UV/Vis. FIG. 48 graphically depicts the pH values of the formulation samples. FIG. 49 graphically depicts the surfactant content of the formulation samples. FIG. 50 graphically depicts the clarity and opalescence of solution (turbidity) of the formulation samples. FIGS. 51A, 51B, and 51C graphically depict the percentage of main peak, HMW species, and LMW species obtained from the SE-HPLC analysis of the formulation samples. FIG. 52A graphically depicts the counts of subvisible particles having a size of at least 2 μm for all formulation samples, where panel (a) illustrates the counts in a range from 0 to 12000 (counts/mL), while panel (b) illustrates the counts in the range of 0-2000 (counts/mL). FIG. 52B graphically depicts the counts of subvisible particles having a size of at least 5 μm (panel a), at least 10 (panel b), and at least 25 (panel (c) for all formulation samples. FIGS. 53A, 53B, and 53C graphically depict the main charge variant (main peak), the acidic variants, and the basic variants of protein measured by icIEF.

### Freeze-Thaw and Shaking Studies

As can be seen in FIGS. 47-50, all formulations subjected to freeze-thaw stress (−65° C. to 25° C.) or shaking stress at ambient temperature and at cool temperature did not show significantly changes in any of the analytical methods compared to the initial non-stressed samples. This indicates that the formulations effectively stabilized the amlitelimab molecule against both freeze-thaw and shaking stress.

No major differences were observed in aggregation and fragmentation by size exclusion chromatography (SE-HPLC). Overall visible and subvisible particle counts were low on shaking and freeze-thaw stress. All aqueous formulations were practically free of visible particles on shaking and freeze-thaw stress. Data indicated that both surfactants, polysorbate 20 and 80, at the tested concentration levels (0.04% w/v at 125 mg/mL and 0.02% at 75 mg/mL) protected formulations against shaking, freezing, and thawing stresses.

### Short-Term Stability Studies

### 1) Aggregation by SE-HPLC

Short-term stability of formulation samples under different thermal stress conditions was analyzed up to 8 weeks. Purity of the antibody was analyzed by size exclusion chromatography (SE-HPLC) to detect the differences in the percentages of high molecular weight species (HMWS), main peak, and low molecular weight species (LMWS) for all formulations samples.

Data showed that the amlitelimab molecule has low to moderate aggregation tendency at 5 and 25° C., being more prominent at 40° C. Following 8 weeks storage at 5° C., all formulations were comparable with maximum 0.2% area loss in main peak purity compared to T0 analyzed by SE-HPLC. Following 8 weeks storage at 25° C., an increase of ≤0.3% area in high molecular weight species (HMWS) was detected for all formulations. After 8 weeks at 40° C., an increase in HMWS between 1.9% to 3.4% was detected in all formulations. Formulations F2a-2 (containing NaCl) and F2a-3 (containing ArgHCl) showed similar extent of HMWS formation, 3.4% and 3.3% respectively, which was higher compared to formulations F2a-1 and F2a-4 at 125 mg/mL and 75 mg/mL, respectively. In particular, an increase in larger HMWS was observed, which was more pronounced compared to F2a-1 and F2a-4.

Formulations F2a-1 and F2a-4, which were additionally analyzed after 16 weeks storage, did not show major changes after 16 weeks storage at 5° C. and 25° C. At 40° C., an increase in the HMWS percentage area of 6.3% and 5.2% compared to T0 was detected for formulations F2a-1 and F2a-4 at 125 mg/mL and 75 mg/mL, respectively. The change in main peak purity was minimally higher for formulation F2a-1 with 8.9% compared to 8.2% area increase for F2a-4.

As can be seen in FIG. 51C, following 8 weeks storage at 40° C., all formulations showed comparable increase in low molecular weight species (LMWS) with 1.6% to 2.0% area increase compared to T0. Slightly lower LMWS formation was detected for F2a-1 and F2a-4 compared to F2a-2 and F2a-3 containing NaCl and ArgHCl as additional excipient, respectively. After 8 weeks storage at 40° C., a shoulder associated to LMWS could be separated for all formulations. Low fragmentation was observed at 2-8° C. and 25° C. After 16 weeks storage at 40° C., formulations F2a-1 and F2a-4 showed a comparable increase in the LMWS percentage area of 2.6% and 2.9%, respectively.

The comparison of F2a-1 and F2a-4 at different protein concentrations after 16 weeks storage revealed comparable aggregation and fragmentation behavior at 125 mg/mL and 75 mg/mL, respectively. Slightly higher formation of HMWS was detected at higher protein concentration, whereas the LMWS were formed independent of the protein content. The change was that main peak purity, HMWS, and LMWS formation showed a non-linear trend dependent on the protein concentration. The results suggest that aggregation and fragmentation rate did not change significantly when the concentration of the protein is above 75 mg/mL.

### 2) Subvisible Particles

As can be seen in FIGS. 52A and 52B, overall initial subvisible particle counts were low to moderate for all formulations. Over the 8 weeks stability study, formulations F2a-2 and F2a-3 containing NaCl and ArgHCl as additional excipients, respectively, showed an increase of subvisible particle counts, which was most pronounced at 40° C. No significant change in subvisible particle counts was detected for formulations F2a-1 and F2a-4 after 16 weeks storage.

### 3) Opalescence of Solution (Turbidity)

As can be seen in FIG. 50, the opalescence value was moderate around 15 NTU for formulations F2a-1 and F2a-4 with protein concentration at 125 and 75 mg/ml respectively. On the other hand, the addition of NaCl (F2a-2) and ArgHCl (F2a-3) increased the turbidity to 30 NTU and 23 NTU. Following 8 weeks storage at 40° C., all formulations showed an increase in turbidity. No changes in turbidity were observed for formulations stored at 2-8° C. and 25° C.

### 4) Charged Variants

FIGS. 53A, 53B, and 53C show that initial levels of charge variants measured by iCIEF chromatograms. Moderate and main charge variant (main peak) is mainly getting into acidic variants at 25° C. and 40° C. after 8 and 16 weeks. All formulations showed comparable loss of main peak purity area between 31.1% and 34.4% and formation of acidic variants area between 31.4% and 34.1% after 8 weeks compared to T0. Data indicated that the addition of NaCl or ArgHCl or the reduction of the protein concentration from 125 mg/mL to 75 mg/mL did not affect the rate of acidic variant formation. Following 16 weeks storage at 40° C., formulations F2a-1 and F2a-4 showed 57.4% and 58.6% change in main peak purity whereas the increase of acidic variant was 51.4% and 53.0%. The iCIEF chromatograms revealed a change of main charge variant and basic variant into acidic variant.

### 5) Purity and Fragmentation by Capillary Electrophoresis

Monomer purity was assessed by chip-based CE-SDS under non-reducing conditions and by reversed phase HPLC (RP-HPLC), respectively. The chip-based CE-SDS results showed loss of monomer peak purity as well as loss of sum of light and heavy chain (LC+HC) mainly at 40° C. Mainly heavy chain cleavage was observed. However, overall fragmentation is moderate for formulations stored at other temperatures or subject to shaking and freeze-thaw stress. Similar results were observed for reversed phase HPLC (RP-HPLC) analysis, suggesting that there is heavy chain cleavage mainly when the formulation was stored at 40° C., comparable for all formulations.

6) PS80 and/or PS20 Quantification

PS80 and PS 20 quantification showed reduction in polysorbate (PS) 20 and 80 over the stability study. PS20-containing formulations (F2a-2, F2a-3) showed comparable loss in surfactant content after 8 weeks at 25° C. and 40° C. On the other hand, loss of PS80 in F2a-1 and F2a-4 was lower at 25° C. compared to PS20.

The study demonstrates that all aqueous formulations prepared and analyzed were stable against freeze-thaw and shaking stress (FIGS. 47-53C). Overall, amlitelimab showed better stability in formulations without NaCl and ArgHCl after 8 weeks storage at 40° C.

Formulations F2a-1 and F2a-4 without NaCl and ArgHCl were additionally analyzed after 16 weeks storage. No major changes were observed in physicochemical properties and main peak purities in formulation (F2a-1) after 16 weeks at 2-8° C. (recommended storage temperature). Aqueous formulation (F2a-1) shows adequate stability over 16 weeks at 25° C., indicating adequate stability at ambient conditions for handling during manufacturing. Temperature stress at 40° C. induces increased physicochemical changes as well as chemical degradation.

Based on the results from the study, a desired aqueous/liquid formulation that agrees with non-GMP and GMP drug product is recommended to comprise 125 mg/mL amlitelimab, 20 mM Histidine/Histidine HCl, 220 mM sucrose, and 0.04% (w/v) polysorbate 80, and water, with a pH at about 6.0.

### Example 7—Exemplary Formulations and Packaged Products

Exemplary aqueous pharmaceutical formulations F3a-1 and F3a-2 were prepared according to the formulation listed in Table 3a-1 below:

TABLE 3a-1
Example Formulations

| Formulation | Formulation |
|---|---|
| F3a-1 | 125 mg/mL amlitelimab |
| | 20 mM L-histidine/histidine hydrochloride |
| | 220 mM sucrose |
| | 0.04% (w/v) polysorbate 80, |
| | water, |
| | pH 6.0 |
| F3a-2 | 75 mg/mL amlitelimab |
| | 20 mM L-histidine/histidine hydrochloride |
| | 220 mM sucrose |
| | 0.04% (w/v) polysorbate 80, |
| | water, |
| | pH 6.0 |
| F3a-3 | 10 mg/mL amlitelimab |
| | 25 mM L-histidine, |
| | 100 mM sodium chloride, |
| | 100 mM mannitol and |
| | 0.02% (weight/volume [w/v]) polysorbate 80 |
| | water, |
| | pH 6.2 |

Containers comprising 1 mL of aqueous formulation F3a-1 or F3a-2 were developed. The containers were 2 mL Type 1 clear glass injection vials, where aqueous formulation F3a-1 or F3a-2 was aseptically filled into such a vial with a nominal fill volume of 1 mL, and the vial was subsequentially sealed with a FLUROTEC® coated stopper and the stopper is crimped over the vial neck with a cap to ensure container closure integrity. As an example, the stopper may be a 13 mm FLUROTEC® injection stopper made by West Company, and the cap may be a 13 mm white FLIP-OFF® overcaps made by West design TruEdge. The formulations F3a-1 and F3a-2 in these glass vials are suitable for s.c. administration to a subject.

The drug product comprising 1 ml of the aqueous formulation, or solution, of formulation F3a-1 or F3a-2 contained in each glass vial is suitable to be used as a pharmaceutical unit dosage form.

SEQUENCE LISTING

| SEQ ID NO: | | | | |
|---|---|---|---|---|
| 33 | 02D10 | | VH Nucleotide | GAGGTGCAGTTGGTGGAGTCTGGGGGAGG |
| | | | Sequence | CTTGGTACAGCCTGGGGGGTCCCTGAGACT |
| | | | | CTCCTGTGCAGCCTCTGGATTCACTTTTAGC |
| | | | | AACTATGCCATGAACTGGGTCCGCCAGGCT |
| | | | | CCAGGGAAGGGGCTGGAGTGGGTCTCAACT |
| | | | | ATTAGCGGAAGTGGTGGTGCCACAAGGTAT |
| | | | | GCAGACTCCGTGAAGGGCCGATTCACCATA |
| | | | | TCCAGAGACAATTCCAGGAACACGGTGTAT |
| | | | | CTGCAAATGAACAGCCTGAGAGTCGAGGA |
| | | | | CACGGCCGTTTTTTACTGTACGAAAGATCG |
| | | | | GCTCATTATGGCTACGGTTCGGGGACCCTA |
| | | | | TTACTACGGTATGGACGTCTGGGGCCAAGG |
| | | | | GACCACGGTCACCGTCTCCTCA |
| 34 | | | VH Amino Acid | EVQLVESGGGLVQPGGSLRLSCAASGFTFSN |
| | | | Sequence | YAMNWVRQAPGKGLEWVSTISGSGGATRYA |
| | | | | DSVKGRFTISRDNSRNTVYLQMNSLRVEDTA |
| | | | | VFYCTKDRLIMATVRGPYYYGMDVWGQGTT |
| | | | | VTVSS |
| 35 | | | HCDR1 Nucleotide | GGATTCACTTTTAGCAACTATGCC |
| | | | Sequence (IMGT) | |
| 36 | | | HCDR1 Amino | GFTFSNYA |
| | | | Acid Sequence | |
| | | | (IMGT) | |
| 37 | | | HCDR2 Nucleotide | ATTAGCGGAAGTGGTGGTGCCACA |
| | | | Sequence (IMGT) | |
| 38 | | | HCDR2 Amino | ISGSGGAT |
| | | | Acid Sequence | |
| | | | (IMGT) | |
| 39 | | | HCDR3 Nucleotide | ACGAAAGATCGGCTCATTATGGCTACGGTT |
| | | | Sequence (IMGT) | CGGGGACCCTATTACTACGGTATGGACGTC |
| 40 | | | HCDR3 Amino | TKDRLIMATVRGPYYYGMDV |
| | | | Acid Sequence | |
| | | | (IMGT) | |
| 41 | | | HCDR1 Nucleotide | AACTATGCCATGAAC |
| | | | Sequence (KABAT) | |
| 42 | | | HCDR1 Amino | NYAMN |
| | | | Acid Sequence | |
| | | | (KABAT) | |
| 43 | | | HCDR2 Nucleotide | ACTATTAGCGGAAGTGGTGGTGCCACAAGG |
| | | | Sequence (KABAT) | TATGCAGACTCCGTGAAGGGC |
| 44 | | | HCDR2 Amino | TISGSGGATRYADSVKG |
| | | | Acid Sequence | |
| | | | (KABAT) | |
| 45 | | | HCDR3 Nucleotide | GATCGGCTCATTATGGCTACGGTTCGGGGA |
| | | | Sequence (KABAT) | CCCTATTACTACGGTATGGACGTC |
| 46 | | | HCDR3 Amino | DRLIMATVRGPYYYGMDV |
| | | | Acid Sequence | |
| | | | (KABAT) | |
| 47 | | | VL Nucleotide | GACATCCAGATGACCCAGTCTCCATCCTCC |
| | | | Sequence | CTGTCTGCATCTGTAGGAGACAGAGTCACC |
| | | | | ATCACTTGCCGGGCAAGTCAGAGCATTAGC |
| | | | | AGCTATTTAAATTGGTATCAGCAGAAACCA |
| | | | | GGGAAAGCCCCTAACCTCCTGATCTATGCT |
| | | | | GCATCCAGTTTGCAAAGTGGGGTCCCATCA |
| | | | | AGGTTCAGTGGCAGTGGATCTGAGACAGAT |
| | | | | TTCACTCTCACCATCAGCAGTCTGCAACCT |
| | | | | GAAGATTTTGCAACTTACTACTGTCAACAG |
| | | | | AGTCACAGTGTCTCATTCACTTTCGGCCCTG |
| | | | | GGACCAAAGTGGATATCAAA |
| 48 | | | VL Amino Acid | DIQMTQSPSSLSASVGDRVTITCRASQSISSYL |
| | | | Sequence | NWYQQKPGKAPNLLIYAASSLQSGVPSRFSG |
| | | | | SGSETDFTLTISSLQPEDFATYYCQQSHSVSFT |
| | | | | FGPGTKVDIK |
| 49 | | | LCDR1 Nucleotide | CAGAGCATTAGCAGCTAT |
| | | | Sequence (IMGT) | |
| 50 | | | LCDR1 Amino | QSISSY |
| | | | Acid Sequence | |
| | | | (IMGT) | |
| 51 | | | LCDR2 Nucleotide | GCTGCATCC |
| | | | Sequence (IMGT) | |
| 52 | | | LCDR2 Amino | AAS |
| | | | Acid Sequence | |
| | | | (IMGT) | |
| 53 | | | LCDR3 Nucleotide | CAACAGAGTCACAGTGTCTCATTCACT |
| | | | Sequence (IMGT) | |
| 54 | | | LCDR3 Amino | QQSHSVSFT |
| | | | Acid Sequence | |
| | | | (IMGT) | |
| 55 | | | LCDR1 Nucleotide | CGGGCAAGTCAGAGCATTAGCAGCTATTTA |
| | | | Sequence (KABAT) | AAT |
| 56 | | | LCDR1 Amino | RASQSISSYLN |
| | | | Acid Sequence | |
| | | | (KABAT) | |
| 57 | | | LCDR2 Nucleotide | GCTGCATCCAGTTTGCAAAGT |
| | | | Sequence (KABAT) | |
| 58 | | | LCDR2 Amino | AASSLOS |
| | | | Acid Sequence | |
| | | | (KABAT) | |
| 59 | | | LCDR3 Nucleotide | CAACAGAGTCACAGTGTCTCATTCACT |
| | | | Sequence (KABAT) | |
| 60 | | | LCDR3 Amino | QQSHSVSFT |
| | | | Acid Sequence | |
| | | | (KABAT) | |
| 61 | | | Heavy Chain | GAAGTGCAACTGGTGGAGTCCGGAGGAGG |
| | | | Nucleotide | CCTGGTGCAGCCTGGAGGAAGCCTGAGGCT |
| | | | Sequence | GAGCTGTGCCGCCAGCGGCTTCACCTTCAG |
| | | | | CAACTACGCCATGAACTGGGTGAGGCAGGC |
| | | | | CCCTGGCAAGGGACTGGAGTGGGTCTCCAC |
| | | | | CATCAGCGGCTCCGGAGGCGCTACACGGTA |
| | | | | CGCCGATAGCGTGAAGGGCCGGTTTACCAT |
| | | | | TTCCCGGGACAACTCCCGGAACACCGTGTA |
| | | | | CCTCCAGATGAACAGCCTGAGGGTGGAGG |
| | | | | ATACCGCCGTGTTCTACTGCACCAAGGACA |
| | | | | GGCTGATTATGGCCACCGTGAGGGGACCTT |
| | | | | ACTACTATGGCATGGATGTGTGGGGCCAGG |
| | | | | GCACAACCGTCACCGTGTCCTCCGCCTCCA |
| | | | | CCAAGGGACCTAGCGTGTTCCCTCTCGCCC |
| | | | | CCTGTTCCAGGTCCACAAGCGAGTCCACCG |
| | | | | CTGCCCTCGGCTGTCTGGTGAAAGACTACT |
| | | | | TTCCCGAGCCCGTGACCGTCTCCTGGAATA |
| | | | | GCGGAGCCCTGACCTCCGGCGTGCACACAT |
| | | | | TTCCCGCCGTGCTGCAGAGCAGCGGACTGT |
| | | | | ATAGCCTGAGCAGCGTGGTGACCGTGCCCA |
| | | | | GCTCCAGCCTCGGCACCAAAACCTACACCT |
| | | | | GCAACGTGGACCACAAGCCCTCCAACACCA |
| | | | | AGGTGGACAAGCGGGTGGAGAGCAAGTAC |
| | | | | GGCCCCCCTTGCCCTCCTTGTCCTGCCCCTG |
| | | | | AGTTCGAGGGAGGACCCTCCGTGTTCCTGT |
| | | | | TTCCCCCCAAACCCAAGGACACCCTGATGA |
| | | | | TCTCCCGGACACCCGAGGTGACCTGTGTGG |
| | | | | TCGTGGACGTCAGCCAGGAGGACCCCGAG |
| | | | | GTGCAGTTCAACTGGTATGTGGACGGCGTG |
| | | | | GAGGTGCACAATGCCAAAACCAAGCCCAG |
| | | | | GGAGGAGCAGTTCAATTCCACCTACAGGGT |
| | | | | GGTGAGCGTGCTGACCGTCCTGCATCAGGA |
| | | | | TTGGCTGAACGGCAAGGAGTACAAGTGCA |
| | | | | AGGTGTCCAACAAGGGACTGCCCAGCTCCA |
| | | | | TCGAGAAGACCATCAGCAAGGCTAAGGGC |
| | | | | CAGCCGAGGGAGCCCCAGGTGTATACCCTG |
| | | | | CCTCCTAGCCAGGAAGAGATGACCAAGAA |
| | | | | CCAAGTGTCCCTGACCTGCCTGGTGAAGGG |
| | | | | ATTCTACCCCTCCGACATCGCCGTGGAGTG |
| | | | | GGAGAGCAATGGCCAGCCCGAGAACAACT |
| | | | | ACAAAACAACCCCTCCCGTGCTCGATAGCG |
| | | | | ACGGCAGCTTCTTTCTCTACAGCCGGCTGA |
| | | | | CAGTGGACAAGAGCAGGTGGCAGGAGGGC |
| | | | | AACGTGTTCTCCTGTTCCGTGATGCACGAG |
| | | | | GCCCTGCACAATCACTACACCCAGAAGAGC |
| | | | | CTCTCCCTGTCCCTGGGCAAG |
| 62 | | | Heavy Chain Amino | EVQLVESGGGLVQPGGSLRLSCAASGFTFSN |
| | | | Acid Sequence | YAMNWVRQAPGKGLEWVSTISGSGGATRYA |
| | | | | DSVKGRFTISRDNSRNTVYLQMNSLRVEDTA |
| | | | | VFYCTKDRLIMATVRGPYYYGMDVWGQGTT |
| | | | | VTVSSASTKGPSVFPLAPCSRSTSESTAALGCL |
| | | | | VKDYFPEPVTVSWNSGALTSGVHTFPAVLQS |
| | | | | SGLYSLSSVVTVPSSSLGTKTYTCNVDHKPSN |
| | | | | TKVDKRVESKYGPPCPPCPAPEFEGGPSVFLF |
| | | | | PPKPKDTLMISRTPEVTCVVVDVSQEDPEVQF |
| | | | | NWYVDGVEVHNAKTKPREEQFNSTYRVVSV |
| | | | | LTVLHQDWLNGKEYKCKVSNKGLPSSIEKTIS |
| | | | | KAKGQPREPQVYTLPPSQEEMTKNQVSLTCL |
| | | | | VKGFYPSDIAVEWESNGQPENNYKTTPPVLD |
| | | | | SDGSFFLYSRLTVDKSRWQEGNVFSCSVMHE |
| | | | | ALHNHYTQKSLSLSLGK |
| 63 | | | Light Chain | GACATCCAGATGACCCAGTCCCCTTCCTCC |
| | | | Nucleotide | CTGAGCGCTAGCGTGGGAGATAGGGTGACC |
| | | | Sequence | ATCACCTGCAGGGCCTCCCAAAGCATCTCC |
| | | | | TCCTACCTGAACTGGTACCAGCAGAAACCC |
| | | | | GGCAAGGCCCCCAACCTGCTGATCTACGCT |
| | | | | GCCTCCTCCCTCCAGTCCGGCGTGCCTAGC |
| | | | | AGGTTTAGCGGCTCCGGAAGCGAGACCGAC |
| | | | | TTCACCCTGACCATCTCCTCCCTGCAGCCCG |
| | | | | AGGACTTCGCCACCTACTACTGCCAGCAAT |
| | | | | CCCACAGCGTGTCCTTCACCTTCGGCCCCG |
| | | | | GCACCAAGGTGGACATCAAGAGGACCGTG |
| | | | | GCCGCCCCCTCCGTGTTCATCTTTCCCCCCT |
| | | | | CCGATGAACAGCTGAAGAGCGGCACCGCT |
| | | | | AGCGTGGTGTGCCTGCTGAACAACTTCTAC |
| | | | | CCCAGGGAGGCCAAGGTGCAGTGGAAGGT |
| | | | | GGACAATGCCCTGCAGTCCGGCAACAGCCA |
| | | | | GGAGAGCGTGACCGAGCAGGACTCCAAGG |
| | | | | ACAGCACCTACAGCCTGTCCTCCACCCTGA |
| | | | | CCCTGTCCAAGGCCGACTACGAGAAGCACA |
| | | | | AAGTGTACGCCTGCGAAGTGACCCATCAGG |
| | | | | GCCTGAGCTCCCCCGTGACCAAGTCCTTTA |
| | | | | ACAGGGGCGAGTGC |
| 64 | | | Light Chain Amino | DIQMTQSPSSLSASVGDRVTITCRASQSISSYL |
| | | | Acid Sequence | NWYQQKPGKAPNLLIYAASSLQSGVPSRFSG |
| | | | | SGSETDFTLTISSLQPEDFATYYCQQSHSVSFT |
| | | | | FGPGTKVDIKRTVAAPSVFIFPPSDEQLKSGTA |
| | | | | SVVCLLNNFYPREAKVQWKVDNALQSGNSQ |
| | | | | ESVTEQDSKDSTYSLSSTLTLSKADYEKHKV |
| | | | | YACEVTHQGLSSPVTKSFNRGEC |
| 65 | 09H04 | | VH Nucleotide | CAGGTGCAGCTGGTGGAGTCTGGGGGAGG |
| | | | Sequence | CTTGGTCAAGCCTGGAGGGTCCCTGAGACT |
| | | | | CTCCTGTGCAGCCTCTCGATTCACCCTCAGT |
| | | | | GACTACTACATGACCTGGATCCGCCAGGCT |
| | | | | CCAGGGAAGGGGCTGGAGTGGGTTTCATAC |
| | | | | ATTAGTAGTAGTGGTAATACCATATACTAC |
| | | | | GCAGACTCTGTGAAGGGCCGATTCACCATC |
| | | | | TCCAGGGACAACGCCAAGAACTCACTGTAT |
| | | | | CTGCAAATGAACAGCCTGAGAGCCGAGGA |
| | | | | CACGGCCGTGTATTACTGTGCGAGAGATCT |
| | | | | GAGTGGGAGCTACTGGGACTACTACTACGG |
| | | | | TATGGACGTCTGGGGCCAAGGGACCACGGT |
| | | | | CACCGTCTCCTCA |
| 66 | | | VH Amino Acid | QVQLVESGGGLVKPGGSLRLSCAASRFTLSD |
| | | | Sequence | YYMTWIRQAPGKGLEWVSYISSSGNTIYYAD |
| | | | | SVKGRFTISRDNAKNSLYLQMNSLRAEDTAV |
| | | | | YYCARDLSGSYWDYYYGMDVWGQGTTVTV |
| | | | | SS |
| 67 | | | HCDR1 Nucleotide | CGATTCACCCTCAGTGACTACTAC |
| | | | Sequence (IMGT) | |
| 68 | | | HCDR1 Amino | RFTLSDYY |
| | | | Acid Sequence | |
| | | | (IMGT) | |
| 69 | | | HCDR2 Nucleotide | ATTAGTAGTAGTGGTAATACCATA |
| | | | Sequence (IMGT) | |
| 70 | | | HCDR2 Amino | ISSSGNTI |
| | | | Acid Sequence | |
| | | | (IMGT) | |
| 71 | | | HCDR3 Nucleotide | GCGAGAGATCTGAGTGGGAGCTACTGGGA |
| | | | Sequence (IMGT) | CTACTACTACGGTATGGACGTC |
| 72 | | | HCDR3 Amino | ARDLSGSYWDYYYGMDV |
| | | | Acid Sequence | |
| | | | (IMGT) | |
| 73 | | | HCDR1 Nucleotide | GACTACTACATGACC |
| | | | Sequence (KABAT) | |
| 74 | | | HCDR1 Amino | DYYMT |
| | | | Acid Sequence | |
| | | | (KABAT) | |
| 75 | | | HCDR2 Nucleotide | TACATTAGTAGTAGTGGTAATACCATATAC |
| | | | Sequence (KABAT) | TACGCAGACTCTGTGAAGGGC |
| 76 | | | HCDR2 Amino | YISSSGNTIYYADSVKG |
| | | | Acid Sequence | |
| | | | (KABAT) | |
| 77 | | | HCDR3 Nucleotide | GATCTGAGTGGGAGCTACTGGGACTACTAC |
| | | | Sequence (KABAT) | TACGGTATGGACGTC |
| 78 | | | HCDR3 Amino | DLSGSYWDYYYGMDV |
| | | | Acid Sequence | |
| | | | (KABAT) | |
| 79 | | | VL Nucleotide | GCCATCCAGTTGACCCAGTCTCCATCCTCCC |
| | | | Sequence | TGTCTACATCTGTAGGAGACAGAGTCACCA |
| | | | | TCGCTTGCCGGGCAAGTCAGGGCATTAACA |
| | | | | ATGCTTTAGCCTGGTATCAGCAGAAACCAG |
| | | | | GGAAAGCTCCTAAGCTCCTGATCTATGATG |
| | | | | CCTCCAGTTTGGAAAGTGGGGTCCCATCAA |
| | | | | GGTTCAGCGGCAGTGGATCTGGGACAGATT |
| | | | | TCACTCTCACCATCAGCAGCCTGCAGCCTG |
| | | | | AAGATTTTGCAACTTATTACTGTCAACAGTT |
| | | | | TAATAGTTACCCTCGGACGTTCGGCCAAGG |
| | | | | GACCAAGGTGGAAATCAAA |
| 80 | | | VL Amino Acid | AIQLTQSPSSLSTSVGDRVTIACRASQGINNAL |
| | | | Sequence | AWYQQKPGKAPKLLIYDASSLESGVPSRFSGS |
| | | | | GSGTDFTLTISSLQPEDFATYYCQQFNSYPRTF |
| | | | | GQGTKVEIK |
| 81 | | | LCDR1 Nucleotide | CAGGGCATTAACAATGCT |
| | | | Sequence (IMGT) | |
| 82 | | | LCDR1 Amino | QGINNA |
| | | | Acid Sequence | |
| | | | (IMGT) | |
| 83 | | | LCDR2 Nucleotide | GATGCCTCC |
| | | | Sequence (IMGT) | |
| 84 | | | LCDR2 Amino | DAS |
| | | | Acid Sequence | |
| | | | (IMGT) | |
| 85 | | | LCDR3 Nucleotide | CAACAGTTTAATAGTTACCCTCGGACG |
| | | | Sequence (IMGT) | |
| 86 | | | LCDR3 Amino | QQFNSYPRT |
| | | | Acid Sequence | |
| | | | (IMGT) | |
| 87 | | | LCDR1 Nucleotide | CGGGCAAGTCAGGGCATTAACAATGCTTTA |
| | | | Sequence (KABAT) | GCC |
| 88 | | | LCDR1 Amino | RASQGINNALA |
| | | | Acid Sequence | |
| | | | (KABAT) | |
| 89 | | | LCDR2 Nucleotide | GATGCCTCCAGTTTGGAAAGT |
| | | | Sequence (KABAT) | |
| 90 | | | LCDR2 Amino | DASSLES |
| | | | Acid Sequence | |
| | | | (KABAT) | |
| 91 | | | LCDR3 Nucleotide | CAACAGTTTAATAGTTACCCTCGGACG |
| | | | Sequence (KABAT) | |
| 92 | | | LCDR3 Amino | QQFNSYPRT |
| | | | Acid Sequence | |
| | | | (KABAT) | |
| 93 | 19H01 | | VH Nucleotide | GAGGTGCAGCTGGTGGAGTCTGGGGGAGG |
| | | | Sequence | CTTGGTAAAGCCTGGGGGGTCCCTTAGACT |
| | | | | CTCCTGTGCAGCCTCTGGATTCACTTTCAGT |
| | | | | AACGCCTGGATGAGCTGGGTCCGCCAGGCT |
| | | | | CCAGGGAAGGGGCTGGAGTGGGTTGGCCG |
| | | | | TATTAAAAGCAAAACTGAAGGTGGGACAA |
| | | | | CAGACTACGCTGCACCCGTGAAAGGCAGAT |
| | | | | TCACCATCTCAAGAGATGATTCAAAAAACA |
| | | | | CGCTGTATCTGCAAATGAACAGCCTGAAAA |
| | | | | CCGAGGACACAGCCGTGTATTACTGTACCA |
| | | | | CAGATTTTCTATGGTTCGGGGAGTTCCCTTT |
| | | | | TGACTACTGGGGCCAGGGAACCCTGGTCAC |
| | | | | CGTCTCCTCA |
| 94 | | | VH Amino Acid | EVOLVESGGGLVKPGGSLRLSCAASGFTFSN |
| | | | Sequence | AWMSWVRQAPGKGLEWVGRIKSKTEGGTT |
| | | | | DYAAPVKGRFTISRDDSKNTLYLQMNSLKTE |
| | | | | DTAVYYCTTDFLWFGEFPFDYWGQGTLVTV |
| | | | | SS |
| 95 | | | HCDR1 Nucleotide | GGATTCACTTTCAGTAACGCCTGG |
| | | | Sequence (IMGT) | |
| 96 | | | HCDR1 Amino | GFTFSNAW |
| | | | Acid Sequence | |
| | | | (IMGT) | |
| 97 | | | HCDR2 Nucleotide | ATTAAAAGCAAAACTGAAGGTGGGACAAC |
| | | | Sequence (IMGT) | A |
| 98 | | | HCDR2 Amino | IKSKTEGGTT |
| | | | Acid Sequence | |
| | | | (IMGT) | |
| 99 | | | HCDR3 Nucleotide | ACCACAGATTTTCTATGGTTCGGGGAGTTC |
| | | | Sequence (IMGT) | CCTTTTGACTAC |
| 100 | | | HCDR3 Amino | TTDFLWFGEFPFDY |
| | | | Acid Sequence | |
| | | | (IMGT) | |
| 101 | | | HCDR1 Nucleotide | AACGCCTGGATGAGC |
| | | | Sequence (KABAT) | |
| 102 | | | HCDR1 Amino | NAWMS |
| | | | Acid Sequence | |
| | | | (KABAT) | |
| 103 | | | HCDR2 Nucleotide | CGTATTAAAAGCAAAACTGAAGGTGGGAC |
| | | | Sequence (KABAT) | AACAGACTACGCTGCACCCGTGAAAGGC |
| 104 | | | HCDR2 Amino | RIKSKTEGGTTDYAAPVKG |
| | | | Acid Sequence | |
| | | | (KABAT) | |
| 105 | | | HCDR3 Nucleotide | GATTTTCTATGGTTCGGGGAGTTCCCTTTTG |
| | | | Sequence (KABAT) | ACTAC |
| 106 | | | HCDR3 Amino | DFLWFGEFPFDY |
| | | | Acid Sequence | |
| | | | (KABAT) | |
| 107 | | | VL Nucleotide | GACATCCAGATGACCCAGTCTCCATCCTCC |
| | | | Sequence | CTGTCTGCATCTGTAGGAGACAGAGTCACC |
| | | | | ATCACTTGCCGGGCGAGTCAGGGCATTAGC |
| | | | | AATTATTTAGCCTGGTATCAGCAGAAACCA |
| | | | | GGGAAAATTCCTAAGCTCCTGATCTATGCT |
| | | | | GCATCCACTTTGCAATCAGGGGTCCCATCT |
| | | | | CGGTTCAGTGGCAGTGGATCTGGGACAGAT |
| | | | | TTCACTCTCACCATCAGCAGCCTGCAGCCT |
| | | | | GAAGATGTTGCAACTTATTACTGTCAAAAG |
| | | | | TATAACAGTGCCCCTCGGACGTTCGGCCAA |
| | | | | GGGACCAAGGTGGAAATCAAA |
| 108 | | | VL Amino Acid | DIQMTQSPSSLSASVGDRVTITCRASQGISNYL |
| | | | Sequence | AWYQQKPGKIPKLLIYAASTLQSGVPSRFSGS |
| | | | | GSGTDFTLTISSLQPEDVATYYCQKYNSAPRT |
| | | | | FGQGTKVEIK |
| 109 | | | LCDR1 Nucleotide | CAGGGCATTAGCAATTAT |
| | | | Sequence (IMGT) | |
| 110 | | | LCDR1 Amino | QGISNY |
| | | | Acid Sequence | |
| | | | (IMGT) | |
| 111 | | | LCDR2 Nucleotide | GCTGCATCC |
| | | | Sequence (IMGT) | |
| 112 | | | LCDR2 Amino | AAS |
| | | | Acid Sequence | |
| | | | (IMGT) | |
| 113 | | | LCDR3 Nucleotide | CAAAAGTATAACAGTGCCCCTCGGACG |
| | | | Sequence (IMGT) | |
| 114 | | | LCDR3 Amino | QKYNSAPRT |
| | | | Acid Sequence | |
| | | | (IMGT) | |
| 115 | | | LCDR1 Nucleotide | CGGGCGAGTCAGGGCATTAGCAATTATTTA |
| | | | Sequence (KABAT) | GCC |
| 116 | | | LCDR1 Amino | RASQGISNYLA |
| | | | Acid Sequence | |
| | | | (KABAT) | |
| 117 | | | LCDR2 Nucleotide | GCTGCATCCACTTTGCAATCA |
| | | | Sequence (KABAT) | |
| 118 | | | LCDR2 Amino | AASTLQS |
| | | | Acid Sequence | |
| | | | (KABAT) | |
| 119 | | | LCDR3 Nucleotide | CAAAAGTATAACAGTGCCCCTCGGACG |
| | | | Sequence (KABAT) | |
| 120 | | | LCDR3 Amino | QKYNSAPRT |
| | | | Acid Sequence | |
| | | | (KABAT) | |
| 121 | Human | IGHG* | Heavy Chain | gcttccaccaagggcccatccgtcttccccctggcgccctgctccagg |
| | IgG4 | 01 | Constant Region | agcacctccgagagcacagccgccctgggctgcctggtcaaggacta |
| | heavy | | Nucleotide | cttccccgaaccggtgacggtgtcgtggaactcaggcgccctgaccag |
| | chain | | Sequence | cggcgtgcacaccttcccggctgtcctacagtcctcaggactctactcc |
| | constant | | | ctcagcagcgtggtgaccgtgccctccagcagcttgggcacgaagac |
| | region | | | ctacacctgcaacgtagatcacaagcccagcaacaccaaggtggaca |
| | #1 | | | agagagttgagtccaaatatggtcccccatgcccatcatgcccagcacc |
| | | | | tgagttcctggggggaccatcagtcttcctgttccccccaaaacccaag |
| | | | | gacactctcatgatctcccggacccctgaggtcacgtgcgtggtggtgg |
| | | | | acgtgagccaggaagaccccgaggtccagttcaactggtacgtggat |
| | | | | ggcgtggaggtgcataatgccaagacaaagccgcgggaggagcagt |
| | | | | tcaacagcacgtaccgtgtggtcagcgtcctcaccgtcctgcaccagg |
| | | | | actggctgaacggcaaggagtacaagtgcaaggtctccaacaaaggc |
| | | | | ctcccgtcctccatcgagaaaaccatctccaaagccaaagggcagccc |
| | | | | cgagagccacaggtgtacaccctgcccccatcccaggaggagatgac |
| | | | | caagaaccaggtcagcctgacctgcctggtcaaaggcttctaccccag |
| | | | | cgacatcgccgtggagtgggagagcaatgggcagccggagaacaac |
| | | | | tacaagaccacgcctcccgtgctggactccgacggctccttcttcctcta |
| | | | | cagcaggctaaccgtggacaagagcaggtggcaggaggggaatgtc |
| | | | | ttctcatgctccgtgatgcatgaggctctgcacaaccactacacacaga |
| | | | | agagcctctccctgtctctgggtaaa |
| 122 | | | Heavy Chain | ASTKGPSVFPLAPCSRSTSESTAALGCLVKDY |
| | | | Constant Region | FPEPVTVSWNSGALTSGVHTFPAVLQSSGLYS |
| | | | Amino Acid | LSSVVTVPSSSLGTKTYTCNVDHKPSNTKVD |
| | | | Sequence | KRVESKYGPPCPSCPAPEFLGGPSVFLFPPKPK |
| | | | | DTLMISRTPEVTCVVVDVSQEDPEVQFNWYV |
| | | | | DGVEVHNAKTKPREEQFNSTYRVVSVLTVLH |
| | | | | QDWLNGKEYKCKVSNKGLPSSIEKTISKAKG |
| | | | | QPREPQVYTLPPSQEEMTKNQVSLTCLVKGF |
| | | | | YPSDIAVEWESNGQPENNYKTTPPVLDSDGSF |
| | | | | FLYSRLTVDKSRWQEGNVFSCSVMHEALHN |
| | | | | HYTQKSLSLSLGK |
| 123 | Human | IGHG* | Heavy Chain | gcttccaccaagggcccatccgtcttccccctggcgccctgctccagg |
| | IgG4 | 02 | Constant Region | agcacctccgagagcacagccgccctgggctgcctggtcaaggacta |
| | heavy | | Nucleotide | cttccccgaaccggtgacggtgtcgtggaactcaggcgccctgaccag |
| | chain | | Sequence | cggcgtgcacaccttcccggctgtcctacagtcctcaggactctactcc |
| | constant | | | ctcagcagcgtggtgaccgtgccctccagcagcttgggcacgaagac |
| | region | | | ctacacctgcaacgtagatcacaagcccagcaacaccaaggtggaca |
| | #2 | | | agagagttgagtccaaatatggtcccccgtgcccatcatgcccagcac |
| | | | | ctgagttcctggggggaccatcagtcttcctgttccccccaaaacccaa |
| | | | | ggacactctcatgatctcccggacccctgaggtcacgtgcgtggtggtg |
| | | | | gacgtgagccaggaagaccccgaggtccagttcaactggtacgtgga |
| | | | | tggcgtggaggtgcataatgccaagacaaagccgcgggaggagcag |
| | | | | ttcaacagcacgtaccgtgtggtcagcgtcctcaccgtcgtgcaccagg |
| | | | | actggctgaacggcaaggagtacaagtgcaaggtctccaacaaaggc |
| | | | | ctcccgtcctccatcgagaaaaccatctccaaagccaaagggcagccc |
| | | | | cgagagccacaggtgtacaccctgcccccatcccaggaggagatgac |
| | | | | caagaaccaggtcagcctgacctgcctggtcaaaggcttctaccccag |
| | | | | cgacatcgccgtggagtgggagagcaatgggcagccggagaacaac |
| | | | | tacaagaccacgcctcccgtgctggactccgacggctccttcttcctcta |
| | | | | cagcaggctaaccgtggacaagagcaggtggcaggaggggaatgtc |
| | | | | ttctcatgctccgtgatgcatgaggctctgcacaaccactacacgcaga |
| | | | | agagcctctccctgtctctgggtaaa |
| 124 | | | Heavy Chain | ASTKGPSVFPLAPCSRSTSESTAALGCLVKDY |
| | | | Constant Region | FPEPVTVSWNSGALTSGVHTFPAVLQSSGLYS |
| | | | Amino Acid | LSSVVTVPSSSLGTKTYTCNVDHKPSNTKVD |
| | | | Sequence | KRVESKYGPPCPSCPAPEFLGGPSVFLFPPKPK |
| | | | | DTLMISRTPEVTCVVVDVSQEDPEVQFNWYV |
| | | | | DGVEVHNAKTKPREEQFNSTYRVVSVLTVV |
| | | | | HQDWLNGKEYKCKVSNKGLPSSIEKTISKAK |
| | | | | GQPREPQVYTLPPSQEEMTKNQVSLTCLVKG |
| | | | | FYPSDIAVEWESNGQPENNYKTTPPVLDSDGS |
| | | | | FFLYSRLTVDKSRWQEGNVFSCSVMHEALHN |
| | | | | HYTQKSLSLSLGK |
| 125 | Human | IGHG* | Heavy Chain | gcttccaccaagggcccatccgtcttccccctggcgccctgctccagg |
| | IgG4 | 03 | Constant Region | agcacctccgagagcacagccgccctgggctgcctggtcaaggacta |
| | heavy | | Nucleotide | cttccccgaaccggtgacggtgtcgtggaactcaggcgccctgaccag |
| | chain | | Sequence | cggcgtgcacaccttcccggctgtcctacagtcctcaggactctactcc |
| | constant | | | ctcagcagcgtggtgaccgtgccctccagcagcttgggcacgaagac |
| | region | | | ctacacctgcaacgtagatcacaagcccagcaacaccaaggtggaca |
| | #3 | | | agagagttgagtccaaatatggtcccccatgcccatcatgcccagcacc |
| | | | | tgagttcctggggggaccatcagtcttcctgttccccccaaaacccaag |
| | | | | gacactctcatgatctcccggacccctgaggtcacgtgcgtggtggtgg |
| | | | | acgtgagccaggaagaccccgaggtccagttcaactggtacgtggat |
| | | | | ggcgtggaggtgcataatgccaagacaaagccgcgggaggagcagt |
| | | | | tcaacagcacgtaccgtgtggtcagcgtcctcaccgtcctgcaccagg |
| | | | | actggctgaacggcaaggagtacaagtgcaaggtctccaacaaaggc |
| | | | | ctcccgtcctccatcgagaaaaccatctccaaagccaaagggcagccc |
| | | | | cgagagccacaggtgtacaccctgcccccatcccaggaggagatgac |
| | | | | caagaaccaggtcagcctgacctgcctggtcaaaggcttctaccccag |
| | | | | cgacatcgccgtggagtgggagagcaatgggcagccggagaacaac |
| | | | | tacaagaccacgcctcccgtgctggactccgacggctccttcttcctcta |
| | | | | cagcaagctcaccgtggacaagagcaggtggcaggaggggaacgtc |
| | | | | ttctcatgctccgtgatgcatgaggctctgcacaaccactacacgcaga |
| | | | | agagcctctccctgtctctgggtaaa |
| 126 | | | Heavy Chain | ASTKGPSVFPLAPCSRSTSESTAALGCLVKDY |
| | | | Constant Region | FPEPVTVSWNSGALTSGVHTFPAVLQSSGLYS |
| | | | Amino Acid | LSSVVTVPSSSLGTKTYTCNVDHKPSNTKVD |
| | | | Sequence | KRVESKYGPPCPSCPAPEFLGGPSVFLFPPKPK |
| | | | | DTLMISRTPEVTCVVVDVSQEDPEVQFNWYV |
| | | | | DGVEVHNAKTKPREEQFNSTYRVVSVLTVLH |
| | | | | QDWLNGKEYKCKVSNKGLPSSIEKTISKAKG |
| | | | | QPREPQVYTLPPSQEEMTKNQVSLTCLVKGF |
| | | | | YPSDIAVEWESNGQPENNYKTTPPVLDSDGSF |
| | | | | FLYSKLTVDKSRWQEGNVFSCSVMHEALHN |
| | | | | HYTQKSLSLSLGK |
| 127 | IgG4 | | Heavy Chain | gcctccaccaagggcccatccgtcttccccctggcgccctgctccagg |
| | heavy | | Constant Region | agcacctccgagagcacggccgccctgggctgcctggtcaaggacta |
| | chain | | Nucleotide | cttccccgaaccagtgacggtgtcgtggaactcaggcgccctgaccag |
| | constant | | Sequence- | cggcgtgcacaccttcccggctgtcctacagtcctcaggactctactcc |
| | region | | Synthetic Version A | ctcagcagcgtggtgaccgtgccctccagcagcttgggcacgaagac |
| | | | | ctacacctgcaacgtagatcacaagcccagcaacaccaaggtggaca |
| | | | | agagagttgagtccaaatatggtcccccatgcccaccatgcccagcgc |
| | | | | ctgaatttgaggggggaccatcagtcttcctgttccccccaaaacccaa |
| | | | | ggacactctcatgatctcccggacccctgaggtcacgtgcgtggtggtg |
| | | | | gacgtgagccaggaagaccccgaggtccagttcaactggtacgtgga |
| | | | | tggcgtggaggtgcataatgccaagacaaagccgcgggaggagcag |
| | | | | ttcaacagcacgtaccgtgtggtcagcgtcctcaccgtcctgcaccagg |
| | | | | actggctgaacggcaaggagtacaagtgcaaggtctccaacaaaggc |
| | | | | ctcccgtcatcgatcgagaaaaccatctccaaagccaaagggcagccc |
| | | | | cgagagccacaggtgtacaccctgcccccatcccaggaggagatgac |
| | | | | caagaaccaggtcagcctgacctgcctggtcaaaggcttctaccccag |
| | | | | cgacatcgccgtggagtgggagagcaatgggcagccggagaacaac |
| | | | | tacaagaccacgcctcccgtgctggactccgacggatccttcttcctcta |
| | | | | cagcaggctaaccgtggacaagagcaggtggcaggaggggaatgtc |
| | | | | ttctcatgctccgtgatgcatgaggctctgcacaaccactacacacaga |
| | | | | agagcctctccctgtctctgggtaaa |
| 128 | IgG4 | | Heavy Chain | ASTKGPSVFPLAPCSRSTSESTAALGCLVKDY |
| | heavy | | Constant Region | FPEPVTVSWNSGALTSGVHTFPAVLQSSGLYS |
| | chain | | Amino Acid | LSSVVTVPSSSLGTKTYTCNVDHKPSNTKVD |
| | constant | | Sequence-Encoded | KRVESKYGPPCPPCPAPEFEGGPSVFLFPPKPK |
| | region- | | by Synthetic | DTLMISRTPEVTCVVVDVSQEDPEVQFNWYV |
| | IgG4-PE | | Version A, B & C | DGVEVHNAKTKPREEQFNSTYRVVSVLTVLH |
| | | | | QDWLNGKEYKCKVSNKGLPSSIEKTISKAKG |
| | | | | QPREPQVYTLPPSQEEMTKNQVSLTCLVKGF |
| | | | | YPSDIAVEWESNGQPENNYKTTPPVLDSDGSF |
| | | | | FLYSRLTVDKSRWQEGNVFSCSVMHEALHN |
| | | | | HYTQKSLSLSLGK |
| 129 | IgG4 | | Heavy Chain | Gcctccaccaagggacctagcgtgttccctctcgccccctgttccaggt |
| | heavy | | Constant Region | ccacaagcgagtccaccgctgccctcggctgtctggtgaaagactactt |
| | chain | | Nucleotide | tcccgagcccgtgaccgtctcctggaatagcggagccctgacctccgg |
| | constant | | Sequence- | cgtgcacacatttcccgccgtgctgcagagcagcggactgtatagcct |
| | region | | Synthetic Version B | gagcagcgtggtgaccgtgcccagctccagcctcggcaccaaaacct |
| | | | | acacctgcaacgtggaccacaagccctccaacaccaaggtggacaag |
| | | | | cgggtggagagcaagtacggccccccttgccctccttgtcctgcccct |
| | | | | gagttcgagggaggaccctccgtgttcctgtttccccccaaacccaagg |
| | | | | acaccctgatgatctcccggacacccgaggtgacctgtgtggtcgtgg |
| | | | | acgtcagccaggaggaccccgaggtgcagttcaactggtatgtggac |
| | | | | ggcgtggaggtgcacaatgccaaaaccaagcccagggaggagcagt |
| | | | | tcaattccacctacagggtggtgagcgtgctgaccgtcctgcatcagga |
| | | | | ttggctgaacggcaaggagtacaagtgcaaggtgtccaacaagggac |
| | | | | tgcccagctccatcgagaagaccatcagcaaggctaagggccagccg |
| | | | | agggagccccaggtgtataccctgcctcctagccaggaagagatgac |
| | | | | caagaaccaagtgtccctgacctgcctggtgaagggattctacccctcc |
| | | | | gacatcgccgtggagtgggagagcaatggccagcccgagaacaact |
| | | | | acaaaacaacccctcccgtgctcgatagcgacggcagcttctttctctac |
| | | | | agccggctgacagtggacaagagcaggtggcaggagggcaacgtgt |
| | | | | tctcctgttccgtgatgcacgaggccctgcacaatcactacacccagaa |
| | | | | gagcctctccctgtccctgggcaag |
| 130 | IgG4 | | Heavy Chain | gccagcaccaagggcccttccgtgttccccctggccccttgcagcagg |
| | heavy | | Constant Region | agcacctccgaatccacagctgccctgggctgtctggtgaaggactact |
| | chain | | Nucleotide | ttcccgagcccgtgaccgtgagctggaacagcggcgctctgacatccg |
| | constant | | Sequence- | gcgtccacacctttcctgccgtcctgcagtcctccggcctctactccctgt |
| | region | | Synthetic Version C | cctccgtggtgaccgtgcctagctcctccctcggcaccaagacctacac |
| | | | | ctgtaacgtggaccacaaaccctccaacaccaaggtggacaaacggg |
| | | | | tcgagagcaagtacggccctccctgccctccttgtcctgcccccgagtt |
| | | | | cgaaggcggacccagcgtgttcctgttccctcctaagcccaaggacac |
| | | | | cctcatgatcagccggacacccgaggtgacctgcgtggtggtggatgt |
| | | | | gagccaggaggaccctgaggtccagttcaactggtatgtggatggcgt |
| | | | | ggaggtgcacaacgccaagacaaagccccgggaagagcagttcaac |
| | | | | tccacctacagggtggtcagcgtgctgaccgtgctgcatcaggactgg |
| | | | | ctgaacggcaaggagtacaagtgcaaggtcagcaataagggactgcc |
| | | | | cagcagcatcgagaagaccatctccaaggctaaaggccagccccgg |
| | | | | gaacctcaggtgtacaccctgcctcccagccaggaggagatgaccaa |
| | | | | gaaccaggtgagcctgacctgcctggtgaagggattctacccttccga |
| | | | | catcgccgtggagtgggagtccaacggccagcccgagaacaattata |
| | | | | agaccacccctcccgtcctcgacagcgacggatccttctttctgtactcc |
| | | | | aggctgaccgtggataagtccaggtggcaggaaggcaacgtgttcag |
| | | | | ctgctccgtgatgcacgaggccctgcacaatcactacacccagaagtc |
| | | | | cctgagcctgtccctgggaaag |
| 131 | IgG4 | | Heavy Chain | gcctccaccaagggcccatccgtcttccccctggcgccctgctccagg |
| | heavy | | Constant Region | agcacctccgagagcacggccgccctgggctgcctggtcaaggacta |
| | chain | | Nucleotide | cttccccgaaccagtgacggtgtcgtggaactcaggcgccctgaccag |
| | constant | | Sequence- | cggcgtgcacaccttcccggctgtcctacagtcctcaggactctactcc |
| | region | | Synthetic Version D | ctcagcagcgtggtgaccgtgccctccagcagcttgggcacgaagac |
| | | | | ctacacctgcaacgtagatcacaagcccagcaacaccaaggtggaca |
| | | | | agagagttgagtccaaatatggtcccccatgcccaccatgcccagcgc |
| | | | | ctccagttgcggggggaccatcagtcttcctgttccccccaaaacccaa |
| | | | | ggacactctcatgatctcccggacccctgaggtcacgtgcgtggtggtg |
| | | | | gacgtgagccaggaagaccccgaggtccagttcaactggtacgtgga |
| | | | | tggcgtggaggtgcataatgccaagacaaagccgcgggaggagcag |
| | | | | ttcaacagcacgtaccgtgtggtcagcgtcctcaccgtcctgcaccagg |
| | | | | actggctgaacggcaaggagtacaagtgcaaggtctccaacaaaggc |
| | | | | ctcccgtcatcgatcgagaaaaccatctccaaagccaaagggcagccc |
| | | | | cgagagccacaggtgtacaccctgcccccatcccaggaggagatgac |
| | | | | caagaaccaggtcagcctgacctgcctggtcaaaggcttctaccccag |
| | | | | cgacatcgccgtggagtgggagagcaatgggcagccggagaacaac |
| | | | | tacaagaccacgcctcccgtgctggactccgacggatccttcttcctcta |
| | | | | cagcaggctaaccgtggacaagagcaggtggcaggaggggaatgtc |
| | | | | ttctcatgctccgtgatgcatgaggctctgcacaaccactacacacaga |
| | | | | agagcctctccctgtctctgggtaaa |
| 132 | | | Heavy Chain | ASTKGPSVFPLAPCSRSTSESTAALGCLVKDY |
| | | | Constant Region | FPEPVTVSWNSGALTSGVHTFPAVLQSSGLYS |
| | | | Amino Acid | LSSVVTVPSSSLGTKTYTCNVDHKPSNTKVD |
| | | | Sequence-encoded | KRVESKYGPPCPPCPAPPVAGGPSVFLFPPKP |
| | | | by Synthetic | KDTLMISRTPEVTCVVVDVSQEDPEVQFNWY |
| | | | Version D | VDGVEVHNAKTKPREEQFNSTYRVVSVLTVL |
| | | | | HQDWLNGKEYKCKVSNKGLPSSIEKTISKAK |
| | | | | GQPREPQVYTLPPSQEEMTKNQVSLTCLVKG |
| | | | | FYPSDIAVEWESNGQPENNYKTTPPVLDSDGS |
| | | | | FFLYSRLTVDKSRWQEGNVFSCSVMHEALHN |
| | | | | HYTQKSLSLSLGK |
| 133 | Human | | Heavy Chain | gcctccaccaagggcccatcggtcttccccctggcaccctcctccaag |
| | IgG1 | | Constant Region | agcacctctgggggcacagcggccctgggctgcctggtcaaggacta |
| | heavy | | Nucleotide | cttccccgaaccggtgacggtgtcgtggaactcaggcgccctgaccag |
| | chain | | Sequence | cggcgtgcacaccttcccggctgtcctacagtcctcaggactctactcc |
| | constant | | | ctcagcagcgtggtgaccgtgccctccagcagcttgggcacccagac |
| | region | | | ctacatctgcaacgtgaatcacaagcccagcaacaccaaggtggacaa |
| | | | | gaaagtggagcccaaatcttgtgacaaaactcacacatgcccaccgtg |
| | | | | cccagcacctgaactcgcgggggcaccgtcagtcttcctcttcccccca |
| | | | | aaacccaaggacaccctcatgatctcccggacccctgaggtcacatgc |
| | | | | gtggtggtggacgtgagccacgaagaccctgaggtcaagttcaactgg |
| | | | | tacgtggacggcgtggaggtgcataatgccaagacaaagccgcggg |
| | | | | aggagcagtacaacagcacgtaccgtgtggtcagcgtcctcaccgtcc |
| | | | | tgcaccaggactggctgaatggcaaggagtacaagtgcaaggtctcca |
| | | | | acaaagccctcccagcccccatcgagaaaaccatctccaaagccaaa |
| | | | | gggcagccccgagaaccacaggtgtacaccctgcccccatcccggg |
| | | | | atgagctgaccaagaaccaggtcagcctgacctgcctggtcaaaggct |
| | | | | tctatcccagcgacatcgccgtggagtgggagagcaatgggcagccg |
| | | | | gagaacaactacaagaccacgcctcccgtgctggactccgacggctc |
| | | | | cttcttcctctacagcaagctcaccgtggacaagagcaggtggcagca |
| | | | | ggggaacgtcttctcatgctccgtgatgcatgaggctctgcacaaccac |
| | | | | tacacgcagaagagcctctccctgtctccgggtaaa |
| 134 | | | Heavy Chain | ASTKGPSVFPLAPSSKSTSGGTAALGCLVKDY |
| | | | Constant Region | FPEPVTVSWNSGALTSGVHTFPAVLQSSGLYS |
| | | | Amino Acid | LSSVVTVPSSSLGTQTYICNVNHKPSNTKVDK |
| | | | Sequence | KVEPKSCDKTHTCPPCPAPELAGAPSVFLFPP |
| | | | | KPKDTLMISRTPEVTCVVVDVSHEDPEVKFN |
| | | | | WYVDGVEVHNAKTKPREEQYNSTYRVVSVL |
| | | | | TVLHQDWLNGKEYKCKVSNKALPAPIEKTIS |
| | | | | KAKGQPREPQVYTLPPSRDELTKNQVSLTCL |
| | | | | VKGFYPSDIAVEWESNGQPENNYKTTPPVLD |
| | | | | SDGSFFLYSKLTVDKSRWQQGNVFSCSVMHE |
| | | | | ALHNHYTQKSLSLSPGK |
| 135 | Human | IGKC* | Cκ Light Chain | cgtacggtggccgctccctccgtgttcatcttcccaccttccgacgagca |
| | Cκ | 01 | Constant Region | gctgaagtccggcaccgcttctgtcgtgtgcctgctgaacaacttctacc |
| | constant | | Nucleotide | cccgcgaggccaaggtgcagtggaaggtggacaacgccctgcagtc |
| | region | | Sequence | cggcaactcccaggaatccgtgaccgagcaggactccaaggacagc |
| | | | | acctactccctgtcctccaccctgaccctgtccaaggccgactacgaga |
| | | | | agcacaaggtgtacgcctgcgaagtgacccaccagggcctgtctagc |
| | | | | cccgtgaccaagtctttcaaccggggcgagtgt |
| 136 | | | Cκ Light Chain | RTVAAPSVFIFPPSDEQLKSGTASVVCLLNNF |
| | | | Constant Region | YPREAKVQWKVDNALQSGNSQESVTEQDSK |
| | | | Amino Acid | DSTYSLSSTLTLSKADYEKHKVYACEVTHQG |
| | | | Sequence | LSSPVTKSFNRGEC |
| 137 | Human | IGKC* | Cκ Light Chain | cgaactgtggctgcaccatctgtcttcatcttcccgccatctgatgagca |
| | Cκ | 02 | Constant Region | gttgaaatctggaactgcctctgttgtgtgcctgctgaataacttctatccc |
| | constant | | Nucleotide | agagaggccaaagtacagtggaaggtggataacgccctccaatcggg |
| | region | | Sequence | taactcccaggagagtgtcacagagcaggagagcaaggacagcacct |
| | | | | acagcctcagcagcaccctgacgctgagcaaagcagactacgagaaa |
| | | | | cacaaagtctacgccggcgaagtcacccatcagggcctgagctcgcc |
| | | | | cgtcacaaagagcttcaacaggggagagtgt |
| 138 | | | Cκ Light Chain | RTVAAPSVFIFPPSDEQLKSGTASVVCLLNNF |
| | | | Constant Region | YPREAKVQWKVDNALQSGNSQESVTEQESK |
| | | | Amino Acid | DSTYSLSSTLTLSKADYEKHKVYAGEVTHQG |
| | | | Sequence | LSSPVTKSFNRGEC |
| 139 | Human | IGKC* | Cκ Light Chain | cgaactgtggctgcaccatctgtcttcatcttcccgccatctgatgagca |
| | Cκ | 03 | Constant Region | gttgaaatctggaactgcctctgttgtgtgcctgctgaataacttctatccc |
| | constant | | Nucleotide | agagaggccaaagtacagcggaaggtggataacgccctccaatcgg |
| | region | | Sequence | gtaactcccaggagagtgtcacagagcaggagagcaaggacagcac |
| | | | | ctacagcctcagcagcaccctgacgctgagcaaagcagactacgaga |
| | | | | aacacaaagtctacgcctgcgaagtcacccatcagggcctgagctcgc |
| | | | | ccgtcacaaagagcttcaacaggggagagtgt |
| 140 | | | Cκ Light Chain | RTVAAPSVFIFPPSDEQLKSGTASVVCLLNNF |
| | | | Constant Region | YPREAKVQRKVDNALQSGNSQESVTEQESKD |
| | | | Amino Acid | STYSLSSTLTLSKADYEKHKVYACEVTHQGL |
| | | | Sequence | SSPVTKSFNRGEC |
| 141 | Human | IGKC* | Cκ Light Chain | cgaactgtggctgcaccatctgtcttcatcttcccgccatctgatgagca |
| | Cκ | 04 | Constant Region | gttgaaatctggaactgcctctgttgtgtgcctgctgaataacttctatccc |
| | constant | | Nucleotide | agagaggccaaagtacagtggaaggtggataacgccctccaatcggg |
| | region | | Sequence | taactcccaggagagtgtcacagagcaggacagcaaggacagcacct |
| | | | | acagcctcagcagcaccctgacgctgagcaaagcagactacgagaaa |
| | | | | cacaaactctacgcctgcgaagtcacccatcagggcctgagctcgccc |
| | | | | gtcacaaagagcttcaacaggggagagtgt |
| 142 | | | Cκ Light Chain | RTVAAPSVFIFPPSDEQLKSGTASVVCLLNNF |
| | | | Constant Region | YPREAKVQWKVDNALQSGNSQESVTEQDSK |
| | | | Amino Acid | DSTYSLSSTLTLSKADYEKHKLYACEVTHQG |
| | | | Sequence | LSSPVTKSFNRGEC |
| 143 | Human | IGKC* | Cκ Light Chain | cgaactgtggctgcaccatctgtcttcatcttcccgccatctgatgagca |
| | Cκ | 05 | Constant Region | gttgaaatctggaactgcctctgttgtgtgcctgctgaataacttctatccc |
| | constant | | Nucleotide | agagaggccaaagtacagtggaaggtggataacgccctccaatcggg |
| | region | | Sequence | taactcccaggagagtgtcacagagcaggacagcaaggacagcacct |
| | | | | acagcctcagcaacaccctgacgctgagcaaagcagactacgagaaa |
| | | | | cacaaagtctacgcctgcgaagtcacccatcagggcctgagctcgccc |
| | | | | gtcacaaagagcttcaacaggggagagtgc |
| 144 | | | Cκ Light Chain | RTVAAPSVFIFPPSDEQLKSGTASVVCLLNNF |
| | | | Constant Region | YPREAKVQWKVDNALQSGNSQESVTEQDSK |
| | | | Amino Acid | DSTYSLSNTLTLSKADYEKHKVYACEVTHQG |
| | | | Sequence | LSSPVTKSFNRGEC |
| 145 | Human | IGCλ1* | Cλ Light Chain | cccaaggccaaccccacggtcactctgttcccgccctcctctgaggag |
| | Cλ | 01 | Constant Region | ctccaagccaacaaggccacactagtgtgtctgatcagtgacttctacc |
| | constant | | Nucleotide | cgggagctgtgacagtggcttggaaggcagatggcagccccgtcaag |
| | region | | Sequence | gcgggagtggagacgaccaaaccctccaaacagagcaacaacaagt |
| | | | | acgcggccagcagctacctgagcctgacgcccgagcagtggaagtc |
| | | | | ccacagaagctacagctgccaggtcacgcatgaagggagcaccgtg |
| | | | | gagaagacagtggcccctacagaatgttca |
| 146 | | | Cλ Light Chain | PKANPTVTLFPPSSEELQANKATLVCLISDFYP |
| | | | Constant Region | GAVTVAWKADGSPVKAGVETTKPSKQSNNK |
| | | | Amino Acid | YAASSYLSLTPEQWKSHRSYSCQVTHEGSTV |
| | | | Sequence | EKTVAPTECS |
| 147 | Human | IGCλ1* | Cλ Light Chain | ggtcagcccaaggccaaccccactgtcactctgttcccgccctcctctg |
| | Cλ | 02 | Constant Region | aggagctccaagccaacaaggccacactagtgtgtctgatcagtgactt |
| | constant | | Nucleotide | ctacccgggagctgtgacagtggcctggaaggcagatggcagcccc |
| | region | | Sequence | gtcaaggcgggagtggagaccaccaaaccctccaaacagagcaaca |
| | | | | acaagtacgcggccagcagctacctgagcctgacgcccgagcagtg |
| | | | | gaagtcccacagaagctacagctgccaggtcacgcatgaagggagca |
| | | | | ccgtggagaagacagtggcccctacagaatgttca |
| 148 | | | Cλ Light Chain | GQPKANPTVTLFPPSSEELQANKATLVCLISD |
| | | | Constant Region | FYPGAVTVAWKADGSPVKAGVETTKPSKQS |
| | | | Amino Acid | NNKYAASSYLSLTPEQWKSHRSYSCQVTHEG |
| | | | Sequence | STVEKTVAPTECS |
| 149 | Human | IGCλ2* | Cλ Light Chain | ggtcagcccaaggccaaccccactgtcactctgttcccgccctcctctg |
| | Cλ | 01 | Constant Region | aggagctccaagccaacaaggccacactagtgtgtctgatcagtgactt |
| | constant | | Nucleotide | ctacccgggagctgtgacagtggcctggaaggcagatggcagcccc |
| | region | | Sequence-Version | gtcaaggcgggagtggagaccaccaaaccctccaaacagagcaaca |
| | | | A | acaagtacgcggccagcagctacctgagcctgacgcccgagcagtg |
| | | | | gaagtcccacagaagctacagctgccaggtcacgcatgaagggagca |
| | | | | ccgtggagaagacagtggcccctacagaatgttca |
| 150 | | | Cλ Light Chain | ggccagcctaaggccgctccttctgtgaccctgttccccccatcctccg |
| | | | Constant Region | aggaactgcaggctaacaaggccaccctcgtgtgcctgatcagcgact |
| | | | Nucleotide | tctaccctggcgccgtgaccgtggcctggaaggctgatagctctcctgt |
| | | | Sequence-Version | gaaggccggcgtggaaaccaccaccccttccaagcagtccaacaaca |
| | | | B | aatacgccgcctcctcctacctgtccctgacccctgagcagtggaagtc |
| | | | | ccaccggtcctacagctgccaagtgacccacgagggctccaccgtgg |
| | | | | aaaagaccgtggctcctaccgagtgctcc |
| 151 | | | Cλ Light Chain | ggccagcctaaagctgcccccagcgtcaccctgtttcctccctccagcg |
| | | | Constant Region | aggagctccaggccaacaaggccaccctcgtgtgcctgatctccgact |
| | | | Nucleotide | tctatcccggcgctgtgaccgtggcttggaaagccgactccagccctgt |
| | | | Sequence-Version | caaagccggcgtggagaccaccacaccctccaagcagtccaacaac |
| | | | C | aagtacgccgcctccagctatctctccctgacccctgagcagtggaagt |
| | | | | cccaccggtcctactcctgtcaggtgacccacgagggctccaccgtgg |
| | | | | aaaagaccgtcgcccccaccgagtgctcc |
| 152 | | | Cλ Light Chain | GQPKANPTVTLFPPSSEELQANKATLVCLISD |
| | | | Constant Region | FYPGAVTVAWKADGSPVKAGVETTKPSKQS |
| | | | Amino Acid | NNKYAASSYLSLTPEQWKSHRSYSCQVTHEG |
| | | | Sequence-Encoded | STVEKTVAPTECS |
| | | | by Version A, B & | |
| | | | C | |
| 153 | Human | IGCλ2* | Cλ Light Chain | ggtcagcccaaggctgccccctcggtcactctgttcccgccctcctctg |
| | Cλ | 02 | Constant Region | aggagcttcaagccaacaaggccacactggtgtgtctcataagtgactt |
| | constant | | Nucleotide | ctacccgggagccgtgacagtggcctggaaggcagatagcagcccc |
| | region | | Sequence | gtcaaggcgggagtggagaccaccacaccctccaaacaaagcaaca |
| | | | | acaagtacgcggccagcagctatctgagcctgacgcctgagcagtgg |
| | | | | aagtcccacagaagctacagctgccaggtcacgcatgaagggagcac |
| | | | | cgtggagaagacagtggcccctacagaatgttca |
| 154 | | | Cλ Light Chain | GQPKAAPSVTLFPPSSEELQANKATLVCLISD |
| | | | Constant Region | FYPGAVTVAWKADSSPVKAGVETTTPSKQSN |
| | | | Amino Acid | NKYAASSYLSLTPEQWKSHRSYSCQVTHEGS |
| | | | Sequence | TVEKTVAPTECS |
| 155 | Human | IG3* | Cλ Light Chain | cccaaggctgccccctcggtcactctgttcccaccctcctctgaggagc |
| | Cλ | Cλ | Constant Region | ttcaagccaacaaggccacactggtgtgtctcataagtgacttctacccg |
| | constant | 01 | Nucleotide | ggagccgtgacagttgcctggaaggcagatagcagccccgtcaaggc |
| | region | | Sequence | gggggtggagaccaccacaccctccaaacaaagcaacaacaagtac |
| | | | | gcggccagcagctacctgagcctgacgcctgagcagtggaagtccca |
| | | | | caaaagctacagctgccaggtcacgcatgaagggagcaccgtggag |
| | | | | aagacagttgcccctacggaatgttca |
| 156 | | | Cλ Light Chain | PKAAPSVTLFPPSSEELQANKATLVCLISDFYP |
| | | | Constant Region | GAVTVAWKADSSPVKAGVETTTPSKQSNNK |
| | | | Amino Acid | YAASSYLSLTPEQWKSHKSYSCQVTHEGSTV |
| | | | Sequence | EKTVAPTECS |
| 157 | Human | IGCλ3* | Cλ Light Chain | ggtcagcccaaggctgccccctcggtcactctgttcccaccctcctctg |
| | Cλ | 02 | Constant Region | aggagcttcaagccaacaaggccacactggtgtgtctcataagtgactt |
| | | | Nucleotide | ctacccggggccagtgacagttgcctggaaggcagatagcagccccg |
| | | | Sequence | tcaaggcgggggtggagaccaccacaccctccaaacaaagcaacaa |
| | | | | caagtacgcggccagcagctacctgagcctgacgcctgagcagtgga |
| | | | | agtcccacaaaagctacagctgccaggtcacgcatgaagggagcacc |
| | | | | gtggagaagacagtggcccctacggaatgttca |
| 158 | constant | | Cλ Light Chain | GQPKAAPSVTLFPPSSEELQANKATLVCLISD |
| | region | | Constant Region | FYPGPVTVAWKADSSPVKAGVETTTPSKQSN |
| | | | Amino Acid | NKYAASSYLSLTPEQWKSHKSYSCQVTHEGS |
| | | | Sequence | TVEKTVAPTECS |
| 159 | Human | IGCλ3* | Cλ Light Chain | ggtcagcccaaggctgccccctcggtcactctgttcccaccctcctctg |
| | Cλ | 03 | Constant Region | aggagcttcaagccaacaaggccacactggtgtgtctcataagtgactt |
| | constant | | Nucleotide | ctacccgggagccgtgacagtggcctggaaggcagatagcagcccc |
| | region | | Sequence | gtcaaggcgggagtggagaccaccacaccctccaaacaaagcaaca |
| | | | | acaagtacgcggccagcagctacctgagcctgacgcctgagcagtgg |
| | | | | aagtcccacaaaagctacagctgccaggtcacgcatgaagggagcac |
| | | | | cgtggagaagacagtggcccctacagaatgttca |
| 160 | | | Cλ Light Chain | GQPKAAPSVTLFPPSSEELQANKATLVCLISD |
| | | | Constant Region | FYPGAVTVAWKADSSPVKAGVETTTPSKQSN |
| | | | Amino Acid | NKYAASSYLSLTPEQWKSHKSYSCQVTHEGS |
| | | | Sequence | TVEKTVAPTECS |
| 161 | Human | IGCλ3* | Cλ Light Chain | ggtcagcccaaggctgccccctcggtcactctgttcccgccctcctctg |
| | Cλ | 04 | Constant Region | aggagcttcaagccaacaaggccacactggtgtgtctcataagtgactt |
| | constant | | Nucleotide | ctacccgggagccgtgacagtggcctggaaggcagatagcagcccc |
| | region | | Sequence | gtcaaggcgggagtggagaccaccacaccctccaaacaaagcaaca |
| | | | | acaagtacgcggccagcagctacctgagcctgacgcctgagcagtgg |
| | | | | aagtcccacagaagctacagctgccaggtcacgcatgaagggagcac |
| | | | | cgtggagaagacagtggcccctacagaatgttca |
| 162 | | | Cλ Light Chain | GQPKAAPSVTLFPPSSEELQANKATLVCLISD |
| | | | Constant Region | FYPGAVTVAWKADSSPVKAGVETTTPSKQSN |
| | | | Amino Acid | NKYAASSYLSLTPEQWKSHRSYSCQVTHEGS |
| | | | Sequence | TVEKTVAPTECS |
| 163 | Human | IGCλ6* | Cλ Light Chain | ggtcagcccaaggctgccccatcggtcactctgttcccgccctcctctg |
| | Cλ | 01 | Constant Region | aggagcttcaagccaacaaggccacactggtgtgcctgatcagtgactt |
| | constant | | Nucleotide | ctacccgggagctgtgaaagtggcctggaaggcagatggcagcccc |
| | region | | Sequence | gtcaacacgggagtggagaccaccacaccctccaaacagagcaaca |
| | | | | acaagtacgcggccagcagctacctgagcctgacgcctgagcagtgg |
| | | | | aagtcccacagaagctacagctgccaggtcacgcatgaagggagcac |
| | | | | cgtggagaagacagtggcccctgcagaatgttca |
| 164 | | | Cλ Light Chain | GQPKAAPSVTLFPPSSEELQANKATLVCLISD |
| | | | Constant Region | FYPGAVKVAWKADGSPVNTGVETTTPSKQS |
| | | | Amino Acid | NNKYAASSYLSLTPEQWKSHRSYSCQVTHEG |
| | | | Sequence | STVEKTVAPAECS |
| 165 | Human | IGCλ7* | Cλ Light Chain | ggtcagcccaaggctgccccatcggtcactctgttcccaccctcctctg |
| | Cλ | 02 | Constant Region | aggagcttcaagccaacaaggccacactggtgtgtctcgtaagtgactt |
| | | | Nucleotide | ctacccgggagccgtgacagtggcctggaaggcagatggcagcccc |
| | | | Sequence | gtcaaggtgggagtggagaccaccaaaccctccaaacaaagcaacaa |
| | | | | caagtatgcggccagcagctacctgagcctgacgcccgagcagtgga |
| | | | | agtcccacagaagctacagctgccgggtcacgcatgaagggagcacc |
| | | | | gtggagaagacagtggcccctgcagaatgctct |
| 166 | constant | | Cλ Light Chain | GQPKAAPSVTLFPPSSEELQANKATLVCLVSD |
| | region | | Constant Region | FYPGAVTVAWKADGSPVKVGVETTKPSKQS |
| | | | Amino Acid | NNKYAASSYLSLTPEQWKSHRSYSCRVTHEG |
| | | | Sequence | STVEKTVAPAECS |
| 167 | Recombinant | | Nucleotide | ATGGGCTGGTCCTGCATCATCCTGTTTCTGG |
| | Human | | Sequence | TGGCCACCGCCACCGGCGTGCACAGCGATT |
| | OX40L | | | ACAAGGATGACGACGATAAGCGTATGAAA |
| | (Leader | | | CAGATCGAAGATAAAATTGAAGAGATCTTG |
| | sequence, | | | AGCAAAATCTATCATATCGAAAACGAAATT |
| | Isoleucine | | | GCGCGTATCAAAAAGCTGATTGGCGAACGT |
| | Zipper | | | GGCGGTGGCAGCGGTGGCGGTAGCGGCGG |
| | and | | | TGGCAGCCAGGTGTCCCACCGATACCCCAG |
| | FLAG | | | GATCCAGTCCATCAAGGTCCAGTTCACCGA |
| | Sequence | | | GTACAAAAAGGAGAAGGGATTCATCCTGA |
| | Included) | | | CCTCCCAAAAGGAGGACGAGATCATGAAG |
| | | | | GTGCAAAACAACTCCGTGATCATCAACTGC |
| | | | | GACGGCTTCTACCTGATCTCCCTGAAGGGC |
| | | | | TACTTCTCCCAGGAGGTGAACATCTCCCTG |
| | | | | CACTACCAGAAGGACGAGGAGCCCCTGTTC |
| | | | | CAGCTGAAGAAGGTGAGGTCCGTGAATTCC |
| | | | | CTGATGGTGGCCAGCCTGACCTACAAGGAC |
| | | | | AAGGTCTACCTGAACGTGACCACCGACAAC |
| | | | | ACCAGCCTGGACGACTTCCATGTCAACGGC |
| | | | | GGCGAGCTGATCCTGATCCATCAGAACCCC |
| | | | | GGCGAGTTTTGCGTCCTG |
| 168 | | | Amino Acid | MGWSCIILFLVATATGVHSDYKDDDDKRMK |
| | | | Sequence | QIEDKIEEILSKIYHIENEIARIKKLIGERGGGS |
| | | | | GGGSGGGSQVSHRYPRIQSIKVQFTEYKKEK |
| | | | | GFILTSQKEDEIMKVQNNSVIINCDGFYLISLK |
| | | | | GYFSQEVNISLHYQKDEEPLFQLKKVRSVNSL |
| | | | | MVASLTYKDKVYLNVTTDNTSLDDFHVNGG |
| | | | | ELILIHQNPGEFCVL |
| 169 | Recombinant | | Nucleotide | ATGGGCTGGTCCTGCATCATCCTGTTTCTGG |
| | Rhesus | | Sequence | TGGCCACCGCCACCGGCGTGCACAGCGATT |
| | OX40L | | | ACAAGGATGACGACGATAAGCGTATGAAA |
| | (Leader | | | CAGATCGAAGATAAAATTGAAGAGATCTTG |
| | Sequence, | | | AGCAAAATCTATCATATCGAAAACGAAATT |
| | FLAG | | | GCGCGTATCAAAAAGCTGATTGGCGAACGT |
| | and | | | GGCGGTGGCAGCGGTGGCGGTAGCGGCGG |
| | Isoleucine | | | TGGCAGCCAGGTGTCCCACCAATACCCCAG |
| | zipper | | | GATCCAGTCCATCAAGGTCCAGTTCACCGA |
| | included) | | | GTACAAAAAGGAGGAGGGATTCATCCTGA |
| | | | | CCTCCCAAAAGGAGGACGAGATCATGAAG |
| | | | | GTGCAAAACAACTCCGTGATCATCAACTGC |
| | | | | GACGGCTTCTACCTGATCTCCCTGAAGGGC |
| | | | | TACTTCTCCCAGGAGGTGAACATCTCCCTG |
| | | | | CACTACCAGAAGGACGAGGAGCCCCTGTTC |
| | | | | CAGCTGAAGAAGGTGAGGTCCGTGAATTCC |
| | | | | CTGATGGTGGCCAGCCTGACCTACAAGGAC |
| | | | | AAGGTCTACCTGAACGTGACCACCGACAAC |
| | | | | ACCAGCCTGGACGACTTCCATGTCAACGGC |
| | | | | GGCGAGCTGATCCTGATCCATCAGAACCCC |
| | | | | GGCGAGTTTTGCGTCCTG |
| 170 | | | Amino Acid | MGWSCIILFLVATATGVHSDYKDDDDKRMK |
| | | | Sequence | QIEDKIEEILSKIYHIENEIARIKKLIGERGGGS |
| | | | | GGGSGGGSQVSHQYPRIQSIKVQFTEYKKEE |
| | | | | GFILTSQKEDEIMKVQNNSVIINCDGFYLISLK |
| | | | | GYFSQEVNISLHYQKDEEPLFQLKKVRSVNSL |
| | | | | MVASLTYKDKVYLNVTTDNTSLDDFHVNGG |
| | | | | ELILIHQNPGEFCVL |
| 171 | Recombinant | | Nucleotide | ATGGGCTGGTCCTGCATCATCCTGTTTCTGG |
| | Human | | Sequence | TGGCCACCGCCACCGGCGTGCACAGCCTGC |
| | OX40R | | | ATTGCGTGGGCGACACCTATCCCTCCAACG |
| | (Leader | | | ACAGGTGCTGCCACGAGTGCAGGCCTGGAA |
| | Sequence | | | ACGGCATGGTGAGCAGGTGCAGCCGGTCCC |
| | and | | | AGAATACCGTGTGTAGGCCCTGCGGCCCCG |
| | Human | | | GCTTTTACAACGACGTGGTGTCCTCCAAGC |
| | Fc | | | CCTGCAAGCCCTGCACATGGTGCAACCTGC |
| | Sequence | | | GGTCCGGCAGCGAGAGGAAGCAGCTCTGC |
| | included) | | | ACAGCCACCCAGGACACCGTCTGTAGGTGT |
| | | | | AGGGCTGGCACCCAGCCTCTGGACTCCTAC |
| | | | | AAGCCCGGCGTGGATTGTGCTCCTTGCCCT |
| | | | | CCCGGCCATTTCTCCCCTGGCGACAACCAG |
| | | | | GCTTGCAAGCCCTGGACCAACTGTACCCTG |
| | | | | GCCGGCAAGCATACACTGCAGCCTGCTTCC |
| | | | | AACTCCTCCGACGCTATCTGCGAGGATAGG |
| | | | | GACCCCCCTGCCACACAACCCCAGGAGACA |
| | | | | CAGGGCCCTCCTGCTAGGCCCATCACAGTC |
| | | | | CAACCCACCGAAGCCTGGCCCAGGACATCC |
| | | | | CAAGGCCCTTCCACCAGGCCTGTGGAAGTG |
| | | | | CCTGGAGGAAGGGCTGTGGCCATTGAAGGT |
| | | | | CGTATGGATGAACCCAAGTCCTGCGACAAG |
| | | | | ACCCACACCTGTCCCCCTTGTCCTGCCCCTG |
| | | | | AACTGCTGGGCGGACCTTCCGTGTTCCTGTT |
| | | | | CCCCCCAAAGCCCAAGGACACCCTGATGAT |
| | | | | CTCCCGGACCCCCGAAGTGACCTGCGTGGT |
| | | | | GGTGGATGTGTCCCACGAGGACCCTGAAGT |
| | | | | GAAGTTCAATTGGTACGTGGACGGCGTGGA |
| | | | | AGTGCACAACGCCAAGACCAAGCCTAGAG |
| | | | | AGGAACAGTACAACTCCACCTACCGGGTGG |
| | | | | TGTCCGTGCTGACCGTGCTGCACCAGGATT |
| | | | | GGCTGAACGGCAAAGAGTACAAGTGCAAG |
| | | | | GTGTCCAACAAGGCCCTGCCTGCCCCCATC |
| | | | | GAAAAGACCATCTCCAAGGCCAAGGGCCA |
| | | | | GCCCCGGGAACCCCAGGTGTACACACTGCC |
| | | | | CCCTAGCAGGGACGAGCTGACCAAGAACC |
| | | | | AGGTGTCCCTGACCTGTCTCGTGAAAGGCT |
| | | | | TCTACCCCTCCGATATCGCCGTGGAATGGG |
| | | | | AGTCCAACGGCCAGCCTGAGAACAACTACA |
| | | | | AGACCACCCCCCCTGTGCTGGACTCCGACG |
| | | | | GCTCATTCTTCCTGTACAGCAAGCTGACAG |
| | | | | TGGACAAGTCCCGGTGGCAGCAGGGCAAC |
| | | | | GTGTTCTCCTGCTCCGTGATGCACGAGGCC |
| | | | | CTGCACAACCACTACACCCAGAAGTCCCTG |
| | | | | TCCCTGAGCCCCTGA |
| 172 | | | Amino Acid | MGWSCIILFLVATATGVHSLHCVGDTYPSND |
| | | | Sequence | RCCHECRPGNGMVSRCSRSQNTVCRPCGPGF |
| | | | | YNDVVSSKPCKPCTWCNLRSGSERKQLCTAT |
| | | | | QDTVCRCRAGTQPLDSYKPGVDCAPCPPGHF |
| | | | | SPGDNQACKPWTNCTLAGKHTLQPASNSSDA |
| | | | | ICEDRDPPATQPQETQGPPARPITVQPTEAWP |
| | | | | RTSQGPSTRPVEVPGGRAVAIEGRMDEPKSC |
| | | | | DKTHTCPPCPAPELLGGPSVFLFPPKPKDTLMI |
| | | | | SRTPEVTCVVVDVSHEDPEVKFNWYVDGVE |
| | | | | VHNAKTKPREEQYNSTYRVVSVLTVLHQDW |
| | | | | LNGKEYKCKVSNKALPAPIEKTISKAKGQPRE |
| | | | | PQVYTLPPSRDELTKNQVSLTCLVKGFYPSDI |
| | | | | AVEWESNGQPENNYKTTPPVLDSDGSFFLYS |
| | | | | KLTVDKSRWQQGNVFSCSVMHEALHNHYTQ |
| | | | | KSLSLSP |
| 173 | Cell | | Nucleotide | ATGGAGAGGGTGCAGCCCCTCGAGGAGAA |
| | Expressed | | Sequence | CGTGGGAAACGCCGCCAGGCCTAGGTTCGA |
| | OX40L | | | GAGGAACAAGCTGCTGCTGGTGGCTTCCGT |
| | (CHO/MEF) | | | GATCCAAGGACTCGGCCTGCTGCTCTGCTT |
| | (Leader | | | CACCTACATCTGCCTCCACTTCAGCGCCCTG |
| | sequence | | | CAGGTGTCCCACCGATACCCCAGGATCCAG |
| | included) | | | TCCATCAAGGTCCAGTTCACCGAGTACAAA |
| | | | | AAGGAGAAGGGATTCATCCTGACCTCCCAA |
| | | | | AAGGAGGACGAGATCATGAAGGTGCAAAA |
| | | | | CAACTCCGTGATCATCAACTGCGACGGCTT |
| | | | | CTACCTGATCTCCCTGAAGGGCTACTTCTCC |
| | | | | CAGGAGGTGAACATCTCCCTGCACTACCAG |
| | | | | AAGGACGAGGAGCCCCTGTTCCAGCTGAAG |
| | | | | AAGGTGAGGTCCGTGAATTCCCTGATGGTG |
| | | | | GCCAGCCTGACCTACAAGGACAAGGTCTAC |
| | | | | CTGAACGTGACCACCGACAACACCAGCCTG |
| | | | | GACGACTTCCATGTCAACGGCGGCGAGCTG |
| | | | | ATCCTGATCCATCAGAACCCCGGCGAGTTT |
| | | | | TGCGTCCTGTAA |
| 174 | | | Amino Acid | MERVQPLEENVGNAARPRFERNKLLLVASVI |
| | | | Sequence | QGLGLLLCFTYICLHFSALQVSHRYPRIQSIKV |
| | | | | QFTEYKKEKGFILTSQKEDEIMKVQNNSVIIN |
| | | | | CDGFYLISLKGYFSQEVNISLHYQKDEEPLFQ |
| | | | | LKKVRSVNSLMVASLTYKDKVYLNVTTDNT |
| | | | | SLDDFHVNGGELILIHQNPGEFC |
| 175 | Cell | | Nucleotide | ATGTGCGTGGGGGCTCGGCGGCTGGGCCGC |
| | Expressed | | Sequence | GGGCCGTGTGCGGCTCTGCTCCTCCTGGGC |
| | OX40 | | | CTGGGGCTGAGCACCGTGACGGGGCTCCAC |
| | receptor | | | TGTGTCGGGGACACCTACCCCAGCAACGAC |
| | (HT1080) | | | CGGTGCTGCCACGAGTGCAGGCCAGGCAAC |
| | | | | GGGATGGTGAGCCGCTGCAGCCGCTCCCAG |
| | | | | AACACGGTGTGCCGTCCGTGCGGGCCGGGC |
| | | | | TTCTACAACGACGTGGTCAGCTCCAAGCCG |
| | | | | TGCAAGCCCTGCACGTGGTGTAACCTCAGA |
| | | | | AGTGGGAGTGAGCGGAAGCAGCTGTGCAC |
| | | | | GGCCACACAGGACACAGTCTGCCGCTGCCG |
| | | | | GGCGGGCACCCAGCCCCTGGACAGCTACAA |
| | | | | GCCTGGAGTTGACTGTGCCCCCTGCCCTCC |
| | | | | AGGGCACTTCTCCCCAGGCGACAACCAGGC |
| | | | | CTGCAAGCCCTGGACCAACTGCACCTTGGC |
| | | | | TGGGAAGCACACCCTGCAGCCGGCCAGCA |
| | | | | ATAGCTCGGACGCAATCTGTGAGGACAGGG |
| | | | | ACCCCCCAGCCACGCAGCCCCAGGAGACCC |
| | | | | AGGGCCCCCCGGCCAGGCCCATCACTGTCC |
| | | | | AGCCCACTGAAGCCTGGCCCAGAACCTCAC |
| | | | | AGGGACCCTCCACCCGGCCCGTGGAGGTCC |
| | | | | CCGGGGGCCGTGCGGTTGCCGCCATCCTGG |
| | | | | GCCTGGGCCTGGTGCTGGGGCTGCTGGGCC |
| | | | | CCCTGGCCATCCTGCTGGCCCTGTACCTGCT |
| | | | | CCGGAGGGACCAGAGGCTGCCCCCCGATGC |
| | | | | CCACAAGCCCCCTGGGGGAGGCAGTTTCCG |
| | | | | GACCCCCATCCAAGAGGAGCAGGCCGACG |
| | | | | CCCACTCCACCCTGGCCAAGATCTGA |
| 176 | | | Amino Acid | MCVGARRLGRGPCAALLLLGLGLSTVTGLHC |
| | | | Sequence | VGDTYPSNDRCCHECRPGNGMVSRCSRSQNT |
| | | | | VCRPCGPGFYNDVVSSKPCKPCTWCNLRSGS |
| | | | | ERKQLCTATQDTVCRCRAGTQPLDSYKPGVD |
| | | | | CAPCPPGHFSPGDNQACKPWTNCTLAGKHTL |
| | | | | QPASNSSDAICEDRDPPATQPQETQGPPARPIT |
| | | | | VQPTEAWPRTSQGPSTRPVEVPGGRAVAAIL |
| | | | | GLGLVLGLLGPLAILLALYLLRRDQRLPPDAH |
| | | | | KPPGGGSFRTPIQEEQADAHSTLAKI |
| 177 | OX40L15B07 | | Amino Acid | EVQLVESGGGLVQAGGSLRLSCAASRSIGRL |
| | | | Sequence of | DRMGWYRHRTGEPRELVATITGGSSINYGDF |
| | | | OX40L15B07 (Seq | VKGRFTISIDNAKNTVYLQMNNLKPEDTAVY |
| | | | ID No 179 in | YCNFNKYVTSRDTWGQGTQVTVSS |
| | | | WO2011/073180, | |
| | | | Table A-1) | |
| 178 | OX40L01B11 | | Amino Acid | EVQLVESGGGLVQAGGSLRLSCVASGRSFST |
| | | | Sequence of | YIMGWFRQAPGKEREFVATISRSGITIRSADS |
| | | | OX40L01B11 (Seq | VKGRFTISRDNAKNTVYLQMNSLKPEDTAVY |
| | | | ID No 180 in | YCAAGPYVEQTLGLYQTLG |
| | | | WO2011/073180, | PWDYWGQGTQVTVSS |
| | | | Table A-1) | |
| 179 | OX40L01E07 | | Amino Acid | EVQLVESGGGLVQAGGSLRLSCAASGRTFSSI |
| | | | Sequence of | YAKGWFRQAPGKEREFVAAISRSGRSTSYAD |
| | | | OX40L01E07 (Seq | SVKGRFTISRDNAKNTVYLQMNSLKPEDTAV |
| | | | ID No 181 in | YYCAAVGGATTVTASEWDYWGLGTQVTVSS |
| | | | WO2011/073180, | |
| | | | Table A-1) | |
| 180 | OX40L01E10 | | Amino Acid | EVQLVESGGGLVQAGDSLRLSCAASGLTFSSF |
| | | | Sequence of | AMGWFRQAPGKEREFVAAISRSGYGTSEADS |
| | | | OX40L01E10 (Seq | VRDRFIISRDNAKNTVTLHLSRLKPEDTAVYY |
| | | | ID No 182 in | CAAEHTLGRPSRSQINYLYWGQGTQVTVSS |
| | | | WO2011/073180, | |
| | | | Table A-1) | |
| 181 | OX40L18E09 | | Amino Acid | EVQLVESGGGLVQAGGSLRLSCAASRNILSL |
| | | | Sequence of | NTMGWYRHAPGKPRELVARISSNSKTDYADS |
| | | | OX40L18E09 (Seq | VKGRFTISRDNAKNTVLLQMNSLKPEDTGVY |
| | | | ID No 183 in | YCNLNVWRTSSDYWGQGTQVTVSS |
| | | | WO2011/073180, | |
| | | | Table A-1) | |
| 182 | OX40L19A07 | | Amino Acid | EVQLVESGGGLVQAGGSLRLSCAASGFTLDD |
| | | | Sequence of | YAIAWFRQAPGKEREGVSRIKISNGRTTYAGS |
| | | | OX40L19A07 (Seq | VKGRFTISSDNAKNTVYLQMNSLNAEDTAVY |
| | | | ID No 184 in | YCAADRSSLLFGSNWDRKARYDYWGQGTQ |
| | | | WO2011/073180, | VTVSS |
| | | | Table A-1) | |
| 183 | OX40L19D08 | | Amino Acid | EVQLVESGGGLVQAGASLRLSCAASGRRFIS |
| | | | Sequence of | NYAMGWFRQAPGQERAFVAAISRSGSITYYT |
| | | | OX40L19D08 (Seq | DSVKGRFSISRDYAKSTVYLQMDNLKPEDTA |
| | | | ID No 185 in | VYYCAADGGAVRDLTTNLPDYWGRGTQVT |
| | | | WO2011/073180, | VSS |
| | | | Table A-1) | |
| 184 | OX40L075 | | Amino Acid | EVQLVESGGGLVQPGGSLRLSCAASGRSFST |
| | | | Sequence of | YIMGWFRQAPGKEREFVATISRSGITTRSADS |
| | | | OX40L075 (Seq ID | VKGRFTISRDNSKNTVYLQMNSLRPEDTAVY |
| | | | No 199 in | YCAAGPYVEQTLG |
| | | | WO2011/073180, | LYQTLGPWDYWGQGTLVTVSS |
| | | | Table A-2) | |
| 185 | OX40L024 | | Amino Acid | EVQLVESGGGLVQPGGSLRLSCAASG |
| | | | Sequence of | RTFSSIYAKGWFRQAPGKEREFV |
| | | | OX40L024 (Seq ID | AAISRSGRSTSYADSVKGRFTISRD |
| | | | No 200 in | NAKNTVYLQMNSLRPEDTAVYYCAA |
| | | | WO2011/073180, | VGGATTVTASEWDYWGLGTLVTVSS |
| | | | Table A-2) | |
| 186 | OX40L025 | | Amino Acid | EVQLVESGGGLVQPGGSLRLSCAASG |
| | | | Sequence of | RTFSSIYAKGWFRQAPGKEREFV |
| | | | OX40L025 (Seq ID | AAISRSGRSTSYADSVKGRFTISRD |
| | | | No 201 in | NSKNTVYLQMNSLRPEDTAVYYCAA |
| | | | WO2011/073180, | VGGATTVTASEWDYWGLGTLVTVSS |
| | | | Table A-2) | |
| 187 | OX40L026 | | Amino Acid | EVQLVESGGGLVQPGGSLRLSCAASG |
| | | | Sequence of | RTFSSIYAKGWFRQAPGKEREFV |
| | | | OX40L026 (Seq ID | AAISRSGRSTSYADSVKGRFTISRD |
| | | | No 202 in | NAKNTVYLQMNSLRPEDTAVYYCAA |
| | | | WO2011/073180, | VGGATTVTASEWDYWGQGTLVTVSS |
| | | | Table A-2) | |
| 188 | OX40L027 | | Amino Acid | EVQLVESGGGLVQPGGSLRLSCAASG |
| | | | Sequence of | RTFSSIYAKGWFRQAPGKEREFV |
| | | | OX40L027 (Seq ID | AAISRSGRSTSYADSVKG |
| | | | No 203 in | RFTISRDNSKNTVYLQM |
| | | | WO2011/073180, | NSLRPEDTAVYYCAA |
| | | | Table A-2) | VGGATTVTASEWDYWGQGTLVTVSS |
| 189 | OX40L028 | | Amino Acid | DVQLVESGGGLVQPGGSLRLSCAASG |
| | | | Sequence of | RTFSSIYAKGWFRQAPGKEREFVAAISRSG |
| | | | OX40L028 (Seq ID | RSTSYADSVKGRFTISRDNAKNTVYLQM |
| | | | No 204 in | NSLRPEDTAVYYCAAVGGATTVTASEWDYW |
| | | | WO2011/073180, | GLGTLVTVSS |
| | | | Table A-2) | |
| 190 | OX40L039 | | Amino Acid | DVQLVESGGGLVQPGGSLRLSCAASG |
| | | | Sequence of | RTFSSIYAKGWFRQAPGKEREFV |
| | | | OX40L039 (Seq ID | AAISRSGRSTSYADSVKGRFTISRD |
| | | | No 205 in | NSKNTVYLQM |
| | | | WO2011/073180, | NSLRPEDTAVYYCAAVGGATTVTASEWDYW |
| | | | Table A-2) | GQGTLVTVSS |
| 191 | OX40L030 | | Amino Acid | DVQLVESGGGLVQAGGSLRLSCAASRSIGRL |
| | | | Sequence of | DRMGWYRHRTGEPRELVATITGGSSINYGD |
| | | | OX40L030 (Seq ID | FVKGRFTISIDNAKNTVYLQMN |
| | | | No 206 in | LKPEDTAVYYCN |
| | | | WO2011/073180, | FNKYVTSRDTWGQGTQVTVSS |
| | | | Table A-2) | |
| 192 | OX40L040 | | Amino Acid | DVQLVESGGGLVQPGGSLRLSCAASRSIGRL |
| | | | Sequence of | DRMGWYRHRTGEPRELVATITGGSSINYGD |
| | | | OX40L040 (Seq ID | FVKGRFTISRDNSKNTVYLQM |
| | | | No 207 in | NSLRPEDTAVYYCNFN |
| | | | WO2011/073180, | KYVTSRDTWGQGTLVTVSS |
| | | | Table A-2) | |
| 193 | OX40L041 | | Amino Acid | DVQLVESGGGLVQPGGSLRLSCAASRSIGRL |
| | | | Sequence of | DRMGWYRHATGEPRELVATITGGSSINYGD |
| | | | OX40L041 (Seq ID | FVKGRFTISRDNSKNTVYLQM |
| | | | No 208 in | NSLRPEDTAVYYCNFN |
| | | | WO2011/073180, | KYVTSRDTWGQGTLVTVSS |
| | | | Table A-2) | |
| 194 | OX40L042 | | Amino Acid | DVQLVESGGGLVQPGGSLRLSCAASRSIGRL |
| | | | Sequence of | DRMGWYRHRPGEPRELVATITGGSSINYGD |
| | | | OX40L042 (Seq ID | FVKGRFTISRDNSKNTVYLQM |
| | | | No 209 in | NSLRPEDTAVYYCNFN |
| | | | WO2011/073180, | KYVTSRDTWGQGTLVTVSS |
| | | | Table A-2) | |
| 195 | OX40L043 | | Amino Acid | DVQLVESGGGLVQPGGSLRLSCAASRSIGRL |
| | | | Sequence of | DRMGWYRHRTGKPRELVATITGGSSINYGD |
| | | | OX40L043 (Seq ID | FVKGRFTISRDNSKNTVYLQM |
| | | | No 210 in | NSLRPEDTAVYYCNFN |
| | | | WO2011/073180, | KYVTSRDTWGQGTLVTVSS |
| | | | Table A-2) | |
| 196 | OX40L044 | | Amino Acid | DVQLVESGGGLVQPGGSLRLSCAASRSIGRL |
| | | | Sequence of | DRMGWYRHAPGEPRELVATITGGSSINYGD |
| | | | OX40L044 (Seq ID | FVKGRFTISRDNSKNTVYLQM |
| | | | No 211 in | NSLRPEDTAVYYCNFN |
| | | | WO2011/073180, | KYVTSRDTWGQGTLVTVSS |
| | | | Table A-2) | |
| 197 | OX40L045 | | Amino Acid | DVQLVESGGGLVQPGGSLRLSCAASRSIG |
| | | | Sequence of | RLDRMGWYRHATGKPRELV |
| | | | OX40L045 (Seq ID | ATITGGSSINYGDFVKG |
| | | | No 212 in | RFTISRDNSKNTVYLQMNSLRPEDTAVYYCN |
| | | | WO2011/073180, | FN |
| | | | Table A-2) | KYVTSRDTWGQGTLVTVSS |
| 198 | OX40L046 | | Amino Acid | DVQLVESGGGLVQPGGSLRLSCAASRSIGRL |
| | | | Sequence of | DRMGWYRHRPGKPRELVATITGGSSINYGD |
| | | | OX40L046 (Seq ID | FVKGRFTISRDNSKNTVYLQM |
| | | | No 213 in | NSLRPEDTAVYYCNFN |
| | | | WO2011/073180, | KYVTSRDTWGQGTLVTVSS |
| | | | Table A-2) | |
| 199 | OX40L047 | | Amino Acid | DVQLVESGGGLVQPGGSLRLSCAASRSIGRL |
| | | | Sequence of | DRMGWYRHAPGKPRELVATITGGSSINYGD |
| | | | OX40L047 (Seq ID | FVKGRFTISRDNSKNTVYLQM |
| | | | No 214 in | NSLRPEDTAVYYCNFN |
| | | | WO2011/073180, | KYVTSRDTWGQGTLVTVSS |
| | | | Table A-2) | |
| 200 | OX40L048 | | Amino Acid | DVQLVESGGGLVQPGGSLRLSCAASRSIGRL |
| | | | Sequence of | DRMGWYRHRTGEPRELVATITGGSSINYADF |
| | | | OX40L048 (Seq ID | VKGRFTISRDNSKNTVYLQM |
| | | | No 215 in | NSLRPEDTAVYYCNFN |
| | | | WO2011/073180, | KYVTSRDTWGQGTLVTVSS |
| | | | Table A-2) | |
| 201 | OX40L049 | | Amino Acid | DVQLVESGGGLVQPGGSLRLSCAASRSIGRL |
| | | | Sequence of | DRMGWYRHRTGEPRELVATITGGSSINYG |
| | | | OX40L049 (Seq ID | DSVKGRFTISRDNSKNTVYLQM |
| | | | No 216 in | NSLRPEDTAVYYCNFN |
| | | | WO2011/073180, | KYVTSRDTWGQGTLVTVSS |
| | | | Table A-2) | |
| 202 | OX40L050 | | Amino Acid | DVQLVESGGGLVQPGGSLRLSCAASRSIGRL |
| | | | Sequence of | DRMGWYRHRTGEPRELVATITGGSSINYADS |
| | | | OX40L050 (Seq ID | VKGRFTISRDNSKNTVYLQM |
| | | | No 217 in | NSLRPEDTAVYYCNFN |
| | | | WO2011/073180, | KYVTSRDTWGQGTLVTVSS |
| | | | Table A-2) | |
| 203 | OX40L053 | | Amino Acid | DVQLVESGGGLVQPGGSLRLSCAASRSIGRL |
| | | | Sequence of | DRMGWYRHRTGEPRELV |
| | | | OX40L053 (Seq ID | ATITGGSSINYGDFVKGRFTISIDNSKNTVYLQ |
| | | | No 218 in | MNSLRPEDTAVYYCNFNK |
| | | | WO2011/073180, | YVTSRDTWGQGTLVTVSS |
| | | | Table A-2) | |
| 1 | OX40L054 | | Amino Acid | DVQLVESGGGLVQPGGSLRLSCAASRSIGRL |
| | | | Sequence of | DRMGWYRHRTGEPRELV |
| | | | OX40L054 (Seq ID | ATITGGSSINYGDFVKG |
| | | | No 219 in | RFTISRDNAKNTVYLQM |
| | | | WO2011/073180, | NSLRPEDTAVYYCNFN |
| | | | Table A-2) | KYVTSRDTWGQGTLVTVSS |
| 2 | OX40L055 | | Amino Acid | DVQLVESGGGLVQPGGSLRLSCAASRSIGRL |
| | | | Sequence of | DRMGWYRHRTGEPRELV |
| | | | OX40L055 (Seq ID | ATITGGSSINYGDFVKGRFTISRDNSKNTVYL |
| | | | No 220 in | QMNNLRPEDTAVYYCNFN |
| | | | WO2011/073180, | KYVTSRDTWGQGTLVTVSS |
| | | | Table A-2) | |
| 3 | OX40L056 | | Amino Acid | DVQLVESGGGLVQPGGSLRLSCAASRSIGRL |
| | | | Sequence of | DRMGWYRHRPGKPRELV |
| | | | OX40L056 (Seq ID | ATITGGSSINYADSVKGRFTISRD |
| | | | No 221 in | NSKNTVYLQMNSLRPEDTAVYYCNFN |
| | | | WO2011/073180, | KYVTSRDTWGQGTLVTVSS |
| | | | Table A-2) | |
| 4 | OX40L069 | | Amino Acid | DVQLVESGGGLVQPGGSLRLSCAASRSIGRL |
| | | | Sequence of | DRMGWYRHRPGKPRELV |
| | | | OX40L069 (Seq ID | ATITGGSSINYADSVKGRFTISI |
| | | | No 222 in | DNSKNTVYLQMNSLRPEDTAVYYCNFNK |
| | | | WO2011/073180, | YVTSRDTWGQGTLVTVSS |
| | | | Table A-2) | |
| 5 | OX40L070 | | Amino Acid | DVQLVESGGGLVQPGGSLRLSCAASRSIGRL |
| | | | Sequence of | DRMGWYRHRPGKPRELV |
| | | | OX40L070 (Seq ID | ATITGGSSINYADSVKGRFTISRD |
| | | | No 223 in | NSKNTVYLQMNLRPEDTAVYYCNFN |
| | | | WO2011/073180, | KYVTSRDTWGQGTLVTVSS |
| | | | Table A-2) | |
| 6 | OX40L071 | | Amino Acid | DVQLVESGGGLVQPGGSLRLSCAASRSIGRL |
| | | | Sequence of | DRMGWYRHRPGKPRELV |
| | | | OX40L071 (Seq ID | ATITGGSSINYADSVKGRFTISI |
| | | | No 224 in | DNSKNTVYLQMNLRPEDTAVYYCNFN |
| | | | WO2011/073180, | KYVTSRDTWGQGTLVTVSS |
| | | | Table A-2) | |
| 7 | OX40L082 | | Amino Acid | EVQLVESGGGLVQPGGSLRLSCAASRSIGRLD |
| | | | Sequence of | RMGWYRHRPGEPRELVA |
| | | | OX40L082 (Seq ID | TITGGSSINYGDSVKGRFTISIDNSKNTVYLQ |
| | | | No 225 in | MNSLRPEDTAVYYCNENKY |
| | | | WO2011/073180, | VTSRDTWGQGTLVTVSS |
| | | | Table A-2) | |
| 8 | OX40L083 | | Amino Acid | EVQLVESGGGLVQPGGSLRLSCAASRSIGRLD |
| | | | Sequence of | RMGWYRHRPGKPRELV |
| | | | OX40L083 (Seq ID | ATITGGSSINYGDSVKGRFTISIDNSKNTVYLQ |
| | | | No 226 in | MNSLRPEDTAVYYCNFNK |
| | | | WO2011/073180, | YVTSRDTWGQGTLVTVSS |
| | | | Table A-2) | |
| 9 | OX40L | | Amino acid | EVQLLESGGGLVQPGGSLRLSCAASGFTFNSY |
| | benchmark | | sequence of OX40L | AMSWVRQAPGKGLEW |
| | antibody | | benchmark antibody | VSIISGSGGFTYYADSVKG |
| | heavy | | heavy chain (Seq ID | RFTISRDNSRTTLYLQMNSLRAEDTAVYYCA |
| | chain | | No: 177 in | KDRLVAPGTFDYWGQGALVTVSSASTKG |
| | | | WO2011/073180, | PSVFPLAPSSKSTSGGTAALG |
| | | | Table A-5) | CLVKDYFPEPVTVSWNSGALTSGVHTFPAVL |
| | | | | QSSGLYSLSSVVTVPSSSL |
| | | | | GTQTYICNVNH |
| | | | | KPSNTKVDKKVEPKSCDKTHTCPPCPAPELL |
| | | | | GGPSVFLF |
| | | | | PPKPKDTLMISRTPEVTCVVVDVSHEDP |
| | | | | EVKFNWYVDGVEVHNAKTKP |
| | | | | REEQYNSTYRVVSVLTVLHQDWLNG |
| | | | | KEYKCKVSNKALPAPIEKTISKAKG |
| | | | | QPREPQVYTLPPSRDELTKNQVSLTCLVKG |
| | | | | FYPSDIAVEWESNGQPEN |
| | | | | YKTTPPVLDSDGSFFLYSKLTVDKSRWQQG |
| | | | | NVFSCSVMHEALHNHYTQ |
| | | | | KSLSLSPGK |
| 10 | OX40L | | Amino acid | DIQMTQSPSSLSASVGDRVTITCRASQGISSW |
| | benchmark | | sequence of OX40L | LAWYQQKPEKAPKSLIY |
| | antibody | | benchmark antibody | AASSLQSGVPSRFSGSGSGTDFTLTISSLQPED |
| | light | | light chain (Seq ID | FATYYCQQYNSYPYTFG |
| | chain | | No: 178 in | QGTKLEIKRTVAAPSVFIFPPSD |
| | | | WO2011/073180, | EQLKSGTASVVCLLNNFYPREAKVQWK |
| | | | Table A-5) | VDNALQSG |
| | | | | NSQESVTEQDSKDSTYSLSSTLTLSKADYEKH |
| | | | | KVYACEVTH |
| | | | | QGLSSPVTKSFNRGEC |
| 11 | κ light | | Amino acid | DIQMTQSPSSLSASVGDRVTITCRASQGISSW |
| | chain | | sequence of kappa | LAWYQQKPEKAPKSLIYAASSLQSGVPSRESG |
| | variable | | light chain variable | SGSGTDFTLTISSLQPEDFATYYCQQYNSYPY |
| | region of | | region of LC.001 | TFGQGTKLEIK |
| | LC.001 | | (Seq ID No: 1 in | |
| | | | WO2006/029879) | |
| 12 | γ heavy | | Amino acid | EVQLLESGGGLVQPGGSLRLSCAASGFTFNSY |
| | chain | | sequence of γ heavy | AMSWVRQAPGKGLEWVSIISGSGGFTYYADS |
| | variable | | chain variable | VKGRFTISRDNSRTTLYLQMNSLRAEDTAVY |
| | region of | | region of LC.001 | YCAKDRLVAPGTFDYWGQGALVTVSS |
| | LC.001 | | (Seq ID No: 2 in | |
| | | | WO2006/029879) | |
| 13 | κ light | | Amino acid | EIVLTQSPGTLSLSPGERATLSCRASQSVSSNY |
| | chain | | sequence of kappa | LAWYQQKPGQAPRLLIYGASSRATGIPDRFSG |
| | variable | | light chain variable | SGSGTDFTLTISRLEPEDFAVYYCQQYGSSFTF |
| | region of | | region of LC.005 | GPGTKVDIK |
| | LC.005 | | (Seq ID No: 3 in | |
| | | | WO2006/029879) | |
| 14 | γ heavy | | Amino acid | QVQLVESGGGVVQPGRSLRLSCAASGFTFSN |
| | chain | | sequence of γ heavy | FGMHWVRQAPGKGLEWVAAIWYDGHDKYY |
| | variable | | chain variable | SYYVKGRFTISRDNSKNTLFLQMNSLRAEDT |
| | region of | | region of LC.005 | AVYYCARDSSSWYRYFDYWGQGTLVTVSS |
| | LC.005 | | (Seq ID No: 4 in | |
| | | | WO2006/029879) | |
| 15 | κ light | | Amino acid | EIVLTQSPGTLSLSPGERATLSCRASQSVSSSY |
| | chain | | sequence of kappa | LAWYQQKPGQAPRLLIYGASSRATGIPDRESG |
| | variable | | light chain variable | SGSGTDFTLTISRLEPEDFAVYYCQQYGSSFTF |
| | region of | | region of LC.010 | GPGTKVDIK |
| | LC.010 | | (Seq ID No: 5 in | |
| | | | WO2006/029879) | |
| 16 | γ heavy | | Amino acid | QVQLVESGGGVVQPGRSLRLSCAASGFTFSN |
| | chain | | sequence of γ heavy | FGMHWVRQAPGKGLEWVAAIWYDGHDKYY |
| | variable | | chain variable | AYYVKGRFTISRDNSKNTLFLQMNSLRAEDT |
| | region of | | region of LC.010 | AVYYCARDSSSWYRYFDYWGQGTLVTVSS |
| | LC.010 | | (Seq ID No: 6 in | |
| | | | WO2006/029879) | |
| 17 | κ light | | Amino acid | MLHPLCKVGSHQGSVAVDLGQISLSPSAACS |
| | chain | | sequence of kappa | LKILQLITVNSIIVSLTFGGGTKVEIK |
| | variable | | light chain variable | |
| | region of | | region of LC.029 | |
| | LC.029 | | (Seq ID No: 7 in | |
| | | | WO2006/029879) | |
| 18 | γ heavy | | Amino acid | QVQLVESGGGVVQPGRSLRLSCAASGFTFSN |
| | chain | | sequence of γ heavy | FGMHWVRQAPGKGLEWVAAIWYDGHDKYY |
| | variable | | chain variable | SYYVKGRFTISRDNSKNTLFLQMNSLRAEDT |
| | region of | | region of LC.029 | AVYYCARDSSSWYRYFDYWGQGTLVTVSS |
| | LC.029 | | (Seq ID No: 8 in | |
| | | | WO2006/029879) | |
| 19 | κ light | | Amino acid | MPPVWKVGSHQGSAAVDLGQISLSPSAACSL |
| | chain | | sequence of kappa | KILQLITVNSLIVTLTFGGGTKVEIK |
| | variable | | light chain variable | |
| | region of | | region of LC.019 | |
| | LC.019 | | (Seq ID No: 9 in | |
| | | | WO2006/029879) | |
| 20 | γ heavy | | Amino acid | QVQLVESGGGVVQPGRSLRLSCAASGFTFSS |
| | chain | | sequence of γ heavy | YTMHWVRQAPGKGLEWVAVIWYDGSNKYY |
| | variable | | chain variable | VDSVKGRFTISRDNSKNTLYLQMNSLRAEDT |
| | region of | | region of LC.019 | AVYYCARKNWSFDFWGQGTLVTVSS |
| | LC.019 | | (Seq ID No: 10 in | |
| | | | WO2006/029879) | |
| 21 | κ light | | Amino acid | EIVLTQSPATLSLSPGERATLSCRASQGVSRYL |
| | chain | | sequence of kappa | AWYQQKPGQAPRLLIYDASNRATGIPARVSG |
| | variable | | light chain variable | SGPGTDFTLTISSLEPEDFAVDYCQQRSNWQY |
| | region of | | region of LC.033 | TFGQGTKLEI |
| | LC.033 | | (Seq ID No: 11 in | |
| | | | WO2006/029879) | |
| 22 | γ heavy | | Amino acid | QKQLVEFGGGVVQPGRSLRLSCAASGFTFSN |
| | chain | | sequence of γ heavy | YGMHWVRQAPGKGLEWVAVIWNDGSNKYY |
| | variable | | chain variable | VDSVKGRFIISRDNSKNTLYLQMNSLRAEDT |
| | region of | | region of LC.033 | AVYYCARDRMGIYYYGMDVWGQGTTVTVS |
| | LC.033 | | (Seq ID No: 12 in | S |
| | | | WO2006/029879) | |
| 23 | mutant κ | | Amino acid | EIVLTQSPATLSLSPGERATLSCRASQSVSSYL |
| | light | | sequence of mutant | AWYQQKPGQAPRLLIYDASNRATGIPARFSG |
| | chain | | kappa light chain | SGSGTDFTLTISSLEPEDFAVYYCQQRSNWTF |
| | variable | | variable region of | GQGTKVEI |
| | region of | | LC.033 (Seq ID No: | |
| | LC.033 | | 16 in | |
| | | | WO2006/029879) | |
| 24 | γ heavy | | Amino acid | EVQLLESGGGLVQPGGSLRLSCAASGFTFSSY |
| | chain | | sequence of γ heavy | AMNWVRQAPGKGLEWVSIISGSGGFTYYAD |
| | variable | | chain variable | SVKGRFTISRDNSKNTLYLQMNRLRAEDTAI |
| | region of | | region of LC.059 | YFCAKDDIPAAGTFDPWGQGTLVTVSS |
| | LC.059 | | (Seq ID No: 17 in | |
| | | | WO2006/029879) | |
| 25 | κ light | | Amino acid | AIQLTQSPSSLSASVGDRVTITCRASQGISSAL |
| | chain | | sequence of kappa | AWYQQKPGKAPKLLIYDVSSLESGVPSRFSGS |
| | variable | | light chain variable | GSGTDFTLTISSLQPEDFATYYCQQFNSYWTF |
| | region of | | region of LC.060 | GQGTKVEIK |
| | LC.060 | | (Seq ID No: 18 in | |
| | | | WO2006/029879) | |
| 26 | γ heavy | | Amino acid | EVQLLESGGGLVQPGGSLRLSCAASGFTESSY |
| | chain | | sequence of γ heavy | AMSWVRQAPGKGLEWVSLISGSGGLTKYAD |
| | variable | | chain variable | SVKGRFTISRDNSKRTLYLQMNSLRAEDTAV |
| | region of | | region of LC.060 | YYCAKDILVTGALDYWGQGTLVTVSS |
| | LC.060 | | (Seq ID No: 19 in | |
| | | | WO2006/029879) | |
| 27 | γ heavy | | Amino acid | EVQLLESGGGLVQPGGSLRLSCAASGFTFSSY |
| | chain | | sequence of γ heavy | AMNWVRQAPGKGLEWVSIISGSGGFTYYAD |
| | variable | | chain variable | SVKGRFTISRDNSKKTLYLQMSRLRAEDTAIY |
| | region | | region of LC.063 | FCAKDDIPAAGTFDPWGQGTLVTVSS |
| | of | | (Seq ID No: 20 in | |
| | LC.063 | | WO2006/029879) | |
| 28 | 8E12 | | Amino acid | DILMTQTPLSLPVSLGDQASISCRSSQSIVHGN |
| | light | | sequence of 8E12 | GNTYLEWHLQKPGQSPKLLIYRVSNRFSGVP |
| | chain | | light chain variable | DRFSGSGSGTDFTLKINRVEAEDLGVYYCFQ |
| | variable | | region (Seq ID No: | GSHVPYTFGGGTKVEIKR |
| | region | | 13 in US7,812,133) | |
| 29 | 8E12 | | Amino acid | DIVMTQTPLSLPVSLGDQASMYCRSSQSPVHS |
| | heavy | | sequence of 8E12 | NGNTYLHWYLQKPGQSPKLLIYKVSNRFSGV |
| | chain | | heavy chain variable | PDRFSGSGSGTDFTLKISRVEAEDLGVYFCSQ |
| | variable | | region (Seq ID No: | STHIPWTFGGGTKVEIKR |
| | region | | 14 in US7,812,133) | |
| 30 | 13G5 | | Amino acid | QVQLQQPGAELVRPGASVKLSCKASGYTFTS |
| | light | | sequence of 13G5 | YWLNWVKQRPGQGLEWIVMIDPSDSETHYN |
| | chain | | light chain variable | QVFKDKATLTVDKSSSTAYMQLSSLTSEDSA |
| | variable | | region (Seq ID No: | VYYCIRGRGNFYGGSHAMEYWGQGTLLTVS |
| | region | | 15 in US7,812,133) | S |
| 31 | 13G5 | | Amino acid | QVQLQQPGAELVKPGTSVKLSCKASGYSFTS |
| | heavy | | sequence of 13G5 | YWMHGVRQRPGQGLEWIGEIDPSNGRTNYN |
| | chain | | heavy chain variable | EKFKSKATLTVDKSSSTAYIQLSSLTSEDSAV |
| | variable | | region (Seq ID No: | YYCTRERSPRYFDVWGAGTTLTVSS |
| | region | | 16 in US7,812,133) | |
| 32 | | | HCDR3 motif | VRGXYYY |

IMGT indicates that CDR is determined using IMGT nomenclature;
KABAT indicates that CDR is determined using Kabat nomenclature.
The numbering in the sequence correlation table takes precedence over any inconsistent numbering elsewhere in this text.

The disclosure includes the following numbered embodiments:

1. An aqueous pharmaceutical formulation comprising:
(a) an anti-OX40 ligand (OX40L) antagonist antibody or antigen binding fragment thereof;
(b) a stabilizer;
(c) a chelator;
(d) a surfactant; and
(e) a buffer comprising histidine or a histidine salt;
wherein the pH of the aqueous pharmaceutical formulation ranges from 5.5 to about 6.5,
wherein the antibody or antigen binding fragment thereof comprises:
   a heavy chain complementarity determining region (HCDR) 1 of SEQ ID NO: 42, an HCDR2 of SEQ ID NO:44, and an HCDR3 of SEQ ID NO:46; and
   a light chain complementarity determining region (LCDR) 1 of SEQ ID NO:56, an LCDR2 of SEQ ID NO:58, and an LCDR3 of SEQ ID NO:60; and
wherein the aqueous pharmaceutical formulation is suitable for parenteral administration to a mammalian subject.

2. An aqueous pharmaceutical formulation comprising:
(a) an anti-OX40 ligand (OX40L) antagonist antibody or antigen binding fragment thereof;
(b) a stabilizer;
(c) a chelator;
(d) a surfactant; and
(e) a buffer comprising histidine or a histidine salt;
wherein the pH of the aqueous pharmaceutical formulation ranges from 5.5 to about 6.5,
wherein the antibody or antigen binding fragment thereof comprises:
   a heavy chain complementarity determining region (HCDR) 1 of SEQ ID NO: 36, an HCDR2 of SEQ ID NO:38, and an HCDR3 of SEQ ID NO:40; and
   a light chain complementarity determining region (LCDR) 1 of SEQ ID NO:50, an LCDR2 of SEQ ID NO:52, and an LCDR3 of SEQ ID NO:54; and
wherein the aqueous pharmaceutical formulation is suitable for parenteral administration to a mammalian subject.

3. An aqueous pharmaceutical formulation comprising:
(a) an anti-OX40 ligand (OX40L) antagonist antibody or antigen binding fragment thereof;
(b) a stabilizer;
(c) a chelator;
(d) a surfactant; and
(e) a buffer comprising histidine or a histidine salt;
wherein the pH of the aqueous pharmaceutical formulation ranges from 5.5 to about 6.5,
   wherein the antibody or antigen binding fragment thereof comprises a VH domain comprising at least 80% identity to the amino acid sequence of SEQ ID NO: 34 and a VL domain having at least 80% identity to the amino acid sequence of SEQ ID NO: 48; and
wherein the aqueous pharmaceutical formulation is suitable for parenteral administration to a mammalian subject.

4. The aqueous pharmaceutical formulation of embodiment 1 or 2, wherein the antibody or antigen binding fragment comprises a VH domain comprising at least 80% identity to the amino acid sequence of SEQ ID NO: 34 and/or comprises a VL domain having at least 80% identity to the amino acid sequence of SEQ ID NO: 48.

5. The aqueous pharmaceutical formulation of any of embodiments 1-4, wherein the antibody or antigen binding fragment comprises a VH domain of SEQ ID NO: 34 and/or a VL domain of SEQ ID NO: 48.

6. The aqueous pharmaceutical formulation of any of embodiments 1-5, wherein the antibody or antigen binding fragment comprises a heavy chain and/or a light chain, wherein the heavy chain amino acid sequence consists of the amino acid sequence of SEQ ID No: 62 and/or the light chain amino acid sequence consists of the amino acid sequence of SEQ ID No: 64.

7. The aqueous pharmaceutical formulation of any of embodiments 1-6, wherein the antibody or antigen binding fragment thereof is amlitelimab or a variant thereof.

8. The aqueous pharmaceutical formulation of any of embodiments 1-7, wherein the antibody or antigen binding fragment thereof is present in a concentration ranging from 28 to 138 mg/mL, or from 31 to 125 mg/mL.

9. The aqueous pharmaceutical formulation of any one of embodiments 1-8, wherein the antibody or antigen binding fragment thereof is present in a concentration of 28 mg/mL, 31 mg/mL, 62.5 mg/mL, 125 mg/mL, or 138 mg/mL.

10. The aqueous pharmaceutical formulation according to any one of embodiments 1-9, wherein the antibody or antigen binding fragment thereof is present at a concentration of about 62.5 mg/mL, preferably 62.5 mg/mL.

11. The aqueous pharmaceutical formulation according to any one of embodiments 1-9, wherein the antibody or antigen binding fragment thereof is present at a concentration of about 125 mg/mL, preferably 125 mg/mL.

12. The aqueous pharmaceutical formulation of any one of embodiments 1-11, wherein the stabilizer is sucrose.

13. The aqueous pharmaceutical formulation of any one of embodiments 1-12, wherein the at least one stabilizer is sucrose, which is present in an amount of 220 mM.

14. The aqueous pharmaceutical formulation of any one of embodiments 1-13, wherein the surfactant is polysorbate 80.

15. The aqueous pharmaceutical formulation of any one of embodiments 1-14, comprising 0.02% (w/v) to 0.1% (w/v) polysorbate 80.

16. The aqueous pharmaceutical formulation of any one of embodiments 1-15, comprising 0.02% (w/v) to 0.06% polysorbate 80.

17. The aqueous pharmaceutical formulation of any one of embodiments 1-16, comprising 0.04% (w/v) or 0.06% polysorbate 80.

18. The aqueous pharmaceutical formulation of any one of embodiments 1-17, wherein the buffer comprises 10 mM L-histidine or histidine hydrochloride.

19. The aqueous pharmaceutical formulation of any one of embodiments 1-18, further comprising a chelator selected from the group consisting of ethylenediaminetetraacetic acid (EDTA), diethylenetriamene pentaacetate (DTPA) and salts thereof, and any combinations thereof.

20. The aqueous pharmaceutical formulation of embodiment 19, wherein the chelator comprises one or both of 10 μM EDTA and 10 μM DTPA.

21. The aqueous pharmaceutical formulation of embodiment 19, wherein the chelator comprises 10 μM EDTA.

22. The aqueous pharmaceutical formulation of any of embodiments 1-7, comprising:
28 mg/mL to 138 mg/mL of the antibody or antigen binding fragment thereof;
10 mM±1.5 mM L-histidine or histidine hydrochloride;
220 mM±33 mM sucrose,
0.06% (w/v) polysorbate 80; and
water;
with the pH of the aqueous pharmaceutical formulation between about 5.8 and about 6.2.

23. The aqueous pharmaceutical formulation of embodiment 22, wherein the antibody or antigen binding fragment thereof is present at a concentration of about 62.5 mg/mL, preferably 62.5 mg/mL.

24. The aqueous pharmaceutical formulation of embodiment 22, wherein the antibody or antigen binding fragment thereof is present at a concentration of about 125 mg/mL, preferably 125 mg/mL.

25. The aqueous pharmaceutical formulation of any one of embodiments 22-24, further comprising 10 μM EDTA or 10 μM DTPA.

26. The aqueous pharmaceutical formulation of any one of embodiments 22-25, wherein the aqueous pharmaceutical formulation is free or essentially free of a chelator.

27. The aqueous pharmaceutical formulation of any one of embodiments 1-26, which is free or essentially free of particles.

28. The aqueous pharmaceutical formulation of any one of embodiments 1-27, which is free or substantially free of sodium chloride.

29. The aqueous pharmaceutical formulation of any one of embodiments 1-28, which is free or substantially free of arginine.

30. The aqueous pharmaceutical formulation of any one of embodiments 1-29, wherein less than 5% of the antibody is detected in an aggregated form after 28 days of storage at 40° C., detected by size exclusion high performance liquid chromatography.

31. The aqueous pharmaceutical formulation of any one of embodiments 1-30, which is stable upon storage at 5° C. or 25° C. for at least 3 months.

32. The aqueous pharmaceutical formulation of any one of embodiments 1-31, which is stable upon storage at 40° C. for at least 2 months.

33. The aqueous pharmaceutical formulation of any one of embodiments 1-32, which is stable for at least 1 year when stored at a temperature of about 5° C.

34. The aqueous pharmaceutical formulation of any one of embodiments 1-33, which is comprised in a container.

35. The aqueous pharmaceutical formulation of any of embodiments 1-34, which is suitable for subcutaneous delivery.

36. A pharmaceutical unit dosage form suitable for parenteral administration to a mammalian subject which comprises the aqueous pharmaceutical formulation of any one of embodiments 1 to 35 in a suitable container.

37. The pharmaceutical unit dosage form of embodiment 36, wherein the aqueous pharmaceutical formulation is suitable for intravenous, subcutaneous, or intramuscular administration.

38. The pharmaceutical unit dosage form of embodiment 36 or 37, wherein the suitable container is a pre-filled syringe.

39. A sealed container comprising the aqueous pharmaceutical formulation of any one of embodiments 1-35.

40. The sealed container of embodiment 39, which is a vial, a syringe, a microinfusor, a pen delivery device, or an autoinjector.

41. The sealed container of embodiment 39 or embodiment 40, which is a single or multi-chambered syringe.

42. The sealed container of embodiment 41, which is a pre-filled syringe containing 2.25 mL of the aqueous pharmaceutical formulation.

43. A pre-filled syringe comprising an aqueous pharmaceutical formulation comprising:
about 28 mg/mL to about 138 mg/mL of an anti-OX40 ligand (OX40L) antagonist antibody or antigen binding fragment thereof;
about 10 mM L-histidine or histidine hydrochloride;
about 220 mM sucrose;
about 0.06% (w/v) polysorbate 80; and
water;
wherein the pH of the aqueous pharmaceutical formulation is about 6.0.

44. A pre-filled pen or autoinjector comprising an aqueous pharmaceutical formulation comprising:
about 28 mg/mL to about 138 mg/mL of an anti-OX40 ligand (OX40L) antagonist antibody or antigen binding fragment thereof;
about 10 mM L-histidine or histidine hydrochloride;
about 220 mM sucrose;
about 0.06% (w/v) polysorbate 80; and
water;
wherein the pH of the aqueous pharmaceutical formulation is about 6.0.

45. The pre-filled syringe of embodiment 43 or the pre-filled pen or autoinjector of embodiment 44, further comprising 10 μM EDTA or 10 μM DTPA.

46. The pre-filled syringe of embodiment 43 or the pre-filled pen or autoinjector of embodiment 44, further comprising 10 μM EDTA.

47. The pre-filled syringe of any one of embodiments 43, 45 or 46 or the pre-filled pen or autoinjector of any one of embodiments 44-46, wherein the antibody or antigen binding fragment thereof is present at a concentration of about 62.5 mg/mL, preferably 62.5 mg/mL.

48. The pre-filled syringe of any one of embodiments 43, 45 or 46 or the pre-filled pen or autoinjector of any one of embodiments 44-46, wherein the antibody or antigen binding fragment thereof is present at a concentration of about 125 mg/mL, preferably 125 mg/mL.

49. The pre-filled syringe of any of embodiments 43 or 45-48 or the prefilled pen or autoinjector of any of embodiments 44-48, wherein the anti-OX40 ligand (OX40L) antagonist antibody or antigen binding fragment thereof, comprises:
(a) a heavy chain complementarity determining region (HCDR) 1 of SEQ ID NO: 42, an HCDR2 of SEQ ID NO:44, and an HCDR3 of SEQ ID NO:46; and a light chain complementarity determining region (LCDR) 1 of SEQ ID NO:56, an LCDR2 of SEQ ID NO:58, and an LCDR3 of SEQ ID NO:60; or
(b) a heavy chain complementarity determining region (HCDR) 1 of SEQ ID NO: 36, an HCDR2 of SEQ ID NO:38, and an HCDR3 of SEQ ID NO:40; and a light chain complementarity determining region (LCDR) 1 of SEQ ID NO:50, an LCDR2 of SEQ ID NO:52, and an LCDR3 of SEQ ID NO:54; or
(c) a VH domain comprising at least 80% identity to the amino acid sequence of SEQ ID NO: 34 and a VL domain having at least 80% identity to the amino acid sequence of SEQ ID NO: 48; or
(d) a VH domain comprising at least 80% identity to the amino acid sequence of SEQ ID NO: 34 and/or comprises a VL domain having at least 80% identity to the amino acid sequence of SEQ ID NO: 48; or
(e) a heavy chain amino acid sequence consisting of the amino acid sequence of SEQ ID No: 62 and/or the light chain amino acid sequence consisting of the amino acid sequence of SEQ ID No: 64

50. The pre-filled syringe of any of embodiments 43 or 45-49 or the pre-filled pen or autoinjector of any of embodiments 44-49, comprising about 31 mg/mL to about 125 mg/mL amlitelimab or a variant thereof.

51. The pre-filled syringe of any of embodiments 43 or 45-50 or the pre-filled pen or autoinjector of any of embodiments 44-50, comprising 62.5 mg/mL amlitelimab or a variant thereof.

52. The pre-filled syringe of any of embodiments 43 or 45-50 or the pre-filled pen or autoinjector of any of embodiments 44-50, comprising 125 mg/mL amlitelimab or a variant thereof.

53. A kit comprising a sealed container of any one of embodiments 39-42, a pre-filled syringe of any one of embodiments 43 or 45-52 or a pre-filled pen or autoinjector of any one of embodiments 44-52.

54. A kit comprising:
a sealed container comprising the aqueous pharmaceutical formulation of any one of embodiment 1-35; and
at least one separate injection device for delivery the aqueous pharmaceutical formulation to a mammalian subject in need thereof.

55. The kit of embodiment 54, wherein the injection device is a syringe, a microinfusor, a pen delivery device, or an autoinjector.

56. The kit of embodiment 55, wherein the injection device is a single or multi-chambered syringes.

57. The aqueous pharmaceutical formulation of any one of embodiments 1-35, the pharmaceutical unit dosage form of any one of embodiments 36-38, the sealed container of any one of embodiments 39-42, the pre-filled syringe of any one of embodiments 43 or 45-52, the pre-filled pen or autoinjector of any one of embodiments 44-52, or the kit of any one of embodiments 53-56 for use in treating a disease or condition in a subject selected from the group consisting of an autoimmune disease or condition, an inflammatory disease or condition, a systemic inflammatory disease or condition, and a transplant rejection disease or condition mediated by hOX40L.

58. The aqueous pharmaceutical formulation of any one of embodiments 1-35, the pharmaceutical unit dosage form of any one of embodiments 36-38, the sealed container of any one of embodiments 39-42, the pre-filled syringe of any one of embodiments 43 or 45-52, the pre-filled pen or autoinjector of any one of embodiments 44-52, or the kit of any one of embodiments 53-56 for use in treating atopic dermatitis.

59. The aqueous pharmaceutical formulation of any one of embodiments 1-35, the pharmaceutical unit dosage form of any one of embodiments 36-38, the sealed container of any one of embodiments 39-42, the pre-filled syringe of any one of embodiments 43 or 45-52, the pre-filled pen or autoinjector of any one of embodiments 44-52, or the kit of any one of embodiments 53-56 for use in treating asthma.

60. Use of the aqueous pharmaceutical formulation of any one of embodiments 1-35, the pharmaceutical unit dosage form of any one of embodiments 36-38, the sealed container of any one of embodiments 39-42, the pre-filled syringe of any one of embodiments 43 or 45-52, the pre-filled pen or autoinjector of any one of embodiments 44-52, or the kit of any one of embodiments 53-56 in the manufacture of a medicament for treating a disease or condition in a subject selected from the group consisting of an autoimmune disease or condition, an inflammatory disease or condition, a systemic inflammatory disease or condition, and a transplant rejection disease or condition mediated by hOX40L.

61. Use of the aqueous pharmaceutical formulation of any one of embodiments 1-35, the pharmaceutical unit dosage form of any one of embodiments 36-38, the sealed container of any one of embodiments 39-42, the pre-filled syringe of any one of embodiments 43 or 45-52, the pre-filled pen or autoinjector of any one of embodiments 44-52, or the kit of any one of embodiments 53-56 in the manufacture of a medicament for treating atopic dermatitis.

62. Use of the aqueous pharmaceutical formulation of any one of embodiments 1-35, the pharmaceutical unit dosage form of any one of embodiments 36-38, the sealed container of any one of embodiments 39-42, the pre-filled syringe of any one of embodiments 43 or 45-52, the pre-filled pen or autoinjector of any one of embodiments 44-52, or the kit of any one of embodiments 53-56 in the manufacture of a medicament for treating asthma.

63. A method of treating a disease or condition in a subject selected from the group consisting of an autoimmune disease or condition, an inflammatory disease or condition, a systemic inflammatory disease or condition, and a transplant rejection disease or condition (e.g. an autoimmune disease or condition, an inflammatory disease or condition, a systemic inflammatory disease or condition, and a transplant rejection disease or condition mediated by hOX40L), comprising administering to the subject an effective amount of the aqueous pharmaceutical formulation of any one of embodiments 1 to 35.

64. A method of treating atopic dermatitis comprising administering to the subject an effective amount of the aqueous pharmaceutical formulation of any one of embodiments 1 to 35.

65. A method of treating asthma comprising administering to the subject an effective amount of the aqueous pharmaceutical formulation of any one of embodiments 1 to 35.

66. An aqueous pharmaceutical formulation comprising:
(a) an anti-OX40 ligand (OX40L) antagonist antibody or antigen binding fragment;
(b) a stabilizer;
(c) a chelator;
(d) a surfactant; and
(e) a buffer comprising histidine or a histidine salt;
wherein the pH of the aqueous pharmaceutical formulation ranges from 5.5 to about 6.5,
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60; and
wherein the aqueous pharmaceutical formulation is suitable for parenteral administration to a mammalian subject.

67. The method of embodiment 66, wherein the VL domain comprises the amino acid sequence of SEQ ID NO:48 and the VH domain comprises the amino acid sequence of SEQ ID NO:34.

68. The method of embodiment 66 or 67, wherein the antibody comprises a heavy chain and a light chain, wherein the heavy chain amino acid sequence consists of the amino acid sequence of SEQ ID NO: 62 and the light chain amino acid sequence consists of the amino acid sequence of SEQ ID NO: 64.

69. A pharmaceutical unit dosage form suitable for parenteral administration to a mammalian subject, comprising
(a) about 62.5 mg (e.g. 62.15 mg) of an anti-OX40 ligand (OX40L) antagonist antibody or antigen binding fragment thereof;
(b) about 1.49 mg of L-Histidine;
(c) about 2.18 mg of L-Histidine hydrochloride (e.g L-histidine hydrochloride monohydrate);
(d) about 150.62 mg sucrose;
(e) about 0.0074 mg EDTA (e.g. EDTA disodium salt dihydrate);
(f) about 1.2 mg of polysorbate 80 (e.g. super refined polysorbate 80); and
(g) optionally water.
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60.

70. A pharmaceutical unit dosage form suitable for parenteral administration to a mammalian subject, comprising
(a) about 125 mg of an anti-OX40 ligand (OX40L) antagonist antibody or antigen binding fragment thereof;
(b) about 1.49 mg of L-Histidine;
(c) about 2.18 mg of L-Histidine hydrochloride (e.g. L-Histidine hydrochloride monohydrate);
(d) about 150.62 mg sucrose;
(e) about 0.0074 mg EDTA (e.g. EDTA disodium salt dihydrate);
(f) about 1.2 mg of super refined polysorbate 80; and
(g) optionally water.
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60.

71. A pharmaceutical unit dosage form suitable for parenteral administration to a mammalian subject, comprising
(a) about 250 mg of an anti-OX40 ligand (OX40L) antagonist antibody or antigen binding fragment thereof;
(b) about 1.49 mg of L-Histidine;
(c) about 2.18 mg of L-Histidine hydrochloride (e.g. L-Histidine hydrochloride monohydrate);
(d) about 150.62 mg sucrose;
(e) about 0.0074 mg EDTA (e.g. EDTA disodium salt dihydrate);
(f) about 1.2 mg of polysorbate 80 (e.g. super refined polysorbate 80); and
(g) optionally water.
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60.

72. A pharmaceutical unit dosage form according to any of embodiments 69-71, wherein the anti-OX40 ligand (OX40L) antagonist antibody or antigen binding fragment thereof is amlitelimab or a variant thereof.

73. A pharmaceutical unit dosage form according to any of embodiments 69-71, wherein the unit dosage form is contained in a suitable container.

74. A pharmaceutical unit dosage form according to embodiment 73, wherein the suitable container is a vial, a syringe (e.g. a pre-filled syringe), a microinfusor, a pen delivery device (e.g. a pre-filled pen delivery device), or an autoinjector.

75. A pharmaceutical unit dosage form according to embodiment 73 or 74, wherein the container is sealed.

The disclosure includes the following numbered aspects:

1. A pharmaceutical formulation comprising:
(a) an anti-OX40 ligand (OX40L) antagonist antibody or antigen binding fragment thereof;
(b) a stabilizer;
(c) a chelator;
(d) a surfactant; and
(e) a buffer comprising histidine or a histidine salt;
wherein the pH of the pharmaceutical formulation ranges from 5.5 to about 6.5,
wherein the antibody or antigen binding fragment thereof comprises:
   a heavy chain complementarity determining region (HCDR) 1 of SEQ ID NO: 42, an HCDR2 of SEQ ID NO:44, and an HCDR3 of SEQ ID NO:46; and
   a light chain complementarity determining region (LCDR) 1 of SEQ ID NO:56, an LCDR2 of SEQ ID NO:58, and an LCDR3 of SEQ ID NO:60; and
wherein the pharmaceutical formulation is suitable for parenteral administration to a mammalian subject.

2. A pharmaceutical formulation comprising:
(a) an anti-OX40 ligand (OX40L) antagonist antibody or antigen binding fragment thereof;
(b) a stabilizer;
(c) a chelator;
(d) a surfactant; and
(e) a buffer comprising histidine or a histidine salt;
wherein the pH of the pharmaceutical formulation ranges from 5.5 to about 6.5,
wherein the antibody or antigen binding fragment thereof comprises:
   a heavy chain complementarity determining region (HCDR) 1 of SEQ ID NO: 36, an HCDR2 of SEQ ID NO:38, and an HCDR3 of SEQ ID NO:40; and
   a light chain complementarity determining region (LCDR) 1 of SEQ ID NO:50, an LCDR2 of SEQ ID NO:52, and an LCDR3 of SEQ ID NO:54; and
wherein the pharmaceutical formulation is suitable for parenteral administration to a mammalian subject.

3. A pharmaceutical formulation comprising:
(a) an anti-OX40 ligand (OX40L) antagonist antibody or antigen binding fragment thereof;
(b) a stabilizer;
(c) a chelator;
(d) a surfactant; and
(e) a buffer comprising histidine or a histidine salt;
wherein the pH of the pharmaceutical formulation ranges from 5.5 to about 6.5,
   wherein the antibody or antigen binding fragment thereof comprises a VH domain comprising at least 80% identity to the amino acid sequence of SEQ ID NO: 34 and a VL domain having at least 80% identity to the amino acid sequence of SEQ ID NO: 48; and
wherein the pharmaceutical formulation is suitable for parenteral administration to a mammalian subject.

4. The pharmaceutical formulation of aspect 1 or 2, wherein the antibody or antigen binding fragment comprises a VH domain comprising at least 80% identity to the amino acid sequence of SEQ ID NO: 34 and/or comprises a VL domain having at least 80% identity to the amino acid sequence of SEQ ID NO: 48.

5. The pharmaceutical formulation of any of aspects 1-4, wherein the antibody or antigen binding fragment comprises a VH domain of SEQ ID NO: 34 and/or a VL domain of SEQ ID NO: 48.

6. The pharmaceutical formulation of any of aspects 1-5, wherein the antibody or antigen binding fragment comprises a heavy chain and/or a light chain, wherein the heavy chain amino acid sequence consists of the amino acid sequence of SEQ ID No: 62 and/or the light chain amino acid sequence consists of the amino acid sequence of SEQ ID No: 64.

7. The pharmaceutical formulation of any of aspects 1-6, wherein the antibody or antigen binding fragment thereof is amlitelimab or a variant thereof.

8. The pharmaceutical formulation of any of aspects 1-7, wherein the antibody or antigen binding fragment thereof is present in a concentration ranging from 28 to 138 mg/mL, or from 31 to 125 mg/mL.

9. The pharmaceutical formulation of any one of aspects 1-8, wherein the antibody or antigen binding fragment thereof is present in a concentration of 28 mg/mL, 31 mg/mL, 62.5 mg/mL, 125 mg/mL, or 138 mg/mL.

10. The pharmaceutical formulation according to any one of aspects 1-9, wherein the antibody or antigen binding fragment thereof is present at a concentration of about 62.5 mg/mL, preferably 62.5 mg/mL.

11. The pharmaceutical formulation according to any one of aspects 1-9, wherein the antibody or antigen binding fragment thereof is present at a concentration of about 125 mg/mL, preferably 125 mg/mL.

12. The pharmaceutical formulation of any one of aspects 1-11, wherein the stabilizer is sucrose.

13. The pharmaceutical formulation of any one of aspects 1-12, wherein the at least one stabilizer is sucrose, which is present in an amount of 220 mM.

14. The pharmaceutical formulation of any one of aspects 1-13, wherein the surfactant is polysorbate 80.

15. The pharmaceutical formulation of any one of aspects 1-14, comprising 0.02% (w/v) to 0.1% (w/v) polysorbate 80.

16. The pharmaceutical formulation of any one of aspects 1-15, comprising 0.02% (w/v) to 0.06% polysorbate 80.

17. The pharmaceutical formulation of any one of aspects 1-16, comprising 0.04% (w/v) or 0.06% polysorbate 80.

18. The pharmaceutical formulation of any one of aspects 1-17, wherein the buffer comprises 10 mM L-histidine or histidine hydrochloride.

19. The pharmaceutical formulation of any one of aspects 1-18, further comprising a chelator selected from the group consisting of ethylenediaminetetraacetic acid (EDTA), diethylenetriamene pentaacetate (DTPA) and salts thereof, and any combinations thereof.

20. The pharmaceutical formulation of aspect 19, wherein the chelator comprises one or both of 10 μM EDTA and 10 μM DTPA.

21. The pharmaceutical formulation of aspect 19, wherein the chelator comprises 10 μM EDTA.

22. The pharmaceutical formulation of any of aspects 1-7, comprising:
28 mg/mL to 138 mg/mL of the antibody or antigen binding fragment thereof;
10 mM±1.5 mM L-histidine or histidine hydrochloride;
220 mM±33 mM sucrose,
0.06% (w/v) polysorbate 80; and
water;
with the pH of the pharmaceutical formulation between about 5.8 and about 6.2.

23. The pharmaceutical formulation of aspect 22, wherein the antibody or antigen binding fragment thereof is present at a concentration of about 62.5 mg/mL, preferably 62.5 mg/mL.

24. The pharmaceutical formulation of aspect 22, wherein the antibody or antigen binding fragment thereof is present at a concentration of about 125 mg/mL, preferably 125 mg/mL.

25. The pharmaceutical formulation of any one of aspects 22-24, further comprising 10 μM EDTA or 10 μM DTPA.

26. The pharmaceutical formulation of any one of aspects 22-25, wherein the pharmaceutical formulation is free or essentially free of a chelator.

27. The pharmaceutical formulation of any one of aspects 1-26, which is free or essentially free of particles.

28. The pharmaceutical formulation of any one of aspects 1-27, which is free or substantially free of sodium chloride.

29. The pharmaceutical formulation of any one of aspects 1-28, which is free or substantially free of arginine.

30. The pharmaceutical formulation of any one of aspects 1-29, wherein less than 5% of the antibody is detected in an aggregated form after 28 days of storage at 40° C., detected by size exclusion high performance liquid chromatography.

31. The pharmaceutical formulation of any one of aspects 1-30, which is stable upon storage at 5° C. or 25° C. for at least 3 months.

32. The pharmaceutical formulation of any one of aspects 1-31, which is stable upon storage at 40° C. for at least 2 months.

33. The pharmaceutical formulation of any one of aspects 1-32, which is stable for at least 1 year when stored at a temperature of about 5° C.

34. The pharmaceutical formulation of any one of aspects 1-33, which is comprised in a container.

35. The pharmaceutical formulation of any of aspects 1-34, which is suitable for subcutaneous delivery.

36. A pharmaceutical unit dosage form suitable for parenteral administration to a mammalian subject which comprises the pharmaceutical formulation of any one of aspects 1 to 35 in a suitable container.

37. The pharmaceutical unit dosage form of aspect 36, wherein the pharmaceutical formulation is suitable for intravenous, subcutaneous, or intramuscular administration.

38. The pharmaceutical unit dosage form of aspect 36 or 37, wherein the suitable container is a pre-filled syringe.

39. A sealed container comprising the pharmaceutical formulation of any one of aspects 1-35.

40. The sealed container of aspect 39, which is a vial, a syringe, a microinfusor, a pen delivery device, or an autoinjector.

41. The sealed container of aspect 39 or aspect 40, which is a single or multi-chambered syringe.

42. The sealed container of aspect 41, which is a pre-filled syringe containing 2.25 mL of the pharmaceutical formulation.

43. A pre-filled syringe comprising a pharmaceutical formulation comprising:
about 28 mg/mL to about 138 mg/mL of an anti-OX40 ligand (OX40L) antagonist antibody or antigen binding fragment thereof;
about 10 mM L-histidine or histidine hydrochloride;
about 220 mM sucrose;
about 0.06% (w/v) polysorbate 80; and
water;
wherein the pH of the pharmaceutical formulation is about 6.0.

44. A pre-filled pen or autoinjector comprising a pharmaceutical formulation comprising:
about 28 mg/mL to about 138 mg/mL of an anti-OX40 ligand (OX40L) antagonist antibody or antigen binding fragment thereof;
about 10 mM L-histidine or histidine hydrochloride;
about 220 mM sucrose;
about 0.06% (w/v) polysorbate 80; and water;
wherein the pH of the pharmaceutical formulation is about 6.0.

45. The pre-filled syringe of aspect 43 or the pre-filled pen or autoinjector of aspect 44, further comprising 10 μM EDTA or 10 μM DTPA.

46. The pre-filled syringe of aspect 43 or the pre-filled pen or autoinjector of aspect 44, further comprising 10 μM EDTA.

47. The pre-filled syringe of any one of aspects 43, 45 or 46 or the pre-filled pen or autoinjector of any one of aspects 44-46, wherein the antibody or antigen binding fragment thereof is present at a concentration of about 62.5 mg/mL, preferably 62.5 mg/mL.

48. The pre-filled syringe of any one of aspects 43, 45 or 46 or the pre-filled pen or autoinjector of any one of aspects 44-46, wherein the antibody or antigen binding fragment thereof is present at a concentration of about 125 mg/mL, preferably 125 mg/mL.

49. The pre-filled syringe of any of aspects 43 or 45-48 or the prefilled pen or autoinjector of any of aspects 44-48, wherein the anti-OX40 ligand (OX40L) antagonist antibody or antigen binding fragment thereof, comprises:
(a) a heavy chain complementarity determining region (HCDR) 1 of SEQ ID NO: 42, an HCDR2 of SEQ ID NO:44, and an HCDR3 of SEQ ID NO:46; and a light chain complementarity determining region (LCDR) 1 of SEQ ID NO:56, an LCDR2 of SEQ ID NO:58, and an LCDR3 of SEQ ID NO:60; or
(b) a heavy chain complementarity determining region (HCDR) 1 of SEQ ID NO: 36, an HCDR2 of SEQ ID NO:38, and an HCDR3 of SEQ ID NO:40; and a light chain complementarity determining region (LCDR) 1 of SEQ ID NO:50, an LCDR2 of SEQ ID NO:52, and an LCDR3 of SEQ ID NO:54; or
(c) a VH domain comprising at least 80% identity to the amino acid sequence of SEQ ID NO: 34 and a VL domain having at least 80% identity to the amino acid sequence of SEQ ID NO: 48; or
(d) a VH domain comprising at least 80% identity to the amino acid sequence of SEQ ID NO: 34 and/or comprises a VL domain having at least 80% identity to the amino acid sequence of SEQ ID NO: 48; or
(e) a heavy chain amino acid sequence consisting of the amino acid sequence of SEQ ID No: 62 and/or the light chain amino acid sequence consisting of the amino acid sequence of SEQ ID No: 64

50. The pre-filled syringe of any of aspects 43 or 45-49 or the pre-filled pen or autoinjector of any of aspects 44-49, comprising about 31 mg/mL to about 125 mg/mL amlitelimab or a variant thereof.

51. The pre-filled syringe of any of aspects 43 or 45-50 or the pre-filled pen or autoinjector of any of aspects 44-50, comprising 62.5 mg/mL amlitelimab or a variant thereof.

52. The pre-filled syringe of any of aspects 43 or 45-50 or the pre-filled pen or autoinjector of any of aspects 44-50, comprising 125 mg/mL amlitelimab or a variant thereof.

53. A kit comprising a sealed container of any one of aspects 39-42, a pre-filled syringe of any one of aspects 43 or 45-52 or a pre-filled pen or autoinjector of any one of aspects 44-52.

54. A kit comprising:
a sealed container comprising the pharmaceutical formulation of any one of aspect 1-35; and
at least one separate injection device for delivery the pharmaceutical formulation to a mammalian subject in need thereof.

55. The kit of aspect 54, wherein the injection device is a syringe, a microinfusor, a pen delivery device, or an autoinjector.

56. The kit of aspect 55, wherein the injection device is a single or multi-chambered syringes.

57. The pharmaceutical formulation of any one of aspects 1-35, the pharmaceutical unit dosage form of any one of aspects 36-38, the sealed container of any one of aspects 39-42, the pre-filled syringe of any one of aspects 43 or 45-52, the pre-filled pen or autoinjector of any one of aspects 44-52, or the kit of any one of aspects 53-56 for use in treating a disease or condition in a subject selected from the group consisting of an autoimmune disease or condition, an inflammatory disease or condition, a systemic inflammatory disease or condition, and a transplant rejection disease or condition mediated by hOX40L.

58. The pharmaceutical formulation of any one of aspects 1-35, the pharmaceutical unit dosage form of any one of aspects 36-38, the sealed container of any one of aspects 39-42, the pre-filled syringe of any one of aspects 43 or 45-52, the pre-filled pen or autoinjector of any one of aspects 44-52, or the kit of any one of aspects 53-56 for use in treating atopic dermatitis.

59. The pharmaceutical formulation of any one of aspects 1-35, the pharmaceutical unit dosage form of any one of aspects 36-38, the sealed container of any one of aspects 39-42, the pre-filled syringe of any one of aspects 43 or 45-52, the pre-filled pen or autoinjector of any one of aspects 44-52, or the kit of any one of aspects 53-56 for use in treating asthma.

60. Use of the pharmaceutical formulation of any one of aspects 1-35, the pharmaceutical unit dosage form of any one of aspects 36-38, the sealed container of any one of aspects 39-42, the pre-filled syringe of any one of aspects 43 or 45-52, the pre-filled pen or autoinjector of any one of aspects 44-52, or the kit of any one of aspects 53-56 in the manufacture of a medicament for treating a disease or condition in a subject selected from the group consisting of an autoimmune disease or condition, an inflammatory disease or condition, a systemic inflammatory disease or condition, and a transplant rejection disease or condition mediated by hOX40L.

61. Use of the pharmaceutical formulation of any one of aspects 1-35, the pharmaceutical unit dosage form of any one of aspects 36-38, the sealed container of any one of aspects 39-42, the pre-filled syringe of any one of aspects 43 or 45-52, the pre-filled pen or autoinjector of any one of aspects 44-52, or the kit of any one of aspects 53-56 in the manufacture of a medicament for treating atopic dermatitis.

62. Use of the pharmaceutical formulation of any one of aspects 1-35, the pharmaceutical unit dosage form of any one of aspects 36-38, the sealed container of any one of aspects 39-42, the pre-filled syringe of any one of aspects 43 or 45-52, the pre-filled pen or autoinjector of any one of aspects 44-52, or the kit of any one of aspects 53-56 in the manufacture of a medicament for treating asthma.

63. A method of treating a disease or condition in a subject selected from the group consisting of an autoimmune disease or condition, an inflammatory disease or condition, a systemic inflammatory disease or condition, and a transplant rejection disease or condition (e.g. an autoimmune disease or condition, an inflammatory disease or condition, a systemic inflammatory disease or condition, and a transplant rejection disease or condition mediated by hOX40L), comprising administering to the subject an effective amount of the pharmaceutical formulation of any one of aspects 1 to 35.

64. A method of treating atopic dermatitis comprising administering to the subject an effective amount of the pharmaceutical formulation of any one of aspects 1 to 35.

65. A method of treating asthma comprising administering to the subject an effective amount of the pharmaceutical formulation of any one of aspects 1 to 35.

66. A pharmaceutical formulation comprising:
(a) an anti-OX40 ligand (OX40L) antagonist antibody or antigen binding fragment;
(b) a stabilizer;
(c) a chelator;
(d) a surfactant; and
(e) a buffer comprising histidine or a histidine salt;
wherein the pH of the pharmaceutical formulation ranges from 5.5 to about 6.5,
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60; and
wherein the pharmaceutical formulation is suitable for parenteral administration to a mammalian subject.

67. The method of aspect 66, wherein the VL domain comprises the amino acid sequence of SEQ ID NO:48 and the VH domain comprises the amino acid sequence of SEQ ID NO:34.

68. The method of aspect 66 or 67, wherein the antibody comprises a heavy chain and a light chain, wherein the heavy chain amino acid sequence consists of the amino acid sequence of SEQ ID NO: 62 and the light chain amino acid sequence consists of the amino acid sequence of SEQ ID NO: 64.

69. A pharmaceutical unit dosage form suitable for parenteral administration to a mammalian subject, comprising
(a) about 62.5 mg (e.g. 62.15 mg) of an anti-OX40 ligand (OX40L) antagonist antibody or antigen binding fragment thereof;
(b) about 1.49 mg of L-Histidine;
(c) about 2.18 mg of L-Histidine hydrochloride (e.g L-histidine hydrochloride monohydrate);
(d) about 150.62 mg sucrose;
(e) about 0.0074 mg EDTA (e.g. EDTA disodium salt dihydrate);
(f) about 1.2 mg of polysorbate 80 (e.g. super refined polysorbate 80); and
(g) optionally water.
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60.

70. A pharmaceutical unit dosage form suitable for parenteral administration to a mammalian subject, comprising
(a) about 125 mg of an anti-OX40 ligand (OX40L) antagonist antibody or antigen binding fragment thereof;
(b) about 1.49 mg of L-Histidine;
(c) about 2.18 mg of L-Histidine hydrochloride (e.g. L-Histidine hydrochloride monohydrate);
(d) about 150.62 mg sucrose;
(e) about 0.0074 mg EDTA (e.g. EDTA disodium salt dihydrate);
(f) about 1.2 mg of super refined polysorbate 80; and
(g) optionally water.
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60.

71. A pharmaceutical unit dosage form suitable for parenteral administration to a mammalian subject, comprising
(a) about 250 mg of an anti-OX40 ligand (OX40L) antagonist antibody or antigen binding fragment thereof;
(b) about 1.49 mg of L-Histidine;
(c) about 2.18 mg of L-Histidine hydrochloride (e.g. L-Histidine hydrochloride monohydrate);
(d) about 150.62 mg sucrose;
(e) about 0.0074 mg EDTA (e.g. EDTA disodium salt dihydrate);
(f) about 1.2 mg of polysorbate 80 (e.g. super refined polysorbate 80); and
(g) optionally water.
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60.

72. A pharmaceutical unit dosage form according to any of aspects 69-71, wherein the anti-OX40 ligand (OX40L) antagonist antibody or antigen binding fragment thereof is amlitelimab or a variant thereof.

73. A pharmaceutical unit dosage form according to any of aspects 69-71, wherein the unit dosage form is contained in a suitable container.

74. A pharmaceutical unit dosage form according to aspect 73, wherein the suitable container is a vial, a syringe (e.g. a pre-filled syringe), a microinfusor, a pen delivery device (e.g. a pre-filled pen delivery device), or an autoinjector.

75. A pharmaceutical unit dosage form according to aspect 73 or 74, wherein the container is sealed.

The disclosure includes the following numbered clauses:

1. An aqueous pharmaceutical formulation comprising:
(a) an antibody that is capable of inhibiting and/or neutralizing the biological signalling activity of OX40, or an antigen binding fragment thereof;
(b) a stabilizer;
(c) a chelator;
(d) a surfactant; and
(e) a buffer comprising histidine or a histidine salt;
wherein the pH of the aqueous pharmaceutical formulation ranges from 5.5 to about 6.5, and
wherein the aqueous pharmaceutical formulation is suitable for parenteral administration to a mammalian subject.

2. The aqueous pharmaceutical formulation of clause 1, wherein the antibody or antigen binding fragment thereof is amlitelimab or a variant thereof.

3. The aqueous pharmaceutical formulation of clause 1 or clause 2, wherein the antibody or antigen binding fragment thereof is present in a concentration ranging from 28 to 138 mg/mL, or from 31 to 125 mg/mL.

4. The aqueous pharmaceutical formulation of any one of clauses 1-3, wherein the antibody or antigen binding fragment thereof is present in a concentration of 28 mg/mL, 31 mg/mL, 62.5 mg/mL, 125 mg/mL, or 138 mg/mL.

5. The aqueous pharmaceutical formulation according to any one of clauses 1-4, wherein the antibody or antigen binding fragment thereof is present at a concentration of about 62.5 mg/mL, preferably 62.5 mg/mL.

6. The aqueous pharmaceutical formulation according to any one of clauses 1-4, wherein the antibody or antigen binding fragment thereof is present at a concentration of about 125 mg/mL, preferably 125 mg/mL.

7. The aqueous pharmaceutical formulation of any one of clauses 1-6, wherein the stabilizer is sucrose.

8. The aqueous pharmaceutical formulation of any one of clauses 1-7, wherein the at least one stabilizer is sucrose, which is present in an amount of 220 mM.

9. The aqueous pharmaceutical formulation of any one of clauses 1-8, wherein the surfactant is polysorbate 80.

10. The aqueous pharmaceutical formulation of any one of clauses 1-9, comprising 0.02% (w/v) to 0.1% (w/v) polysorbate 80.

11. The aqueous pharmaceutical formulation of any one of clauses 1-10, comprising 0.02% (w/v) to 0.06% polysorbate 80.

12. The aqueous pharmaceutical formulation of any one of clauses 1-11, comprising 0.04% (w/v) or 0.06% polysorbate 80.

13. The aqueous pharmaceutical formulation of any one of clauses 1-12, wherein the buffer comprises 10 mM L-histidine or histidine hydrochloride.

14. The aqueous pharmaceutical formulation of any one of clauses 1-13, further comprising a chelator selected from the group consisting of ethylenediaminetetraacetic acid (EDTA), diethylenetriamene pentaacetate (DTPA) and salts thereof, and any combinations thereof.

15. The aqueous pharmaceutical formulation of clause 14, wherein the chelator comprises one or both of 10 μM EDTA and 10 μM DTPA.

16. The aqueous pharmaceutical formulation of clause 14, wherein the chelator comprises 10 μM EDTA.

17. The aqueous pharmaceutical formulation of any of clauses 1 or 2, comprising:
28 mg/mL to 138 mg/mL of the antibody or antigen binding fragment thereof;
10 mM±1.5 mM L-histidine or histidine hydrochloride;
220 mM±33 mM sucrose,
0.06% (w/v) polysorbate 80; and
water;
with the pH of the aqueous pharmaceutical formulation between about 5.8 and about 6.2.

18. The aqueous pharmaceutical formulation of clause 17, wherein the antibody or antigen binding fragment thereof is present at a concentration of about 62.5 mg/mL, preferably 62.5 mg/mL.

19. The aqueous pharmaceutical formulation of clause 17, wherein the antibody or antigen binding fragment thereof is present at a concentration of about 125 mg/mL, preferably 125 mg/mL.

20. The aqueous pharmaceutical formulation of any of clauses 17-19, further comprising 10 μM EDTA or 10 μM DTPA.

21. The aqueous pharmaceutical formulation of any of clauses 17-19, wherein the aqueous pharmaceutical formulation is free or essentially free of a chelator.

22. The aqueous pharmaceutical formulation of any one of clauses 1-21 which is free or essentially free of particles.

23. The aqueous pharmaceutical formulation of any one of clauses 1-22, which is free or substantially free of sodium chloride.

24. The aqueous pharmaceutical formulation of any one of clauses 1-23, which is free or substantially free of arginine.

25. The aqueous pharmaceutical formulation of any one of clauses 1-24, wherein less than 5% of the antibody is detected in an aggregated form after 28 days of storage at 40° C., detected by size exclusion high performance liquid chromatography.

26. The aqueous pharmaceutical formulation of any one of clauses 1-25, which is stable upon storage at 5° C. or 25° C. for at least 3 months.

27. The aqueous pharmaceutical formulation of any one of clauses 1-26, which is stable upon storage at 40° C. for at least 2 months.

28. The aqueous pharmaceutical formulation of any one of clauses 1-27, which is stable for at least 1 year when stored at a temperature of about 5° C.

29. The aqueous pharmaceutical formulation of any one of clauses 1-28, which is comprised in a container.

30. The aqueous pharmaceutical formulation of any of clauses 1-29, which is suitable for subcutaneous delivery.

31. A pharmaceutical unit dosage form suitable for parenteral administration to a mammalian subject which comprises the aqueous pharmaceutical formulation of any one of clauses 1 to 30 in a suitable container.

32. The pharmaceutical unit dosage form of clause 31, wherein the aqueous pharmaceutical formulation is suitable for intravenous, subcutaneous, or intramuscular administration.

33. The pharmaceutical unit dosage form of clause 31 or 32, wherein the suitable container is a pre-filled syringe.

34. A sealed container comprising the aqueous pharmaceutical formulation of any one of clauses 1-30.

35. The sealed container of clause 34, which is a vial, a syringe, a microinfusor, a pen delivery device, or an autoinjector.

36. The sealed container of clause 34 or clause 35, which is a single or multi-chambered syringe.

37. The sealed container of clause 36, which is a pre-filled syringe containing 2.25 mL of the aqueous pharmaceutical formulation.

38. A pre-filled syringe comprising an aqueous pharmaceutical formulation comprising:
about 28 mg/mL to about 138 mg/mL of an antibody that is capable of inhibiting and/or neutralizing the biological signalling activity of OX40, or an antigen binding fragment thereof;
about 10 mM L-histidine or histidine hydrochloride;
about 220 mM sucrose;
about 0.06% (w/v) polysorbate 80; and
water;
wherein the pH of the aqueous pharmaceutical formulation is about 6.0.

39. A pre-filled pen or autoinjector comprising an aqueous pharmaceutical formulation comprising:
about 28 mg/mL to about 138 mg/mL of an antibody that is capable of inhibiting and/or neutralizing the biological signalling activity of OX40, or an antigen binding fragment thereof;
about 10 mM L-histidine or histidine hydrochloride;
about 220 mM sucrose;
about 0.06% (w/v) polysorbate 80; and
water;
wherein the pH of the aqueous pharmaceutical formulation is about 6.0.

40. The pre-filled syringe of clause 38 or the pre-filled pen or autoinjector of clause 39, further comprising 10 μM EDTA or 10 μM DTPA.

41. The pre-filled syringe of clause 38 or the pre-filled pen or autoinjector of clause 39, further comprising 10 μM EDTA.

42. The pre-filled syringe of any of clauses 38, 40 or 41 or the pre-filled pen or autoinjector of any of clauses 39-41, comprising about 31 mg/mL to about 125 mg/mL amlitelimab or a variant thereof.

43. The pre-filled syringe of any of clauses 38, 40-42 or the pre-filled pen or autoinjector of any of clauses 39-42, comprising 62.5 mg/mL amlitelimab or a variant thereof.

44. The pre-filled syringe of any of clauses 38, 40-42 or the pre-filled pen or autoinjector of any of clauses 39-42, comprising 125 mg/mL amlitelimab or a variant thereof.

45. A kit comprising a sealed container of any one of clauses 34-37, a pre-filled syringe of any one of clauses 38 or 40-44 or a pre-filled pen or autoinjector of any one of clauses 39-44.

46. A kit comprising:
a sealed container comprising the aqueous pharmaceutical formulation of any one of clauses 1-30; and
at least one separate injection device for delivery the aqueous pharmaceutical formulation to a mammalian subject in need thereof.

47. The kit of clause 46, wherein the injection device is a syringe, a microinfusor, a pen delivery device, or an autoinjector.

48. The kit of clause 47, wherein the injection device is a single or multi-chambered syringes.

49. The aqueous pharmaceutical formulation of any one of clauses 1-30, the pharmaceutical unit dosage form of any one of clauses 31-33, the sealed container of any one of clauses 34-37, the pre-filled syringe of any one of clauses 38 or 40-44, the pre-filled pen or autoinjector of any one of clauses 39-44, or the kit of any one of clauses 45-48 for use in treating a disease or condition in a subject selected from the group consisting of an autoimmune disease or condition, an inflammatory disease or condition, a systemic inflammatory disease or condition, and a transplant rejection disease or condition mediated by hOX40L.

50. The aqueous pharmaceutical formulation of any one of clauses 1-30, the pharmaceutical unit dosage form of any one of clauses 31-33, the sealed container of any one of clauses 34-37, the pre-filled syringe of any one of clauses 38 or 40-44, the pre-filled pen or autoinjector of any one of clauses 39-44, or the kit of any one of clauses 45-48 for use in treating atopic dermatitis.

51. The aqueous pharmaceutical formulation of any one of clauses 1-30, the pharmaceutical unit dosage form of any one of clauses 31-33, the sealed container of any one of clauses 34-37, the pre-filled syringe of any one of clauses 38 or 40-44, the pre-filled pen or autoinjector of any one of clauses 39-44, or the kit of any one of clauses 45-48 for use in treating asthma.

52. Use of the aqueous pharmaceutical formulation of any one of clauses 1-30, the pharmaceutical unit dosage form of any one of clauses 31-33, the sealed container of any one of clauses 34-37, the pre-filled syringe of any one of clauses 38 or 40-44, the pre-filled pen or autoinjector of any one of clauses 39-44, or the kit of any one of clauses 45-48 in the manufacture of a medicament for treating a disease or condition in a subject selected from the group consisting of an autoimmune disease or condition, an inflammatory disease or condition, a systemic inflammatory disease or condition, and a transplant rejection disease or condition mediated by hOX40L.

53. Use of the aqueous pharmaceutical formulation of any one of clauses 1-30, the pharmaceutical unit dosage form of any one of clauses 31-33, the sealed container of any one of clauses 34-37, the pre-filled syringe of any one of clauses 38 or 40-44, the pre-filled pen or autoinjector of any one of clauses 39-44, or the kit of any one of clauses 45-48 in the manufacture of a medicament for treating atopic dermatitis.

54. Use of the aqueous pharmaceutical formulation of any one of clauses 1-30, the pharmaceutical unit dosage form of any one of clauses 31-33, the sealed container of any one of clauses 34-37, the pre-filled syringe of any one of clauses 38 or 40-44, the pre-filled pen or autoinjector of any one of clauses 39-44, or the kit of any one of clauses 45-48 in the manufacture of a medicament for treating asthma.

55. A method of treating a disease or condition in a subject selected from the group consisting of an autoimmune disease or condition, an inflammatory disease or condition, a systemic inflammatory disease or condition, and a transplant rejection disease or condition (e.g. an autoimmune disease or condition, an inflammatory disease or condition, a systemic inflammatory disease or condition, and a transplant rejection disease or condition mediated by hOX40L), comprising administering to the subject an effective amount of the aqueous pharmaceutical formulation of any one of clauses 1 to 30.

56. A method of treating atopic dermatitis comprising administering to the subject an effective amount of the aqueous pharmaceutical formulation of any one of clauses 1 to 30.

57. A method of treating asthma comprising administering to the subject an effective amount of the aqueous pharmaceutical formulation of any one of clauses 1 to 30.

58. An aqueous pharmaceutical formulation comprising:
(a) antibody that is capable of inhibiting and/or neutralizing the biological signalling activity of OX40, or an antigen binding fragment thereof;
(b) a stabilizer;
(c) a chelator;
(d) a surfactant; and
(e) a buffer comprising histidine or a histidine salt;
wherein the pH of the aqueous pharmaceutical formulation ranges from 5.5 to about 6.5, and
wherein the aqueous pharmaceutical formulation is suitable for parenteral administration to a mammalian subject.

59. A pharmaceutical unit dosage form suitable for parenteral administration to a mammalian subject, comprising
(a) about 62.5 mg of an antibody that is capable of inhibiting and/or neutralizing the biological signalling activity of OX40, or an antigen binding fragment thereof;
(b) about 1.49 mg of L-Histidine;
(c) about 2.18 mg of L-Histidine hydrochloride (e.g L-histidine hydrochloride monohydrate);
(d) about 150.62 mg sucrose;
(e) about 0.0074 mg EDTA (e.g. EDTA disodium salt dihydrate);
(f) about 1.2 mg of polysorbate 80 (e.g. super refined polysorbate 80); and
(g) optionally water.

60. A pharmaceutical unit dosage form suitable for parenteral administration to a mammalian subject, comprising
(a) about 125 mg of an antibody that is capable of inhibiting and/or neutralizing the biological signalling activity of OX40, or an antigen binding fragment thereof;
(b) about 1.49 mg of L-Histidine;
(c) about 2.18 mg of L-Histidine hydrochloride (e.g. L-Histidine hydrochloride monohydrate);
(d) about 150.62 mg sucrose;
(e) about 0.0074 mg EDTA (e.g. EDTA disodium salt dihydrate);
(f) about 1.2 mg of super refined polysorbate 80; and
(g) optionally water.

61. A pharmaceutical unit dosage form suitable for parenteral administration to a mammalian subject, comprising
(a) about 250 mg of an antibody that is capable of inhibiting and/or neutralizing the biological signalling activity of OX40, or an antigen binding fragment thereof;
(b) about 1.49 mg of L-Histidine;
(c) about 2.18 mg of L-Histidine hydrochloride (e.g. L-Histidine hydrochloride monohydrate);
(d) about 150.62 mg sucrose;
(e) about 0.0074 mg EDTA (e.g. EDTA disodium salt dihydrate);
(f) about 1.2 mg of polysorbate 80 (e.g. super refined polysorbate 80); and
(g) optionally water.

62. A pharmaceutical unit dosage form according to any of clauses 59-61, wherein the antibody or antigen binding fragment thereof is amlitelimab or a variant thereof.

63. A pharmaceutical unit dosage form according to any of clauses 59-62, wherein the unit dosage form is contained in a suitable container.

64. A pharmaceutical unit dosage form according to clause 63, wherein the suitable container is a vial, a syringe (e.g. a pre-filled syringe), a microinfusor, a pen delivery device (e.g. a pre-filled pen delivery device), or an autoinjector.

69. A pharmaceutical unit dosage form according to clause 63 or 64, wherein the container is sealed.

The disclosure includes the following numbered elements:

1. An aqueous pharmaceutical formulation comprising:
(a) an antibody that is capable of blocking binding or substantially reducing binding of OX40 to OX40L, or an antigen binding fragment thereof;
(b) a stabilizer;
(c) a chelator;
(d) a surfactant; and
(e) a buffer comprising histidine or a histidine salt;
wherein the pH of the aqueous pharmaceutical formulation ranges from 5.5 to about 6.5, and
wherein the aqueous pharmaceutical formulation is suitable for parenteral administration to a mammalian subject.

2. The aqueous pharmaceutical formulation of element 1, wherein the antibody or antigen binding fragment thereof is amlitelimab or a variant thereof.

3. The aqueous pharmaceutical formulation of element 1 or element 2, wherein the antibody or antigen binding fragment thereof is present in a concentration ranging from 28 to 138 mg/mL, or from 31 to 125 mg/mL.

4. The aqueous pharmaceutical formulation of any one of elements 1-3, wherein the antibody or antigen binding fragment thereof is present in a concentration of 28 mg/mL, 31 mg/mL, 62.5 mg/mL, 125 mg/mL, or 138 mg/mL.

5. The aqueous pharmaceutical formulation according to any one of elements 1-4, wherein the antibody or antigen binding fragment thereof is present at a concentration of about 62.5 mg/mL, preferably 62.5 mg/mL.

6. The aqueous pharmaceutical formulation according to any one of elements 1-4, wherein the antibody or antigen binding fragment thereof is present at a concentration of about 125 mg/mL, preferably 125 mg/mL.

7. The aqueous pharmaceutical formulation of any one of elements 1-6, wherein the stabilizer is sucrose.

8. The aqueous pharmaceutical formulation of any one of elements 1-7, wherein the at least one stabilizer is sucrose, which is present in an amount of 220 mM.

9. The aqueous pharmaceutical formulation of any one of elements 1-8, wherein the surfactant is polysorbate 80.

10. The aqueous pharmaceutical formulation of any one of elements 1-9, comprising 0.02% (w/v) to 0.1% (w/v) polysorbate 80.

11. The aqueous pharmaceutical formulation of any one of elements 1-10, comprising 0.02% (w/v) to 0.06% polysorbate 80.

12. The aqueous pharmaceutical formulation of any one of elements 1-11, comprising 0.04% (w/v) or 0.06% polysorbate 80.

13. The aqueous pharmaceutical formulation of any one of elements 1-12, wherein the buffer comprises 10 mM L-histidine or histidine hydrochloride.

14. The aqueous pharmaceutical formulation of any one of elements 1-13, further comprising a chelator selected from the group consisting of ethylenediaminetetraacetic acid (EDTA), diethylenetriamene pentaacetate (DTPA) and salts thereof, and any combinations thereof.

15. The aqueous pharmaceutical formulation of element 14, wherein the chelator comprises one or both of 10 μM EDTA and 10 μM DTPA.

16. The aqueous pharmaceutical formulation of element 14, wherein the chelator comprises 10 μM EDTA.

17. The aqueous pharmaceutical formulation of any of elements 1 or 2, comprising:
28 mg/mL to 138 mg/mL of the antibody or antigen binding fragment thereof;
10 mM±1.5 mM L-histidine or histidine hydrochloride;
220 mM±33 mM sucrose,
0.06% (w/v) polysorbate 80; and
water;
with the pH of the aqueous pharmaceutical formulation between about 5.8 and about 6.2.

18. The aqueous pharmaceutical formulation of element 17, wherein the antibody or antigen binding fragment thereof is present at a concentration of about 62.5 mg/mL, preferably 62.5 mg/mL.

19. The aqueous pharmaceutical formulation of element 17, wherein the antibody or antigen binding fragment thereof is present at a concentration of about 125 mg/mL, preferably 125 mg/mL.

20. The aqueous pharmaceutical formulation of any of elements 17-19, further comprising 10 μM EDTA or 10 μM DTPA.

21. The aqueous pharmaceutical formulation of any of elements 17-19, wherein the aqueous pharmaceutical formulation is free or essentially free of a chelator.

22. The aqueous pharmaceutical formulation of any one of elements 1-21 which is free or essentially free of particles.

23. The aqueous pharmaceutical formulation of any one of elements 1-22, which is free or substantially free of sodium chloride.

24. The aqueous pharmaceutical formulation of any one of elements 1-23, which is free or substantially free of arginine.

25. The aqueous pharmaceutical formulation of any one of elements 1-24, wherein less than 5% of the antibody is detected in an aggregated form after 28 days of storage at 40° C., detected by size exclusion high performance liquid chromatography.

26. The aqueous pharmaceutical formulation of any one of elements 1-25, which is stable upon storage at 5° C. or 25° C. for at least 3 months.

27. The aqueous pharmaceutical formulation of any one of elements 1-26, which is stable upon storage at 40° C. for at least 2 months.

28. The aqueous pharmaceutical formulation of any one of elements 1-27, which is stable for at least 1 year when stored at a temperature of about 5° C.

29. The aqueous pharmaceutical formulation of any one of elements 1-28, which is comprised in a container.

30. The aqueous pharmaceutical formulation of any of elements 1-29, which is suitable for subcutaneous delivery.

31. A pharmaceutical unit dosage form suitable for parenteral administration to a mammalian subject which comprises the aqueous pharmaceutical formulation of any one of elements 1 to 30 in a suitable container.

32. The pharmaceutical unit dosage form of element 31, wherein the aqueous pharmaceutical formulation is suitable for intravenous, subcutaneous, or intramuscular administration.

33. The pharmaceutical unit dosage form of element 31 or 32, wherein the suitable container is a pre-filled syringe.

34. A sealed container comprising the aqueous pharmaceutical formulation of any one of elements 1-30.

35. The sealed container of element 34, which is a vial, a syringe, a microinfusor, a pen delivery device, or an autoinjector.

36. The sealed container of element 34 or element 35, which is a single or multi-chambered syringe.

37. The sealed container of element 36, which is a pre-filled syringe containing 2.25 mL of the aqueous pharmaceutical formulation.

38. A pre-filled syringe comprising an aqueous pharmaceutical formulation comprising:
about 28 mg/mL to about 138 mg/mL of an antibody that is capable of blocking binding or substantially reducing binding of OX40 to OX40L, or an antigen binding fragment thereof;
about 10 mM L-histidine or histidine hydrochloride;
about 220 mM sucrose;
about 0.06% (w/v) polysorbate 80; and
water;
wherein the pH of the aqueous pharmaceutical formulation is about 6.0.

39. A pre-filled pen or autoinjector comprising an aqueous pharmaceutical formulation comprising:
about 28 mg/mL to about 138 mg/mL of an antibody that is capable of blocking binding or substantially reducing binding of OX40 to OX40L, or an antigen binding fragment thereof;
about 10 mM L-histidine or histidine hydrochloride;
about 220 mM sucrose;
about 0.06% (w/v) polysorbate 80; and water;
wherein the pH of the aqueous pharmaceutical formulation is about 6.0.

40. The pre-filled syringe of element 38 or the pre-filled pen or autoinjector of element 39, further comprising 10 μM EDTA or 10 μM DTPA.

41. The pre-filled syringe of element 38 or the pre-filled pen or autoinjector of element 39, further comprising 10 μM EDTA.

42. The pre-filled syringe of any of elements 38, 40 or 41 or the pre-filled pen or autoinjector of any of elements 39-41, comprising about 31 mg/mL to about 125 mg/mL amlitelimab or a variant thereof.

43. The pre-filled syringe of any of elements 38, 40-42 or the pre-filled pen or autoinjector of any of elements 39-42, comprising 62.5 mg/mL amlitelimab or a variant thereof.

44. The pre-filled syringe of any of elements 38, 40-42 or the pre-filled pen or autoinjector of any of elements 39-42, comprising 125 mg/mL amlitelimab or a variant thereof.

45. A kit comprising a sealed container of any one of elements 34-37, a pre-filled syringe of any one of elements 38 or 40-44 or a pre-filled pen or autoinjector of any one of elements 39-44.

46. A kit comprising: a sealed container comprising the aqueous pharmaceutical formulation of any one of elements 1-30; and at least one separate injection device for delivery the aqueous pharmaceutical formulation to a mammalian subject in need thereof.

47. The kit of element 46, wherein the injection device is a syringe, a microinfusor, a pen delivery device, or an autoinjector.

48. The kit of element 47, wherein the injection device is a single or multi-chambered syringes.

49. The aqueous pharmaceutical formulation of any one of elements 1-30, the pharmaceutical unit dosage form of any one of elements 31-33, the sealed container of any one of elements 34-37, the pre-filled syringe of any one of elements 38 or 40-44, the pre-filled pen or autoinjector of any one of elements 39-44, or the kit of any one of elements 45-48 for use in treating a disease or condition in a subject selected from the group consisting of an autoimmune disease or condition, an inflammatory disease or condition, a systemic inflammatory disease or condition, and a transplant rejection disease or condition mediated by hOX40L.

50. The aqueous pharmaceutical formulation of any one of elements 1-30, the pharmaceutical unit dosage form of any one of elements 31-33, the sealed container of any one of elements 34-37, the pre-filled syringe of any one of elements 38 or 40-44, the pre-filled pen or autoinjector of any one of elements 39-44, or the kit of any one of elements 45-48 for use in treating atopic dermatitis.

51. The aqueous pharmaceutical formulation of any one of elements 1-30, the pharmaceutical unit dosage form of any one of elements 31-33, the sealed container of any one of elements 34-37, the pre-filled syringe of any one of elements 38 or 40-44, the pre-filled pen or autoinjector of any one of elements 39-44, or the kit of any one of elements 45-48 for use in treating asthma.

52. Use of the aqueous pharmaceutical formulation of any one of elements 1-30, the pharmaceutical unit dosage form of any one of elements 31-33, the sealed container of any one of elements 34-37, the pre-filled syringe of any one of elements 38 or 40-44, the pre-filled pen or autoinjector of any one of elements 39-44, or the kit of any one of elements 45-48 in the manufacture of a medicament for treating a disease or condition in a subject selected from the group consisting of an autoimmune disease or condition, an inflammatory disease or condition, a systemic inflammatory disease or condition, and a transplant rejection disease or condition mediated by hOX40L.

53. Use of the aqueous pharmaceutical formulation of any one of elements 1-30, the pharmaceutical unit dosage form of any one of elements 31-33, the sealed container of any one of elements 34-37, the pre-filled syringe of any one of elements 38 or 40-44, the pre-filled pen or autoinjector of any one of elements 39-44, or the kit of any one of elements 45-48 in the manufacture of a medicament for treating atopic dermatitis.

54. Use of the aqueous pharmaceutical formulation of any one of elements 1-30, the pharmaceutical unit dosage form of any one of elements 31-33, the sealed container of any one of elements 34-37, the pre-filled syringe of any one of elements 38 or 40-44, the pre-filled pen or autoinjector of any one of elements 39-44, or the kit of any one of elements 45-48 in the manufacture of a medicament for treating asthma.

55. A method of treating a disease or condition in a subject selected from the group consisting of an autoimmune disease or condition, an inflammatory disease or condition, a systemic inflammatory disease or condition, and a transplant rejection disease or condition (e.g. an autoimmune disease or condition, an inflammatory disease or condition, a systemic inflammatory disease or condition, and a transplant rejection disease or condition mediated by hOX40L), comprising administering to the subject an effective amount of the aqueous pharmaceutical formulation of any one of elements 1 to 30.

56. A method of treating atopic dermatitis comprising administering to the subject an effective amount of the aqueous pharmaceutical formulation of any one of elements 1 to 30.

57. A method of treating asthma comprising administering to the subject an effective amount of the aqueous pharmaceutical formulation of any one of elements 1 to 30.

58. An aqueous pharmaceutical formulation comprising:
(a) antibody that is capable of blocking binding or substantially reducing binding of OX40 to OX40L, or an antigen binding fragment thereof;
(b) a stabilizer;
(c) a chelator;
(d) a surfactant; and
(e) a buffer comprising histidine or a histidine salt;
wherein the pH of the aqueous pharmaceutical formulation ranges from 5.5 to about 6.5, and
wherein the aqueous pharmaceutical formulation is suitable for parenteral administration to a mammalian subject.

59. A pharmaceutical unit dosage form suitable for parenteral administration to a mammalian subject, comprising
(a) about 62.5 mg of an antibody that is capable of blocking binding or substantially reducing binding of OX40 to OX40L, or an antigen binding fragment thereof;
(b) about 1.49 mg of L-Histidine;
(c) about 2.18 mg of L-Histidine hydrochloride (e.g L-histidine hydrochloride monohydrate);
(d) about 150.62 mg sucrose;
(e) about 0.0074 mg EDTA (e.g. EDTA disodium salt dihydrate);
(f) about 1.2 mg of polysorbate 80 (e.g. super refined polysorbate 80); and
(g) optionally water.

60. A pharmaceutical unit dosage form suitable for parenteral administration to a mammalian subject, comprising
(a) about 125 mg of an antibody that is capable of blocking binding or substantially reducing binding of OX40 to OX40L, or an antigen binding fragment thereof;
(b) about 1.49 mg of L-Histidine;
(c) about 2.18 mg of L-Histidine hydrochloride (e.g. L-Histidine hydrochloride monohydrate);
(d) about 150.62 mg sucrose;
(e) about 0.0074 mg EDTA (e.g. EDTA disodium salt dihydrate);
(f) about 1.2 mg of super refined polysorbate 80; and
(g) optionally water.

61. A pharmaceutical unit dosage form suitable for parenteral administration to a mammalian subject, comprising
(a) about 250 mg of an antibody that is capable of blocking binding or substantially reducing binding of OX40 to OX40L, or an antigen binding fragment thereof;
(b) about 1.49 mg of L-Histidine;
(c) about 2.18 mg of L-Histidine hydrochloride (e.g. L-Histidine hydrochloride monohydrate);
(d) about 150.62 mg sucrose;
(e) about 0.0074 mg EDTA (e.g. EDTA disodium salt dihydrate);
(f) about 1.2 mg of polysorbate 80 (e.g. super refined polysorbate 80); and
(g) optionally water.

62. A pharmaceutical unit dosage form according to any of elements 59-61, wherein the antibody or antigen binding fragment thereof is amlitelimab or a variant thereof.

63. A pharmaceutical unit dosage form according to any of elements 59-62, wherein the unit dosage form is contained in a suitable container.

64. A pharmaceutical unit dosage form according to element 63, wherein the suitable container is a vial, a syringe (e.g. a pre-filled syringe), a microinfusor, a pen delivery device (e.g. a pre-filled pen delivery device), or an autoinjector.

69. A pharmaceutical unit dosage form according to element 63 or 64, wherein the container is sealed.

The disclosure includes the following numbered paragraphs:

1. An aqueous pharmaceutical formulation comprising:
(a) an anti-OX40 ligand (OX40L) antagonist antibody or antigen binding fragment thereof;
(b) a stabilizer;
(c) a surfactant; and
(d) a buffer comprising histidine or a histidine salt;
wherein the pH of the aqueous pharmaceutical formulation ranges from 5.5 to about 6.5,
wherein the antibody or antigen binding fragment thereof comprises:
   a heavy chain complementarity determining region (HCDR) 1 of SEQ ID NO: 42, an HCDR2 of SEQ ID NO:44, and an HCDR3 of SEQ ID NO:46; and
   a light chain complementarity determining region (LCDR) 1 of SEQ ID NO:56, an LCDR2 of SEQ ID NO:58, and an LCDR3 of SEQ ID NO:60; and
wherein the aqueous pharmaceutical formulation is suitable for parenteral administration to a mammalian subject.

2. The aqueous pharmaceutical formulation of paragraph 1, wherein the antibody or antigen binding fragment thereof is amlitelimab or a variant thereof.

3. The aqueous pharmaceutical formulation of paragraph 1 or paragraph 2, wherein the antibody or antigen binding fragment thereof is present in a concentration ranging from 75 mg/mL to 125 mg/mL, from 125 mg/mL to 138 mg/mL, or from 112.5 mg/mL to 137.5 mg/mL.

4. The aqueous pharmaceutical formulation of any one of paragraphs 1-3, wherein the antibody or antigen binding fragment thereof is present in a concentration of 125 mg/mL to 138 mg/mL.

5. The aqueous pharmaceutical formulation of any one of paragraphs 1-4, comprising 125 mg/mL, 120.5 mg/mL, or 124.4 mg/mL of the antibody or antigen binding fragment thereof.

6. The aqueous pharmaceutical formulation of any one of paragraphs 1-5, wherein the stabilizer is sucrose.

7. The aqueous pharmaceutical formulation of any one of paragraphs 1-6, wherein the at least one stabilizer is sucrose, which is present in an amount of 220 mM.

8. The aqueous pharmaceutical formulation of any one of paragraphs 1-7, wherein the surfactant is polysorbate 80.

9. The aqueous pharmaceutical formulation of any one of paragraphs 1-8, comprising 0.01% (w/v) to 0.1% (w/v) polysorbate 80.

10. The aqueous pharmaceutical formulation of any one of paragraphs 1-9, comprising 0.02% (w/v) to 0.06% (w/v) polysorbate 80.

11. The aqueous pharmaceutical formulation of any one of paragraphs 1-10, comprising 0.04% (w/v) polysorbate 80.

12. The aqueous pharmaceutical formulation of any one of paragraphs 1-11, wherein the buffer comprises 20 mM L-histidine or histidine hydrochloride.

13. The aqueous pharmaceutical formulation of paragraph 1, comprising:
125 mg/mL to 138 mg/mL of the antibody or antigen binding fragment thereof;
20 mM L-histidine or histidine hydrochloride;
220 mM sucrose,
0.04% (w/v) polysorbate 80; and
water;
with the pH of the aqueous pharmaceutical formulation between about 5.8 and about 6.2.

14. The aqueous pharmaceutical formulation of any one of paragraphs 1-13, which is free or essentially free of particles.

15. The aqueous pharmaceutical formulation of any one of paragraphs 1-14, which is free or substantially free of sodium chloride.

16. The aqueous pharmaceutical formulation of any one of paragraphs 1-15, which is free or substantially free of arginine.

17. The aqueous pharmaceutical formulation of any one of paragraphs 1-16, wherein at least 91% of the antibody has native conformation after 28 days at 40° C., measured by size exclusion chromatography.

18. The aqueous pharmaceutical formulation of any one of paragraphs 1-17, wherein at least 94% of the antibody has native conformation after 8 weeks at 25° C., measured by size exclusion chromatography.

19. The aqueous pharmaceutical formulation of any one of paragraphs 1-18, wherein at least 98% of the antibody has native conformation after 16 weeks at 5° C. or at −65° C., measured by size exclusion chromatography.

20. The aqueous pharmaceutical formulation of any one of paragraphs 1-19, wherein at least 55% of the antibody is the main charge variant of the antibody after 28 days at 40° C., measured by imaged capillary isoelectric focusing (iCIEF).

21. The aqueous pharmaceutical formulation of any one of paragraphs 1-20, wherein at least 65% of the antibody is the main charge variant of the antibody after 16 weeks at 25° C., measured by iCIEF.

22. The aqueous pharmaceutical formulation of any one of paragraphs 1-21, wherein at least 70% of the antibody is the main charge variant of the antibody after 16 weeks at 5° C., measured by iCIEF.

23. The aqueous pharmaceutical formulation of any one of paragraphs 1-22, wherein at least 70% of the antibody is the main charge variant of the antibody after 16 weeks at −65° C., measured by iCIEF.

24. The aqueous pharmaceutical formulation of any one of paragraphs 1-23, which is stable upon storage at −65° C., 5° C., or 25° C. for at least 16 weeks.

25. The aqueous pharmaceutical formulation of any one of paragraphs 1-24, which is stable upon storage at 40° C. for at least 8 weeks.

26. The aqueous pharmaceutical formulation of any one of paragraphs 1-25, which is stable upon storage at 5° C. for at least 1 year.

27. The aqueous pharmaceutical formulation of any one of paragraphs 1-26, which is stable upon freezing and thawing.

28. The aqueous pharmaceutical formulation of any one of paragraphs 1-27, which is provided in a container.

29. A pharmaceutical unit dosage form suitable for parenteral administration to a mammalian subject which comprises the aqueous pharmaceutical formulation of any one of claims 1 to 27 in a suitable container.

30. The pharmaceutical unit dosage form of claim 29, wherein the aqueous pharmaceutical formulation is suitable for intravenous, subcutaneous, or intramuscular administration.

31. The pharmaceutical unit dosage form of claim 29 or 30, wherein the suitable container is a pre-filled syringe.

32. A sealed container comprising the aqueous pharmaceutical formulation of any one of paragraphs 1-27.

33. The sealed container of paragraph 32, which is a vial, a syringe, a microinfusor, a pen delivery device, or an autoinjector.

34. The sealed container of paragraph 32 or paragraph 33, which is a single or multi-chambered syringe.

35. The sealed container of paragraph 34, which is a pre-filled syringe containing 2.25 mL of the aqueous pharmaceutical formulation.

36. A pre-filled syringe comprising an aqueous pharmaceutical formulation comprising:
125 mg/mL to 138 mg/mL of amlitelimab or a variant thereof;
20 mM L-histidine or histidine hydrochloride;
220 mM sucrose,
0.04% (w/v) polysorbate 80, and
water;
wherein the pH of the aqueous pharmaceutical formulation is 6.0.

37. A kit comprising a sealed container of any one of paragraphs 32-35 or a pre-filled syringe of paragraph 36.

38. A kit comprising:
a sealed container comprising the aqueous pharmaceutical formulation of any one of paragraphs 1-27, and
at least one separate injection device for delivery the aqueous pharmaceutical formulation to a mammalian subject in need thereof.

39. The kit of paragraph 38, wherein the injection device is a syringe, a microinfusor, a pen delivery device, or an autoinjector.

40. The kit of paragraph 39, wherein the injection device is a single or multi-chambered syringes.

41. The aqueous pharmaceutical formulation of any one of paragraphs 1-28, the pharmaceutical unit dosage form of any one of paragraphs 29-31, the sealed container of any one of paragraphs 32-35, the pre-filled syringe of paragraph 36, or the kit of any one of paragraphs 38-40 for use in treating a disease or condition in a subject selected from the group consisting of an autoimmune disease or condition, an inflammatory disease or condition, and a transplant rejection disease or condition mediated by hOX40L.

42. A method of treating a disease or condition in a subject selected from the group consisting of an autoimmune disease or condition, an inflammatory disease or condition, and a transplant rejection disease or condition, comprising administering to the subject an effective amount of the aqueous pharmaceutical formulation of any one of paragraphs 1 to 27.

43. A method of treating atopic dermatitis comprising administering to the subject an effective amount of the aqueous pharmaceutical formulation of any one of paragraphs 1 to 27.

44. A method of treating asthma comprising administering to the subject an effective amount of the aqueous pharmaceutical formulation of any one of paragraphs 1 to 27.

45. An aqueous pharmaceutical formulation comprising:
(a) an anti-OX40 ligand (Ox40L) antagonist antibody or antigen binding fragment;
(b) a stabilizer;
(c) a surfactant; and
(d) a buffer comprising histidine or a histidine salt;
wherein the pH of the aqueous pharmaceutical formulation ranges from 5.5 to about 6.5,
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60; and
wherein the aqueous pharmaceutical formulation is suitable for parenteral administration to a mammalian subject.

46. The method of paragraph 45, wherein the VL domain comprises the amino acid sequence of SEQ ID NO:48 and the VH domain comprises the amino acid sequence of SEQ ID NO:34.

47. The method of paragraph 45 or 46, wherein the antibody comprises a heavy chain and a light chain, wherein the heavy chain amino acid sequence consists of the amino acid sequence of SEQ ID NO: 62 and the light chain amino acid sequence consists of the amino acid sequence of SEQ ID NO: 64

The disclosure includes the following numbered passages:

1. A pharmaceutical formulation comprising:
(a) an anti-OX40 ligand (OX40L) antagonist antibody or antigen binding fragment thereof;
(b) a stabilizer;
(c) a surfactant; and
(d) a buffer comprising histidine or a histidine salt;
wherein the pH of the pharmaceutical formulation ranges from 5.5 to about 6.5,
wherein the antibody or antigen binding fragment thereof comprises:
   a heavy chain complementarity determining region (HCDR) 1 of SEQ ID NO: 42, an HCDR2 of SEQ ID NO:44, and an HCDR3 of SEQ ID NO:46; and
   a light chain complementarity determining region (LCDR) 1 of SEQ ID NO:56, an LCDR2 of SEQ ID NO:58, and an LCDR3 of SEQ ID NO:60; and
wherein the pharmaceutical formulation is suitable for parenteral administration to a mammalian subject.

2. The pharmaceutical formulation of passage 1, wherein the antibody or antigen binding fragment thereof is amlitelimab or a variant thereof.

3. The pharmaceutical formulation of passage 1 or passage 2, wherein the antibody or antigen binding fragment thereof is present in a concentration ranging from 75 mg/mL to 125 mg/mL, from 125 mg/mL to 138 mg/mL, or from 112.5 mg/mL to 137.5 mg/mL.

4. The pharmaceutical formulation of any one of passages 1-3, wherein the antibody or antigen binding fragment thereof is present in a concentration of 125 mg/mL to 138 mg/mL.

5. The pharmaceutical formulation of any one of passages 1-4, comprising 125 mg/mL, 120.5 mg/mL, or 124.4 mg/mL of the antibody or antigen binding fragment thereof.

6. The pharmaceutical formulation of any one of passages 1-5, wherein the stabilizer is sucrose.

7. The pharmaceutical formulation of any one of passages 1-6, wherein the at least one stabilizer is sucrose, which is present in an amount of 220 mM.

8. The pharmaceutical formulation of any one of passages 1-7, wherein the surfactant is polysorbate 80.

9. The pharmaceutical formulation of any one of passages 1-8, comprising 0.01% (w/v) to 0.1% (w/v) polysorbate 80.

10. The pharmaceutical formulation of any one of passages 1-9, comprising 0.02% (w/v) to 0.06% (w/v) polysorbate 80.

11. The pharmaceutical formulation of any one of passages 1-10, comprising 0.04% (w/v) polysorbate 80.

12. The pharmaceutical formulation of any one of passages 1-11, wherein the buffer comprises 20 mM L-histidine or histidine hydrochloride.

13. The pharmaceutical formulation of passage 1, comprising:
125 mg/mL to 138 mg/mL of the antibody or antigen binding fragment thereof;
20 mM L-histidine or histidine hydrochloride;
220 mM sucrose,
0.04% (w/v) polysorbate 80; and
water;
with the pH of the pharmaceutical formulation between about 5.8 and about 6.2.

14. The pharmaceutical formulation of any one of passages 1-13, which is free or essentially free of particles.

15. The pharmaceutical formulation of any one of passages 1-14, which is free or substantially free of sodium chloride.

16. The pharmaceutical formulation of any one of passages 1-15, which is free or substantially free of arginine.

17. The pharmaceutical formulation of any one of passages 1-16, wherein at least 91% of the antibody has native conformation after 28 days at 40° C., measured by size exclusion chromatography.

18. The pharmaceutical formulation of any one of passages 1-17, wherein at least 94% of the antibody has native conformation after 8 weeks at 25° C., measured by size exclusion chromatography.

19. The pharmaceutical formulation of any one of passages 1-18, wherein at least 98% of the antibody has native conformation after 16 weeks at 5° C. or at −65° C., measured by size exclusion chromatography.

20. The pharmaceutical formulation of any one of passages 1-19, wherein at least 55% of the antibody is the main charge variant of the antibody after 28 days at 40° C., measured by imaged capillary isoelectric focusing (iCIEF).

21. The pharmaceutical formulation of any one of passages 1-20, wherein at least 65% of the antibody is the main charge variant of the antibody after 16 weeks at 25° C., measured by iCIEF.

22. The pharmaceutical formulation of any one of passages 1-21, wherein at least 70% of the antibody is the main charge variant of the antibody after 16 weeks at 5° C., measured by iCIEF.

23. The pharmaceutical formulation of any one of passages 1-22, wherein at least 70% of the antibody is the main charge variant of the antibody after 16 weeks at −65° C., measured by iCIEF.

24. The pharmaceutical formulation of any one of passages 1-23, which is stable upon storage at −65° C., 5° C., or 25° C. for at least 16 weeks.

25. The pharmaceutical formulation of any one of passages 1-24, which is stable upon storage at 40° C. for at least 8 weeks.

26. The pharmaceutical formulation of any one of passages 1-25, which is stable upon storage at 5° C. for at least 1 year.

27. The pharmaceutical formulation of any one of passages 1-26, which is stable upon freezing and thawing.

28. The pharmaceutical formulation of any one of passages 1-27, which is provided in a container.

29. A pharmaceutical unit dosage form suitable for parenteral administration to a mammalian subject which comprises the pharmaceutical formulation of any one of claims 1 to 27 in a suitable container.

30. The pharmaceutical unit dosage form of claim 29, wherein the pharmaceutical formulation is suitable for intravenous, subcutaneous, or intramuscular administration.

31. The pharmaceutical unit dosage form of claim 29 or 30, wherein the suitable container is a pre-filled syringe.

32. A sealed container comprising the pharmaceutical formulation of any one of passages 1-27.

33. The sealed container of passage 32, which is a vial, a syringe, a microinfusor, a pen delivery device, or an autoinjector.

34. The sealed container of passage 32 or passage 33, which is a single or multi-chambered syringe.

35. The sealed container of passage 34, which is a pre-filled syringe containing 2.25 mL of the pharmaceutical formulation.

36. A pre-filled syringe comprising a pharmaceutical formulation comprising:
125 mg/mL to 138 mg/mL of amlitelimab or a variant thereof;
20 mM L-histidine or histidine hydrochloride;
220 mM sucrose,
0.04% (w/v) polysorbate 80, and
water;
wherein the pH of the pharmaceutical formulation is 6.0.

37. A kit comprising a sealed container of any one of passages 32-35 or a pre-filled syringe of passage 36.

38. A kit comprising:
a sealed container comprising the pharmaceutical formulation of any one of passages 1-27, and
at least one separate injection device for delivery the pharmaceutical formulation to a mammalian subject in need thereof.

39. The kit of passage 38, wherein the injection device is a syringe, a microinfusor, a pen delivery device, or an autoinjector.

40. The kit of passage 39, wherein the injection device is a single or multi-chambered syringes.

41. The pharmaceutical formulation of any one of passages 1-28, the pharmaceutical unit dosage form of any one of passages 29-31, the sealed container of any one of passages 32-35, the pre-filled syringe of passage 36, or the kit of any one of passages 38-40 for use in treating a disease or condition in a subject selected from the group consisting of an autoimmune disease or condition, an inflammatory disease or condition, and a transplant rejection disease or condition mediated by hOX40L.

42. A method of treating a disease or condition in a subject selected from the group consisting of an autoimmune disease or condition, an inflammatory disease or condition, and a transplant rejection disease or condition, comprising administering to the subject an effective amount of the pharmaceutical formulation of any one of passages 1 to 27.

43. A method of treating atopic dermatitis comprising administering to the subject an effective amount of the pharmaceutical formulation of any one of passages 1 to 27.

44. A method of treating asthma comprising administering to the subject an effective amount of the pharmaceutical formulation of any one of passages 1 to 27.

45. A pharmaceutical formulation comprising:
(a) an anti-OX40 ligand (Ox40L) antagonist antibody or antigen binding fragment;
(b) a stabilizer;
(c) a surfactant; and
(d) a buffer comprising histidine or a histidine salt;
wherein the pH of the pharmaceutical formulation ranges from 5.5 to about 6.5,
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60; and
wherein the pharmaceutical formulation is suitable for parenteral administration to a mammalian subject.

46. The method of passage 45, wherein the VL domain comprises the amino acid sequence of SEQ ID NO:48 and the VH domain comprises the amino acid sequence of SEQ ID NO:34.

47. The method of passage 45 or 46, wherein the antibody comprises a heavy chain and a light chain, wherein the heavy chain amino acid sequence consists of the amino acid sequence of SEQ ID NO: 62 and the light chain amino acid sequence consists of the amino acid sequence of SEQ ID NO: 64

The disclosure includes the following numbered items:

1. An aqueous pharmaceutical formulation comprising:
(a) an antibody that is capable of inhibiting and/or neutralizing the biological signalling activity of OX40 or an antigen binding fragment thereof;
(b) a stabilizer;
(c) a surfactant; and
(d) a buffer comprising histidine or a histidine salt;
wherein the pH of the aqueous pharmaceutical formulation ranges from 5.5 to about 6.5, and
wherein the aqueous pharmaceutical formulation is suitable for parenteral administration to a mammalian subject.

2. The aqueous pharmaceutical formulation of item 1, wherein the antibody or antigen binding fragment thereof is amlitelimab or a variant thereof.

3. The aqueous pharmaceutical formulation of item 1 or item 2, wherein the antibody or antigen binding fragment thereof is present in a concentration ranging from 75 mg/mL to 125 mg/mL, from 125 mg/mL to 138 mg/mL, or from 112.5 mg/mL to 137.5 mg/mL.

4. The aqueous pharmaceutical formulation of any one of items 1-3, wherein the antibody or antigen binding fragment thereof is present in a concentration of 125 mg/mL to 138 mg/mL.

5. The aqueous pharmaceutical formulation of any one of items 1-4, comprising 125 mg/mL, 120.5 mg/mL, or 124.4 mg/mL of the antibody or antigen binding fragment thereof.

6. The aqueous pharmaceutical formulation of any one of items 1-5, wherein the stabilizer is sucrose.

7. The aqueous pharmaceutical formulation of any one of items 1-6, wherein the at least one stabilizer is sucrose, which is present in an amount of 220 mM.

8. The aqueous pharmaceutical formulation of any one of items 1-7, wherein the surfactant is polysorbate 80.

9. The aqueous pharmaceutical formulation of any one of items 1-8, comprising 0.01% (w/v) to 0.1% (w/v) polysorbate 80.

10. The aqueous pharmaceutical formulation of any one of items 1-9, comprising 0.02% (w/v) to 0.06% (w/v) polysorbate 80.

11. The aqueous pharmaceutical formulation of any one of items 1-10, comprising 0.04% (w/v) polysorbate 80.

12. The aqueous pharmaceutical formulation of any one of items 1-11, wherein the buffer comprises 20 mM L-histidine or histidine hydrochloride.

13. The aqueous pharmaceutical formulation of item 1, comprising:
125 mg/mL to 138 mg/mL of the antibody or antigen binding fragment thereof;
20 mM L-histidine or histidine hydrochloride;
220 mM sucrose,
0.04% (w/v) polysorbate 80; and
water;
with the pH of the aqueous pharmaceutical formulation between about 5.8 and about 6.2.

14. The aqueous pharmaceutical formulation of any one of items 1-13, which is free or essentially free of particles.

15. The aqueous pharmaceutical formulation of any one of items 1-14, which is free or substantially free of sodium chloride.

16. The aqueous pharmaceutical formulation of any one of items 1-15, which is free or substantially free of arginine.

17. The aqueous pharmaceutical formulation of any one of items 1-16, wherein at least 91% of the antibody has native conformation after 28 days at 40° C., measured by size exclusion chromatography.

18. The aqueous pharmaceutical formulation of any one of items 1-17, wherein at least 94% of the antibody has native conformation after 8 weeks at 25° C., measured by size exclusion chromatography.

19. The aqueous pharmaceutical formulation of any one of items 1-18, wherein at least 98% of the antibody has native conformation after 16 weeks at 5° C. or at −65° C., measured by size exclusion chromatography.

20. The aqueous pharmaceutical formulation of any one of items 1-19, wherein at least 55% of the antibody is the main charge variant of the antibody after 28 days at 40° C., measured by imaged capillary isoelectric focusing (iCIEF).

21. The aqueous pharmaceutical formulation of any one of items 1-20, wherein at least 65% of the antibody is the main charge variant of the antibody after 16 weeks at 25° C., measured by iCIEF.

22. The aqueous pharmaceutical formulation of any one of items 1-21, wherein at least 70% of the antibody is the main charge variant of the antibody after 16 weeks at 5° C., measured by iCIEF.

23. The aqueous pharmaceutical formulation of any one of items 1-22, wherein at least 70% of the antibody is the main charge variant of the antibody after 16 weeks at −65° C., measured by iCIEF.

24. The aqueous pharmaceutical formulation of any one of items 1-23, which is stable upon storage at −65° C., 5° C., or 25° C. for at least 16 weeks.

25. The aqueous pharmaceutical formulation of any one of items 1-24, which is stable upon storage at 40° C. for at least 8 weeks.

26. The aqueous pharmaceutical formulation of any one of items 1-25, which is stable upon storage at 5° C. for at least 1 year.

27. The aqueous pharmaceutical formulation of any one of items 1-26, which is stable upon freezing and thawing.

28. The aqueous pharmaceutical formulation of any one of items 1-27, which is provided in a container.

29. A pharmaceutical unit dosage form suitable for parenteral administration to a mammalian subject which comprises the aqueous pharmaceutical formulation of any one of claims 1 to 27 in a suitable container.

30. The pharmaceutical unit dosage form of claim 29, wherein the aqueous pharmaceutical formulation is suitable for intravenous, subcutaneous, or intramuscular administration.

31. The pharmaceutical unit dosage form of claim 29 or 30, wherein the suitable container is a pre-filled syringe.

32. A sealed container comprising the aqueous pharmaceutical formulation of any one of items 1-27.

33. The sealed container of item 32, which is a vial, a syringe, a microinfusor, a pen delivery device, or an autoinjector.

34. The sealed container of item 32 or item 33, which is a single or multi-chambered syringe.

35. The sealed container of item 34, which is a pre-filled syringe containing 2.25 mL of the aqueous pharmaceutical formulation.

36. A pre-filled syringe comprising an aqueous pharmaceutical formulation comprising:
125 mg/mL to 138 mg/mL of amlitelimab or a variant thereof;
20 mM L-histidine or histidine hydrochloride;
220 mM sucrose,
0.04% (w/v) polysorbate 80, and
water;
wherein the pH of the aqueous pharmaceutical formulation is 6.0.

37. A kit comprising a sealed container of any one of items 32-35 or a pre-filled syringe of item 36.

38. A kit comprising:
a sealed container comprising the aqueous pharmaceutical formulation of any one of items 1-27, and
at least one separate injection device for delivery the aqueous pharmaceutical formulation to a mammalian subject in need thereof.

39. The kit of item 38, wherein the injection device is a syringe, a microinfusor, a pen delivery device, or an autoinjector.

40. The kit of item 39, wherein the injection device is a single or multi-chambered syringes.

41. The aqueous pharmaceutical formulation of any one of items 1-28, the pharmaceutical unit dosage form of any one of items 29-31, the sealed container of any one of items 32-35, the pre-filled syringe of item 36, or the kit of any one of items 38-40 for use in treating a disease or condition in a subject selected from the group consisting of an autoimmune disease or condition, an inflammatory disease or condition, and a transplant rejection disease or condition mediated by hOX40L.

42. A method of treating a disease or condition in a subject selected from the group consisting of an autoimmune disease or condition, an inflammatory disease or condition, and a transplant rejection disease or condition, comprising administering to the subject an effective amount of the aqueous pharmaceutical formulation of any one of items 1 to 27.

43. A method of treating atopic dermatitis comprising administering to the subject an effective amount of the aqueous pharmaceutical formulation of any one of items 1 to 27.

44. A method of treating asthma comprising administering to the subject an effective amount of the aqueous pharmaceutical formulation of any one of items 1 to 27.

45. An aqueous pharmaceutical formulation comprising:
(a) an antibody that is capable of inhibiting and/or neutralizing the biological signalling activity of OX40 or an antigen binding fragment thereof;
(b) a stabilizer;
(c) a surfactant; and
(d) a buffer comprising histidine or a histidine salt;
wherein the pH of the aqueous pharmaceutical formulation ranges from 5.5 to about 6.5, and
wherein the aqueous pharmaceutical formulation is suitable for parenteral administration to a mammalian subject.

The disclosure includes the following numbered features:

1. An aqueous pharmaceutical formulation comprising:
(a) an antibody that is capable of blocking binding or substantially reducing binding of OX40 to OX40L, or an antigen binding fragment thereof;
(b) a stabilizer;
(c) a surfactant; and
(d) a buffer comprising histidine or a histidine salt;
wherein the pH of the aqueous pharmaceutical formulation ranges from 5.5 to about 6.5, and
wherein the aqueous pharmaceutical formulation is suitable for parenteral administration to a mammalian subject.

2. The aqueous pharmaceutical formulation of feature 1, wherein the antibody or antigen binding fragment thereof is amlitelimab or a variant thereof.

3. The aqueous pharmaceutical formulation of feature 1 or feature 2, wherein the antibody or antigen binding fragment thereof is present in a concentration ranging from 75 mg/mL to 125 mg/mL, from 125 mg/mL to 138 mg/mL, or from 112.5 mg/mL to 137.5 mg/mL.

4. The aqueous pharmaceutical formulation of any one of features 1-3, wherein the antibody or antigen binding fragment thereof is present in a concentration of 125 mg/mL to 138 mg/mL.

5. The aqueous pharmaceutical formulation of any one of features 1-4, comprising 125 mg/mL, 120.5 mg/mL, or 124.4 mg/mL of the antibody or antigen binding fragment thereof.

6. The aqueous pharmaceutical formulation of any one of features 1-5, wherein the stabilizer is sucrose.

7. The aqueous pharmaceutical formulation of any one of features 1-6, wherein the at least one stabilizer is sucrose, which is present in an amount of 220 mM.

8. The aqueous pharmaceutical formulation of any one of features 1-7, wherein the surfactant is polysorbate 80.

9. The aqueous pharmaceutical formulation of anyone of features 1-8, comprising 0.01% (w/v) to 0.1% (w/v) polysorbate 80.

10. The aqueous pharmaceutical formulation of any one of features 1-9, comprising 0.02% (w/v) to 0.06% (w/v) polysorbate 80.

11. The aqueous pharmaceutical formulation of any one of features 1-10, comprising 0.04% (w/v) polysorbate 80.

12. The aqueous pharmaceutical formulation of any one of features 1-11, wherein the buffer comprises 20 mM L-histidine or histidine hydrochloride.

13. The aqueous pharmaceutical formulation of feature 1, comprising:
125 mg/mL to 138 mg/mL of the antibody or antigen binding fragment thereof;
20 mM L-histidine or histidine hydrochloride;
220 mM sucrose,
0.04% (w/v) polysorbate 80; and
water;
with the pH of the aqueous pharmaceutical formulation between about 5.8 and about 6.2.

14. The aqueous pharmaceutical formulation of any one of features 1-13, which is free or essentially free of particles.

15. The aqueous pharmaceutical formulation of any one of features 1-14, which is free or substantially free of sodium chloride.

16. The aqueous pharmaceutical formulation of any one of features 1-15, which is free or substantially free of arginine.

17. The aqueous pharmaceutical formulation of any one of features 1-16, wherein at least 91% of the antibody has native conformation after 28 days at 40° C., measured by size exclusion chromatography.

18. The aqueous pharmaceutical formulation of any one of features 1-17, wherein at least 94% of the antibody has native conformation after 8 weeks at 25° C., measured by size exclusion chromatography.

19. The aqueous pharmaceutical formulation of any one of features 1-18, wherein at least 98% of the antibody has native conformation after 16 weeks at 5° C. or at −65° C., measured by size exclusion chromatography.

20. The aqueous pharmaceutical formulation of any one of features 1-19, wherein at least 55% of the antibody is the main charge variant of the antibody after 28 days at 40° C., measured by imaged capillary isoelectric focusing (iCIEF).

21. The aqueous pharmaceutical formulation of any one of features 1-20, wherein at least 65% of the antibody is the main charge variant of the antibody after 16 weeks at 25° C., measured by iCIEF.

22. The aqueous pharmaceutical formulation of any one of features 1-21, wherein at least 70% of the antibody is the main charge variant of the antibody after 16 weeks at 5° C., measured by iCIEF.

23. The aqueous pharmaceutical formulation of any one of features 1-22, wherein at least 70% of the antibody is the main charge variant of the antibody after 16 weeks at −65° C., measured by iCIEF.

24. The aqueous pharmaceutical formulation of any one of features 1-23, which is stable upon storage at −65° C., 5° C., or 25° C. for at least 16 weeks.

25. The aqueous pharmaceutical formulation of any one of features 1-24, which is stable upon storage at 40° C. for at least 8 weeks.

26. The aqueous pharmaceutical formulation of any one of features 1-25, which is stable upon storage at 5° C. for at least 1 year.

27. The aqueous pharmaceutical formulation of any one of features 1-26, which is stable upon freezing and thawing.

28. The aqueous pharmaceutical formulation of any one of features 1-27, which is provided in a container.

29. A pharmaceutical unit dosage form suitable for parenteral administration to a mammalian subject which comprises the aqueous pharmaceutical formulation of any one of claims 1 to 27 in a suitable container.

30. The pharmaceutical unit dosage form of claim 29, wherein the aqueous pharmaceutical formulation is suitable for intravenous, subcutaneous, or intramuscular administration.

31. The pharmaceutical unit dosage form of claim 29 or 30, wherein the suitable container is a pre-filled syringe.

32. A sealed container comprising the aqueous pharmaceutical formulation of any one of features 1-27.

33. The sealed container of feature 32, which is a vial, a syringe, a microinfusor, a pen delivery device, or an autoinjector.

34. The sealed container of feature 32 or feature 33, which is a single or multi-chambered syringe.

35. The sealed container of feature 34, which is a pre-filled syringe containing 2.25 mL of the aqueous pharmaceutical formulation.

36. A pre-filled syringe comprising an aqueous pharmaceutical formulation comprising:
125 mg/mL to 138 mg/mL of amlitelimab or a variant thereof;
20 mM L-histidine or histidine hydrochloride;
220 mM sucrose,
0.04% (w/v) polysorbate 80, and
water;
wherein the pH of the aqueous pharmaceutical formulation is 6.0.

37. A kit comprising a sealed container of any one of features 32-35 or a pre-filled syringe of feature 36.

38. A kit comprising:
a sealed container comprising the aqueous pharmaceutical formulation of any one of features 1-27, and
at least one separate injection device for delivery the aqueous pharmaceutical formulation to a mammalian subject in need thereof.

39. The kit of feature 38, wherein the injection device is a syringe, a microinfusor, a pen delivery device, or an autoinjector.

40. The kit of feature 39, wherein the injection device is a single or multi-chambered syringes.

41. The aqueous pharmaceutical formulation of any one of features 1-28, the pharmaceutical unit dosage form of any one of features 29-31, the sealed container of any one of features 32-35, the pre-filled syringe of feature 36, or the kit of any one of features 38-40 for use in treating a disease or condition in a subject selected from the group consisting of an autoimmune disease or condition, an inflammatory disease or condition, and a transplant rejection disease or condition mediated by hOX40L.

42. A method of treating a disease or condition in a subject selected from the group consisting of an autoimmune disease or condition, an inflammatory disease or condition, and a transplant rejection disease or condition, comprising administering to the subject an effective amount of the aqueous pharmaceutical formulation of any one of features 1 to 27.

43. A method of treating atopic dermatitis comprising administering to the subject an effective amount of the aqueous pharmaceutical formulation of any one of features 1 to 27.

44. A method of treating asthma comprising administering to the subject an effective amount of the aqueous pharmaceutical formulation of any one of features 1 to 27.

45. An aqueous pharmaceutical formulation comprising:
(a) an antibody that is capable of blocking binding or substantially reducing binding of OX40 to OX40L, or an antigen binding fragment thereof;
(b) a stabilizer;
(c) a surfactant; and
(d) a buffer comprising histidine or a histidine salt;
wherein the pH of the aqueous pharmaceutical formulation ranges from 5.5 to about 6.5, and
wherein the aqueous pharmaceutical formulation is suitable for parenteral administration to a mammalian subject.

The disclosure includes the following numbered segments:

1. An aqueous pharmaceutical formulation comprising:
(a) an anti-OX40 ligand (OX40L) antagonist antibody or antigen binding fragment thereof;
(b) L-histidine or histidine hydrochloride;
(c) sucrose;
(d) ethylenediaminetetraacetic acid (EDTA) (e) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60.

2. An aqueous pharmaceutical formulation comprising:
(a) about 62.5 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60.

3. An aqueous pharmaceutical formulation comprising:
(a) about 125 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60.

4. An aqueous pharmaceutical formulation comprising:
(a) an anti-OX40 ligand (OX40L) antagonist antibody or antigen binding fragment thereof;
(b) L-histidine or histidine hydrochloride;
(c) sucrose;
(d) ethylenediaminetetraacetic acid (EDTA)
(e) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises: a VH domain comprising at least 80% identity to the amino acid sequence of SEQ ID NO: 34 and a VL domain having at least 80% identity to the amino acid sequence of SEQ ID NO: 48.

5. An aqueous pharmaceutical formulation comprising:
(a) about 62.5 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises: a VH domain comprising at least 80% identity to the amino acid sequence of SEQ ID NO: 34 and a VL domain having at least 80% identity to the amino acid sequence of SEQ ID NO: 48.

6. An aqueous pharmaceutical formulation comprising:
(a) about 125 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises: a VH domain comprising at least 80% identity to the amino acid sequence of SEQ ID NO: 34 and a VL domain having at least 80% identity to the amino acid sequence of SEQ ID NO: 48.

7. An aqueous pharmaceutical formulation comprising:
(a) an anti-OX40 ligand (OX40L) antagonist antibody or antigen binding fragment thereof;
(b) L-histidine or histidine hydrochloride;
(c) sucrose;
(d) ethylenediaminetetraacetic acid (EDTA)
(e) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises a VH domain of SEQ ID NO: 34 and a VL domain of SEQ ID NO: 48.

8. An aqueous pharmaceutical formulation comprising:
(a) about 62.5 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises a VH domain of SEQ ID NO: 34 and a VL domain of SEQ ID NO: 48.

9. An aqueous pharmaceutical formulation comprising:
(a) about 125 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA) (e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises a VH domain of SEQ ID NO: 34 and a VL domain of SEQ ID NO: 48.

10. An aqueous pharmaceutical formulation comprising:
(a) an anti-OX40 ligand (OX40L) antagonist antibody or antigen binding fragment thereof;
(b) L-histidine or histidine hydrochloride;
(c) sucrose;
(d) ethylenediaminetetraacetic acid (EDTA)
(e) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises a heavy chain having an amino acid sequence of SEQ ID NO: 62 and a light chain having an amino acid sequence of SEQ ID NO: 64.

11. An aqueous pharmaceutical formulation comprising:
(a) about 62.5 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises a heavy chain having an amino acid sequence of SEQ ID NO: 62 and a light chain having an amino acid sequence of SEQ ID NO: 64.

12. An aqueous pharmaceutical formulation comprising:
(a) about 125 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises a heavy chain having an amino acid sequence of SEQ ID NO: 62 and a light chain having an amino acid sequence of SEQ ID NO: 64.

13. A pre-filled syringe comprising an aqueous pharmaceutical formulation comprising:
(a) an anti-OX40 ligand (OX40L) antagonist antibody or antigen binding fragment thereof;
(b) L-histidine or histidine hydrochloride;
(c) sucrose;
(d) ethylenediaminetetraacetic acid (EDTA)
(e) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60.

14. A pre-filled syringe comprising an aqueous pharmaceutical formulation comprising:
(a) about 62.5 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60.

15. A pre-filled syringe comprising an aqueous pharmaceutical formulation comprising:
(a) about 125 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60.

16. A pre-filled syringe comprising an aqueous pharmaceutical formulation comprising:
(a) an anti-OX40 ligand (OX40L) antagonist antibody or antigen binding fragment thereof;
(b) L-histidine or histidine hydrochloride;
(c) sucrose;
(d) ethylenediaminetetraacetic acid (EDTA) (e) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises:
a VH domain comprising at least 80% identity to the amino acid sequence of SEQ ID NO: 34 and a VL domain having at least 80% identity to the amino acid sequence of SEQ ID NO: 48.

17. A pre-filled syringe comprising an aqueous pharmaceutical formulation comprising:
(a) about 62.5 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises: a VH domain comprising at least 80% identity to the amino acid sequence of SEQ ID NO: 34 and a VL domain having at least 80% identity to the amino acid sequence of SEQ ID NO: 48.

18. A pre-filled syringe comprising an aqueous pharmaceutical formulation comprising:
(a) about 125 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA) (e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises: a VH domain comprising at least 80% identity to the amino acid sequence of SEQ ID NO: 34 and a VL domain having at least 80% identity to the amino acid sequence of SEQ ID NO: 48.

19. A pre-filled syringe comprising an aqueous pharmaceutical formulation comprising:
(a) an anti-OX40 ligand (OX40L) antagonist antibody or antigen binding fragment thereof;
(b) L-histidine or histidine hydrochloride;
(c) sucrose;
(d) ethylenediaminetetraacetic acid (EDTA)
(e) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises a VH domain of SEQ ID NO: 34 and a VL domain of SEQ ID NO: 48.

20. A pre-filled syringe comprising an aqueous pharmaceutical formulation comprising:
(a) about 62.5 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises a VH domain of SEQ ID NO: 34 and a VL domain of SEQ ID NO: 48.

21. A pre-filled syringe comprising an aqueous pharmaceutical formulation comprising:
(a) about 125 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises a VH domain of SEQ ID NO: 34 and a VL domain of SEQ ID NO: 48.

22. A pre-filled syringe comprising an aqueous pharmaceutical formulation comprising:
(a) an anti-OX40 ligand (OX40L) antagonist antibody or antigen binding fragment thereof;
(b) L-histidine or histidine hydrochloride;
(c) sucrose;
(d) ethylenediaminetetraacetic acid (EDTA) (e) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises a heavy chain having an amino acid sequence of SEQ ID NO: 62 and a light chain having an amino acid sequence of SEQ ID NO: 64.

23. A pre-filled syringe comprising an aqueous pharmaceutical formulation comprising:
(a) about 62.5 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA) (e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises a heavy chain having an amino acid sequence of SEQ ID NO: 62 and a light chain having an amino acid sequence of SEQ ID NO: 64.

24. A pre-filled syringe comprising an aqueous pharmaceutical formulation comprising:
(a) about 125 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA) (e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises a heavy chain having an amino acid sequence of SEQ ID NO: 62 and a light chain having an amino acid sequence of SEQ ID NO: 64.

25. A pre-filled pen or autoinjector comprising an aqueous pharmaceutical formulation comprising:
(a) an anti-OX40 ligand (OX40L) antagonist antibody or antigen binding fragment thereof;
(b) L-histidine or histidine hydrochloride;
(c) sucrose;
(d) ethylenediaminetetraacetic acid (EDTA) (e) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60.

26. A pre-filled pen or autoinjector comprising an aqueous pharmaceutical formulation comprising:
(a) about 62.5 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60.

27. A pre-filled pen or autoinjector comprising an aqueous pharmaceutical formulation comprising:
(a) about 125 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises:
   a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
   a VL domain which comprises the LCDR1 sequence of SEQ ID NO:50 or 56, the LCDR2 sequence of SEQ ID NO:52 or 58, and the LCDR3 sequence of SEQ ID NO:54 or 60.

28. A pre-filled pen or autoinjector comprising an aqueous pharmaceutical formulation comprising:
(a) an anti-OX40 ligand (OX40L) antagonist antibody or antigen binding fragment thereof;
(b) L-histidine or histidine hydrochloride;
(c) sucrose;
(d) ethylenediaminetetraacetic acid (EDTA)
(e) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises:
a VH domain comprising at least 80% identity to the amino acid sequence of SEQ ID NO: 34 and a VL domain having at least 80% identity to the amino acid sequence of SEQ ID NO: 48.

29. A pre-filled pen or autoinjector comprising an aqueous pharmaceutical formulation comprising:
(a) about 62.5 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises:
a VH domain comprising at least 80% identity to the amino acid sequence of SEQ ID NO: 34 and a VL domain having at least 80% identity to the amino acid sequence of SEQ ID NO: 48.

30. A pre-filled pen or autoinjector comprising an aqueous pharmaceutical formulation comprising:
(a) about 125 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises:
a VH domain comprising at least 80% identity to the amino acid sequence of SEQ ID NO: 34 and a VL domain having at least 80% identity to the amino acid sequence of SEQ ID NO: 48.

31. A pre-filled pen or autoinjector comprising an aqueous pharmaceutical formulation comprising:
(a) an anti-OX40 ligand (OX40L) antagonist antibody or antigen binding fragment thereof;
(b) L-histidine or histidine hydrochloride;
(c) sucrose;
(d) ethylenediaminetetraacetic acid (EDTA)
(e) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises a VH domain of SEQ ID NO: 34 and a VL domain of SEQ ID NO: 48.

32. A pre-filled pen or autoinjector comprising an aqueous pharmaceutical formulation comprising:
(a) about 62.5 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises a VH domain of SEQ ID NO: 34 and a VL domain of SEQ ID NO: 48.

33. A pre-filled pen or autoinjector comprising an aqueous pharmaceutical formulation comprising:
(a) about 125 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises a VH domain of SEQ ID NO: 34 and a VL domain of SEQ ID NO: 48.

34. A pre-filled pen or autoinjector comprising an aqueous pharmaceutical formulation comprising:
(a) an anti-OX40 ligand (OX40L) antagonist antibody or antigen binding fragment thereof;
(b) L-histidine or histidine hydrochloride;
(c) sucrose;
(d) ethylenediaminetetraacetic acid (EDTA) (e) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises a heavy chain having an amino acid sequence of SEQ ID NO: 62 and a light chain having an amino acid sequence of SEQ ID NO: 64.

35. A pre-filled pen or autoinjector comprising an aqueous pharmaceutical formulation comprising:
(a) about 62.5 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises a heavy chain having an amino acid sequence of SEQ ID NO: 62 and a light chain having an amino acid sequence of SEQ ID NO: 64.

36. A pre-filled pen or autoinjector comprising an aqueous pharmaceutical formulation comprising:
(a) about 125 mg/mL of an anti-OX40 ligand (OX40L) antagonist antibody or antigen binding fragment thereof;
(b) about 10 mM L-histidine or histidine hydrochloride;
(c) about 220 mM sucrose;
(d) about 10 μM ethylenediaminetetraacetic acid (EDTA)
(e) about 0.06% (w/v) polysorbate 80; and
(f) water;
wherein the pH of the aqueous pharmaceutical formulation ranges from 5.5 to about 6.5, preferably wherein the pH of the aqueous pharmaceutical formulation is about 6,
wherein the antibody or antigen binding fragment thereof comprises a heavy chain having an amino acid sequence of SEQ ID NO: 62 and a light chain having an amino acid sequence of SEQ ID NO: 64.

37. An aqueous pharmaceutical formulation according to any one of segments 1-12, a pre-filled syringe according to any one of segments 13-24, or a pre-filled pen or autoinjector according to any one of segments 25-36, wherein the aqueous pharmaceutical formulation may be in a volume of about 1 mL or about 2 mL, preferably 1 mL or 2 mL.

38. An aqueous pharmaceutical formulation according to any one of segments 1-12 and 37, a pre-filled syringe according to any one of segments 13-24 and 37, or a pre-filled pen or autoinjector according to any one of segments 25-37, wherein the aqueous pharmaceutical formulation is suitable for parenteral administration to a mammalian subject.

39. An aqueous pharmaceutical formulation according to any one of segments 1-12 and 38, a pre-filled syringe according to any one of segments 13-24 and 38, or a pre-filled pen or autoinjector according to any one of segments 25-38, wherein the antibody or antigen binding fragment thereof is amlitelimab or a variant thereof.

40. An aqueous pharmaceutical formulation according to any one of segments 1-12, 37-39, a pre-filled syringe according to any one of segments 13-24, 37-39, or a pre-filled pen or autoinjector according to any one of segments 25-39, for use in treating a disease or condition in a subject selected from the group consisting of an autoimmune disease or condition, an inflammatory disease or condition, a systemic inflammatory disease or condition, and a transplant rejection disease or condition mediated by hOX40L.

41. An aqueous pharmaceutical formulation according to any one of segments 1-12 and 37-40 a pre-filled syringe according to any one of segments 13-24 and 37-40, or a pre-filled pen or autoinjector according to any one of segments 25-40, for use in treating atopic dermatitis or asthma.

1. An aqueous pharmaceutical formulation comprising:
(a) an anti-OX40 ligand (OX40L) antagonist antibody or antigen binding fragment thereof,
(b) a stabilizer;
(c) a chelator;
(d) a surfactant; and
(e) a buffer comprising histidine or a histidine salt;
wherein the pH of the aqueous pharmaceutical formulation ranges from 5.5 to about 6.5,
wherein:
(i) the antibody or antigen binding fragment thereof comprises:
the heavy chain complementarity determining region (HCDR) 1 of SEQ ID NO: 42, the HCDR2 of SEQ ID NO:44, and the HCDR3 of SEQ ID NO: 46; and
the light chain complementarity determining region (LCDR) 1 of SEQ ID NO:56, the LCDR2 of SEQ ID NO:58, and the LCDR3 of SEQ ID NO: 60; or
(ii) the antibody or antigen binding fragment thereof comprises:
the heavy chain complementarity determining region (HCDR) 1 of SEQ ID NO: 36, the HCDR2 of SEQ ID NO: 38, and the HCDR3 of SEQ ID NO: 40; and
the light chain complementarity determining region (LCDR) 1 of SEQ ID NO: 50, the LCDR2 of amino acid sequence AAS, and the LCDR3 of SEQ ID NO: 54, or
(iii) the antibody or antigen binding fragment thereof comprises a variable heavy (VH) domain comprising at least 80% identity to the amino acid sequence of SEQ ID NO: 34 and a variable light (VL) domain having at least 80% identity to the amino acid sequence of SEQ ID NO: 48; or
(iv) the antibody or antigen binding fragment thereof comprises:
a VH domain which comprises the HCDR1 sequence of SEQ ID NO: 36 or 42, the HCDR2 sequence of SEQ ID NO: 38 or 44, and the HCDR3 sequence of SEQ ID NO: 40 or 46; and
a VL domain which comprises the LCDR1 sequence of SEQ ID NO: 50 or 56, the LCDR2 sequence of SEQ ID NO: 52 or 58, and the LCDR3 sequence of SEQ ID NO: 54 or 60; or
(v) the VL domain comprises the amino acid sequence of SEQ ID NO:48 and the VH domain comprises the amino acid sequence of SEQ ID NO:34; or
(vi) the antibody comprises a heavy chain and a light chain, wherein the heavy chain amino acid sequence consists of the amino acid sequence of SEQ ID NO: 62 and the light chain amino acid sequence consists of the amino acid sequence of SEQ ID NO: 64,
wherein the aqueous pharmaceutical formulation is suitable for parenteral administration to a mammalian subject.

2. (canceled)

3. (canceled)

4. The aqueous pharmaceutical formulation of claim 1, wherein:
the antibody or antigen binding fragment comprises a VH domain comprising at least 80% or 100% sequence identity to the amino acid sequence of SEQ ID NO: 34 and/or comprises a VL domain comprising at least 80% or 100% sequence identity to the amino acid sequence of SEQ ID NO: 48; or
the antibody or antigen binding fragment comprises the heavy chain of SEQ ID NO: 62 and the light chain of SEQ ID NO: 64; or
the antibody is amlitelimab or a variant thereof.

5-7. (canceled)

8. The aqueous pharmaceutical formation of claim 1, wherein the aqueous pharmaceutical formulation is suitable for parenteral administration to a mammalian subject, optionally wherein the aqueous pharmaceutical formulation is suitable for subcutaneously delivery.

9. The aqueous pharmaceutical formulation of claim 1, wherein the antibody or antigen binding fragment thereof is present at a concentration of 62.5 mg/mL or 125 mg/mL.

10. (canceled)

11. The aqueous pharmaceutical formulation of claim 1, wherein the stabilizer is sucrose, optionally wherein the sucrose is present in an amount of 220 mM.

12. (canceled)

13. The aqueous pharmaceutical formulation of claim 1, wherein the surfactant is polysorbate 80, optionally wherein the polysorbate 80 is present in a concentration of from 0.02% (w/v) to 0.1% (w/v), from 0.02% (w/v) to 0.06%, or from 0.04% (w/v) or 0.06% (w/v).

14-16. (canceled)

17. The aqueous pharmaceutical formulation of claim 1, wherein the buffer comprises 10 mM L-histidine or histidine hydrochloride.

18. The aqueous pharmaceutical formulation of claim 1, wherein the chelator is selected from the group consisting of ethylenediaminetetraacetic acid (EDTA), diethylenetriamene pentaacetate (DTPA) and salts thereof, and any combinations thereof, optionally wherein the chelator comprises one or both of 10 μM EDTA and 10 μM DTPA.

19. (canceled)

20. The aqueous pharmaceutical formulation of claim 1, comprising:
28 mg/mL to 138 mg/mL of the antibody or antigen binding fragment thereof;
10 mM±1.5 mM L-histidine or histidine hydrochloride;
220 mM±33 mM sucrose,
0.06% (w/v) polysorbate 80; and
water,
wherein the pH of the aqueous pharmaceutical formulation is between about 5.8 and about 6.2.

21. The aqueous pharmaceutical formulation of claim 20, further comprising 10 μM EDTA or 10 μM DTPA, optionally wherein the aqueous pharmaceutical formulation is housed in a container, or suitable for subcutaneously delivery.

22-24. (canceled)

25. A pharmaceutical unit dosage form suitable for parenteral administration to a mammalian subject which comprises the aqueous pharmaceutical formulation of claim 1 in a suitable container, optionally wherein the aqueous pharmaceutical formulation is suitable for intravenous, subcutaneous, or intramuscular administration, or optionally wherein the suitable container is a pre-filled syringe.

26. (canceled)

27. (canceled)

28. A sealed container comprising the aqueous pharmaceutical formulation of claim 1.

29. The sealed container of claim 28, which is a vial, a syringe, a microinfusor, a pen delivery device, or an autoinjector, optionally wherein the sealed container is a single or multi-chambered syringe, or optionally wherein the sealed container is a pre-filled syringe or pen.

30. (canceled)

31. A container comprising an aqueous pharmaceutical formulation comprising:
about 28 mg/mL to about 138 mg/mL of an anti-OX40 ligand (OX40L) antagonist antibody or antigen binding fragment thereof,
about 10 mM L-histidine or histidine hydrochloride;
about 220 mM sucrose;
about 0.06% (w/v) polysorbate 80;
about 10 μM EDTA; and
water,
wherein the pH of the aqueous pharmaceutical is about 6.0, and wherein the container is a pre-filled syringe, a pre-filled pen, or an autoinjector, optionally wherein the antibody or antigen binding fragment thereof is present at a concentration of 62.5 mg/mL or 125 mg/mL.

32-34. (canceled)

35. The container of claim 31, wherein the anti-OX40 ligand (OX40L) antagonist antibody or antigen binding fragment thereof comprises:
(a) the heavy chain complementarity determining region (HCDR) 1 of SEQ ID NO: 42, the HCDR2 of SEQ ID NO:44, and the HCDR3 of SEQ ID NO:46; and the light chain complementarity determining region (LCDR) 1 of SEQ ID NO:56, the LCDR2 of SEQ ID NO:58, and the LCDR3 of SEQ ID NO:60; or
(b) the heavy chain complementarity determining region (HCDR) 1 of SEQ ID NO: 36, the HCDR2 of SEQ ID NO:38, and the HCDR3 of SEQ ID NO:40; and the light chain complementarity determining region (LCDR) 1 of SEQ ID NO:50, the LCDR2 of SEQ ID NO:52, and the LCDR3 of SEQ ID NO:54; or
(c) the VH domain comprising at least 80% identity to the amino acid sequence of SEQ ID NO: 34 and the VL domain having at least 80% identity to the amino acid sequence of SEQ ID NO: 48; or
(d) the VH domain comprising at least 80% identity to the amino acid sequence of SEQ ID NO: 34 and/or comprises the VL domain having at least 80% identity to the amino acid sequence of SEQ ID NO: 48; or
(e) the heavy chain amino acid sequence consisting of the amino acid sequence of SEQ ID NO: 62 and/or the light chain amino acid sequence consisting of the amino acid sequence of SEQ ID NO: 64.

36. The container of claim 31, wherein the antibody is amlitelimab or a variant thereof, wherein the container comprises about 31 mg/mL to about 125 mg/mL of amlitelimab or a variant thereof, optionally about 62.5 mg/mL or about 125 mg/mL of amlitelimab or a variant thereof.

37. A kit comprising a sealed container of claim 28, optionally comprising: at least one separate injection device for delivery of the aqueous pharmaceutical formulation to a mammalian subject in need thereof, optionally wherein the injection device is a syringe, a microinfusor, a pen delivery device, or an autoinjector; or optionally wherein the injection device is a single or multi-chambered syringes.

38-46. (canceled)

47. A method of treating:
a disease or condition in a subject selected from the group consisting of an autoimmune disease or condition, an inflammatory disease or condition, a systemic inflammatory disease or condition, and a transplant rejection disease or condition mediated by human OX40L (hOX40L),
comprising administering to the subject an effective amount of the aqueous pharmaceutical formulation of claim 1.

48-49. (canceled)

50. An aqueous pharmaceutical formulation comprising:
(a) an anti-OX40 ligand (OX40L) antagonist antibody or antigen binding fragment;
(b) a stabilizer;
(c) a chelator;
(d) a surfactant; and
(e) a buffer comprising histidine or a histidine salt;
wherein the pH of the aqueous pharmaceutical formulation ranges from 5.5 to about 6.5,
wherein the antibody or antigen binding fragment thereof comprises:
a VH domain which comprises the HCDR1 sequence of SEQ ID NO:36 or 42, the HCDR2 sequence of SEQ ID NO:38 or 44, and the HCDR3 sequence of SEQ ID NO:40 or 46, and
a VL domain which comprises the LCDR1 sequence of SEQ ID NO:50 or 56, the LCDR2 sequence of SEQ ID NO:52 or 58, and the LCDR3 sequence of SEQ ID NO:54 or 60; and
wherein the aqueous pharmaceutical formulation is suitable for parenteral administration to a mammalian subject.

51-52. (canceled)

53. The method of claim 47, wherein the disease or condition is atopic dermatitis or asthma.
